(19) United States
(12) Patent Application Publication
(10) Pub. No.: US 2013/0005721 A1
(43) Pub. Date: Jan. 3, 2013

(54) INDOLIZINE INHIBITORS OF 5-LIPOXYGENASE

(75) Inventors: Brian Andrew Stearns, Encinitas, CA (US); Jeffrey Roger Roppe, Temecula, CA (US); Timothy Andrew Parr, La Mesa, CA (US); Nicholas Simon Stock, San Diego, CA (US); Deborah Volkots, La Jolla, CA (US); John Howard Hutchinson, San Diego, CA (US)

(73) Assignee: PANMIRA PHARMACEUTICALS, LLC, San Diego, CA (US)

(21) Appl. No.: 13/497,517

(22) PCT Filed: Sep. 23, 2010
(86) PCT No.: PCT/US10/49954

§ 371 Date: Aug. 8, 2012

Related U.S. Application Data
(60) Provisional application No. 61/245,198, filed on Sep. 23, 2009.

(51) Int. Cl. A61K 31/437; A61K 31/5377; A61K 31/4709; A61K 31/444; A61K 31/506; A61P 29/00; A61P 11/00; A61P 9/00; A61P 11/06; A61P 9/10; A61P 9/12; A61P 25/00; A61P 11/02; A61P 19/02; A61P 35/00; A61P 3/00; C07D 471/04

(52) U.S. Cl. 514/233.2; 546/112; 514/299; 546/121; 514/300; 544/127; 546/167; 514/314; 544/333; 514/256

(57) ABSTRACT

Described herein are compounds and pharmaceutical compositions containing such compounds, which inhibit the activity of 5-lipoxygenase (5-LO). Also described herein are methods of using such 5-LO inhibitors, alone and in combination with other compounds, for treating respiratory, cardiovascular, and other leukotriene-dependent or leukotriene mediated conditions, diseases, or disorders.

### RELATED APPLICATIONS

This application claims the benefit of U.S. provisional patent application No. 61/245,198 entitled “INDOLIZINE INHIBITORS OF 5-LIPOXYGENASE” filed on Sep. 23, 2009, which is incorporated by reference in its entirety.

### FIELD OF THE INVENTION

Described herein are compounds, methods of making such compounds, pharmaceutical compositions and medicaments that include such compounds and methods of using such compounds to treat or prevent diseases or conditions associated with 5-lipoxygenase activity.

### BACKGROUND OF THE INVENTION

The protein 5-lipoxygenase (5-LO) is associated with the pathway of leukotriene synthesis. Upon presentation of inflammatory stimuli from the exterior of the cell, calcium is released and binds to phospholipase A₂ (PLA2) and 5-lipoxygenase. Cell activation results in the translocation of PLA₂ and 5-lipoxygenase from the cytoplasm to the endoplasmic reticulum and/or nuclear membranes, where in the presence of 5-lipoxygenase-activating protein (FLAP), 5-lipoxygenase can then catalyze the two-step oxygenation and dehydration of arachidonic acid, converting it into the intermediate compound 5-HPETE (5hydroperoxyeicosatetraenoic acid), and in the presence of FLAP convert the 5-HPETE to Leukotriene A₄ (LTA₄).

Leukotrienes are biological compounds formed from arachidonic acid in the leukotriene synthesis pathway (Samuelsson et al., Science, 220, 568-575, 1983; Cooper, The Cell, A Molecular Approach, 2nd Ed. Sinauer Associates, Inc., Sunderland (MA), 2000). They are synthesized primarily by eosinophils, neutrophils, mast cells, basophils, dendritic cells, macrophages and monocytes. Leukotrienes have been implicated in biological actions including, by way of example only, smooth muscle contraction, leukocyte activation, cytokine secretion, mucous secretion, and vascular function.

### SUMMARY OF THE INVENTION

Presented herein are methods, compounds, pharmaceutical compositions, and medicaments for (a) diagnosing, preventing, or treating allergic and non-allergic inflammation, (b) controlling signs and symptoms that are associated with inflammation, and/or (c) controlling proliferative or metabolic disorders. These disorders may arise from genetic, iatrogenic, immunological, infectious, metabolic, oncologic, toxic, and/or traumatic etiology. In one aspect, the methods, compounds, pharmaceutical compositions, and medicaments described herein include 5-lipoxygenase inhibitors described herein.

Compounds, pharmaceutically acceptable salts, pharmaceutically acceptable N-oxides, pharmaceutically active metabolites, pharmaceutically acceptable prodrugs, and/or pharmaceutically acceptable solvates thereof, which antagonize or inhibit 5-lipoxygenase and may be used to treat patients suffering from leukotriene-dependent conditions or diseases, including, but not limited to, asthma, chronic obstructive pulmonary disease, pulmonary hypertension, interstitial lung fibrosis, rhinitis, arthritis, allergy, psoriasis, inflammatory bowel disease, adult respiratory distress syndrome, myocardial infarction, aneurysm, stroke, cancer, endotoxic shock, proliferative disorders, inflammatory conditions and painful conditions, are provided.

Compounds described herein, or pharmaceutically acceptable salts, pharmaceutically acceptable N-oxides, pharmaceutically active metabolites, pharmaceutically acceptable prodrugs, and pharmaceutically acceptable solvates thereof may be used to treat leukotriene dependent or leukotriene-mediated diseases, disorders, or conditions in a patient, or 5-lipoxygenase dependent or 5-lipoxygenase-mediated diseases, disorders, or condition in a patient.

In one aspect, provided herein are compounds that have a structure represented by Formula (I), or a pharmaceutically acceptable salt, or a pharmaceutically acceptable N-oxide thereof

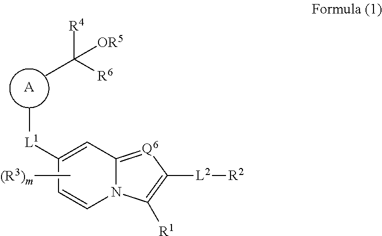

wherein:
A is a 5-membered N-containing heteroaryl;
Q⁶ is N or CR⁷;
R¹ is H, —C(═O)R⁸, substituted or unsubstituted C₁-C₆alkyl, substituted or unsubstituted C₁-C₆-fluoroalkyl, substituted or unsubstituted C₃-C₆cycloalkyl, substituted or unsubstituted C₁-C₆heteroalkyl, substituted or unsubstituted C₂-C₆cycloheteroalkyl, substituted or unsubstituted aryl, substituted or unsubstituted heteroaryl;
R² is halo, —CO₂R⁹, tetrazole, —C(═O)N(R⁹)₂, —CN, —CH(═O), —SR⁸, —S(O)R⁸, —S(O)₂R⁸, —S(O)₂N(R⁹)₂, —OR⁸, —N(R⁹)₂, —C(═O)R⁸, substituted or unsubstituted C₁-C₆alkyl, substituted or unsubstituted C₃-C₆cycloalkyl, substituted or unsubstituted C₁-C₆heteroalkyl, substituted or unsubstituted C₂-C₆cycloheteroalkyl, substituted or unsubstituted aryl, or substituted or unsubstituted heteroaryl;
each R³ is independently halo, —CO₂R⁹, —C(═O)N(R⁹)₂, —CN, tetrazole, —CH(═O), —SR⁸, —S(O)R⁸, —S(O)₂R⁸, substituted or unsubstituted alkyl, substituted or unsubstituted heteroalkyl, —OR⁸, —SR⁸, —N(R⁹)₂, or —C(═O)R⁸;
R⁴ is C₁-C₆haloalkyl, C₁-C₆alkyl, C₃-C₆cycloalkyl, or C₁-C₆heteroalkyl;
R⁵ is H, C₁-C₆alkyl, or —C(═O)R¹⁰;
R⁶ is H, C₁-C₆alkyl, C₃-C₆cycloalkyl, C₁-C₆heteroalkyl, or C₁-C₆haloalkyl;
R⁷ is H, halo, —CO₂R⁹, —C(═O)R⁸, —C(═O)N(R⁹)₂, —CN, —CH(═O), —SR⁸, —S(O)R⁸, —S(O)₂R⁸, —OR⁸, —N(R⁹)₂, substituted or unsubstituted C₁-C₆alkyl, substituted or unsubstituted C₁-C₆heteroalkyl, substituted or unsubstituted aryl, or substituted or unsubstituted heteroaryl;
each R⁸ is independently substituted or unsubstituted C₁-C₆alkyl, substituted or unsubstituted C₁-C₆-fluoroalkyl, substituted or unsubstituted C₁-C₆heteroalkyl, substituted or unsubstituted aryl, or substituted or unsubstituted heteroaryl;
each R⁹ is independently H, substituted or unsubstituted C₁-C₆alkyl, substituted or unsubstituted C₁-C₆-fluoroalkyl, substituted or unsubstituted C₁-C₆heteroalkyl, substituted or unsubstituted aryl, or substituted or unsubstituted heteroaryl; or two R⁹ taken together with the nitrogen to which they are bound form a substituted or unsubstituted heterocycle;
each R¹⁰ substituted or unsubstituted C₁-C₆alkyl, substituted or unsubstituted C₁-C₆heteroalkyl, substituted or unsubstituted aryl, or substituted or unsubstituted heteroaryl;
   or two R¹⁰ taken together with the carbon to which they are bound form a substituted or unsubstituted carbocycle, or substituted or unsubstituted heterocycle;
L¹ is —O—, —NR⁸—, —S—, —C₁-C₃alkylene-, —O—, —C₁-C₃alkylene-, —C₁-C₃alkylene-O—, —C₁-C₃alkylene-NR⁸—, —NR⁸—C₁-C₃alkylene-, —C₁-C₃alkylene-S—, —S—C₁-C₃alkylene-, or —C₁-C₃heteroalkylene-;
L² is a bond, C₁-C₆alkylene, or C₁-C₆heteroalkylene;
m is 0, 1, 2 or 3.

For any and all of the embodiments, substituents are selected from among from a subset of the listed alternatives. For example, in some embodiments, Q⁶ is N. In some embodiments, Q⁶ is CR⁷.

In some embodiments, A is triazolyl, oxadiazolyl or thiazolyl. In some embodiments, A is triazolyl. In some embodiments, A is oxadiazolyl. In some embodiments, A is thiazolyl.

In some embodiments, R¹ is H, —C(═O)R⁸, C₁-C₆alkyl, C₃-C₆cycloalkyl, C₁-C₆heteroalkyl, substituted or unsubstituted C₂-C₆cycloheteroalkyl, substituted or unsubstituted phenyl, substituted or unsubstituted naphthyl, substituted or unsubstituted monocyclic heteroaryl, substituted or unsubstituted bicyclic heteroaryl. In some embodiments, R¹ is H, —C(═O)R⁸, C₁-C₆alkyl, C₃-C₆cycloalkyl, substituted or unsubstituted phenyl, substituted or unsubstituted naphthyl, substituted or unsubstituted monocyclic heteroaryl, substituted or unsubstituted bicyclic heteroaryl. In some embodiments, R¹ is H, —C(═O)R⁸, C₁-C₆alkyl, C₃-C₆cycloalkyl, substituted or unsubstituted phenyl, substituted or unsubstituted monocyclic heteroaryl. In some embodiments, R¹ is C₁-C₆alkyl, C₃-C₆cycloalkyl, substituted or unsubstituted phenyl, substituted or unsubstituted monocyclic heteroaryl. In some embodiments, R¹ is C₁-C₆alkyl or C₃-C₆cycloalkyl. In some embodiments, R¹ is substituted or unsubstituted phenyl. In some embodiments, R¹ is substituted or unsubstituted monocyclic heteroaryl. In some embodiments, R¹ is —C(═O)R⁸. In some embodiments, R¹ is H.

In some embodiments, L¹ is —O—, —S—, —C₁-C₃alkylene-, —C₁-C₃alkylene-NH—, or —NH—C₁-C₃alkylene-. In some embodiments, L¹ is —S—. In some embodiments, L¹ is —C₁-C₃alkylene-. In some embodiments, L¹ is —C₁-C₃alkylene-NH—, or —NH—C₁-C₃alkylene-.

In some embodiments, L² is a bond, C₁-C₄alkylene, or C₁-C₄heteroalkylene. In some embodiments, L² is a bond. In some embodiments, L² is a bond or C₁-C₄alkylene. In some embodiments, L² is C₁-C₄alkylene. In some embodiments, L² is a C₁-C₄alkylene, or C₁-C₄heteroalkylene.

In some embodiments, A is pyrrolyl, oxazolyl, thiazolyl, imidazolyl, pyrazolyl, isoxazolyl, isothiazolyl, triazolyl, or thiadiazolyl; R⁴ is C₁-C₄-fluoroalkyl, C₁-C₄alkyl, or C₃-C₆cycloalkyl; R⁵ is H, C₁-C₄alkyl, or —C(═O)R¹⁰; R⁶ is C₁-C₆alkyl, C₃-C₆cycloalkyl, or C₁-C₄-fluoroalkyl; m is 0 or 1.

In some embodiments, A is triazolyl, oxadiazolyl or thiazolyl; R¹ is H, —C(═O)R⁸, C₁-C₆alkyl, C₃-C₆cycloalkyl, C₁-C₆heteroalkyl, substituted or unsubstituted C₂-C₆cycloheteroalkyl, substituted or unsubstituted phenyl, substituted or unsubstituted naphthyl, substituted or unsubstituted monocyclic heteroaryl, substituted or unsubstituted bicyclic heteroaryl; L¹ is —O—, —S—, —C₁-C₃alkylene-, —C₁-C₃alkylene-NH—, or —NH—C₁-C₃alkylene-; L² is a bond, C₁-C₄alkylene, or C₁-C₄heteroalkylene.

In some embodiments, A is

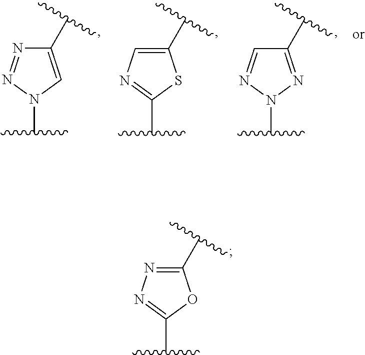 [SMILES: *c1cn(*)nn1 ; *c1cnc(*)s1 ; *c1cnn(*)n1 ; *c1nnc(*)o1]

L² is a bond, C₁-C₄alkylene, or C₁-C₄heteroalkylene; R² is halo, —CO₂R⁹, tetrazole, —C(═O)N(R⁹)₂, —CN, —CH(═O), —SR⁸, —S(O)R⁸, —S(O)₂R⁸, —S(O)₂N(R⁹)₂, —OR⁸, —N(R⁹)₂, or —C(═O)R⁸.

In some embodiments, R² is halo, —CO₂R⁹, tetrazole, —C(═O)N(R⁹)₂, —CN, —CH(═O), —SR⁸, —S(O)R⁸, —S(O)₂R⁸, —S(O)₂N(R⁹)₂, —OR⁸, —N(R⁹)₂, or —C(═O)R⁸. In some embodiments, R² is —CO₂R⁹, tetrazole, —C(═O)N(R⁹)₂, —CN, —CH(═O), —S(O)R⁸, —S(O)₂R⁸, or —C(═O)R⁸.

In some embodiments, A is

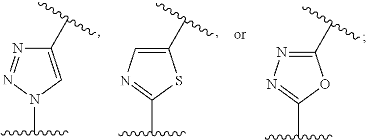 [SMILES: *c1cn(*)nn1 ; *c1cnc(*)s1 ; *c1nnc(*)o1]

R⁴ is C₁-C₄-fluoroalkyl; R⁶ is C₁-C₄alkyl.

In some embodiments, the compound has one of the following structures:

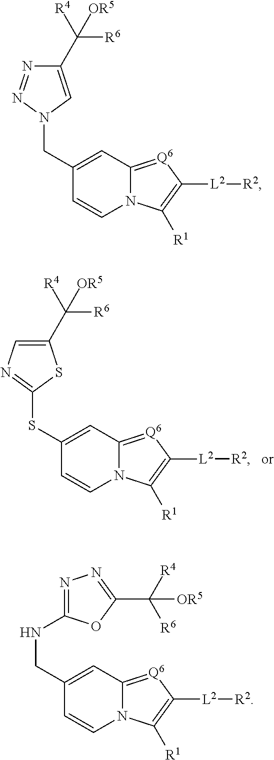

In some embodiments, R¹ is H, —C(═O)R⁸, C₁-C₆alkyl, C₃-C₆cycloalkyl, C₁-C₆heteroalkyl, substituted or unsubstituted C₂-C₆cycloheteroalkyl, substituted or unsubstituted phenyl, substituted or unsubstituted naphthyl, substituted or unsubstituted monocyclic heteroaryl, substituted or unsubstituted bicyclic heteroaryl; L² is a bond, —CH₂—, —CH₂CH₂—, —CH═CH—, or —CH₂NH(CH₂)₂—.

In some embodiments, L² is a bond, —CH₂—, —CH₂CH₂—, —CH═CH—, or —CH₂NH(CH₂)₂—.

In some embodiments, R¹ is C₁-C₆alkyl or C₃-C₆cycloalkyl; R² is —CO₂R⁹, —C(═O)N(R⁹)₂, —CN, —S(O)₂R⁸, —S(O)₂N(R⁹)₂, or —C(═O)R⁸.

In some embodiments, R¹ is C₁-C₆alkyl or C₃-C₆cycloalkyl.

In some embodiments, R² is —CO₂R⁹, —C(═O)N(R⁹)₂, —CN, —S(O)₂R⁸, —S(O)₂N(R⁹)₂, or —C(═O)R⁸.

In some embodiments, R¹ is H, —C(═O)R⁸, —CH₃, —CH₂CH₃, —CH(CH₃)₂, —C(═CH₂)CH₃, cyclopropyl, cyclobutyl, cyclopentyl, cyclopentenyl, cyclohexyl, cyclohexenyl, substituted or unsubstituted phenyl, substituted or unsubstituted naphthyl, substituted or unsubstituted monocyclic heteroaryl selected from substituted or unsubstituted furanyl, substituted or unsubstituted thienyl, substituted or unsubstituted pyrrolyl, substituted or unsubstituted oxazolyl, substituted or unsubstituted thiazolyl, substituted or unsubstituted imidazolyl, substituted or unsubstituted triazolyl, substituted or unsubstituted tetrazolyl, substituted or unsubstituted pyrazolyl, substituted or unsubstituted isoxazolyl, substituted or unsubstituted isothiazolyl, substituted or unsubstituted 1,3,4-thiadiazolyl, substituted or unsubstituted pyridinyl, substituted or unsubstituted pyridazinyl, substituted or unsubstituted pyrimidinyl, substituted or unsubstituted pyrazinyl or substituted or unsubstituted quinolinyl; R² is —CO₂R⁹, —C(═O)N(R⁹)₂, —CN, —S(O)₂R⁸, —S(O)₂N(R⁹)₂, or —C(═O)R⁸.

In some embodiments, R¹ is a substituted or unsubstituted phenyl or a substituted or unsubstituted monocyclic heteroaryl; R² is —CO₂R⁹, —C(═O)N(R⁹)₂, —CN, —S(O)₂R⁸, —S(O)₂N(R⁹)₂, or —C(═O)R⁸.

In some embodiments, R¹ is a substituted or unsubstituted phenyl, substituted or unsubstituted pyridinyl, substituted or unsubstituted pyridazinyl, substituted or unsubstituted pyrimidinyl or substituted or unsubstituted pyrazinyl.

In some embodiments, R¹ is a substituted or unsubstituted phenyl or a substituted or unsubstituted pyridinyl.

In some embodiments, Q⁶ is N; L² is a bond, —CH₂—, —CH₂CH₂—, or —CH═CH—; R⁴ is C₁-C₄-fluoroalkyl; R⁶ is C₁-C₆alkyl; R⁸ is C₁-C₆alkyl; each R⁹ is independently H or C₁-C₆alkyl.

In some embodiments, Q⁶ is CR⁷; L² is a bond, —CH₂—, —CH₂CH₂—, or —CH═CH—; R⁴ is C₁-C₄-fluoroalkyl; R⁶ is C₁-C₆alkyl; R⁷ is H, halo, —C(═O)R⁸, or C₁-C₆alkyl; R⁸ is C₁-C₆alkyl; each R⁹ is independently H or C₁-C₆alkyl.

Any of the combinations of the groups described above for the various variables is contemplated herein. It is understood that substituents and substitution patterns on the compounds provided herein can be selected by one of ordinary skill in the art to provide compounds that are chemically stable and that can be synthesized by techniques known in the art, as well as those set forth herein.

In another aspect, provided are pharmaceutical compositions that include a compound, pharmaceutically acceptable salt, pharmaceutically acceptable N-oxide, pharmaceutically active metabolite, pharmaceutically acceptable prodrug, or pharmaceutically acceptable solvate of any compound described herein. In a further aspect, provided are compositions further including a pharmaceutically acceptable diluent, excipient or binder. In a further aspect, provided are compositions further including a second pharmaceutically active ingredient.

In certain embodiments, provided herein is a pharmaceutical composition containing: i) a physiologically acceptable carrier, diluent, and/or excipient; and ii) one or more compounds provided herein.

In another aspect, described herein is a pharmaceutical composition that includes a therapeutically effective amount of a compound described herein and a pharmaceutically acceptable excipient.

In another aspect, described herein is a method for treating inflammation in a mammal comprising administering a therapeutically effective amount of a compound described herein to the mammal in need.

In another aspect, described herein is a method for treating respiratory disease in a mammal comprising administering a therapeutically effective amount of a compound described herein to the mammal in need. In one aspect, the respiratory disease is asthma.

In another aspect, described herein is a method for treating cardiovascular disease in a mammal comprising administering a therapeutically effective amount of a compound described herein to the mammal in need.

In another aspect, described herein is a method for treating pain in a mammal by administering a therapeutically effective amount of a compound described herein to the mammal in need. In one aspect, the pain is associated with arthritis, including rheumatoid arthritis and osteaoarthritis and ankylosing spondylitis.

In one aspect, provided herein are methods for treating a patient by administering a compound provided herein. In some embodiments, provided herein is a method of inhibiting the activity of 5-LO or of treating a disease, disorder, or condition, which would benefit from inhibition of 5-LO activity in a patient, which includes administering to the patient a therapeutically effective amount of at least one of any of the compounds herein, or pharmaceutically acceptable salt, pharmaceutically acceptable N-oxide, pharmaceutically active metabolite, pharmaceutically acceptable prodrug, or pharmaceutically acceptable solvate.

In another aspect, provided herein are methods of treating a leukotriene dependent or leukotriene-mediated disease or condition in a patient, that includes administering to the patient a therapeutically effective amount of the compound, pharmaceutically acceptable salt, pharmaceutically acceptable N-oxide, pharmaceutically active metabolite, pharmaceutically acceptable prodrug, or pharmaceutically acceptable solvate of any compound described herein, which inhibits the activity of 5-LO. In a further embodiment, the disease or condition is respiratory disease or cardiovascular disease or diseases in which pain occurs. In an alternative embodiment, the disease or condition is asthma, chronic obstructive pulmonary disease, pulmonary hypertension, interstitial lung fibrosis, rhinitis, aortic aneurysm, myocardial infarction, stroke or arthritis. In an alternative embodiment, the disease or condition is asthma. In an alternative embodiment, the disease or condition is osteoarthritis. In an alternative embodiment, the disease or condition is cancer or a non-cancerous disorder. In an alternative embodiment, the disease or condition is a non-cancerous disorder involving the skin or lymphatic tissues. In an alternative embodiment, the disease or disorder is a metabolic disorder. In an alternative embodiment, the disease or disorder relates to bone remodeling, loss or gain. In an alternative embodiment, the disease or condition is iatrogenic.

In some embodiments, the inflammatory conditions to be treated with the compounds described herein include, but are not limited to, asthma, chronic obstructive pulmonary disease, pulmonary hypertension, interstitial lung fibrosis, rhinitis, aortic aneurysm, myocardial infarction, stroke and arthritis. In other embodiments the proliferative disorders include, but are not limited to, cancer and non-cancerous disorders, including, but not limited to, those involving the skin or lymphatic tissues. In other embodiments the metabolic disorders include, but are not limited to, bone remodeling, loss or gain. In additional embodiments, such conditions are iatrogenic and increases in, or abnormal localization of, leukotrienes may be induced by other therapies or medical or surgical procedures.

In other embodiments, the methods, compounds, pharmaceutical compositions, and medicaments described herein may be used to treat NSAID-induced GI tract lesions in a mammal.

In other embodiments, the methods, compounds, pharmaceutical compositions, and medicaments described herein may be used to treat inflammatory diseases associated with fibrosis including pain post chemotherapy or post radio-therapy in a mammal.

In other embodiments, the methods, compounds, pharmaceutical compositions, and medicaments described herein may be used to prevent the cellular activation of 5-lipoxygenase, while in other embodiments the methods, compounds, pharmaceutical compositions, and medicaments described herein may be used to limit the formation of leukotrienes. In other embodiments, such methods, compounds, pharmaceutical compositions, and medicaments include compounds disclosed herein, which inhibit the activity of 5-lipoxygenase, for the treatment of asthma by: (a) lowering the concentrations of leukotrienes in certain tissue(s) of the body or in the entire body of a patient, (b) modulating the activity of enzymes or proteins in a patient wherein such enzymes or proteins are involved in the leukotriene pathway such as, by way of example, 5-lipoxygenase, or (c) combining the effects of (a) and (b). In yet other embodiments, the methods, compounds, pharmaceutical compositions, and medicaments described herein may be used in combination with other medical treatments or surgical modalities.

In some embodiments, provided herein are methods for reducing/inhibiting the leukotriene synthetic activity of 5-lipoxygenase in a mammal comprising administering to the mammal at least once an effective amount of a compound described herein.

In other embodiments, provided herein are methods for modulating, including reducing and/or inhibiting, the activity of 5-lipoxygenase, directly or indirectly, in a mammal comprising administering to the mammal at least once an effective amount of at least one compound described herein.

In further embodiments, provided herein are methods for treating leukotriene-dependent or leukotriene mediated conditions or diseases, comprising administering to the mammal at least once an effective amount of at least one compound described herein.

In yet further embodiments, provided herein are methods for treating inflammation comprising administering to the mammal at least once an effective amount of at least one compound described herein.

In other embodiments, provided herein are methods for treating respiratory diseases comprising administering to the mammal at least once an effective amount of at least one compound described herein. In a further embodiment of this aspect, the respiratory disease is asthma. In a further embodiment of this aspect, the respiratory disease includes, but is not limited to, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma or seasonal asthma.

In yet other embodiments, provided herein are methods for preventing chronic obstructive pulmonary disease comprising administering to the mammal at least once an effective amount of at least one compound described herein. In a further embodiment of this aspect, chronic obstructive pulmonary disease includes, but is not limited to, chronic bronchitis or emphysema, pulmonary hypertension, interstitial lung fibrosis and/or airway inflammation and cystic fibrosis.

In yet other embodiments, provided herein are methods for preventing pain in diseases including, but not limited to, arthritis, comprising administering to the mammal at least once an effective amount of at least one compound described herein. In a further embodiment of this aspect, the painful disease includes, but is not limited to, osteoarthritis, rheumatoid arthritis and ankylosing spondilitis. In another aspect the disease is any musculoseletal disease and the pain associated with these diseases. In another aspect the disease is endometriosis and pain associated with endometriosis and menstruation. In another embodiment the pain is associated with sickle cell disease. In another aspect, the disease is cancer and the pain associated with cancer. In another aspect the disease is fibromyalgia and the pain associated with fibromyalgia. In another embodiment the pain is associated with herpes virus infection of the skin in the disease commonly known as shingles.

In some embodiments, provided herein are methods for preventing increased mucosal secretion and/or edema in a disease or condition comprising administering to the mammal at least once an effective amount of at least one compound described herein.

Yet in other embodiments, provided herein are methods for preventing or treating vasoconstriction, atherosclerosis and its sequelae myocardial ischemia, myocardial infarction, aortic aneurysm, vasculitis and stroke comprising administering to the mammal an effective amount of a compound described herein.

Additional embodiments provided herein include methods for reducing organ reperfusion injury following organ ischemia and/or endotoxic shock comprising administering to the mammal at least once an effective amount of at least one compound described herein.

Still further embodiments provided herein include methods for reducing the constriction of blood vessels in a mammal comprising administering to the mammal at least once an effective amount of at least one compound described herein.

Some other embodiments are methods for lowering or preventing an increase in blood pressure of a mammal comprising administering to the mammal at least once an effective amount of at least one compound described herein.

Other embodiments provided herein include methods for preventing eosinophil and/or basophil and/or dendritic cell and/or neutrophil and/or monocyte recruitment comprising administering to the mammal at least once an effective amount of at least one compound described herein.

Some further embodiments provided herein include methods for the prevention or treatment of abnormal bone remodeling, loss or gain, including diseases or conditions as, by way of example, osteopenia, osteoporosis, Paget's disease, cancer and other diseases comprising administering to the mammal at least once an effective amount of at least one compound described herein.

Additional embodiments provided herein include methods for preventing ocular inflammation and allergic conjunctivitis, vernal keratoconjunctivitis, and papillary conjunctivitis comprising administering to the mammal at least once an effective amount of at least one compound described herein.

Still further embodiments provided herein include methods for preventing CNS disorders comprising administering to the mammal at least once an effective amount of at least one compound described herein. CNS disorders include, but are not limited to, multiple sclerosis, Parkinson's disease, Alzheimer's disease, stroke, cerebral ischemia, retinal ischemia, post-surgical cognitive dysfunction, migraine, peripheral neuropathy/neuropathic pain, spinal cord injury, cerebral edema and head injury.

Some additional embodiments provided herein include methods for the treatment of cancer comprising administering to the mammal at least once an effective amount of at least one compound described herein. The type of cancer may include, but is not limited to, pancreatic cancer and other solid or hematological tumors.

In some embodiments, provided herein are methods for preventing endotoxic shock and septic shock comprising administering to the mammal at least once an effective amount of at least one compound described herein.

In further embodiments, provided herein are methods for preventing rheumatoid arthritis and osteoarthritis comprising administering to the mammal at least once an effective amount of at least one compound described herein.

In other embodiments, methods for preventing increased GI diseases comprising administering to the mammal at least once an effective amount of at least one compound described herein, are provided. Such diseases include, by way of example only, chronic gastritis, eosinophilic gastroenteritis, and gastric motor dysfunction.

In further embodiments, provided herein are methods for treating kidney diseases comprising administering to the mammal at least once an effective amount of at least one compound described herein. Such diseases include, by way of example only, glomerulonephritis, cyclosporine nephrotoxicity renal ischemia reperfusion.

In still further embodiments, provided herein are methods for preventing or treating acute or chronic renal insufficiency comprising administering to the mammal at least once an effective amount of at least one compound described herein.

In yet further embodiments, provided herein are methods for treating type II diabetes comprising administering to the mammal at least once an effective amount of at least one compound described herein.

In other embodiments, provided herein are methods to diminish the inflammatory aspects of acute infections within one or more solid organs or tissues such as the kidney with acute pyelonephritis comprising administering to the mammal at least once an effective amount of at least one compound described herein.

In further embodiments, provided herein are methods for preventing or treating acute or chronic disorders involving recruitment or activation of eosinophils comprising administering to the mammal at least once an effective amount of at least one compound described herein.

In another aspect, provided herein are methods for preventing or treating acute or chronic erosive disease or motor dysfunction of the gastrointestinal tract caused by non-steroidal anti-inflammatory drugs (including selective or non-selective cyclooxygenase-1 or -2 inhibitors) comprising administering to the mammal at least once an effective amount of at least one compound described herein.

Other embodiments provided herein include methods for the prevention or treatment of rejection or dysfunction in a transplanted organ or tissue comprising administering to the mammal at least once an effective amount of at least one compound described herein.

Further embodiments provided herein include methods for treating inflammatory responses of the skin comprising administering to the mammal at least once an effective amount of at least one compound described herein. Such inflammatory responses of the skin include, by way of example, dermatitis, contact dermatitis, eczema, urticaria, rosacea, and scarring. In another aspect are methods for reducing psoriatic lesions in the skin, joints, or other tissues or organs, comprising administering to the mammal an effective amount of a compound described herein.

In another aspect, provided herein are methods for the treatment of cystitis, including, by way of example only, interstitial cystitis, comprising administering to the mammal at least once an effective amount of at least one compound described herein.

Other further embodiments provided herein include methods for the treatment of metabolic syndromes such as Familial Mediterranean Fever or inflammatory pancreatitis comprising administering to the mammal at least once an effective amount of at least one compound described herein.

Additional further embodiments provided herein include methods to treat hepatorenal syndrome comprising administering to the mammal at least once an effective amount of at least one compound described herein.

In another embodiment, compounds described herein are used in the manufacture of a medicament for treating an inflammatory disease or condition in an animal in which the activity of at least one leukotriene protein contributes to the pathology and/or symptoms of the disease or condition. In one embodiment of this aspect, the leukotriene pathway protein is 5-lipoxygenase. In another or further embodiment of this aspect, the inflammatory disease or conditions are respiratory, cardiovascular, arthritic, or proliferative diseases. In one embodiment, the respiratory disease is asthma.

In any of the aforementioned embodiments are further embodiments in which administration is enteral, parenteral, or both, and wherein (a) the effective amount of the compound is systemically administered to the mammal; (b) the effective amount of the compound is administered orally to the mammal; (c) the effective amount of the compound is intravenously administered to the mammal; (d) the effective amount of the compound administered by inhalation; (e) the effective amount of the compound is administered by nasal administration; or (f) the effective amount of the compound is administered by injection to the mammal; (g) the effective amount of the compound is administered topically (dermal) to the mammal; (h) the effective amount of the compound is administered by ophthalmic administration; or (i) the effective amount of the compound is administered rectally to the mammal.

In any of the aforementioned embodiments are further embodiments in which the mammal is a human, including embodiments wherein (a) the human has an asthmatic condition or trait selected from the group consisting of allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, or seasonal asthma, or chronic obstructive pulmonary disease, or pulmonary hypertension or interstitial lung fibrosis. In any of the aforementioned embodiments are further embodiments in which the mammal is an animal model for pulmonary inflammation, examples of which are provided herein.

In any of the aforementioned embodiments are further embodiments comprising single administrations of the effective amount of the compound, including further embodiments in which (i) the compound is administered once; (ii) the compound is administered to the mammal multiple times over the span of one day; (iii) continually; or (iv) continuously.

In any of the aforementioned embodiments are further embodiments comprising multiple administrations of the effective amount of the compound, including further embodiments in which (i) the compound is administered in a single dose; (ii) the time between multiple administrations is every 6 hours; (iii) the compound is administered to the mammal every 8 hours. In further or alternative embodiments, the method comprises a drug holiday, wherein the administration of the compound is temporarily suspended or the dose of the compound being administered is temporarily reduced; at the end of the drug holiday, dosing of the compound is resumed. The length of the drug holiday can vary from 2 days to 1 year.

In any of the aforementioned embodiments involving the treatment of leukotriene dependent diseases or conditions are further embodiments comprising administering at least one additional agent, including, by way of example, an anti-inflammatory agent, a different compound having the structure of Formula (I) or (II), a CysLT₁ receptor antagonist, or a CysLT₁/CysLT₂ dual receptor antagonist. In further or alternative embodiments, the CysLT₁ antagonist is selected from montelukast (Singulair®: [1-[[1-[3-[2-[(7-chloro-2-quinolyl)]vinyl]phenyl]-3-[2-(1-hydroxy-1-methyl-ethyl)phenyl]-propyl]sulfanylmethyl]cyclopropyl]acetic acid), zafirlukast (Accolate® 3-[[2-methoxy-4-(o-tolylsulfonylcarbamoyl)phenyl]methyl]-1-methyl-1H-indol-5-yl]aminoformic acid cyclopentyl ester) or pranlukast (Onon: 4-oxo-8-[p-(4-phenylbutyloxy)benzoylamino]-2-tetrazol-5-yl)-4H-1-benzopyran).

In further or alternative embodiments, the anti-inflammatory agent includes, but is not limited to, non-steroidal anti-inflammatory drugs such as a cyclooxygenase inhibitor (COX-1 and/or COX-2), lipoxygenase inhibitors and steroids such as prednisone or dexamethasone. In further or alternative embodiments, the anti-inflammatory agent is selected from the group consisting of Arthrotec® (diclofenac and misoprostol), Asacol® (5-aminosalicyclic acid), Auralgan® (antipyrine and benzocaine), Azulfidine® (sulfasalazine), Daypro® (oxaprozin), etodolac, Ponstan® (mefenamic acid), Salofalk® (5-aminosalicylic acid), Solu-Medrol® (methylprednisolone), aspirin, Indocin® (indomethacin), Vioxx® (rofecoxib), Celebrex® (celecoxib), Bextra® (valdecoxib), diclofenac, etodolac (Lodine®), ketoprofen, Mobic® (meloxicam), nabumetone, naproxen, Feldene® (piroxicam), Celestone® (betamethasone), Deltasone® (prednisone), or any generic equivalent thereof.

In any of the aforementioned embodiments involving the treatment of pain, including but not limited to arthritic pain, are further embodiments comprising administering at least one additional agent selected from the group consisting of opiod drugs like morphine, fentanyl, bupivacaine and anesthetic compounds such as lidocaine

In any of the aforementioned embodiments involving the treatment of proliferative disorders, including cancer, are further embodiments comprising administering at least one additional agent selected from the group consisting of alemtuzumab, arsenic trioxide, asparaginase (pegylated or non-), bevacizumab, cetuximab, platinum-based compounds such as cisplatin, cladribine, daunorubicin/doxorubicin/idarubicin, irinotecan, fludarabine, 5-fluorouracil, gemtuzumab, methotrexate, paclitaxel (Taxol™), temozolomide, thioguanine, or classes of drugs including hormones (an antiestrogen, an antiandrogen, or gonadotropin releasing hormone analogues, interferons such as alpha interferon, nitrogen mustards such as busulfan or melphalan or mechlorethamine, retinoids such as tretinoin, topoisomerase inhibitors such as irinotecan or topotecan, tyrosine kinase inhibitors such as gefinitinib or imatinib, or agents to treat signs or symptoms induced by such therapy including allopurinol, filgrastim, granisetron/ondansetron/palonosetron, dronabinol.

In any of the aforementioned embodiments involving the therapy of transplanted organs or tissues or cells are further embodiments that include administering at least one additional agent selected from the group consisting of azathioprine, a corticosteroid, cyclophosphamide, cyclosporin, dacluzimab, mycophenolate mofetil, OKT3, rapamycin, tacrolimus, or thymoglobulin.

In any of the aforementioned embodiments involving the therapy of interstitial cystitis are further embodiments that include administering at least one additional agent selected from dimethylsulfoxide, omalizumab, and pentosan polysulfate.

In any of the aforementioned embodiments involving the therapy of disorders of bone are further embodiments that include administering at least one additional agent selected from the group consisting of minerals, vitamins, bisphosphonates, anabolic steroids, parathyroid hormone or analogs, and cathepsin K inhibitors, dronabinol.

In any of the aforementioned embodiments involving the prevention or treatment of inflammation are further embodiments comprising: (a) monitoring inflammation in a mammal; (b) measuring bronchoconstriction in a mammal; (c) measuring eosinophil and/or basophil and/or dendritic cell and/or neutrophil and/or monocyte and/or lymphocyte recruitment in a mammal; (d) monitoring mucosal secretion in a mammal; (e) measuring mucosal edema in a mammal; (e) measuring levels of LTB₄ in the calcium ionophore-challenged blood of a mammal; (f) measuring levels of LTE₄ in the urinary excretion of a mammal; or (g) identifying a patient by measuring leukotriene-driven inflammatory biomarkers such as LTB₄, LTC₄, IL-6, CRP, SAA, MPO, EPO, MCP-1, MIP-α, sICAMs, IL-4, IL-13.

In any of the aforementioned embodiments the leukotriene-dependent or leukotriene mediated diseases or conditions include, but are not limited to, asthma, chronic obstructive pulmonary disease, pulmonary hypertension, interstitial lung fibrosis, rhinitis, arthritis, allergy, inflammatory bowel disease, adult respiratory distress syndrome, myocardial infarction, aneurysm, stroke, cancer, and endotoxic shock.

In some embodiments, compounds provided herein are administered to a human.

In some embodiments, compounds provided herein are orally administered. In some embodiments, compounds provided herein are topically administered.

In some embodiments, compounds provided herein are used for inhibiting the activity of 5-LO. In some embodiments, compounds provided herein are used for inhibiting the activity of 5-LO or for the treatment of a disease or condition that would benefit from inhibition of 5-LO activity.

In other embodiments, compounds provided herein are used for the formulation of a medicament for the inhibition of 5-LO activity.

Articles of manufacture containing packaging material, a compound described herein, or composition or pharmaceutically acceptable derivative thereof, which is effective for inhibiting the activity of 5-LO, within the packaging material, and a label that indicates that the compound or composition, or pharmaceutically acceptable salt, pharmaceutically active metabolite, pharmaceutically acceptable prodrug, or pharmaceutically acceptable solvate thereof, is used for inhibiting the activity of 5-LO, are provided.

Other objects, features and advantages of the methods, compounds, and compositions described herein will become apparent from the following detailed description. It should be understood, however, that the detailed description and the specific examples, while indicating specific embodiments, are given by way of illustration only, since various changes and modifications within the spirit and scope of the disclosure will become apparent to those skilled in the art from this detailed description.

### DETAILED DESCRIPTION

Described herein are compounds, methods of making such compounds, pharmaceutical compositions and medicaments that include such compounds, and methods of using such compounds to treat or prevent diseases or conditions associated with 5-lipoxygenase activity. Described herein are compounds that inhibit the activity of 5-lipoxygenase (5-LO).

Leukotrienes (LTs) are potent contractile and inflammatory mediators produced by release of arachidonic acid from cell membranes and conversion to leukotrienes by the action of 5-lipoxygenase, 5-lipoxygenase-activating protein, LTA₄ hydrolase and LTC₄ synthase. The leukotriene synthesis pathway, or 5-lipoxygenase pathway, involves a series of enzymatic reactions in which arachidonic acid is converted to leukotriene LTB₄, or the cysteinyl leukotrienes, LTC₄, LTD₄, and LTE₄. The pathway occurs mainly at the nuclear envelope and has been described. See, e.g., Wood, J W et al., J. Exp. Med., 178: 1935-1946, 1993; Peters-Golden, Am. J. Respir. Crit. Care Med. 157:S227-S232, 1998; Drazen, et al., ed. Five-Lipoxygenase Products in Asthma, Lung Biology in Health and Disease Series, Vol. 120, Chs. 1, 2, and 7, Marcel Dekker, Inc. N.Y., 1998. Protein components dedicated to the leukotriene synthesis pathway include 5-lipoxygenase (5-LO), 5-lipoxygenase-activating protein (FLAP), LTA₄ hydrolase, and LTC₄ synthase. The synthesis of leukotrienes has been described in the literature, e.g., by Samuelsson et al., Science, 220, 568-575, 1983; Peters-Golden, “Cell Biology of the 5-Lipoxygenase Pathway” Am J Respir Crit. Care Med 157:S227-S232 (1998). Leukotrienes are synthesized directly from arachidonic acid by different cells including eosinophils, neutrophils, basophils, lymphocytes, macrophages, monocytes and mast cells. Excess LTA₄, for example from an activated neutrophil, may enter a cell by a transcellular pathway. Most cells in the body have LTA₄ hydrolase so can produce LTB₄. Platelets and endothelial cells have LTC₄ synthase, so can make LTC₄ when presented with LTA₄ by a transcellular pathway.

Arachidonic acid is a polyunsaturated fatty acid and is present mainly in the membranes of the body's cells. Upon presentation of inflammatory stimuli from the exterior of the cell, calcium is released and binds to phospholipase A₂ (PLA2) and 5-LO. Cell activation results in the translocation of PLA₂ and 5-LO from the cytoplasm to the endoplasmic reticulum and/or nuclear membranes, where in the presence of FLAP, the released arachidonic acid is converted via a 5-HPETE intermediate to the epoxide LTA₄. Depending on the cell type, the LTA₄ may be immediately converted to LTC₄ by the nuclear-bound LTC₄ synthase or to LTB₄ by the action of cytosolic LTA₄ hydrolase. LTB₄ is exported from cells by an as yet uncharacterized transporter and may activate other cells, or the cell it was made in, via high affinity binding to one of two G protein-coupled receptors (GPCRs), namely BLT₁R or BLT₂R. LTC₄ is exported to the blood via the MRP-1 anion pump and rapidly converted to LTD₄ by the action of γ-glutamyl transpeptidase and LTD₄ and is then converted to LTE₄ by the action of dipeptidases. LTC₄, LTD₄ and LTE₄, which are collectively referred to as the cysteinyl leukotrienes (or previously as slow reacting substance of anaphylaxis, SRS-A). The cysteinyl leukotrienes activate other cells, or the cells they are made in, via high affinity binding to one of two GPCRs, namely CysLT₁R or CysLT₂R. CysLT₁ receptors are found in the human airway eosinophils, neutrophils, macrophages, mast cells, B-lymphocytes and smooth muscle and induce bronchoconstriction. Zhu et al., Am. J. Respir. Cell Mol. Biol. Epub. August 25 (2005). CysLT₂ receptors are located in human airway eosinophils, macrophages, mast cells the human pulmonary vasculature (Figueroa et al., Clin. Exp Allergy 33:1380-1388; 2003).

5-Lipoxygenase-activating protein has been shown to form two distinct multimeric complexes that regulate the formation of leukotrienes in RBL-2H3 cells; Mandal et al, Proc Natl Acad. Sci., 101, 6587-6592 (2004). The first complex is the formation of homodimers or homotrimers of 5-lipoxygenase-activating protein, the second is the formation of heterodimers or heterotrimers involving 5-lipoxygenase-activating protein and LTC₄ synthase. The tight association of LTC₄ synthase with 5-lipoxygenase-activating protein and the low expression level of LTC₄ synthase implies that all the LTC₄ synthase is tied up in the heteromultimers with 5-lipoxygenase-activating protein. The formation of LTC₄ is likely regulated through the heterodimer or heterotrimer while the homodimer or homotrimer of 5-lipoxygenase-activating protein regulates the generation of LTA₄ that is then available for the conversion to LTB₄. Inhibition of 5-lipoxygenase results in the complete downstream inhibition of the formation of leukotrienes. In contrast, the existence of different multimeric complexes of 5-lipoxygenase-activating protein offers the possibility of differentially regulating the inhibition of the production of LTB₄ or the cysteinyl leukotrienes LTC₄, LTD₄ and LTE₄ through the preparation of 5-lipoxygenase-activating protein inhibitors selective for each multimeric complex.

### Involvement of Leukotrienes in Diseases or Conditions

The involvement of leukotrienes in disease is described in detail in the literature. See e.g., Busse, Clin. Exp. Allergy 26:868-79, 1996; O'Byrne, Chest 111 (Supp. 2): 27S-34S, 1977; Sheftell, F. D., et al., Headache, 40:158-163, 2000; Klickstein et al., J. Clin. Invest., 66:1166-1170, 1950; Davidson et al., Ann. Rheum. Dis., 42:677-679, 1983. Leukotrienes produce marked inflammatory responses in human skin. Evidence for the involvement of leukotrienes in a human disease is found in psoriasis, in which leukotrienes have been detected in psoriatic lesions (Kragballe et al., Arch. Dermatol., 119:548-552, 1983).

For example, inflammatory responses have been suggested to reflect three types of changes in the local blood vessels. The primary change is an increase in vascular diameter, which results in an increase in local blood flow and leads to an increased temperature, redness and a reduction in the velocity of blood flow, especially along the surfaces of small blood vessels. The second change is the activation of endothelial cells lining the blood vessel to express adhesion molecules that promote the binding of circulating leukocytes. The combination of slowed blood flow and induced adhesion molecules allows leukocytes to attach to the endothelium and migrate into the tissues, a process known as extravasation. These changes are initiated by cytokines and leukotrienes produced by activated macrophages. Once inflammation has begun, the first cells attracted to the site of infection are generally neutrophils. They are followed by monocytes, which differentiate into more tissue macrophages. In the latter stages of inflammation, other leukocytes, such as eosinophils and lymphocytes also enter the infected site. The third major change in the local blood vessels is an increase in vascular permeability. Instead of being tightly joined together, the endothelial cells lining the blood vessel walls become separated, leading to exit of fluid and proteins from the blood and their local accumulation in the tissue. (See Janeway, et al., Immunobiology: the immune system in health and disease, 5th ed., Garland Publishing, New York, 2001).

LTB₄ produces relatively weak contractions of isolated trachea and lung parenchyma, and these contractions are blocked in part by inhibitors of cyclooxygenase, suggesting that the contraction are secondary to the release of prostaglandins. However, LTB₄ has been shown to be a potent chemotactic agent for eosinophils and progenitors of mast cells; and the LTB₄ receptor BLT1−/− knockout mouse is protected from eosinophilic inflammation and T-cell mediated allergic airway hyperreactivity. (Miyahara et al., J. Immunol. 174:4979-4784; Weller et al J Exp Med 201:1961-1971 (2005)). LTB₄ has also been shown to be elevated in synovial fluid in rheumatoid arthritis (Davidson et al., Annals Rheum. Diseases 42:677-679 (1983) and to be involved in arthritic joint pain in both rats (Castro da Rocha et al., Eur. J. Pharm. 497:81-86 (2004) and mice (Guerrero et al., J. Leuk. Biol 83:122-130 (2008). In addition, LTB₄ mediates the itch-associated pain and inflammation responses of intradermal nocioceptin (Andoh et al., J. Invest. Dermatol. 123:196-201 (2004). LTB₄ enhances atherosclerotic progression in two atherosclerotic mouse models, namely low density receptor lipoprotein receptor deficient (LDLr−/−) and apolipoprotein E-deficient (ApoE−/−) mice (Aiello et al., Arterioscler. Thromb. Vasc. Biol. 22:443-449 (2002); Subbarao et al., Arterioscler. Thromb. Vasc. Bio/0.24:369-375 (2004); Heller et al., Circulation 112:578-586 (2005). LTB₄ has also been shown to increase human monocyte chemoattractant protein (MCP-1) a known enhancer of atherosclerotic progression (Huang et al., Arterioscler. Thromb. Vasc. Bio/0.24:1783-1788, 2004).

Leukotrienes C₄ and D₄ are potent smooth muscle contractile agents, promoting bronchoconstriction in a variety of species, including humans (Dahlen et al., Nature, 288:484-486, 1980). These compounds have profound hemodynamic effects, constricting coronary blood vessels, and resulting in a reduction of cardiac output efficiency (Marone et al., in Biology of Leukotrienes, ed. By R. Levi and R. D. Krell, Ann. New York Acad. Sci. 524:321-333, 1988). Leukotrienes also act as vasoconstrictors, however, marked differences exist for different vascular beds. There are reports suggesting that leukotrienes contribute to cardiac reperfusion injury following myocardial ischemia (Barst and Mullane, Eur. J. Pharmacol., 114: 383-387, 1985; Sasaki et al., Cardiovasc. Res., 22: 142-148, 1988). LTC₄ and LTD₄ directly increase vascular permeability probably by promoting retraction of capillary endothelial cells via activation of the CysLT₂ receptor and possibly other as yet undefined CysLT receptors [Lotzer et al Arterioscler Thromb Vasc Biol 23: e32-36. (2003)]. Urinary LTE₄ is a measurement of production of cysteinyl leukotrienes in disease and has been shown to be elevated in respiratory disease (Taylor et al., Lancet 584-588 (1989), cardiovascular disease (Carry et al., Circulation 85:230-236 (1992) and in sickle cell disease (Field et al., Am. J. Hematol. 84:231-233 (2009).

The role of 5-lipoxygenase in the leukotriene synthesis pathway is significant because 5-lipoxygenase in concert with 5-lipoxygenase-activating protein performs the first step in the pathway for the synthesis of leukotrienes. Therefore, the leukotriene synthesis pathway provides a number of targets for compounds useful in the treatment of leukotriene-dependent or leukotriene mediated diseases or conditions, including, by way of example, vascular and inflammatory disorders, proliferative diseases, and non-cancerous disorders.

Leukotriene-dependent or leukotriene mediated conditions treated using the methods, compounds, pharmaceutical compositions and medicaments described herein, include, but are not limited to, bone diseases and disorder, cardiovascular diseases and disorders, inflammatory diseases and disorders, dermatological diseases and disorders, ocular diseases and disorders, cancer and other proliferative diseases and disorders, respiratory diseases and disorders, such as, for example, asthma, and non-cancerous disorders.

### Treatment Options

Leukotrienes are known to contribute to the inflammation of the airways of patients with asthma. CysLT₁ receptor antagonists such as montelukast (Singulair®) have been shown to be efficacious in asthma and allergic rhinitis [Reiss et al. Arch Intern Med 158:1213-1220 (1998); Phillip et al. Clin. Exp Allergy 32:1020-1028 (2002)]. CysLT₁R antagonists pranlukast (Onon) and zafirlukast (Accolate®) have also been shown to be efficacious in asthma.

Several inhibitors of 5-lipoxygenase have been described: Zyflo® (zileuton; approved by the FDA for the prevention and treatment of asthma, Israel et al. Ann Intern Med 119:1059-1066); ZD2138 (6-[(3-fluoro-5-[4-methoxy-3,4,5,6-tetrahydro-2H-pyran-4-yl])phenoxy methyl]-1-methyl-2-quinolone), which has shown efficacy in inhibiting the fall of FEV1 resulting from aspirin-induced asthma (Nasser et al, Thorax, 49, 749-756, 1994); CJ-13,610 (Mano et al, Chem. Pharm. Bull., 53, 965-973, 2005); MK0633; ABT-761 (atreleuton; Stewart et al, J. Med. Chem., 1997, 40, 1955-1968); AZD-4407 [5-((4-((2S,4R)-tetrahydro-4-hydroxy-2-methyl-2H-pyran-4-yl)thiophen-2-yl)sulfanyl)-1-methylindolin-2-one] (European Patent EP 623614); L-739,010 ([1S,5R]-3-cyano-1-(3-furyl)-6-{6-[3-(3-hydroxy-6,8-dioxabicyclo[3.2.1]octanyl)]pyridin-2-yl-methoxyl}naphthalene) (Hamel et al, J. Med. Chem., 40, 2866-2875, 1997); Wy-50,295 ((S)-2-(2-((quinolin-2-yl)methoxy)naphthalen-7-yl)propanoate tromethamine) (Musser and Kreft, Drugs of the Future, 15, 73-80, 1990) and TMK688 (Tohda et al, Clin. Exp. Allergy, 27, 110-118, 1997). See also Young, Eur. J. Med. Chem., 34, 671-685, 1999 and Werz Expert Opin. Ther. Patents, 15, 505-519, 2005. Several inhibitors of 5-lipoxygenase-activating protein have also been described: MK886 (2-((1-(4-chlorobenzyl)-3-(tert-butylthio)-5-isopropyl-1H-indol-2-yl)methyl)-2-methylpropanoic acid) (Gillard et al, Can. J. Physiol. Pharmacol., 67, 456-464, 1989); MK591 (2-((5-((quinolin-2-yl)methoxy)-1-(4-chlorobenzyl)-3-(tert-butylthio)-1H-indol-2-yl)methyl)-2-methylpropanoic acid) (Brideau et al, Can. J. Physiol. Pharmacol.); BAY X1005 ((R)-2-(4-((quinolin-2-yl)methoxy)phenyl)-2-cyclopentylacetic acid) (Fruchtmann et al, Agents Action, 38, 188-195, 1993); VML-530 (Abt-080; Kolasa et al, J. Med. Chem., 43, 3322-3334, 2000); and ETH615 (Kirstein et al, Pharm. Toxicol., 68, 125-130, 1991). See also: Musser et al, J. Med. Chem., 35, 2501-2524, 1992; Brooks et al. J. Med. Chem., 1996, Vol. 39, No. 14, 2629-2654; Steinhilber, Curr. Med. Chem. 6(1):71-85, 1999; Riendeau, Bioorg Med Chem Lett., 15(14):3352-5, 2005; Flamand, et al., Mol. Pharmacol. 62(2):250-6, 2002; Folco, et al., Am. J. Respir. Crit. Care Med. 161 (2 Pt 2):S112-6, 2000; Hakonarson, JAMA, 293(18):2245-56, 2005). AM103 (GSK2190914) Hutchinson et al, J. Med. Chem., 2009; AM803 (GSK2190915), AM679, AM643; CJ-13610 . . . .

5-Lipoxygenase inhibition will decrease LTB₄ from monocytes, neutrophils and other cells involved in vascular inflammation and thereby decrease atherosclerotic progression. The FLAP inhibitor MK-886 has been shown to decrease the post-angioplasty vasoconstrictive response in a porcine carotid injury model. Provost et al., Brit. J. Pharmacol. 123: 251-258 (1998). MK-886 has also been shown to suppress femoral artery intimal hyperplasia in a rat photochemical model of endothelial injury. Kondo et al. Thromb. Haemost. 79:635-639 (1998). The 5-lipoxygenase inhibitor zileuton has been shown to reduce renal ischemia in a mouse model. Nimesh et al., Mol. Pharm. 66:220-227 (2004). The 5-lipoxygenase inhibitor CJ-13610 has been shown to reduce chronic inflammatory pain in a rat model of osteoarthritis (Cortes-Burgos et al., Eur. J. Pharm. 617:59-67 (2009).

Leukotriene pathway modulators have been used for the treatment of a variety of diseases or conditions, including, by way of example only, (i) inflammation (see e.g. Leff A R et al., Ann. Allergy Asthma Immunol., 2001, 86 (Suppl. 1) 4-8; Riccioni G, et al., Ann. Clin. Lab Sci. 2004, 34(4): 379-870); (ii) respiratory diseases including asthma, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma (see e.g. Riccioni et al., Ann. Clin. Lab. Sci., v 34, 379-387 (2004)); (iii) chronic obstructive pulmonary disease, including chronic bronchitis or emphysema, pulmonary hypertension, interstitial lung fibrosis and/or airway inflammation and cystic fibrosis (see e.g. Kostikas K et al., Chest 2004; 127:1553-9); (iv) increased mucosal secretion and/or edema in a disease or condition (see e.g. Shahab, R., et al., J. Laryngol. Otol., 2004; 118; 500-7); arthritic disease and pain associated with arthritic disease (see Castro da Rocha et al., Eur. J. Pharm. 497:81-86 (2004); Guerrero et al., J. Leuk. Biol 83:122-130 (2008); Cortes-Burgos et al., Eur. J. Pharm. 617:59-67 (2009); cardiovascular disease including vasoconstriction, atherosclerosis and its sequelae, myocardial ischemia, myocardial infarction, aortic aneurysm, vasculitis and stroke (see e.g. Jala et al., Trends in Immunol., v 25, 315-322 (2004) and Mehrabian et al., Curr. Opin. Lipidol., v 14, 447-457 (2003)); (vi) reducing organ reperfusion injury following organ ischemia and/or endotoxic shock (see e.g. Matsui N et al., Planta Med. 2005 August; 71(8):717-20); (vii) reducing the constriction of blood vessels (see e.g. Stanke-Labesque F et al., Br J Pharmacol. 2003 September; 140(1):186-94); (viii) lowering or preventing an increase in blood pressure (see e.g. Stanke-Labesque F et al., Br J. Pharmacol. 2003 September; 140(1):186-94, and Walch L, et al. Br J Pharmacol. 2002 December; 137(8):1339-45); (ix) preventing eosinophil and/or basophil and/or dendritic cell and/or neutrophil and/or monocyte recruitment (see e.g. Miyahara N, et al. Immunol. 2005 Apr. 15; 174(8):4979-84); (x) abnormal bone remodeling, loss or gain, including osteopenia, osteoporosis, Paget's disease, cancer and other diseases (see e.g. Anderson G I, et al., Biomed Mater Res. 2001; 58(4):406-140; (xi) ocular inflammation and allergic conjunctivitis, vernal keratoconjunctivitis, and papillary conjunctivitis (see e.g. Lambiase et al., Arch. Opthalmol., v 121, 615-620 (2003)); (xii) CNS disorders, including, but are not limited to, multiple sclerosis, Parkinson's disease, Alzheimer's disease, stroke, cerebral ischemia, retinal ischemia, post-surgical cognitive dysfunction, migraine (see e.g. de Souza Carvalho D, et al. Headache, 2002, November-December; 42(10): 1044-7; Sheftell F, et al., Headache, 2000, February; 40(2): 158-63); (xiii) peripheral neuropathy/neuropathic pain, spinal cord injury (see e.g. Akpek E A, et al., Spine, 1999, Jan. 15, 24(2): 128-32), cerebral edema and head injury; (xiv) cancer, including, but is not limited to, pancreatic cancer and other solid or hematological tumors, (see e.g. Poff and Balazy, Curr. Drug Targets Inflamm. Allergy, v 3, 19-33 (2004) and Steele et al., Cancer Epidemiology & Prevention, v 8, 467-483 (1999); (xv) endotoxic shock and septic shock (see e.g. Leite M S, et al., Shock. 2005 February; 23(2):173-8); (xvi) rheumatoid arthritis and osteoarthritis (see e.g. Alien R, et al., Ann. Rheum. Dis. 2004 February; 63(2):170-6); (xvii) preventing increased GI diseases, including, by way of example only, chronic gastritis, eosinophilic gastroenteritis, and gastric motor dysfunction, (see e.g. Gyomber et al., J Gastroenterol Hepatol., v 11, 922-927 (1996); Quack I et al., BMC Gastroenterol v 18, 24 (2005); Cuzzocrea S, et al., Lab Invest. 2005 June; 85(6):808-22); (xviii) kidney diseases, including, by way of example only, glomerulonephritis, cyclosporine nephrotoxicity renal ischemia reperfusion. (see e.g. Guasch et al Kidney Int., v 56, 261-267; Butterly et al., v 57, 2586-2593 (2000); Guasch A et al., Kidney Int. 1999; 56:261-7; Butterly D W et al., Kidney Int. 2000; 57:2586-93); (xix) preventing or treating acute or chronic renal insufficiency (see e.g. Maccarrone M, et al., J Am Soc Nephrol. 1999; 10:1991-6); (xx) type II diabetes (see e.g. Valdivielso J M, et al., J. Nephrol. 2003, 16(1):85-94; Parlapiano C, et al., Diabetes Res. Clin. Pract., 1999, October, 46(1): 43-5); (xxi) diminish the inflammatory aspects of acute infections within one or more solid organs or tissues such as the kidney with acute pyelonephritis (see e.g. Tardif M, et al., Antimicrob. Agents Chemother., 1994, July, 38(7):1555-60); (xxii) preventing or treating acute or chronic disorders involving recruitment or activation of eosinophils (see e.g. Quack I, et al., BMC Gastroenterol., 2005; 5:24); (xxiii) preventing or treating acute or chronic erosive disease or motor dysfunction of the gastrointestinal tract caused by non-steroidal anti-inflammatory drugs (including selective or non-selective cyclooxygenase-1 or -2 inhibitors) (see e.g. Marusova I B, et al., Eksp. Klin. Farmakol., 2002, 65:16-8 and Gyomber E, et al., J. Gastroenterol. Hepatol., 1996, 11, 922-7; Martin St et al., Eur. J. Gastroenterol. Hepatol., 2005, 17: 983-6); (xxiv) treatment of metabolic syndromes, including, by way of example only, Familial Mediterranean Fever (see e.g. Bentancur A G, et al., Clin. Exp. Rheumatol., 2004, July-August, 22(4 Suppl. 34): S56-8; and (xxv) treat hepatorenal syndrome [see e.g. Capella G L., Prostaglandins Leukot. Essent. Fatty Acids. 2003 April; 68(4): 263-5].

### Identification of Leukotriene Synthesis Pathway Inhibitors

The development and testing of novel 5-lipoxygenase inhibitors, which are effective either alone or in combination with other drugs, and which result in minimal negative side effects would be beneficial for treating leukotriene-dependent or leukotriene mediated diseases, disorders, or conditions Inhibitors of the leukotriene synthesis pathway described herein may target any step of the pathway to prevent or reduce the formation of leukotrienes. Such leukotriene synthesis inhibitors can, by way of example, inhibit at the level of 5-lipoxygenase, or 5-lipoxygenase-activating protein, thus minimizing the formation of various products in the leukotriene pathway, thereby decreasing the amounts of such compounds available in the cell. Leukotriene synthesis inhibitors can be identified based on their ability to bind to proteins in the leukotriene synthesis pathway. For example, 5-lipoxygenase inhibitors can be identified based on the inhibition of formation of the intermediate product 5-HPETE/5-HETE in cytosol fractions or purified 5-lipoxygenase, with product measured by HPLC or spectrophotometry, or by the inhibition of LTB₄ production from stimulated human leukocytes or by the inhibition of LTB₄ production from stimulated human blood (with product LTB₄ measured in both cases by LTB₄ specific ELISA.

### Compounds

Described herein are compounds that inhibit the activity of 5-lipoxygenase. Also described herein are pharmaceutically acceptable salts, pharmaceutically acceptable N-oxides, pharmaceutically active metabolites and pharmaceutically acceptable prodrugs of such compounds. Pharmaceutical compositions that include at least one such compound or a pharmaceutically acceptable salt, pharmaceutically acceptable N-oxide, pharmaceutically active metabolite or pharmaceutically acceptable prodrug of such compound are provided.

In one aspect, provided herein are compounds of Formula (I). Formula (I) is as follows:

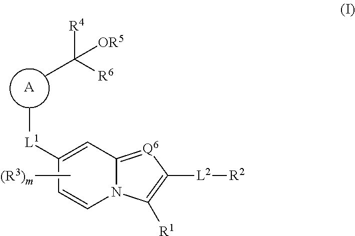

wherein:
A is a substituted or unsubstituted heteraryl or cycloheteroalkyl;
R¹ is H, COR⁸, substituted or unsubstituted alkyl, substituted or unsubstituted cycloalkyl, substituted or unsubstituted heteroalkyl, substituted or unsubstituted cycloheteroalkyl, substituted or unsubstituted aryl, substituted or unsubstituted heteroaryl;
R² is CO₂R⁸, CONR⁹₂, CN, CHO, S(O)ₙR⁸, substituted or unsubstituted alkyl, substituted or unsubstituted cycloalkyl, substituted or unsubstituted heteroalkyl, substituted or unsubstituted cycloheteroalkyl, substituted or unsubstituted aryl, substituted or unsubstituted heteroaryl, OR⁸, SR⁸, NR⁹₂, or COR⁸;
each R³ is independently halo, CO₂R⁸, CONR⁹₂, CN, tetrazole, CHO, S(O)ₙR⁸, substituted or unsubstituted alkyl, substituted or unsubstituted heteroalkyl, OR⁸, SR⁸, NR⁹₂, or COR⁸;
R⁴ is haloalkyl, alkyl, cycloalkyl, or heteroalkyl;
R⁵ is H, alkyl, or COR¹⁰;
R⁶ is H, alkyl, cycloalkyl, heteroalkyl, or haloalkyl;
each R⁷ is independently H, halo, CO₂R⁸, CONR⁹₂, CN, CHO, S(O)ₙR⁸, substituted or unsubstituted alkyl, substituted or unsubstituted heteroalkyl, substituted or unsubstituted aryl, substituted or unsubstituted heteroaryl, OR⁸, SR⁸, NR⁹₂, or COR⁸;
each R⁸ is independently H, substituted or unsubstituted alkyl (e.g., fluoroalkyl), substituted or unsubstituted heteroalkyl, substituted or unsubstituted aryl, or substituted or unsubstituted heteroaryl;
each R⁹ is independently H, substituted or unsubstituted alkyl, substituted or unsubstituted heteroalkyl, substituted or unsubstituted aryl, or substituted or unsubstituted heteroaryl;
   or two R⁹ taken together with the nitrogen to which they are bound form a substituted or unsubstituted heterocycle;
each R¹⁰ is substituted or unsubstituted alkyl, substituted or unsubstituted heteroalkyl, substituted or unsubstituted aryl, or substituted or unsubstituted heteroaryl;
   or two R¹⁰ taken together with the carbon to which they are bound form a substituted or unsubstituted carbocycle, or substituted or unsubstituted heterocycle;
L¹ is O, NR⁸, S, C₁-C₃ alkylene, or C₁-C₃ heteroalkylene;
L² is a bond, C₁-C₆ alkylene, or C₁-C₆ heteroalkylene;
m is 0-3;
each n is independently 0-3.

In specific embodiments, A is a five membered heteroaryl. In other specific embodiments, A is a five membered cycloheteroaryl. In some embodiments A is

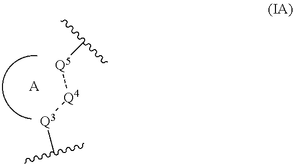

Q³ is C or N;
Q⁵ is C
Q⁴ is CR⁷, S, O, NR⁸, or N; and
each bond represented by ———— is independently a single or double bond.

In specific embodiments, the bond connecting Q³ and Q⁴ is a double bond, the bond connecting Q⁴ and Q⁵ is a double bond, or Q⁴ is O or S.

In some embodiments, the compound has a structure represented by Formula (II):

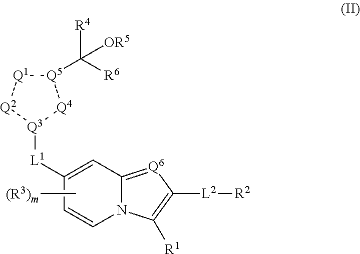

wherein:

Q¹ and Q² are independently N, NR⁷, CR⁷, O, C═X, or S;

Q³ is C or N;

Q⁵ is C

Q⁴ is CR⁷, S, O, NR⁸, or N;
Q⁶ is N or CR⁷;
each X is independently O, NR⁸,
R¹ is H, COR⁸, substituted or unsubstituted alkyl, substituted or unsubstituted cycloalkyl, substituted or unsubstituted heteroalkyl, substituted or unsubstituted cycloheteroalkyl, substituted or unsubstituted aryl, substituted or unsubstituted heteroaryl;
R² is CO₂R⁸, CONR⁹₂, CN, CHO, S(O)ₙR⁸, substituted or unsubstituted alkyl, substituted or unsubstituted cycloalkyl, substituted or unsubstituted heteroalkyl, substituted or unsubstituted cycloheteroalkyl, substituted or unsubstituted aryl, substituted or unsubstituted heteroaryl, OR⁸, SR⁸, NR⁹₂, or COR⁸;
each R³ is independently halo, CO₂R⁸, CONR⁹₂, CN, tetrazole, CHO, S(O)ₙR⁸, substituted or unsubstituted alkyl, substituted or unsubstituted heteroalkyl, OR⁸, SR⁸, NR⁹₂, or COR⁸;
R⁴ is haloalkyl, alkyl, cycloalkyl, or heteroalkyl;
R⁵ is H, alkyl, or COR¹⁰;
R⁶ is H, alkyl, cycloalkyl, heteroalkyl, or haloalkyl;
each R⁷ is independently H, halo, CO₂R⁸, CONR⁹₂, CN, CHO, S(O)ₙR⁸, substituted or unsubstituted alkyl, substituted or unsubstituted heteroalkyl, substituted or unsubstituted aryl, substituted or unsubstituted heteroaryl, OR⁸, SR⁸, NR⁹₂, or COR⁸;
each R⁸ is independently H, substituted or unsubstituted alkyl (e.g., fluoroalkyl), substituted or unsubstituted heteroalkyl, substituted or unsubstituted aryl, or substituted or unsubstituted heteroaryl;
each R⁹ is independently H, substituted or unsubstituted alkyl, substituted or unsubstituted heteroalkyl, substituted or unsubstituted aryl, or substituted or unsubstituted heteroaryl;
   or two R⁹ taken together with the nitrogen to which they are bound form a substituted or unsubstituted heterocycle;
each R¹⁰ is substituted or unsubstituted alkyl, substituted or unsubstituted heteroalkyl, substituted or unsubstituted aryl, or substituted or unsubstituted heteroaryl;
   or two R¹⁰ taken together with the carbon to which they are bound form a substituted or unsubstituted carbocycle, or substituted or unsubstituted heterocycle;
L¹ is O, NR⁸, S, C₁-C₃ alkylene, or C₁-C₃ heteroalkylene;
L² is a bond, C₁-C₆ alkylene, or C₁-C₆ heteroalkylene;
m is 0-3;
each n is independently 0-3; and
each bond represented by ———— is independently a single or double bond.

In some embodiments, the ring formed with Q¹, Q², Q³, Q⁴, and Q⁵ is a cycloheteroalkyl, or a heteroaryl. In certain embodiments, Q¹ is N, CR⁷, or O. In some embodiments, Q¹ is N. In some embodiments, Q¹ is CH. In certain embodiments, Q¹ is O. In some embodiments, Q² is N, or C═X. In certain embodiments, Q² is N. In some embodiments, Q² is C═O. In certain embodiments, Q³ is C. In some embodiments, Q³ is N. In certain embodiments, Q⁴ is N, CR⁷, S, or O. In some embodiments, Q⁴ is CH. In certain embodiments, Q⁴ is N. In some embodiments, Q⁴ is S. In certain embodiments, Q⁴ is O.

In certain embodiments, the group (IIA):

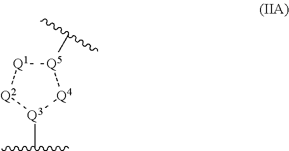

has one of the following structures:

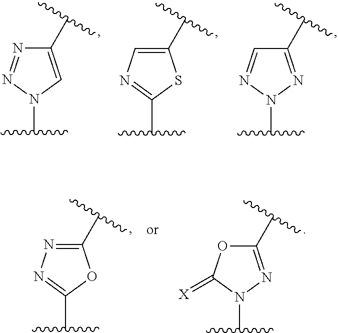

In some embodiments, A is the group (IA) or (IIA).

In more specific embodiments, ring A (or the group (IA) or the group (IIA)) has one of the

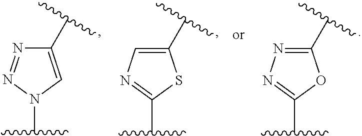 [SMILES: *c1cn(*)nn1 ; *c1cnc(*)s1 ; *c1nnc(*)o1]

In still more specific embodiments, ring A (or the group (IA) or the group (IIA)) has the

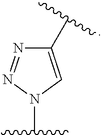 [SMILES: *c1cn(*)nn1]

In other specific embodiments, ring A (or the group (IA) or the group (IIA)) has the

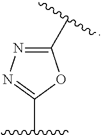 [SMILES: *c1nnc(*)o1]

In some embodiments, R⁴ is haloalkyl. In certain embodiments, R⁴ is fluoroalkyl. In some embodiments, R⁴ is fluorinated methyl. In certain embodiments, R⁴ is CF₃.

In some embodiments, R⁵ is H. In certain embodiments, R⁵ is C₁-C₃ alkyl. In some embodiments, R⁵ is COR¹⁰ wherein R¹⁰ of the COR¹⁰ is C₁-C₃ alkyl.

In certain embodiments, R⁶ is C₁-C₃ alkyl, C₁-C₃ heteroalkyl, or C₁-C₃ haloalkyl. In some embodiments, R⁶ is C₁-C₃ alkyl. In certain embodiments, R⁶ is ethyl or haloethyl. In some embodiments, R⁶ is ethyl.

In certain embodiments, L¹ is S, C₁-C₃ alkylene, or C₁-C₃ heteroalkylene. In some embodiments, L¹ is —CH₂— or —CH₂NH—. In certain embodiments, L¹ is —CH₂—. In some embodiments, L¹ is —CH₂NH—.

In some embodiments, Q⁶ is N. In certain embodiments, Q⁶ is CR⁷. In some embodiments, Q⁶ is CR⁷ and R⁷ of the CR⁷ is halo.

In some embodiments, m is 0, 1, or 2. In certain embodiments, m is 0.

In some embodiments, each R³ is independently C₁-C₃ alkyl.

In certain embodiments, L² is a bond. In some embodiments, L² is C₁-C₆alkylene, or C₁-C₆ heteroalkylene. In certain embodiments, L² is CH₂NH(CH₂)₂. In some embodiments, L² is CH₂. In certain embodiments, L² is —CH═CH—.

In some embodiments, R² is CO₂R⁸. In certain embodiments, R² is CO₂Et. In some embodiments, R² is CONR⁹₂. In certain embodiments, R² is CONH₂. In some embodiments, R² is CONR⁹₂, wherein one R⁹ of the CONR⁹₂ group is H. In certain embodiments, R² is CONR⁹₂, wherein one R⁹ of the CONR⁹₂ group is alkyl and the other is heteroalkyl. In some embodiments, R² is CONR⁹₂, wherein one R⁹ of the CONR⁹₂ group is CH₃ and the other is OCH₃. In certain embodiments, R² is CONR⁹₂, wherein the two R⁹ of the CONR⁹₂ group taken together form a heterocylic ring. In some embodiments, R² is CONR⁹₂, wherein the two R⁹ of the CONR⁹₂ group taken together form —CH₂—S(O)ₙ—(CH₂)₂—. In certain embodiments, R² is CN. In some embodiments, R² is tetrazolyl. In certain embodiments, R² is morpholino. In some embodiments, R² is a hydroxy substituted alkyl.

In certain embodiments, R¹ is H. In some embodiments, R¹ is substituted or unsubstituted alkyl. In certain embodiments, R¹ is C₁₋₅ alkyl. In some embodiments, R¹ is substituted or unsubstituted cycloalkyl. In certain embodiments, R¹ is substituted or unsubstituted aryl. In some embodiments, R¹ is substituted or unsubstituted phenyl. In certain embodiments, R¹ is halo substituted phenyl. In some embodiments, R¹ is fluoro substituted phenyl. In certain embodiments, R¹ is alkyl substituted phenyl. In some embodiments, R¹ is alkyl-sulfonyl substituted phenyl. In certain embodiments, R¹ is phenyl. In some embodiments, R¹ is substituted or unsubstituted heteroaryl. In certain embodiments, R¹ is substituted or unsubstituted pyridyl. In some embodiments, R¹ is unsubstituted pyridyl. In certain embodiments, R¹ is heteroalkyl substituted pyridyl. In some embodiments, R¹ is methoxy substituted pyridyl. In certain embodiments, R¹ is halo substituted pyridyl. In some embodiments, R¹ is alkyl substituted pyridyl. In certain embodiments, R¹ is methyl substituted pyridyl. In some embodiments, R¹ is substituted or unsubstituted N-alkyl-pyridonyl. In certain embodiments, R¹ is N-methyl-pyridonyl. In some embodiments, R¹ is substituted or unsubstituted quinolinyl. In certain embodiments, R¹ is substituted or unsubstituted isoquinolinyl. In some embodiments, R¹ is substituted or unsubstituted quinolinonyl. In certain embodiments, R¹ is COR⁸. In some embodiments, R¹ is COR⁸ and the R⁸ of the COR⁸ group is alkyl. In certain embodiments, R¹ is COCH₃. In some embodiments, each n is independently 0, 1, or 2. In certain embodiments, each n is 2.

In some embodiments, R¹ is as defined in Table 1. In some embodiments, -L²-R² is as defined in Table 1. In some embodiments, -L¹- is as defined in Table 1. In some embodiments, ring A is as defined in Table 1. In some embodiments, Q⁶ is as defined in Table 1.

In specific embodiments, the compounds of Table 1 are provided herein. In some embodiments, compounds of either Formula I or II comprise any one or two, or any other combination of substituents set forth in Table 1.

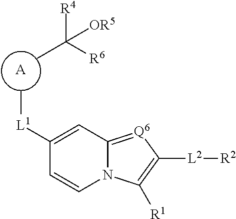

TABLE 1

| | 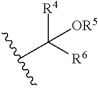 | A | L¹ | Q⁶ | —L²—R² | R¹ | [M + H]* |
|---|---|---|---|---|---|---|---|
| 1 | 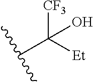 [SMILES: *C(O)(CC)C(F)(F)F] | 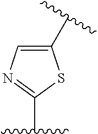 [SMILES: *c1cnc(*)s1] | S | CH | —CO₂Et | H | 431 |
| 2 | 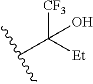 [SMILES: *C(O)(CC)C(F)(F)F] | 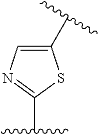 [SMILES: *c1cnc(*)s1] | S | CH | —CO₂H | H | 403 |
| 3 | 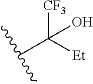 [SMILES: *C(O)(CC)C(F)(F)F] | 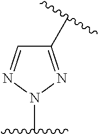 [SMILES: *c1cnn(*)n1] | CH₂ | CH | —CO₂Et | 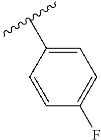 [SMILES: *c1ccc(F)cc1] | 491 |
| 4 | 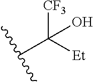 [SMILES: *C(O)(CC)C(F)(F)F] | 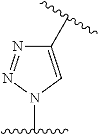 [SMILES: *c1cn(*)nn1] | CH₂ | CH | —CO₂Et | 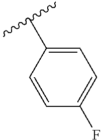 [SMILES: *c1ccc(F)cc1] | 491 |
| 5 | 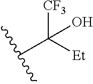 [SMILES: *C(O)(CC)C(F)(F)F] | 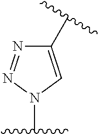 [SMILES: *c1cn(*)nn1] | CH₂ | CH | —CO₂H | 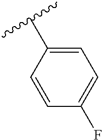 [SMILES: *c1ccc(F)cc1] | 463 |
| 6 | 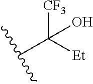 [SMILES: *C(O)(CC)C(F)(F)F] | 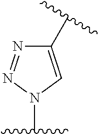 [SMILES: *c1cn(*)nn1] | CH₂ | CH | —CONH₂ | 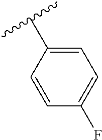 [SMILES: *c1ccc(F)cc1] | 462 |
| 7 | 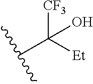 [SMILES: *C(O)(CC)C(F)(F)F] | 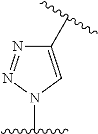 [SMILES: *c1cn(*)nn1] | CH₂ | CH | —C(CH₃)₂OH | 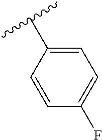 [SMILES: *c1ccc(F)cc1] | 477 |
| 8 | 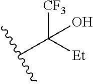 [SMILES: *C(O)(CC)C(F)(F)F] | 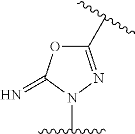 [SMILES: *c1nn(*)c(=N)o1] | CH₂ | CH | —CO₂Et | 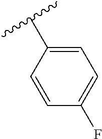 [SMILES: *c1ccc(F)cc1] | 507 |
| 9 (10) | 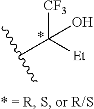 | 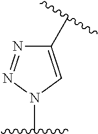 [SMILES: *c1cn(*)nn1] | CH₂ | CH | —CONH₂ | 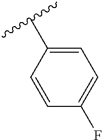 [SMILES: *c1ccc(F)cc1] | 462 |
| 11 | 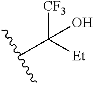 [SMILES: *C(O)(CC)C(F)(F)F] | 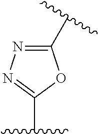 [SMILES: *c1nnc(*)o1] | CH₂NH | CH | —CO₂Et | 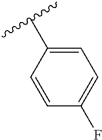 [SMILES: *c1ccc(F)cc1] | 507 |
| 12 | 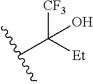 [SMILES: *C(O)(CC)C(F)(F)F] | 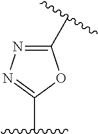 [SMILES: *c1nnc(*)o1] | CH₂NH | N | —CO₂Et | 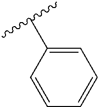 [SMILES: *c1ccccc1] | 490 |
| 13 | 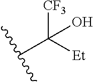 [SMILES: *C(O)(CC)C(F)(F)F] | 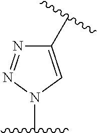 [SMILES: *c1cn(*)nn1] | CH₂ | N | —CO₂Et | 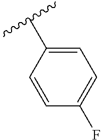 [SMILES: *c1ccc(F)cc1] | 492 |
| 14 | 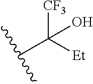 [SMILES: *C(O)(CC)C(F)(F)F] | 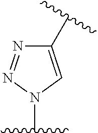 [SMILES: *c1cn(*)nn1] | CH₂ | N | —CONH₂ | 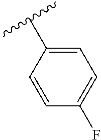 [SMILES: *c1ccc(F)cc1] | 463 |
| 15 | 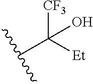 [SMILES: *C(O)(CC)C(F)(F)F] | 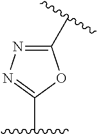 [SMILES: *c1nnc(*)o1] | CH₂NH | CBr | —CO₂Et | 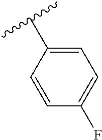 [SMILES: *c1ccc(F)cc1] | 585 |
| 16 (22) | 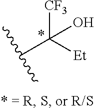 | 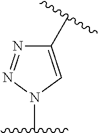 [SMILES: *c1cn(*)nn1] | CH₂ | CH | —CN | 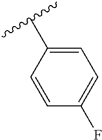 [SMILES: *c1ccc(F)cc1] | 444 |
| 17 | 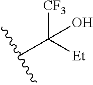 [SMILES: *C(O)(CC)C(F)(F)F] | 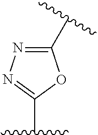 [SMILES: *c1nnc(*)o1] | CH₂NH | CBr | —CONH₂ | 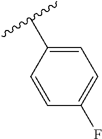 [SMILES: *c1ccc(F)cc1] | 557 |
| 18 | 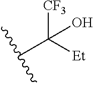 [SMILES: *C(O)(CC)C(F)(F)F] | 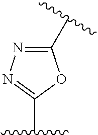 [SMILES: *c1nnc(*)o1] | CH₂NH | CBr | —CN | 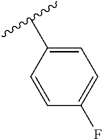 [SMILES: *c1ccc(F)cc1] | 538 |
| 19 (20, 21) | 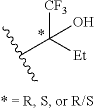 | 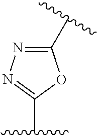 [SMILES: *c1nnc(*)o1] | CH₂NH | CH | —CONH₂ | 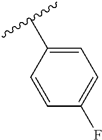 [SMILES: *c1ccc(F)cc1] | 478 |
| 23 | 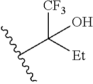 [SMILES: *C(O)(CC)C(F)(F)F] | 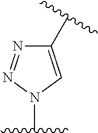 [SMILES: *c1cn(*)nn1] | CH₂ | CH | 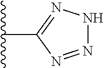 [SMILES: *c1nn[nH]n1] | 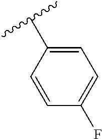 [SMILES: *c1ccc(F)cc1] | 487 |
| 24 | 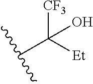 [SMILES: *C(O)(CC)C(F)(F)F] | 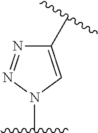 [SMILES: *c1cn(*)nn1] | CH₂ | CH | —CO₂Et | 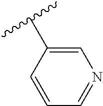 [SMILES: *c1cccnc1] | 474 |
| 25 | 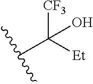 [SMILES: *C(O)(CC)C(F)(F)F] | 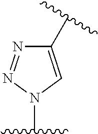 [SMILES: *c1cn(*)nn1] | CH₂ | CH | —CONH₂ | 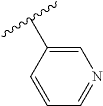 [SMILES: *c1cccnc1] | 445 |
| 26 | 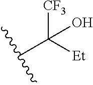 [SMILES: *C(O)(CC)C(F)(F)F] | 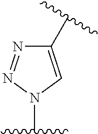 [SMILES: *c1cn(*)nn1] | CH₂ | CH | —CN | 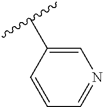 [SMILES: *c1cccnc1] | 427 |
| 27 | 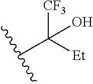 [SMILES: *C(O)(CC)C(F)(F)F] | 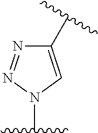 [SMILES: *c1cn(*)nn1] | CH₂ | CH | —CO₂Et | 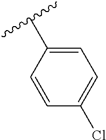 [SMILES: *c1ccc(Cl)cc1] | 507 |
| 28 | 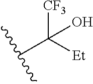 [SMILES: *C(O)(CC)C(F)(F)F] | 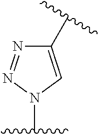 [SMILES: *c1cn(*)nn1] | CH₂ | CH | —CONH₂ | 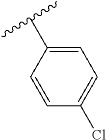 [SMILES: *c1ccc(Cl)cc1] | 478 |
| 29 | 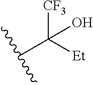 [SMILES: *C(O)(CC)C(F)(F)F] | 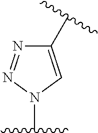 [SMILES: *c1cn(*)nn1] | CH₂ | CH | —CO₂Et | 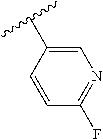 [SMILES: *c1ccc(F)nc1] | 491 |
| 30 | 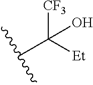 [SMILES: *C(O)(CC)C(F)(F)F] | 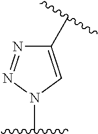 [SMILES: *c1cn(*)nn1] | CH₂ | CH | —CH₂OH | 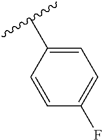 [SMILES: *c1ccc(F)cc1] | 449 |
| 31 | 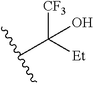 [SMILES: *C(O)(CC)C(F)(F)F] | 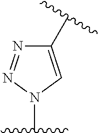 [SMILES: *c1cn(*)nn1] | CH₂ | CH | —CONH₂ | 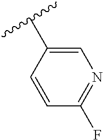 [SMILES: *c1ccc(F)nc1] | 463 |
| 32 | 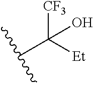 [SMILES: *C(O)(CC)C(F)(F)F] | 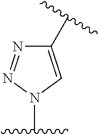 [SMILES: *c1cn(*)nn1] | CH₂ | CH | —CN | 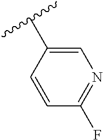 [SMILES: *c1ccc(F)nc1] | 445 |
| 33 | 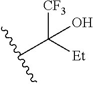 [SMILES: *C(O)(CC)C(F)(F)F] | 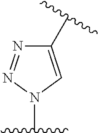 [SMILES: *c1cn(*)nn1] | CH₂ | CH | —CHO | 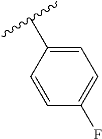 [SMILES: *c1ccc(F)cc1] | 447 |
| 34 | 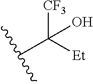 [SMILES: *C(O)(CC)C(F)(F)F] | 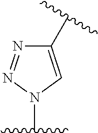 [SMILES: *c1cn(*)nn1] | CH₂ | CH | —CH₂SO₂CH₃ | 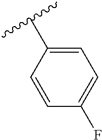 [SMILES: *c1ccc(F)cc1] | 511 |
| 35 | 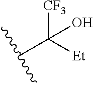 [SMILES: *C(O)(CC)C(F)(F)F] | 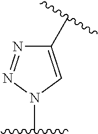 [SMILES: *c1cn(*)nn1] | CH₂ | CH | —CONH₂ | 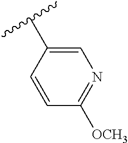 [SMILES: *c1ccc(OC)nc1] | 475 |
| 36 | 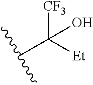 [SMILES: *C(O)(CC)C(F)(F)F] | 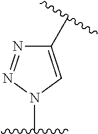 [SMILES: *c1cn(*)nn1] | CH₂ | CH | —CO₂Et | 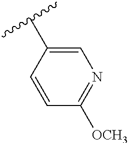 [SMILES: *c1ccc(OC)nc1] | 505 |
| 37 | 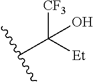 [SMILES: *C(O)(CC)C(F)(F)F] | 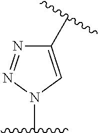 [SMILES: *c1cn(*)nn1] | CH₂ | CH | —CH₂NH—CH₂CH₂CO₂Et | 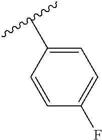 [SMILES: *c1ccc(F)cc1] | 548 |
| 38 | 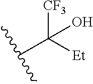 [SMILES: *C(O)(CC)C(F)(F)F] | 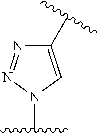 [SMILES: *c1cn(*)nn1] | CH₂ | CH | 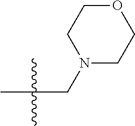 [SMILES: *CN1CCOCC1] | 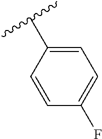 [SMILES: *c1ccc(F)cc1] | 518 |
| 39 | 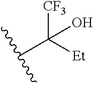 [SMILES: *C(O)(CC)C(F)(F)F] | 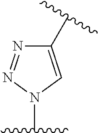 [SMILES: *c1cn(*)nn1] | CH₂ | CH | 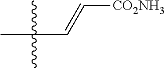 | 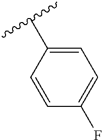 [SMILES: *c1ccc(F)cc1] | 503 |
| 40 | 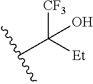 [SMILES: *C(O)(CC)C(F)(F)F] | 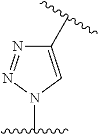 [SMILES: *c1cn(*)nn1] | CH₂ | CH | —CH₂NH—CH₂CH₂CO₂H | 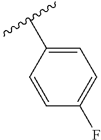 [SMILES: *c1ccc(F)cc1] | 520 |
| 41 | 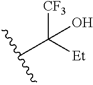 [SMILES: *C(O)(CC)C(F)(F)F] | 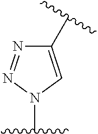 [SMILES: *c1cn(*)nn1] | CH₂ | CH | 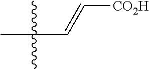 [SMILES: */C=C/C(=O)O] | 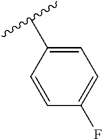 [SMILES: *c1ccc(F)cc1] | 489 |
| 42 | 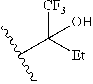 [SMILES: *C(O)(CC)C(F)(F)F] | 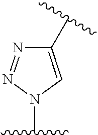 [SMILES: *c1cn(*)nn1] | CH₂ | CH | —CH₂NH—CH₂CH₂CN | 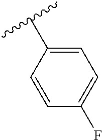 [SMILES: *c1ccc(F)cc1] | 501 |
| 43 | 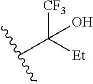 [SMILES: *C(O)(CC)C(F)(F)F] | 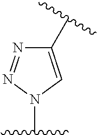 [SMILES: *c1cn(*)nn1] | CH₂ | CH | 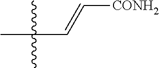 [SMILES: */C=C/C(N)=O] | 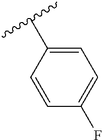 [SMILES: *c1ccc(F)cc1] | 488 |
| 44 | 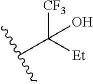 [SMILES: *C(O)(CC)C(F)(F)F] | 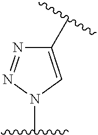 [SMILES: *c1cn(*)nn1] | CH₂ | CH | —CH₂OCH₃ | 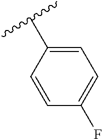 [SMILES: *c1ccc(F)cc1] | 463 |
| 45 | 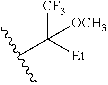 [SMILES: *C(CC)(OC)C(F)(F)F] | 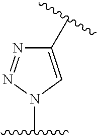 [SMILES: *c1cn(*)nn1] | CH₂ | CH | —CH₂OCH₃ | 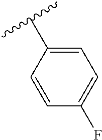 [SMILES: *c1ccc(F)cc1] | 477 |
| 46 | 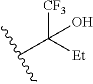 [SMILES: *C(O)(CC)C(F)(F)F] | 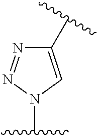 [SMILES: *c1cn(*)nn1] | CH₂ | CH | —CO₂Et | 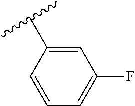 [SMILES: *c1cccc(F)c1] | 491 |
| 47 | 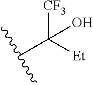 [SMILES: *C(O)(CC)C(F)(F)F] | 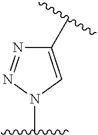 [SMILES: *c1cn(*)nn1] | CH₂ | CH | —CO₂Et | 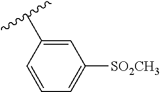 [SMILES: *c1cccc(S(C)(=O)=O)c1] | 551 |
| 48 | 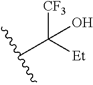 [SMILES: *C(O)(CC)C(F)(F)F] | 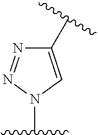 [SMILES: *c1cn(*)nn1] | CH₂ | CH | —CONH₂ | 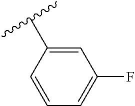 [SMILES: *c1cccc(F)c1] | 462 |
| 49 | 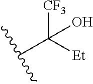 [SMILES: *C(O)(CC)C(F)(F)F] | 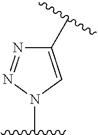 [SMILES: *c1cn(*)nn1] | CH₂ | CH | —CN | 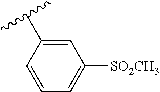 [SMILES: *c1cccc(S(C)(=O)=O)c1] | 504 |
| 50 | 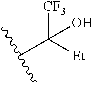 [SMILES: *C(O)(CC)C(F)(F)F] | 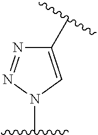 [SMILES: *c1cn(*)nn1] | CH₂ | CH | 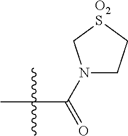 [SMILES: *C(=O)N1CCS(=O)(=O)C1] | 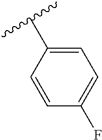 [SMILES: *c1ccc(F)cc1] | 565 |
| 51 | 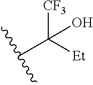 [SMILES: *C(O)(CC)C(F)(F)F] | 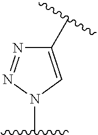 [SMILES: *c1cn(*)nn1] | CH₂ | CH | —CON(CH₃)—OCH₃ | 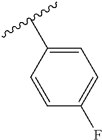 [SMILES: *c1ccc(F)cc1] | 505 |
| 52 | 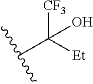 [SMILES: *C(O)(CC)C(F)(F)F] | 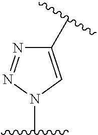 [SMILES: *c1cn(*)nn1] | CH₂ | CH | —CO₂Et | 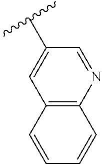 [SMILES: *c1cnc2ccccc2c1] | 524 |
| 53 | 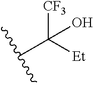 [SMILES: *C(O)(CC)C(F)(F)F] | 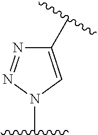 [SMILES: *c1cn(*)nn1] | CH₂ | CH | —CN | 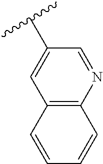 [SMILES: *c1cnc2ccccc2c1] | 477 |
| 54 | 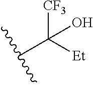 [SMILES: *C(O)(CC)C(F)(F)F] | 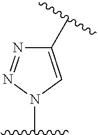 [SMILES: *c1cn(*)nn1] | CH₂ | CH | —COCH₃ | 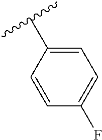 [SMILES: *c1ccc(F)cc1] | 460 |
| 55 | 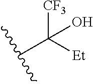 [SMILES: *C(O)(CC)C(F)(F)F] | 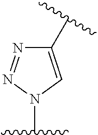 [SMILES: *c1cn(*)nn1] | CH₂ | CH | —CONHEt | 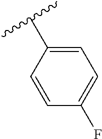 [SMILES: *c1ccc(F)cc1] | 489 |
| 56 | 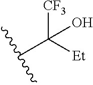 [SMILES: *C(O)(CC)C(F)(F)F] | 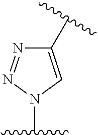 [SMILES: *c1cn(*)nn1] | CH₂ | CH | —CO₂Et | 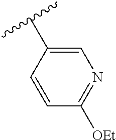 [SMILES: *c1ccc(OCC)nc1] | 518 |
| 57 | 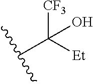 [SMILES: *C(O)(CC)C(F)(F)F] | 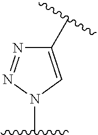 [SMILES: *c1cn(*)nn1] | CH₂ | CH | —CN | 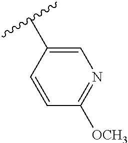 [SMILES: *c1ccc(OC)nc1] | 457 |
| 58 | 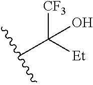 [SMILES: *C(O)(CC)C(F)(F)F] | 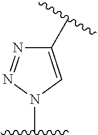 [SMILES: *c1cn(*)nn1] | CH₂ | CH | —CONH₂ | 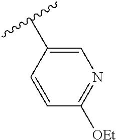 [SMILES: *c1ccc(OCC)nc1] | 489 |
| 59 | 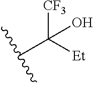 [SMILES: *C(O)(CC)C(F)(F)F] | 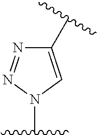 [SMILES: *c1cn(*)nn1] | CH₂ | CH | —CN | 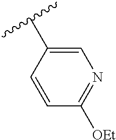 [SMILES: *c1ccc(OCC)nc1] | 471 |
| 60 | 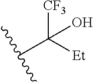 [SMILES: *C(O)(CC)C(F)(F)F] | 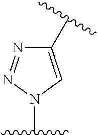 [SMILES: *c1cn(*)nn1] | CH₂ | CH | —CH₂NH—CH₂CH₃ | 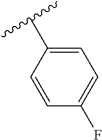 [SMILES: *c1ccc(F)cc1] | 476 |
| 61 | 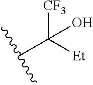 [SMILES: *C(O)(CC)C(F)(F)F] | 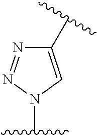 [SMILES: *c1cn(*)nn1] | CH₂ | CCl | —CONH₂ | 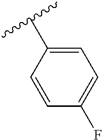 [SMILES: *c1ccc(F)cc1] | 496 |
| 62 | 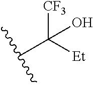 [SMILES: *C(O)(CC)C(F)(F)F] | 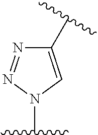 [SMILES: *c1cn(*)nn1] | CH₂ | CH | —CO₂Et | 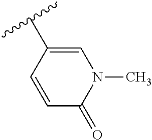 [SMILES: *c1ccc(=O)n(C)c1] | 504 |
| 63 | 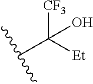 [SMILES: *C(O)(CC)C(F)(F)F] | 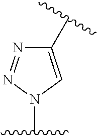 [SMILES: *c1cn(*)nn1] | CH₂ | CH | —CO₂Et | 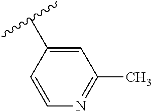 [SMILES: *c1ccnc(C)c1] | 488 |
| 64 | 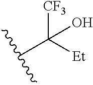 [SMILES: *C(O)(CC)C(F)(F)F] | 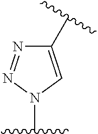 [SMILES: *c1cn(*)nn1] | CH₂ | CH | —CONH₂ | 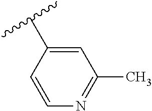 [SMILES: *c1ccnc(C)c1] | 459 |
| 65 | 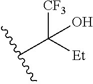 [SMILES: *C(O)(CC)C(F)(F)F] | 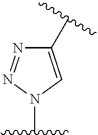 [SMILES: *c1cn(*)nn1] | CH₂ | CH | —CN | 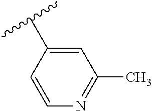 [SMILES: *c1ccnc(C)c1] | 441 |
| 66 | 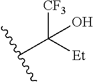 [SMILES: *C(O)(CC)C(F)(F)F] | 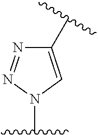 [SMILES: *c1cn(*)nn1] | CH₂ | CH | —CO₂CH₃ | —COCH₃ | 425 |
| 67 | 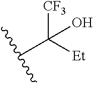 [SMILES: *C(O)(CC)C(F)(F)F] | 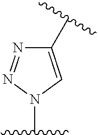 [SMILES: *c1cn(*)nn1] | CH₂ | CH | —CONH₂ | —COCH₃ | 410 |
| 68 | 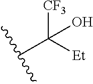 [SMILES: *C(O)(CC)C(F)(F)F] | 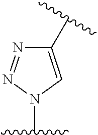 [SMILES: *c1cn(*)nn1] | CH₂ | CH | —CONH₂ | 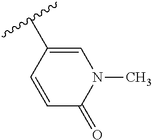 [SMILES: *c1ccc(=O)n(C)c1] | 475 |
| 69 | 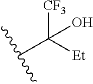 [SMILES: *C(O)(CC)C(F)(F)F] | 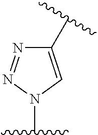 [SMILES: *c1cn(*)nn1] | CH₂ | CH | —CO₂Et | 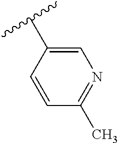 [SMILES: *c1ccc(C)nc1] | 488 |
| 70 | 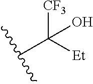 [SMILES: *C(O)(CC)C(F)(F)F] | 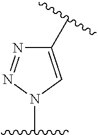 [SMILES: *c1cn(*)nn1] | CH₂ | CH | —CN | 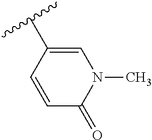 [SMILES: *c1ccc(=O)n(C)c1] | 457 |
| 71 | 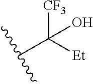 [SMILES: *C(O)(CC)C(F)(F)F] | 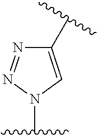 [SMILES: *c1cn(*)nn1] | CH₂ | CH | —CONH₂ | 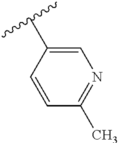 [SMILES: *c1ccc(C)nc1] | 459 |
| 72 | 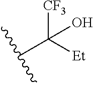 [SMILES: *C(O)(CC)C(F)(F)F] | 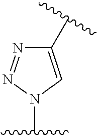 [SMILES: *c1cn(*)nn1] | CH₂ | CH | —CN | 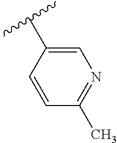 [SMILES: *c1ccc(C)nc1] | 441 |
| 73 | 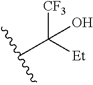 [SMILES: *C(O)(CC)C(F)(F)F] | 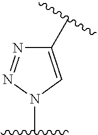 [SMILES: *c1cn(*)nn1] | CH₂ | CH | —CO₂Et | 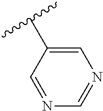 [SMILES: *c1cncnc1] | 475 |
| 74 | 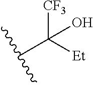 [SMILES: *C(O)(CC)C(F)(F)F] | 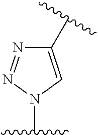 [SMILES: *c1cn(*)nn1] | CH₂ | CH | —CO₂Et | 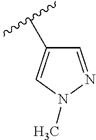 [SMILES: *c1cnn(C)c1] | 477 |
| 75 | 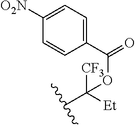 [SMILES: *C(CC)(OC(=O)c1ccc([N+](=O)[O-])cc1)C(F)(F)F] | 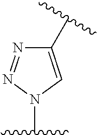 [SMILES: *c1cn(*)nn1] | CH₂ | CH | —CONH₂ | 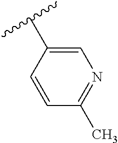 [SMILES: *c1ccc(C)nc1] | 608 |
| 76 | 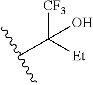 [SMILES: *C(O)(CC)C(F)(F)F] | 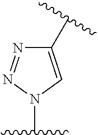 [SMILES: *c1cn(*)nn1] | CH₂ | CH | —CN | 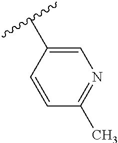 [SMILES: *c1ccc(C)nc1] | 441 |
| 77 | 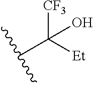 [SMILES: *C(O)(CC)C(F)(F)F] | 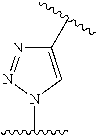 [SMILES: *c1cn(*)nn1] | CH₂ | CH | —CO₂Et | —CH(CH₃)₂ | 439 |
| 78 | 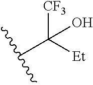 [SMILES: *C(O)(CC)C(F)(F)F] | 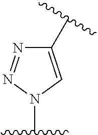 [SMILES: *c1cn(*)nn1] | CH₂ | CH | —CONH₂ | 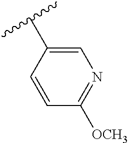 [SMILES: *c1ccc(OC)nc1] | 475 |
| 79 | 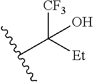 [SMILES: *C(O)(CC)C(F)(F)F] | 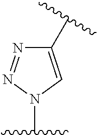 [SMILES: *c1cn(*)nn1] | CH₂ | CH | —CN | 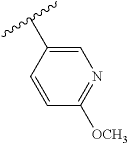 [SMILES: *c1ccc(OC)nc1] | 457 |
| 80 | 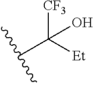 [SMILES: *C(O)(CC)C(F)(F)F] | 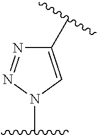 [SMILES: *c1cn(*)nn1] | CH₂ | CH | —CN | 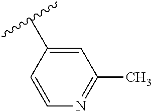 [SMILES: *c1ccnc(C)c1] | 441 |
| 81 | 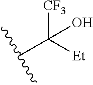 [SMILES: *C(O)(CC)C(F)(F)F] | 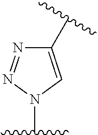 [SMILES: *c1cn(*)nn1] | CH₂ | CH | —CN | —CH(CH₃)₂ | 392 |
| 82 | 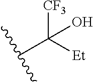 [SMILES: *C(O)(CC)C(F)(F)F] | 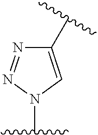 [SMILES: *c1cn(*)nn1] | CH₂ | C—(COCF₃) | —CONH₂ | 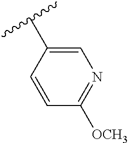 [SMILES: *c1ccc(OC)nc1] | 571 |
| 83 | 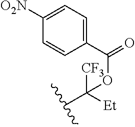 [SMILES: *C(CC)(OC(=O)c1ccc([N+](=O)[O-])cc1)C(F)(F)F] | 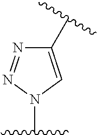 [SMILES: *c1cn(*)nn1] | CH₂ | CH | —CONH₂ | 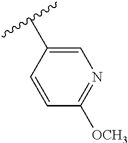 [SMILES: *c1ccc(OC)nc1] | 624 |
| 84 | 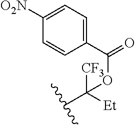 [SMILES: *C(CC)(OC(=O)c1ccc([N+](=O)[O-])cc1)C(F)(F)F] | 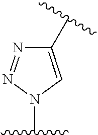 [SMILES: *c1cn(*)nn1] | CH₂ | C—(COCF₃) | —CO₂Et | 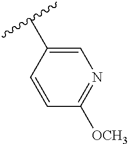 [SMILES: *c1ccc(OC)nc1] | 749 |
| 85 | 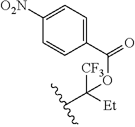 [SMILES: *C(CC)(OC(=O)c1ccc([N+](=O)[O-])cc1)C(F)(F)F] | 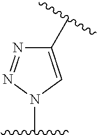 [SMILES: *c1cn(*)nn1] | CH₂ | CH | —CO₂Et | 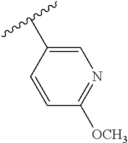 [SMILES: *c1ccc(OC)nc1] | 653 |
| 86 | 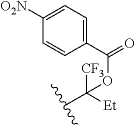 [SMILES: *C(CC)(OC(=O)c1ccc([N+](=O)[O-])cc1)C(F)(F)F] | 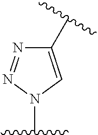 [SMILES: *c1cn(*)nn1] | CH₂ | CH | —CO₂Et | H | 546 |
| 87 | 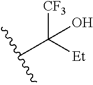 [SMILES: *C(O)(CC)C(F)(F)F] | 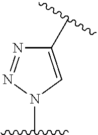 [SMILES: *c1cn(*)nn1] | CH₂ | CH | —CO₂Et | Cl | 431 |
| 88 | 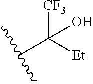 [SMILES: *C(O)(CC)C(F)(F)F] | 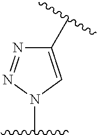 [SMILES: *c1cn(*)nn1] | CH₂ | CH | —CN | Cl | 384 |
| 89 | 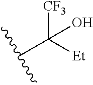 [SMILES: *C(O)(CC)C(F)(F)F] | 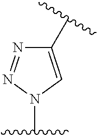 [SMILES: *c1cn(*)nn1] | CH₂ | CH | —CONH₂ | Cl | 402 |

*mass spectrometric data

In some embodiments, a compound provided herein is 7-[5-(1-Hydroxy-1-trifluoromethyl-propyl)-thiazol-2-ylsulfanyl]-indolizine-2-carboxylic acid ethyl ester (Compound 1-1); 7-[5-(1-Hydroxy-1-trifluoromethyl-propyl)-thiazol-2-ylsulfanyl]-indolizine-2-carboxylic acid (Compound 1-2); 3-(4-Fluoro-phenyl)-7-[4-(1-hydroxy-1-trifluoromethyl-propyl)-[1,2,3]triazol-2-ylmethyl]-indolizine-2-carboxylic acid ethyl ester (Compound 1-3); 3-(4-Fluoro-phenyl)-7-[4-(1-hydroxy-1-trifluoromethyl-propyl)-[1,2,3]triazol-1-ylmethyl]-indolizine-2-carboxylic acid ethyl ester (Compound 1-4); 3-(4-Fluoro-phenyl)-7-[4-(1-hydroxy-1-trifluoromethyl-propyl)-[1,2,3]triazol-1-ylmethyl]-indolizine-2-carboxylic acid (Compound 1-5); 3-(4-Fluoro-phenyl)-7-[4-(1-hydroxy-1-trifluoromethyl-propyl)-[1,2,3]triazol-1-ylmethyl]-indolizine-2-carboxylic acid amide (Compound 1-6); 1,1,1-Trifluoro-2-{1-[3-(4fluoro-phenyl)-2-(1-hydroxy-1-methyl-ethyl)-indolizin-7-ylmethyl]-1H-[1,2,3]triazol-4-yl}-butan-2-ol (Compound 1-7); 3-(4-Fluoro-phenyl)-7-[5-(1-hydroxy-1-trifluoromethyl-propyl)-2-imino-[1,3,4]oxadiazol-3-ylmethyl]-indolizine-2-carboxylic acid ethyl ester (Compound 1-8); 3-(4-Fluoro-phenyl)-7-[4-(1-hydroxy-1-trifluoromethyl-propyl)-[1,2,3]triazol-1-ylmethyl]-indolizine-2-carboxylic acid amide (Compound 1-9); 3-(4-Fluoro-phenyl)-7-[4-(1-hydroxy-1-trifluoromethyl-propyl)-[1,2,3]triazol-1-ylmethyl]-indolizine-2-carboxylic acid amide (Compound 1-10); 3-(4-Fluoro-phenyl)-7-{[5-(1-hydroxy-1-trifluoromethyl-propyl)-[1,3,4]oxadiazol-2-ylamino]-methyl}-indolizine-2-carboxylic acid ethyl ester (Compound 1-11); 7-{[5-(1-Hydroxy-1-trifluoromethyl-propyl)-[1,3,4]oxadiazol-2-ylamino]-methyl}-3-phenyl-imidazo[1,2-a]pyridine-2-carboxylic acid ethyl ester (Compound 1-12); 3-(4-Fluoro-phenyl)-7-[4-(1-hydroxy-1-trifluoromethyl-propyl)-[1,2,3]triazol-1-ylmethyl]-imidazo[1,2-a]pyridine-2-carboxylic acid ethyl ester (Compound 1-13); 3-(4-Fluoro-phenyl)-7-[4-(1-hydroxy-1-trifluoromethyl-propyl)-[1,2,3]triazol-1-ylmethyl]-imidazo[1,2-a]pyridine-2-carboxylic acid amide (Compound 1-14); 1-Bromo-3-(4-fluoro-phenyl)-7-{[5-(1-hydroxy-1-trifluoromethyl-propyl)-[1,3,4]oxadiazol-2-ylamino]-methyl}-indolizine-2-carboxylic acid ethyl ester (Compound 1-15); 3-(4-Fluoro-phenyl)-7-[4-(1-hydroxy-1-trifluoromethyl-propyl)-[1,2,3]triazol-1-ylmethyl]-indolizine-2-carbonitrile (Compound 1-16); 1-Bromo-3-(4-fluoro-phenyl)-7-{[5-(1-hydroxy-1-trifluoromethyl-propyl)-[1,3,4]oxadiazol-2-ylamino]-methyl}-indolizine-2-carboxylic acid amide (Compound 1-17); 1-Bromo-3-(4-fluoro-phenyl)-7-{[5-(1-hydroxy-1-trifluoromethyl-propyl)-[1,3,4]oxadiazol-2-ylamino]-methyl}-indolizine-2-carbonitrile (Compound 1-18); 3-(4-Fluoro-phenyl)-7-{[5-(1-hydroxy-1-trifluoromethyl-propyl)-[1,3,4]oxadiazol-2-ylamino]-methyl}-indolizine-2-carboxylic acid amide (Compound 1-19); 3-(4-Fluoro-phenyl)-7-{[5-(1-hydroxy-1-trifluoromethyl-propyl)-[1,3,4]oxadiazol-2-ylamino]-methyl}-indolizine-2-carboxylic acid amide (Compound 1-20); 3-(4-Fluoro-phenyl)-7-{[5-(1-hydroxy-1-trifluoromethyl-propyl)-[1,3,4]oxadiazol-2-ylamino]-methyl}-indolizine-2-carboxylic acid amide (Compound 1-21); 3-(4-Fluoro-phenyl)-7-[4-(1-hydroxy-1-trifluoromethyl-propyl)-[1,2,3]triazol-1-ylmethyl]-indolizine-2-carbonitrile (Compound 1-22); 1,1,1-Trifluoro-2-{1-[3-(4-fluoro-phenyl)-2-(2H-tetrazol-5-yl)-indolizin-7-ylmethyl]-1H-[1,2,3]triazol-4-yl}-butan-2-ol (Compound 1-23); 7-[4-(1-Hydroxy-1-trifluoromethyl-propyl)-[1,2,3]triazol-1-ylmethyl]-3-pyridin-3-yl-indolizine-2-carboxylic acid ethyl ester (Compound 1-24); 7-[4-(1-Hydroxy-1-trifluoromethyl-propyl)-[1,2,3]triazol-1-ylmethyl]-3-pyridin-3-yl-indolizine-2-carboxylic acid amide (Compound 1-25); 7-[4-(1-Hydroxy-1-trifluoromethyl-propyl)-[1,2,3]triazol-1-ylmethyl]-3-pyridin-3-yl-indolizine-2-carbonitrile (Compound 1-26); 3-(4-Chloro-phenyl)-7-[4-(1-hydroxy-1-trifluoromethyl-propyl)-[1,2,3]triazol-1-ylmethyl]-indolizine-2-carboxylic acid ethyl ester (Compound 1-27); 3-(4-Chloro-phenyl)-7-[4-(1-hydroxy-1-trifluoromethyl-propyl)-[1,2,3]triazol-1-ylmethyl]-indolizine-2-carboxylic acid amide (Compound 1-28); 3-(6-Fluoro-pyridin-3-yl)-7-[4-(1-hydroxy-1-trifluoromethyl-propyl) [1,2,3]triazol-1-ylmethyl]-indolizine-2-carb oxylic acid ethyl ester (Compound 1-29); 1,1,1-Trifluoro-2-{1-[3-(4-fluoro-phenyl)-2-hydroxymethyl-indolizin-7-ylmethyl]-1H-[1,2,3]triazol-4-yl}-butan-2-ol (Compound 1-30); 3-(6-Fluoro-pyridin-3-yl)-7-[4-(1-hydroxy-1-trifluoromethyl-propyl)-[1,2,3]triazol-1-ylmethyl]-indolizine-2-carboxylic acid amide (Compound 1-31); 3-(6-Fluoro-pyridin-3-yl)-7-[4-(1-hydroxy-1-trifluoromethyl-propyl)-[1,2,3]triazol-1-ylmethyl]-indolizine-2-carbonitrile (Compound 1-32); 3-(4-Fluoro-phenyl)-7-[4-(1-hydroxy-1-trifluoromethyl-propyl)-[1,2,3]triazol-1-ylmethyl]-indolizine-2-carbaldehyde (Compound 1-33); 1,1,1-Trifluoro-2-{1-[3-(4-fluoro-phenyl)-2-methanesulfonylmethyl-indolizin-7-ylmethyl]-1H-[1,2,3]triazol-4-yl}-butan-2-ol (Compound 1-34); 7-[4-(1-Hydroxy-1-trifluoromethyl-propyl)-[1,2,3]triazol-1-ylmethyl]-3-(6-methoxy-pyridin-3-yl)-indolizine-2-carboxylic acid amide (Compound 1-35); 7-[4-(1-Hydroxy-1-trifluoromethyl-propyl)-[1,2,3]triazol-1-ylmethyl]-3-(6-methoxy-pyridin-3-yl)-indolizine-2-carboxylic acid ethyl ester (Compound 1-36); 3-({3-(4-Fluoro-phenyl)-7-[4-(1-hydroxy-1-trifluoromethyl-propyl)-[1,2,3]triazol-1-ylmethyl]-indolizin-2-ylmethyl}-amino)-propionic acid ethyl ester (Compound 1-37); 1,1,1-Trifluoro-2-{1-[3-(4-fluoro-phenyl)-2-morpholin-4-ylmethyl-indolizin-7-ylmethyl]-1H-[1,2,3]triazol-4-yl}-butan-2-ol (Compound 1-38); (E)-3-{3-(4-Fluoro-phenyl)-7-[4-(1-hydroxy-1-trifluoromethyl-propyl)-[1,2,3]triazol-1-ylmethyl]-indolizin-2-yl}-acrylic acid methyl ester (Compound 1-39); 3-({3-(4-Fluoro-phenyl)-7-[4-(1-hydroxy-1-trifluoromethyl-propyl)-[1,2,3]triazol-1-ylmethyl]-indolizin-2-ylmethyl}-amino)-propionic acid (Compound 1-40); (E)-3-{3-(4-Fluoro-phenyl)-7-[4-(1-hydroxy-1-trifluoromethyl-propyl)-[1,2,3]triazol-1-ylmethyl]-indolizin-2-yl}-acrylic acid (Compound 1-41); 3-({3-(4-Fluoro-phenyl)-7-[4-(1-hydroxy-1-trifluoromethyl-propyl)-[1,2,3]triazol-1-ylmethyl]-indolizin-2-ylmethyl}-amino)-propionitrile (Compound 1-42); (E)-3-{3-(4-Fluoro-phenyl)-7-[4-(1-hydroxy-1-trifluoromethyl-propyl)-[1,2,3]triazol-1-ylmethyl]-indolizin-2-yl}-acrylamide (Compound 1-43); 1,1,1-Trifluoro-2-{1-[3-(4-fluoro-phenyl)-2-methoxymethyl-indolizin-7-ylmethyl]-1H-[1,2,3]triazol-4-yl}-butan-2-ol (Compound 1-44); 3-(4-Fluoro-phenyl)-2-methoxymethyl-7-[4-(1-methoxy-1-trifluoromethyl-propyl)-[1,2,3]triazol-1-ylmethyl]-indolizine (Compound 1-45); 3-(3-Fluoro-phenyl)-7-[4-(1-hydroxy-1-trifluoromethyl-propyl)-[1,2,3]triazol-1-ylmethyl]-indolizine-2-carboxylic acid ethyl ester (Compound 1-46); 7-[4-(1-Hydroxy-1-trifluoromethyl-propyl)-[1,2,3]triazol-1-ylmethyl]-3-(3-methanesulfonyl-phenyl)-indolizine-2-carboxylic acid ethyl ester (Compound 1-47); 3-(3-Fluoro-phenyl)-7-[4-(1-hydroxy-1-trifluoromethyl-propyl)-[1,2,3]triazol-1-ylmethyl]-indolizine-2-carboxylic acid amide (Compound 1-48); 7-[4-(1-Hydroxy-1-trifluoromethyl-propyl)-[1,2,3]triazol-1-ylmethyl]-3-(3-methanesulfonyl-phenyl)-indolizine-2-carbonitrile (Compound 1-49); (1,1-Dioxo-1λ⁶-thiazolidin-3-yl)-{3-(4-fluoro-phenyl)-7-[4-(1-hydroxy-1-trifluoromethyl-propyl)-[1,2,3]triazol-1-ylmethyl]-indolizin-2-yl}-methanone (Compound 1-50); 3-(4-Fluoro-phenyl)-7-[4-(1-hydroxy-1-trifluoromethyl-propyl)-[1,2,3]triazol-1-ylmethyl]-indolizine-2-carboxylic acid methoxy-methyl-amide (Compound 1-51); 7-[4-(1-Hydroxy-1-trifluoromethyl-propyl) [1,2,3]triazol-1-ylmethyl]-3-quinolin-3-yl-indolizine-2-carboxylic acid ethyl ester (Compound 1-52); 7-[4-(1-Hydroxy-1-trifluoromethyl-propyl)-[1,2,3]triazol-1-ylmethyl]-3-quinolin-3-yl-indolizine-2-carbonitrile (Compound 1-53); 1-{3-(4-Fluoro-phenyl)-7-[4-(1-hydroxy-1-trifluoromethyl-propyl)-[1,2,3]triazol-1-ylmethyl]-indolizin-2-yl}-ethanone (Compound 1-54); 3-(4-Fluoro-phenyl)-7-[4-(1-hydroxy-1-trifluoromethyl-propyl)-[1,2,3]triazol-1-ylmethyl]-indolizine-2-carboxylic acid ethylamide (Compound 1-55); 3-(6-Ethoxy-pyridin-3-yl)-7-[4-(1-hydroxy-1-trifluoromethyl-propyl)-[1,2,3]triazol-1-ylmethyl]-indolizine-2-carboxylic acid ethyl ester (Compound 1-56); 7-[4-(1-Hydroxy-1-trifluoromethyl-propyl)-[1,2,3]triazol-1-ylmethyl]-3-(6-methoxy-pyridin-3-yl)-indolizine-2-carbonitrile (Compound 1-57); 3-(6-Ethoxy-pyridin-3-yl)-7-[4-(1-hydroxy-1-trifluoromethyl-propyl)-[1,2,3]triazol-1-ylmethyl]-indolizine-2-carboxylic acid amide (Compound 1-58); 3-(6-Ethoxy-pyridin-3-yl)-7-[4-(1-hydroxy-1-trifluoromethyl-propyl)-[1,2,3]triazol-1-ylmethyl]-indolizine-2-carbonitrile (Compound 1-59); 2-{1-[2-Ethylaminomethyl-3-(4-fluoro-phenyl)-indolizin-7-ylmethyl]-1H-[1,2,3]triazol-4-yl}-1,1,1-trifluoro-butan-2-ol (Compound 1-60); 1-Chloro-3-(4-fluoro-phenyl)-7-[4-(1-hydroxy-1-trifluoromethyl-propyl)-[1,2,3]triazol-1-ylmethyl]-indolizine-2-carboxylic acid amide (Compound 1-61); 7-[4-(1-Hydroxy-1-trifluoromethyl-propyl)-[1,2,3]triazol-1-ylmethyl]-3-(1-methyl-6-oxo-1,6-dihydro-pyridin-3-yl)-indolizine-2-carboxylic acid ethyl ester (Compound 1-62); 7-[4-(1-Hydroxy-1-trifluoromethyl-propyl)-[1,2,3]triazol-1-ylmethyl]-3-(2-methyl-pyridin-4-yl)-indolizine-2-carboxylic acid ethyl ester (Compound 1-63); 7-[4-(1-Hydroxy-1-trifluoromethyl-propyl)-[1,2,3]triazol-1-ylmethyl]-3-(2-methyl-pyridin-4-yl)-indolizine-2-carboxylic acid amide (Compound 1-64); 7-[4-(1-Hydroxy-1-trifluoromethyl-propyl)-[1,2,3]triazol-1-ylmethyl]-3-(2-methyl-pyridin-4-yl)-indolizine-2-carbonitrile (Compound 1-65); 3-Acetyl-7-[4-(1-hydroxy-1-trifluoromethyl-propyl)-[1,2,3]triazol-1-ylmethyl]-indolizine-2-carboxylic acid methyl ester (Compound 1-66); 3-Acetyl-7-[4-(1-hydroxy-1-trifluoromethyl-propyl)-[1,2,3]triazol-1-ylmethyl]-indolizine-2-carboxylic acid amide (Compound 1-67); 7-[4-(1-Hydroxy-1-trifluoromethyl-propyl)-[1,2,3]triazol-1-ylmethyl]-3-(1-methyl-6-oxo-1,6-dihydro-pyridin-3-yl)-indolizine-2-carboxylic acid amide (Compound 1-68); 7-[4-(1-Hydroxy-1-trifluoromethyl-propyl)-[1,2,3]triazol-1-ylmethyl]-3-(6-methyl-pyridin-3-yl)-indolizine-2-carboxylic acid ethyl ester (Compound 1-69); 7-[4-(1-Hydroxy-1-trifluoromethyl-propyl)-[1,2,3]triazol-1-ylmethyl]-3-(1-methyl-6-oxo-1,6-dihydro-pyridin-3-yl)-indolizine-2-carbonitrile (Compound 1-70); 7-[4-(1-Hydroxy-1-trifluoromethyl-propyl)-[1,2,3]triazol-1-ylmethyl]-3-(6-methyl-pyridin-3-yl)-indolizine-2-carboxylic acid amide (Compound 1-71); 7-[4-(1-Hydroxy-1-trifluoromethyl-propyl)-[1,2,3]triazol-1-ylmethyl]-3-(6-methyl-pyridin-3-yl)-indolizine-2-carbonitrile (Compound 1-72); 7-[4-(1-Hydroxy-1-trifluoromethyl-propyl)-[1,2,3]triazol-1-ylmethyl]-3-pyrimidin-5-yl-indolizine-2-carboxylic acid ethyl ester (Compound 1-73); 7-[4-(1-Hydroxy-1-trifluoromethyl-propyl)-[1,2,3]triazol-1-ylmethyl]-3-(1-methyl-1H-pyrazol-4-yl)-indolizine-2-carboxylic acid ethyl ester (Compound 1-74); 4-Nitro-benzoic acid 1-{1-[2-carbamoyl-3-(6-methyl-pyridin-3-yl)-indolizin-7-ylmethyl]-1H-[1,2,3]triazol-4-yl}-1-trifluoromethyl-propyl ester (Enantiomer B) (Compound 1-75); 7-[4-(1-Hydroxy-1-trifluoromethyl-propyl)-[1,2,3]triazol-1-ylmethyl]-3-(6-methyl-pyridin-3-yl)-indolizine-2-carbonitrile (Enantiomer B) (Compound 1-76); 7-[4-(1-Hydroxy-1-trifluoromethyl-propyl)-[1,2,3]triazol-1-ylmethyl]-3-isopropyl-indolizine-2-carboxylic acid ethyl ester (Enantiomer B) (Compound 1-77); 7-[4-(1-Hydroxy-1-trifluoromethyl-propyl)-[1,2,3]triazol-1-ylmethyl]-3-(6-methoxy-pyridin-3-yl)-indolizine-2-carboxylic acid amide (Enantiomer B) (Compound 1-78); 7-[4-(1-Hydroxy-1-trifluoromethyl-propyl)-[1,2,3]triazol-1-ylmethyl]-3-(6-methoxy-pyridin-3-yl)-indolizine-2-carbonitrile (Enantiomer B) (Compound 1-79); 7-[4-(1-Hydroxy-1-trifluoromethyl-propyl)-[1,2,3]triazol-1-ylmethyl]-3-(2-methyl-pyridin-4-yl)-indolizine-2-carbonitrile (Enantiomer B) (Compound 1-80); 7-[4-(1-Hydroxy-1-trifluoromethyl-propyl)-[1,2,3]triazol-1-ylmethyl]-3-isopropyl-indolizine-2-carbonitrile (Enantiomer B) (Compound 1-81); 7-[4-(1-Hydroxy-1-trifluoromethyl-propyl)-[1,2,3]triazol-1-ylmethyl]-3-(6-methoxy-pyridin-3-yl)-1-(2,2,2-trifluoro-acetyl)-indolizine-2-carboxylic acid amide (Enantiomer B) (Compound 1-82); 4-Nitro-benzoic acid 1-{1-[2-carbamoyl-3-(6-methoxy-pyridin-3-yl)-indolizin-7-ylmethyl]-1H-[1,2,3]triazol-4-yl}-1-trifluoromethyl-propyl ester (Enantiomer B) (Compound 1-83); 3-(6-Methoxy-pyridin-3-yl)-7-{4-[1-(4-nitro-benzoyloxy)-1-trifluoromethyl-propyl]-[1,2,3]triazol-1-ylmethyl}-1-(2,2,2-trifluoro-acetyl)-indolizine-2-carboxylic acid ethyl ester (Enantiomer B) (Compound 1-84); 3-(6-Methoxy-pyridin-3-yl)-7-{4-[1-(4-nitro-benzoyloxy)-1-trifluoromethyl-propyl]-[1,2,3]triazol-1-ylmethyl}-indolizine-2-carboxylic acid ethyl ester (Enantiomer B) (Compound 1-85); 7-{4-[1-(4-Nitro-benzoyloxy)-1-trifluoromethyl-propyl]-[1,2,3]triazol-1-ylmethyl}-indolizine-2-carboxylic acid ethyl ester (Enantiomer B) (Compound 1-86); 3-Chloro-7-[4-(1-hydroxy-1-trifluoromethyl-propyl)-[1,2,3]triazol-1-ylmethyl]-indolizine-2-carboxylic acid ethyl ester (Enantiomer B) (Compound 1-87); 3-Chloro-7-[4-(1-hydroxy-1-trifluoromethyl-propyl)-[1,2,3]triazol-1-ylmethyl]-indolizine-2-carbonitrile (Enantiomer B) (Compound 1-88); or 3-Chloro-7-[4-(1-hydroxy-1-trifluoromethyl-propyl)-[1,2,3]triazol-1-ylmethyl]-indolizine-2-carboxylic acid amide (Enantiomer B) (Compound 1-89). In some embodiments, provided herein is a pharmaceutically acceptable salt, pharmaceutically acceptable N-oxide, pharmaceutically active metabolite, pharmaceutically acceptable prodrug, or pharmaceutically acceptable solvate of one of such compounds.

Throughout the specification, groups and substituents thereof can be chosen by one skilled in the field to provide stable moieties and compounds.

### Further Forms of Compounds

Compounds described herein may possess one or more stereocenters and each center may exist in the R or S configuration. The compounds presented herein include all diastereomeric, enantiomeric, and epimeric forms as well as the appropriate mixtures thereof. Separation of steroisomers may be performed by chromatography. Alternatively, individual stereoisomers may be obtained by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds described herein, dissociable complexes are also possible (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chiral chromatography, or by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981, herein incorporated by reference for such disclosure. Stereoisomers may also be obtained by stereoselective synthesis.

In some situations, compounds may exist as tautomers. All tautomers are included within the formulas described herein.

In some cases, cyclic compounds described herein may be in equilibrium with open chain forms. Closed cyclic forms as well as the corresponding open chain forms, which are in equilibrium with the closed cyclic forms, are considered part of the present disclosure.

The methods and formulations described herein include the use of N-oxides, crystalline forms (also known as polymorphs), or pharmaceutically acceptable salts of compounds described herein, as well as active metabolites of these compounds having the same type of activity. In some situations, compounds may exist as tautomers. All tautomers are included within the scope of the compounds presented herein. In addition, the compounds described herein can exist in unsolvated as well as solvated forms with pharmaceutically acceptable solvents such as water, ethanol, and the like. The solvated forms of the compounds presented herein are also considered to be disclosed herein.

Compounds of any of Formula (I) or (II) in unoxidized form can be prepared from N-oxides of compounds of any of Formula (I) or (II), by treating with a reducing agent, such as, but not limited to, sulfur, sulfur dioxide, triphenyl phosphine, lithium borohydride, sodium borohydride, phosphorus trichloride, phosphorus tribromide, or the like in a suitable inert organic solvent, such as, but not limited to, acetonitrile, ethanol, aqueous dioxane, or the like at 0 to 80° C.

In some embodiments, compounds described herein are prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

To produce a prodrug, a pharmaceutically active compound is modified such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Saulnier et al., (1994), Bioorganic and Medicinal Chemistry Letters, Vol. 4, p. 1985).

Prodrug forms of the herein described compounds, wherein the prodrug is metabolized in vivo to produce a derivative as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. Prodrugs may be designed as reversible drug derivatives, for use as modifiers to enhance drug transport to site-specific tissues. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure.

Sites on the aromatic ring portion of compounds described herein can be susceptible to various metabolic reactions, therefore incorporation of appropriate substituents on the aromatic ring structures, such as, by way of example only, halogens can reduce, minimize or eliminate this metabolic pathway.

The compounds described herein may be labeled isotopically (e.g. with a radioisotope) or by other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, ²H, ³H, ¹³C, ¹⁴C, ¹⁵N, ¹⁸O, ¹⁷O, ³⁵S, ¹⁸F, ³⁶Cl, respectively. Certain isotopically-labeled compounds described herein, for example those into which radioactive isotopes such as ³H and ¹⁴C are incorporated, are useful in drug and/or substrate tissue distribution assays. Further, substitution with isotopes such as deuterium, i.e., ²H, can afford certain therapeutic advantages resulting from greater metabolic stability, for example increased in vivo half-life or reduced dosage requirements.

In additional or further embodiments, the compounds described herein are metabolized upon administration to an organism in need to produce a metabolite that is then used to produce a desired effect, including a desired therapeutic effect.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, nitric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; (2) salts formed when an acidic proton present in the parent compound either is replaced by a metal ion, e.g., an alkali metal ion (e.g. lithium, sodium, potassium), an alkaline earth ion (e.g. magnesium, or calcium), or an aluminum ion; or coordinates with an organic base. Acceptable organic bases include ethanolamine, diethanolamine, triethanolamine, tromethamine, N-methylglucamine, dicyclohexylamine, tris(hydroxymethyl)methylamine, and salts with amino acids such as arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

Compounds described herein may be in various forms, including but not limited to, amorphous forms, milled forms and nano-particulate forms. In addition, compounds described herein include crystalline forms, also known as polymorphs. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The screening and characterization of the pharmaceutically acceptable salts, polymorphs and/or solvates may be accomplished using a variety of techniques including, but not limited to, thermal analysis, x-ray diffraction, spectroscopy, vapor sorption, and microscopy. Thermal analysis methods address thermo chemical degradation or thermo physical processes including, but not limited to, polymorphic transitions, and such methods are used to analyze the relationships between polymorphic forms, determine weight loss, to find the glass transition temperature, or for excipient compatibility studies. Such methods include, but are not limited to, Differential scanning calorimetry (DSC), Modulated Differential Scanning calorimetry (MDCS), Thermogravimetric analysis (TGA), and Thermogravi-metric and Infrared analysis (TG/IR). X-ray diffraction methods include, but are not limited to, single crystal and powder diffractometers and synchrotron sources. The various spectroscopic techniques used include, but are not limited to, Raman, FTIR, UV-VIS, and NMR (liquid and solid state). The various microscopy techniques include, but are not limited to, polarized light microscopy, Scanning Electron Microscopy (SEM) with Energy Dispersive X-Ray Analysis (EDX), Environmental Scanning Electron Microscopy with EDX (in gas or water vapor atmosphere), IR microscopy, and Raman microscopy.

Throughout the specification, groups and substituents thereof can be chosen by one skilled in the field to provide stable moieties and compounds.

### Preparation of Compounds

The synthesis of compounds described herein may be accomplished using means described in the chemical literature, using the methods described herein, or by a combination thereof.

Compounds described herein may be synthesized using standard synthetic techniques known to those of skill in the art or using methods known in the art in combination with methods described herein. In addition, solvents, temperatures, reagents and other reaction conditions presented herein may vary according to those of skill in the art.

The starting material used for the synthesis of the compounds described herein may be synthesized or can be obtained from commercial sources, such as, but not limited to, Aldrich Chemical Co. (Milwaukee, Wis.), or Sigma Chemical Co. (St. Louis, Mo.). The compounds described herein, and other related compounds having different substituents can be synthesized using techniques and materials described herein as well as those that are known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999) (all of which are incorporated by reference for such disclosure). General methods for the preparation of compound as disclosed herein may be derived from known reactions in the field, and the reactions may be modified by the use of appropriate reagents and conditions, as would be recognized by the skilled person, for the introduction of the various moieties found in the formulae as provided herein. As a guide the synthetic method of Scheme 1 may be utilized to prepare a compound described herein.

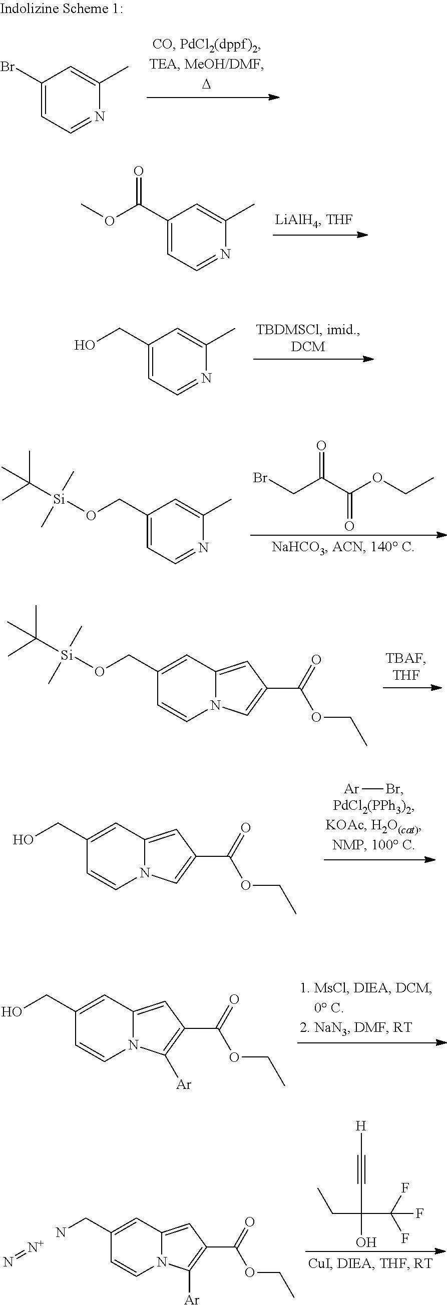

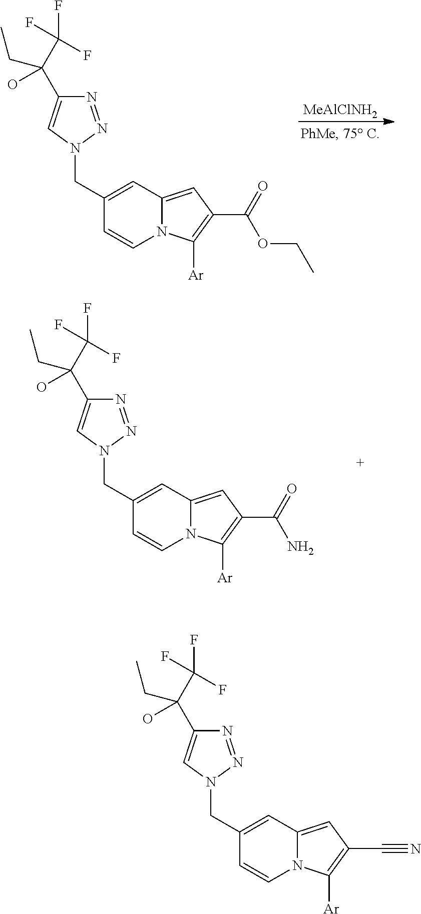

In some embodiments, a non-aromatic substituent at the R¹ position of a compound of either of Formula I or II may be prepared by reacting the indolizine with another desired electrophile under suitable conditions instead of reacting the indolizine with Ar—Br, as described in Scheme 1.

In some embodiments, the synthesis of compounds described herein begins with the synthetic steps outlined in Scheme 2.

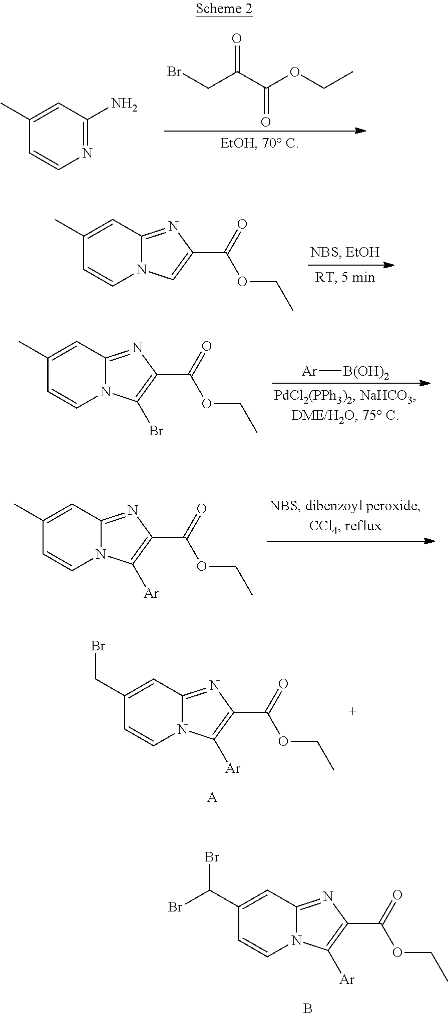

In some embodiments, compounds of structure A are used to prepare compounds of Formula (I) as outlined in Scheme 3.

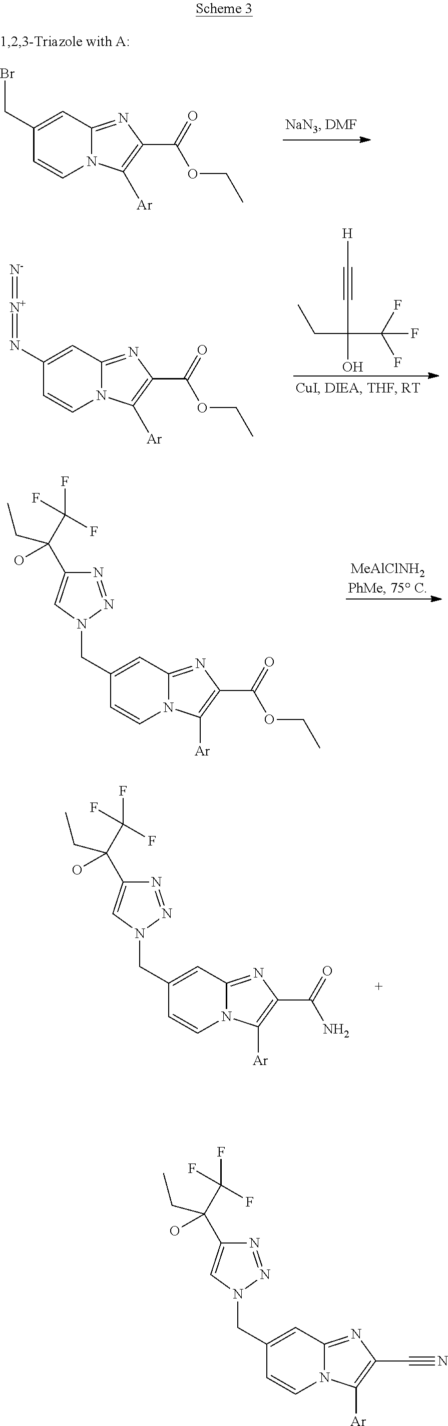

In some embodiments, compounds of structure B are used to prepare compounds of Formula (I) as outlined in Scheme 4.

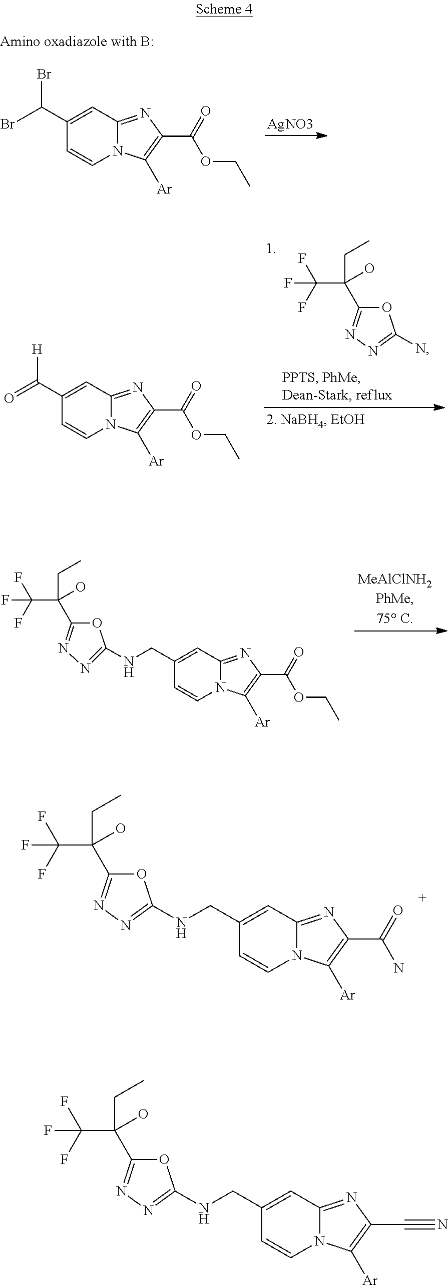

Throughout the specification, groups and substituents thereof can be chosen by one skilled in the field to provide stable moieties and compounds.

### CERTAIN TERMINOLOGY

Unless defined otherwise, all technical and scientific terms used herein have the same meaning as is commonly understood by one of skill in the art to which the claimed subject matter belongs. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless otherwise stated. Furthermore, use of the term “including” as well as other forms, such as “include”, “includes,” and “included,” is not limiting.

Definition of standard chemistry terms may be found in reference works, including Carey and Sundberg “ADVANCED ORGANIC CHEMISTRY 4^{Th} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein.

As used herein, C₁-Cₓ includes C₁-C₂, C₁-C₃ . . . C₁-Cₓ.

An “alkyl” group refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched or straight chain. Depending on the structure, an alkyl group can be a monoradical or a diradical (i.e., an alkylene group).

In certain instances, the “alkyl” moiety may have 1 to 10 carbon atoms (whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated). The alkyl group could also be a “lower alkyl” having 1 to 6 carbon atoms. The alkyl group of the compounds described herein may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, ethenyl, propenyl, butenyl, and the like.

An “alkoxy” group refers to a (alkyl)O— group, where alkyl is as defined herein. A “lower alkoxy” has 1 to 6 carbon atoms.

The term “alkenyl” refers to a type of alkyl group in which the first two atoms of the alkyl group form a double bond that is not part of an aromatic group. That is, an alkenyl group begins with the atoms —C(R)═CR₂, wherein R refers to the remaining portions of the alkenyl group, which may be the same or different. Non-limiting examples of an alkenyl group include —CH═CH₂, —C(CH₃)═CH₂, —CH═CH═CH₃ and —C(CH₃)═CH═CH₃. Depending on the structure, an alkenyl group can be a monoradical or a diradical (i.e., an alkenylene group). In some embodiments, an alkynyl is a C₂-C₆alkenyl.

The term “alkynyl” refers to a type of alkyl group in which the first two atoms of the alkyl group form a triple bond. That is, an alkynyl group begins with the atoms —C≡C—R, wherein R refers to the remaining portions of the alkynyl group, which may be the same or different. Non-limiting examples of an alkynyl group include —C≡CH, —C≡CCH₃ and —C≡CCH₂CH₃. The “R” portion of the alkynyl moiety may be branched, straight chain, or cyclic. Depending on the structure, an alkynyl group can be a monoradical or a diradical (i.e., an alkynylene group). In some embodiments, an alkynyl is a C₂-C₆alkynyl.

The term “alkylamine” refers to the —N(alkyl)ₓH_{y} group, where alkyl is as defined herein and x and y are selected from the group x=1, y=1 and x=2, y=0. When x=2, the alkyl groups taken together with the nitrogen atom to which they are attached can optionally form a heterocyclic ring system.

An “amide” is a chemical moiety with formula —C(═O)NHR or —NHC(═O)R, where R is selected from the group consisting of alkyl, cycloalkyl, aryl, heteroaryl (bonded through a ring carbon) and heteroalicyclic (bonded through a ring carbon). An amide may be an amino acid or a peptide molecule attached to a compound described herein, such as, for example, a compound of Formula (I), thereby forming a prodrug. Any amine, or carboxyl side chain on the compounds described herein can be amidified. The procedures and specific groups to make such amides are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference for such disclosure.

The term “ester” refers to a chemical moiety with formula —COOR, where R is selected from the group consisting of alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference for such disclosure.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

As used herein, the term “ring system” refers to one, or more than one ring.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “fused” refers to structures in which two or more rings share one or more bonds.

The term “carbocyclic” or “carbocycle” refers to a ring wherein each of the atoms forming the ring is a carbon atom. Carbocycle includes aryl and cycloalkyl. The term thus distinguishes carbocycle from heterocycle (“heterocyclic”) in which the ring backbone contains at least one atom which is different from carbon (i.e a heteroatom). Heterocycle includes heteroaryl and heterocycloalkyl. Carbocycles and heterocycles can be optionally substituted.

The term “aromatic” refers to a planar ring having a delocalized π-electron system containing 4n+2π electrons, where n is an integer. Aromatic rings can be formed from five, six, seven, eight, nine, or more than nine atoms. Aromatics can be optionally substituted. The term “aromatic” includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

The term “cycloalkyl” refers to a monocyclic or polycyclic non-aromatic radical, wherein each of the atoms forming the ring (i.e. skeletal atoms) is a carbon atom. Cycloalkyls may be saturated, or partially unsaturated. Cycloalkyl groups include groups having from 3 to 10 ring atoms. Illustrative examples of cycloalkyl groups include the following moieties:

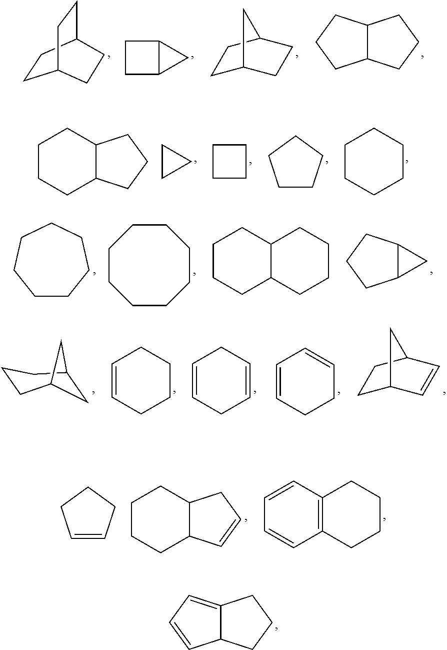

and the like.
Depending on the structure, a cycloalkyl group can be a monoradical or a diradical (e.g., an cycloalkylene group). Monocyclic cycloalkyls include, but are not limited to, cyclopropyl, cyclobutyl, cyclopentyl, cyclohexyl, cycloheptyl, and cyclooctyl. In some embodiments, a cycloalkyl is a C₃-C₈cycloalkyl.

A “cycloalkylalkyl” refers to an alkyl, as defined herein, substituted with a cycloalkyl, as defined herein. Cycloalkylalkyls include, but are not limited to, cyclopropylmethyl, cyclobutylmethyl, cyclopentylmethyl, cyclohexylmethyl, cycloheptylmethyl, and cyclooctylmethyl.

A “cycloalkoxy” refers to —O-(cycloalkyl), where cycloalkyl is as defined herein.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. The polycyclic heteroaryl group may be fused or non-fused. Illustrative examples of heteroaryl groups include the following moieties:

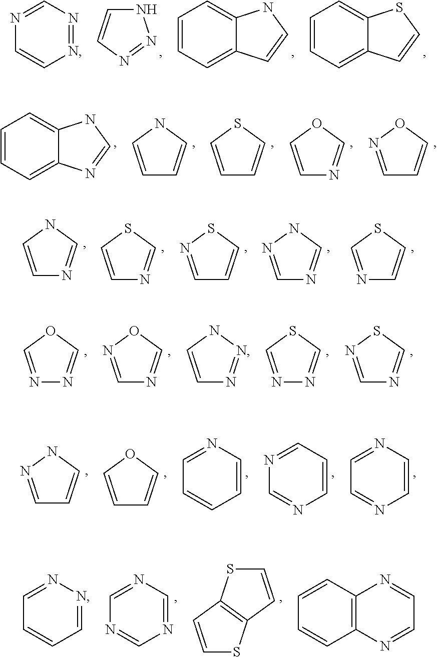

and the like. Depending on the structure, a heteroaryl group can be a monoradical or a diradical (i.e., a heteroarylene group). In some embodiments, a monocyclic heteroaryl is pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, or furazanyl. In one aspect, a heteroaryl contains 0-3 N atoms. In another aspect, a heteroaryl contains 1-3 N atoms. In another aspect, a heteroaryl contains 0-3 N atoms, 0-1 O atoms, and 0-1 S atoms. In another aspect, a heteroaryl is a monocyclic or bicyclic heteroaryl. In one aspect, heteroaryl is a C₁-C₉heteroaryl. In one aspect, monocyclic heteroaryl is a C₁-C₅heteroaryl. In one aspect, monocyclic heteroaryl is a 5-membered or 6-membered heteroaryl. In one aspect, bicyclic heteroaryl is a C₆-C₉heteroaryl.

The term “heterocycle” or “cycloheteroalkyl” refers to heteroaromatic and heteroalicyclic groups containing one to four heteroatoms each selected from O, S and N, wherein each heterocyclic group has from 4 to 10 atoms in its ring system, and with the proviso that the ring of said group does not contain two adjacent O or S atoms. Non-aromatic heterocyclic groups include groups having only 3 atoms in their ring system, but aromatic heterocyclic groups must have at least 5 atoms in their ring system. The heterocyclic groups include benzo-fused ring systems. An example of a 3-membered heterocyclic group is aziridinyl. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one. Depending on the structure, a heterocycle group can be a monoradical or a diradical (i.e., a heterocyclene group).

A “heteroalicyclic” or “heterocycloalkyl” group refers to a cycloalkyl group that includes at least one ring atom that is not a carbon, i.e. at least one ring atom is a heteroatom selected from nitrogen, oxygen and sulfur. The heterocycloalkyl radicals may be fused with an aryl or heteroaryl. Illustrative examples of heterocycloalkyl groups, also referred to as non-aromatic heterocycles, include:

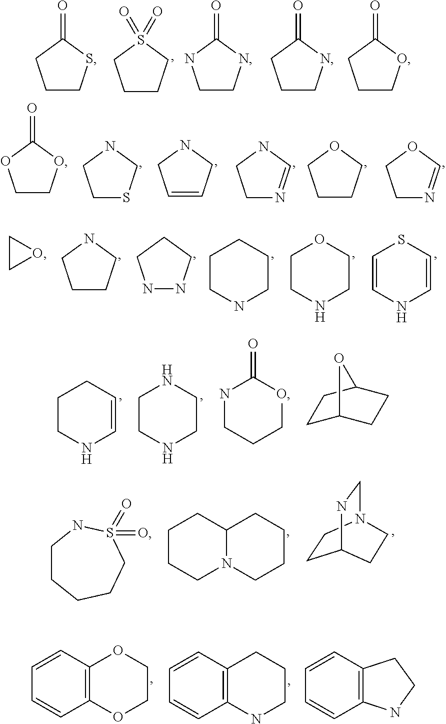

and the like. The term heteroalicyclic also includes all ring forms of the carbohydrates, including but not limited to the monosaccharides, the disaccharides and the oligosaccharides. Heterocycloalkyls have from 2 to 10 carbons in the ring. In some embodiments, a heterocycloalkyl has 2 to 8 ring carbon atoms. It is understood that when referring to the number of carbon atoms in a heterocycloalkyl, the number of carbon atoms in the heterocycloalkyl is not the same at the total number of atoms (including the heteroatoms) that make up the heterocycloalkyl (i.e skeletal atoms of the heterocycloalkyl ring). In one aspect, a heterocycloalkyl contains 0-2 N atoms. In another aspect, a heterocycloalkyl contains 0-2 N atoms, 0-2 O atoms or 0-1 S atoms.

The terms “halo”, “halide”, and “halogen” mean fluoro, chloro, bromo and iodo.

The terms “haloalkyl,” “haloalkenyl,” “haloalkynyl” and “haloalkoxy” include alkyl, alkenyl, alkynyl and alkoxy structures that are substituted with one or more halogens. The halogens may the same or they may be different. In some embodiments, a haloalkyl has 1 to 6 carbon atoms in the chain. In some embodiments, a haloalkenyl has 2 to 6 carbon atoms in the chain. In some embodiments, a haloalkynyl has 2 to 6 carbon atoms in the chain. In some embodiments, a haloalkoxy has 1 to 6 carbon atoms in the chain.

The terms “fluoroalkyl” and “fluoroalkoxy” include haloalkyl and haloalkoxy groups, respectively, in which the halo is fluorine. In some embodiments, a fluoroalkyl and a fluoroalkoxy have 1 to 6 carbon atoms in the chain.

The terms “heteroalkyl” “heteroalkenyl” and “heteroalkynyl” include optionally substituted alkyl, alkenyl and alkynyl radicals and which have one or more skeletal chain atoms selected from an atom other than carbon, e.g., oxygen, nitrogen, sulfur, phosphorus, silicon, or combinations thereof. The heteroatom(s) may be placed at any interior position of the heteroalkyl group. As with “alkyl” groups, “heteroalkyl” groups may include saturated and/or unsaturated groups, as well as straight chain, branched and/or cyclic groups. Examples include, but are not limited to, —O—CH₃, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃. A “lower heteroalkyl” has 1 to 6 carbon atoms in the chain. In some embodiments, a heteroalkyl has 1 to 6 carbon atoms in the chain. In some embodiments, a heteroalkenyl has 2 to 6 carbon atoms in the chain. In some embodiments, a heteroalkynyl has 2 to 6 carbon atoms in the chain.

The term “bond” or “single bond” refers to a chemical bond between two atoms, or two moieties when the atoms joined by the bond are considered to be part of larger substructure.

The term “moiety” refers to a specific segment or functional group of a molecule. Chemical moieties are often recognized chemical entities embedded in or appended to a molecule.

A “cyano” group refers to a —CN group.

An “isocyanato” group refers to a —NCO group.

An “isothiocyanato” group refers to a —NCS group.

“Acyl” refers to a RC(═O)— group.

“Acyloxy” refers to a RC(═O)O— group.

“Sulfanyl” refers to a —S— moiety.

“Sulfinyl” or “sulfoxide” refers to a —S(═O)— moiety.

“Sulfonyl” refers to a —S(═O)₂— moiety.

A “mercaptyl” group or “thioalkoxy” or “alkylthio” refers to a (alkyl)S— group.

A “thiocyanato” group refers to a —CNS group.

As used herein, the substituent “R” appearing by itself and without a number designation refers to a substituent selected from among from alkyl, cycloalkyl, aryl, heteroaryl (bonded through a ring carbon) and non-aromatic heterocycle (bonded through a ring carbon).

The term “optionally substituted” or “substituted” means that the referenced group may be substituted with one or more additional group(s) individually and independently selected from C₁-C₆alkyl, C₃-C₈cycloalkyl, aryl, heteroaryl, C₂-C₆heteroalicyclic, hydroxy, C₁-C₆alkoxy, aryloxy, C₁-C₆alkylthio, arylthio, C₁-C₆alkylsulfoxide, arylsulfoxide, C₁-C₆alkylsulfone, arylsulfone, cyano, halo, C₂-C₈acyl, C₂-C₈acyloxy, nitro, C₁-C₆haloalkyl, C₁-C₆-fluoroalkyl, and amino, including C₁-C₆alkylamino, and the protected derivatives thereof. By way of example, an optional substituents may be L^{s}R^{s}, wherein each L^{s} is independently selected from a bond, —O—, —C(═O)—, —S—, —S(═O)—, —S(═O)₂—, —NH—, —NHC(═O)—, —C(═O)NH—, S(═O)₂NH—, —NHS(═O)₂—, —OC(═O)NH—, —NHC(═O)O—, —(C₁-C₆alkyl)-, or —(C₂-C₆alkenyl)-; and each R^{s} is independently selected from H, (C₁-C₄alkyl), (C₃-C₈cycloalkyl), heteroaryl, aryl, and C₁-C₆heteroalkyl. In some embodiments, an optional substituents is halogen, —CN, —NH₂, —OH, —NH(CH₃), —N(CH₃)₂, —CH₃, —CH₂CH₃, —CF₃, —OCH₃, —OCH₂CH₃, —OCF₃, —SO₂CH₃, —CO₂CH₃, —CO₂CH₂CH₃, —CO₂H, or —CONH₂. In some embodiments, a substituted group is substituted with one or more of the preceding groups. In some embodiments, a substituted group is substituted with one or two of the preceding groups. Optionally substituted non-aromatic groups may be substituted with one or more oxo (═O). The protecting groups that may form the protective derivatives of the above substituents are known to those of skill in the art and may be found in references such as Greene and Wuts, above.

The compounds presented herein may possess one or more stereocenters and each center may exist in the R or S configuration. The compounds presented herein include all diastereomeric, enantiomeric, and epimeric forms as well as the appropriate mixtures thereof. Stereoisomers may be obtained, if desired, by methods known in the art such as, for example, the separation of individual stereoisomers by chiral chromatographic columns or by stereoselective synthesis.

The methods and formulations described herein include the use of N-oxides, crystalline forms (also known as polymorphs), or pharmaceutically acceptable salts of compounds having the structure of any compound described herein, e.g., of Formula (I), Formula (II), or Table 1, as well as active metabolites of these compounds having the same type of activity. In some situations, compounds may exist as tautomers. All tautomers are included within the scope of the compounds presented herein. In addition, the compounds described herein can exist in unsolvated as well as solvated forms with pharmaceutically acceptable solvents such as water, ethanol, and the like. The solvated forms of the compounds presented herein are also considered to be disclosed herein.

The term “acceptable” with respect to a formulation, composition or ingredient, as used herein, means having no persistent detrimental effect on the general health of the subject being treated.

As used herein, the term “target protein” refers to a protein or a portion of a protein capable of being bound by a selective binding compound. In certain embodiments, a target protein is 5-LO.

As used herein, the term “selective binding compound” refers to a compound that selectively binds to any portion of one or more target proteins.

As used herein, the term “selectively binds” refers to the ability of a selective binding compound to bind to a target protein, such as, for example, 5-LO, with greater affinity than it binds to a non-target protein. In certain embodiments, specific binding refers to binding to a target with an affinity that is at least 10, 50, 100, 250, 500, 1000 or more times greater than the affinity for a non-target.

As used herein, amelioration of the symptoms of a particular disease, disorder or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

As used herein, the term “modulator” refers to a compound that alters an activity of a molecule. For example, a modulator can cause an increase or decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

As used herein, the term “target activity” refers to a biological activity capable of being modulated by a selective modulator. Certain exemplary target activities include, but are not limited to, binding affinity, signal transduction, enzymatic activity, tumor growth, inflammation or inflammation-related processes, and amelioration of one or more symptoms associated with a disease or condition.

As used herein, the term “agonist” refers to a compound, the presence of which results in a biological activity of a protein that is the same as the biological activity resulting from the presence of a naturally occurring ligand for the protein, such as, for example, 5-LO.

As used herein, the term “antagonist” refers to a compound, the presence of which results in a decrease in the magnitude of a biological activity of a protein. In certain embodiments, the presence of an antagonist results in complete inhibition of a biological activity of a protein, such as, for example, 5-LO. In certain embodiments, an antagonist is an inhibitor.

The terms “inhibits”, “inhibiting”, or “inhibitor” of 5-LO, as used herein, refer to inhibition of 5-lipoxygenase activity.

The term “asthma” as used herein refers to any disorder of the lungs characterized by variations in pulmonary gas flow associated with airway constriction of whatever cause (intrinsic, extrinsic, or both; allergic or non-allergic). The term asthma may be used with one or more adjectives to indicate cause.

The term “bone disease,’ as used herein, refers to a disease or condition of the bone, including, but not limited to, inappropriate bone remodeling, loss or gain, osteopenia, osteomalacia, osteofibrosis, and Paget's disease [Garcia, “Leukotriene B4 stimulates osteoclastic bone resorption both in intro and in vivo”, J Bone Miner Res. 1996; 11:1619-27].

The term “cardiovascular disease,” as used herein refers to diseases affecting the heart or blood vessels or both, including but not limited to: arrhythmia; atherosclerosis and its sequelae; angina; myocardial ischemia; myocardial infarction; cardiac or vascular aneurysm; vasculitis, stroke; peripheral obstructive arteriopathy of a limb, an organ, or a tissue; reperfusion injury following ischemia of the brain, heart or other organ or tissue; endotoxic, surgical., or traumatic shock; hypertension, valvular heart disease, heart failure, abnormal blood pressure; shock; vasoconstriction (including that associated with migraines); vascular abnormality, inflammation, insufficiency limited to a single organ or tissue. Lotzer K et al., “The 5-lipoxygenase pathway in arterial wall biology and atherosclerosis”, Biochim. Biophys. Acta, 1736:30-7, 2005; Helgadottir, A, et al., Nat. Genet., 233-9, 2004; Heise C E, Evans J F et al., J Biol. Chem. 30531-30536, 2000].

The term “cancer,’ as used herein refers to an abnormal growth of cells, which tend to proliferate in an uncontrolled way and, in some cases, to metastasize (spread). The types of cancer include, but is not limited to, solid tumors (such as those of the bladder, bowel, brain, breast, endometrium, heart, kidney, lung, lymphatic tissue (lymphoma), ovary, pancreas or other endocrine organ (thyroid), prostate, skin (melanoma) or hematological tumors (such as the leukemias) [Ding X Z et al., Anticancer Drugs. 2005 June; 16(5):467-73. Review; Chen X et al., Clin Cancer Res. 2004 Oct. 1; 10(19):6703-9].

The term “dermatological disorder,” as used herein refers to a skin disorder. Such dermatological disorders include, but are not limited to, proliferative or inflammatory disorders of the skin such as, atopic dermatitis, bullous disorders, collagenoses, contact dermatitis eczema, Kawasaki Disease, rosacea, Sjogren-Larsso Syndrome, urticaria [Wedi, B, et al., Bio Drugs, 2001, 15(11): 729-43].

The terms “fibrosis” or “fibrosing disorder,” as used herein, refers to conditions that follow acute or chronic inflammation and are associated with the abnormal accumulation of cells and/or collagen and include but are not limited to fibrosis of individual organs or tissues such as the heart, kidney, joints, lung, or skin, and includes such disorders as idiopathic pulmonary fibrosis and cryptogenic fibrosing alveolitis [Charbeneau R P et al., Clin Sci (Lond). 2005 June; 108(6):479-91].

The term “iatrogenic” means a leukotriene-dependent or leukotriene-mediated condition, disorder, or disease created or worsened by medical or surgical therapy.

The term “inflammatory disorders” refers to those diseases or conditions that are characterized by one or more of the signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and loss of function (functio laesa, which may be partial or complete, temporary or permanent). Inflammation takes many forms and includes, but is not limited to, inflammation that is one or more of the following: acute, adhesive, atrophic, catarrhal., chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative. Inflammatory disorders further include, without being limited to those affecting the blood vessels (polyarteritis, temporal arteritis); joints (arthritis: crystalline, osteo-, psoriatic, reactive, rheumatoid, Reiter's); gastrointestinal tract (Crohn's Disease, ulcerative colitis); skin (dermatitis); or multiple organs and tissues (systemic lupus erythematosus) [Harrison's Principles of Internal Medicine, 16^{th} Edition, Kasper D L, et al., Editors; McGraw-Hill, publishers].

The term “interstitial cystitis” refers to a disorder characterized by lower abdominal discomfort, frequent and sometimes painful urination that is not caused by anatomical abnormalites, infection, toxins, trauma or tumors [Bouchelouche K et al., J. Urol. 166:1734, 2001].

The terms “neurogenerative disease” or “nervous system disorder,” as used herein, refers to conditions that alter the structure or function of the brain, spinal cord or peripheral nervous system, including but not limited to Alzheimer's Disease, cerebral edema, cerebral ischemia, multiple sclerosis, neuropathies, Parkinson's Disease, those found after blunt or surgical trauma (including post-surgical cognitive dysfunction and spinal cord or brain stem injury), as well as the neurological aspects of disorders such as degenerative disk disease and sciatica. The acronym “CNS” refers to disorders of the central nervous system, i.e., brain and spinal cord [Sugaya K, et al., Jpn. J. Pharmacol., 2000, February, 82(2): 85-94; Yu, G L, et al., Pharmacology, 2005, January, 73(1):31-40. Epub 2004 Sep. 27; Zhang W P, et al., Acta Pharmacol. Sin., 2002, October, 23(10): 871-7].

The terms “ocular disease” or “ophthalmic disease,” as used herein, refer to diseases which affect the eye or eyes and potentially the surrounding tissues as well. Ocular or ophthalmic diseases include, but are not limited to, conjunctivitis, retinitis, scleritis, uveitis, allergic conjunctivitis, vernal conjunctivitis, papillary conjunctivitis [Toriyama S., Nippon Ganka Gakkai Zasshi. 2000 June; 104(6):396-40; [Chen F, et al., Ophthalmic Res. 1991; 23(2):84-91].

The term “respiratory disease,” as used herein, refers to diseases affecting the organs that are involved in breathing, such as the nose, throat, larynx, trachea, bronchi, and lungs. Respiratory diseases include, but are not limited to, asthma, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma, seasonal allergic rhinitis, perennial allergic rhinitis, chronic obstructive pulmonary disease, including chronic bronchitis or emphysema, pulmonary hypertension, interstitial lung fibrosis and/or airway inflammation and cystic fibrosis, and hypoxia [Evans J F, “The Cysteinyl Leukotriene (CysLT) Pathway in Allergic Rhinitis”, Allergology International 2005; 54:187-90); Kemp J P., “Leukotriene receptor antagonists for the treatment of asthma”, I Drugs. 2000 April; 3(4):430-41; Riccioni G, et al., Allergy Asthma Proc. 2004 November-December; 25(6):445-8].

The term “leukotriene-driven mediators,” as used herein, refers to molecules able to be produced in a patient that may result from excessive production of leukotriene stimulation of cells, such as, by way of example only, LTB₄, LTC₄, LTE₄, cysteinyl leukotrienes, monocyte inflammatory protein (MIP-1α), interleukin-8 (IL-8), interleukin-4 (IL-4), interleukin-13 (IL-13), monocyte chemoattractant protein (MCP-1), soluble intracellular adhesion molecule (sICAM; soluble ICAM), myeloperoxidase (MPO), eosinophil peroxidase (EPO), and general inflammation molecules such as interleukin-6 (IL-6), C-reactive protein (CRP), and serum amyloid A protein (SAA).

The term “leukotriene-dependent”, as used herein, refers to conditions or disorders that would not occur, or would not occur to the same extent, in the absence of one or more leukotrienes.

The term “leukotriene-mediated”, as used herein, refers to refers to conditions or disorders that might occur in the absence of leukotrienes but can occur in the presence of one or more leukotrienes.

The term “leukotriene-responsive patient,” as used herein, refers to a patient who has been identified by either genotyping of FLAP haplotypes, or genotyping of LTA₄ hydrolase haplotypes or genotyping of one or more other genes in the leukotriene pathway and/or, by phenotyping of patients either by previous positive clinical response to another leukotriene modulator, including, by way of example only, zileuton (Zyflo®), montelukast (Singulair®), pranlukast, zafirlukast (Accolate®), and/or by their profile of leukotriene-driven mediators that indicate excessive leukotriene stimulation of inflammatory cells, as likely to respond favorably to leukotriene modulator therapy.

The term “carrier,” as used herein, refers to relatively nontoxic chemical compounds or agents that facilitate the incorporation of a compound into cells or tissues.

The terms “co-administration” or the like, as used herein, are meant to encompass administration of the selected therapeutic agents to a single patient, and are intended to include treatment regimens in which the agents are administered by the same or different route of administration or at the same or different time.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “enzymatically cleavable linker,” as used herein refers to unstable or degradable linkages which may be degraded by one or more enzymes.

The terms “kit” and “article of manufacture” are used as synonyms.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyl transferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds. Both methods are well known in the art.

By “pharmaceutically acceptable,” as used herein, refers a material., such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a formulation of a compound that does not cause significant irritation to an organism to which it is administered and does not abrogate the biological activity and properties of the compound.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound of any of Formulas (I) or (II), and a co-agent, are both administered to a patient simultaneously in the form of a single entity or dosage. The term “non-fixed combination” means that the active ingredients, e.g. a compound of any of Formulas (I) or (II), and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutical composition” refers to a mixture of a compound of any of Formulas (I) or (II), with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral., aerosol, parenteral., ophthalmic, pulmonary and topical administration.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Composition/Formulation

For convenience, the pharmaceutical compositions and formulations described in this section and other parts herein use a single formula, such as “Formula (I),” by way of example. In addition, the pharmaceutical compositions and formulations described herein apply equally well to all formulae presented herein that fall within the scope of Formula (I). For example, the pharmaceutical compositions and formulations described herein can be applied to compounds having the structure of any of Formulas (I) or (II), as well as to all of the specific compounds that fall within the scope of these generic formulae and any compounds specifically identifed herein.

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

Provided herein are pharmaceutical compositions that include a compound described herein, such as a compound of Formula (I) and a pharmaceutically acceptable diluent(s), excipient(s), and/or carrier(s). In addition, the compounds described herein can be administered as pharmaceutical compositions in which compounds described herein, such as compounds of Formula (I), are mixed with other active ingredients, as in combination therapy.

A pharmaceutical composition, as used herein, refers to a mixture of a compound described herein, such as a compound of Formula (I) with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein, such as compounds of Formula (I) provided herein are administered in a pharmaceutical composition to a mammal having a disease or condition to be treated. Preferably, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

For intravenous injections, compounds described herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations may include aqueous or nonaqueous solutions, preferably with physiologically compatible buffers or excipients. Such excipients are generally known in the art.

For oral administration, compounds described herein can be formulated readily by combining the active compounds with pharmaceutically acceptable carriers or excipients well known in the art. Such carriers enable the compounds described herein to be formulated as tablets, powders, pills, dragees, capsules, liquids, gels, syrups, elixirs, slurries, suspensions and the like, for oral ingestion by a patient to be treated.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, but not limited to, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal or sublingual administration, the compositions may take the form of tablets, lozenges, or gels formulated in a conventional manner. Parental injections may involve bolus injection or continuous infusion. Formulations for injection may be presented in unit dosage form, e.g., in ampoules or in multi-dose containers, with an added preservative. The pharmaceutical composition of the compounds described herein may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compound(s) in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

The compounds described herein, such as compounds of Formula (I), can be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

Formulations suitable for transdermal administration of compounds described herein may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of the compounds described herein can be accomplished by means of iontophoretic patches and the like. Additionally, transdermal patches can provide controlled delivery of the compounds described herein. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

For administration by inhalation, the compounds described herein may be in a form as an aerosol, a mist, or a powder. Pharmaceutical compositions of compounds described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide, or other suitable gas. In the case of a pressurized aerosol the dosage unit may be determined by providing a valve to deliver a metered amount. Capsules and cartridges of, such as, by way of example only, gelatin for use in an inhaler or insufflator may be formulated containing a powder mix of the compound and a suitable powder base such as lactose or starch.

The compounds described herein may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. Pharmaceutical compositions that include a compound described herein may be manufactured in a conventional manner, such as, by way of example only, by means of conventional mixing, dissolving, granulating, dragee-making, levigating, emulsifying, encapsulating, entrapping or compression processes.

The pharmaceutical compositions will include at least one pharmaceutically acceptable carrier, diluent and/or excipient and a compound described herein, such as a compound of Formula (I) as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds may exist as tautomers. All tautomers are included within the scope of the compounds presented herein. In some embodiments, cyclic compounds described herein may exist in equilibrium with open chain forms. Both forms, cyclic and open form, are included. Additionally, the compounds described herein can exist in unsolvated as well as solvated forms with pharmaceutically acceptable solvents such as water, ethanol, and the like. The solvated forms of the compounds presented herein are also considered to be disclosed herein. In addition, the pharmaceutical compositions may include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions can also contain other therapeutically valuable substances.

Methods for the preparation of compositions that include the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions that include a compound described herein, or a solution containing liposomes, micelles, or nanoparticles that include a compound as disclosed herein. Semi-solid compositions include, but are not limited to, gels, suspensions and creams. The compositions may be in liquid solutions or suspensions, solid forms suitable for solution or suspension in a liquid prior to use, or as emulsions. These compositions may also contain minor amounts of nontoxic, auxiliary substances, such as wetting or emulsifying agents, pH buffering agents, and so forth.

A composition that includes a compound described herein, such as a compound of Formula (I) can illustratively take the form of a liquid where the agents are present in solution, in suspension, or both. Typically when the composition is administered as a solution or suspension, a first portion of the compound is present in solution and a second portion of the compound is present in particulate form, in suspension in a liquid matrix. In some embodiments, a liquid composition may include a gel formulation. In other embodiments, the liquid composition is aqueous.

Aqueous suspensions can also contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Useful compositions can also include a mucoadhesive polymer, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran.

Compositions may also include solubilizing agents to aid in the solubility of a compound described herein, such as a compound of Formula (I). The term “solubilizing agent” generally includes agents that result in formation of a micellar solution or a true solution of the agent. Certain acceptable nonionic surfactants, for example polysorbate 80, can be useful as solubilizing agents, as can ophthalmically acceptable glycols, polyglycols, e.g., polyethylene glycol 400, and glycol ethers.

Compositions may also include one or more pH adjusting agents or buffering agents, including acids such as acetic acid, boric acid, citric acid, lactic acid, phosphoric acid and hydrochloric acid; bases such as sodium hydroxide, sodium carbonate, sodium bicarbonate, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other compositions may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other compositions may include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other compositions may include one or more antioxidants to enhance chemical stability where required. Suitable antioxidants include, by way of example only, ascorbic acid and sodium metabisulfite.

Aqueous suspension compositions can be packaged in single-dose non-reclosable containers. Alternatively, multiple-dose reclosable containers can be used, in which case it is typical to include a preservative in the composition.

Alternatively, other delivery systems for hydrophobic pharmaceutical compounds may be employed. Liposomes and emulsions are well known examples of delivery vehicles or carriers for hydrophobic drugs. Certain organic solvents such as N-methylpyrrolidone also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds over the course of 4-24 hours. Depending on the chemical nature and the biological stability of the therapeutic reagent, additional strategies for protein stabilization may be employed.

All of the formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

### Methods of Dosing and Treatment Regimens

For convenience, the methods of dosing and treatment regimens described in this section and other parts herein use a single formula, such as “Formula (I),” by way of example. In addition, the methods of dosing and treatment regimens described herein apply equally well to all formulae presented herein that fall within the scope of Formula (I). For example, the methods of dosing and treatment regimens described herein can be applied to compounds having the structure of any of Formulas (I) or (II), as well as to all of the specific compounds that fall within the scope of these generic formulae and any compounds specifically identifed herein.

The compounds described herein, such as compounds of Formula (I), can be used in the preparation of medicaments for the treatment of leukotriene-dependent or leukotriene mediated diseases or conditions. In addition, a method for treating any of the diseases or conditions described herein in a subject in need of such treatment, involves administration of pharmaceutical compositions containing at least one compound described herein, such as a compound of Formula (I), or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In the case wherein the patient's condition does not improve, upon the doctor's discretion the administration of the compounds described herein may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds described herein may be given continuously; alternatively, the dose of the compounds described herein being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, the dosage or the frequency of administration, or both, can be reduced, as a function of the symptoms, to a level at which the improved state of the disease, disorder or condition is maintained. Patients can, however, require intermittent treatment on a long-term basis upon any recurrence of symptoms.

The amount of a given agent that will correspond to such an amount will vary depending upon factors such as the particular compound, disease condition and its severity, the identity (e.g., age, weight, gender, etc.) of the subject or host in need of treatment, but can nevertheless be determined in a manner known in the art according to the particular circumstances surrounding the case, including, e.g., the specific agent being administered, the route of administration, the condition being treated, and the subject or host being treated. In general, however, doses employed for adult human treatment will typically be in the range of 0.02-5000 mg per day, in some embodiments 1-1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The daily dosages appropriate for the compounds described herein are from about 0.01 to 2.5 mg/kg per body weight. An indicated daily dosage in the larger mammal, including, but not limited to, humans, is in the range from about 0.5 mg to about 100 mg, conveniently administered in divided doses, including, but not limited to, up to four times a day or in extended release form. Suitable unit dosage forms for oral administration include from about 1 mg to about 50 mg active ingredient. The foregoing ranges are merely suggestive, as the number of variables in regard to an individual treatment regime is large, and considerable excursions from these recommended values are not uncommon Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

### Combination Treatments

For convenience, the combination treatments described in this section and other parts herein use a single formula, such as “Formula (I),” by way of example. In addition, the combination treatments described herein apply equally well to all formulae presented herein that fall within the scope of Formula (I). For example, the combination treatments described herein can be applied to compounds having the structure of any of Formulas (I) or (II), as well as to all of the specific compounds that fall within the scope of these generic formulae and any compounds specifically identifed herein.

In certain instances, it may be appropriate to administer at least one compound of Formula (I) in combination with another therapeutic agent. By way of example only, if one of the side effects experienced by a patient upon receiving one of the compounds herein is inflammation, then it may be appropriate to administer an anti-inflammatory agent in combination with the initial therapeutic agent. Or, by way of example only, the therapeutic effectiveness of one of the compounds described herein may be enhanced by administration of an adjuvant (i.e., by itself the adjuvant may have minimal therapeutic benefit, but in combination with another therapeutic agent, the overall therapeutic benefit to the patient is enhanced). Or, by way of example only, the benefit experienced by a patient may be increased by administering one of the compounds described herein with another therapeutic agent (which also includes a therapeutic regimen) that also has therapeutic benefit. By way of example only, in a treatment for asthma involving administration of one of the compounds described herein, increased therapeutic benefit may result by also providing the patient with other therapeutic agents or therapies for asthma. In any case, regardless of the disease, disorder or condition being treated, the overall benefit experienced by the patient may simply be additive of the two therapeutic agents or the patient may experience a synergistic benefit.

It is known to those of skill in the art that therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature.

A combination treatment regimen may encompasses treatment regimens in which administration of a 5-lipoxygenase inhibitor described herein is initiated prior to, during, or after treatment with a second agent described above, and continues until any time during treatment with the second agent or after termination of treatment with the second agent. It also includes treatments in which a 5-lipoxygenase inhibitor described herein and the second agent being used in combination are administered simultaneously or at different times and/or at decreasing or increasing intervals during the treatment period. Combination treatment further includes periodic treatments that start and stop at various times to assist with the clinical management of the patient. For example, a 5-lipoxygenase inhibitor described herein in the combination treatment can be administered weekly at the onset of treatment, decreasing to biweekly, and decreasing further as appropriate.

Compositions and methods for combination therapy are provided herein. In accordance with one aspect, the pharmaceutical compositions disclosed herein are used to treat leukotriene-dependent or leukotriene mediated conditions. In accordance with another aspect, the pharmaceutical compositions disclosed herein are used to treat respiratory diseases, where treatment with a 5-lipoxygenase inhibitor is indicated, in particular asthma, and to induce bronchodilation in a subject. In one embodiment, pharmaceutical compositions disclosed herein are used to treat a subject suffering from a vascular inflammation-driven disorder. In one embodiment, the pharmaceutical compositions disclosed herein are used to treat a subject susceptible to myocardial infarction (MI).

Combination therapies described herein can be used as part of a specific treatment regimen intended to provide a beneficial effect from the co-action of a 5-lipoxygenase inhibitors described herein and a concurrent treatment. It is understood that the dosage regimen to treat, prevent, or ameliorate the condition(s) for which relief is sought, can be modified in accordance with a variety of factors. These factors include the type of respiratory disorder and the type of bronchodilation from which the subject suffers, as well as the age, weight, sex, diet, and medical condition of the subject. Thus, the dosage regimen actually employed can vary widely and therefore can deviate from the dosage regimens set forth herein.

For combination therapies described herein, dosages of the co-administered compounds will of course vary depending on the type of co-drug employed, on the specific compound employed, on the disease or condition being treated and so forth. In addition, when co-administered with one or more biologically active agents, the compound provided herein may be administered either simultaneously with the biologically active agent(s), or sequentially. If administered sequentially, the attending physician will decide on the appropriate sequence of administering the compound described herein, such as a compound of Formula (I), in combination with the biologically active agent(s).

In any case, the multiple therapeutic agents (one of which is one of the compounds described herein, such as a compound of Formula (I) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents; the use of multiple therapeutic combinations are also envisioned.

In addition, the compounds described herein, such as compounds of Formula (I), may also be used in combination with procedures that may provide additional or synergistic benefit to the patient. By way of example only, patients are expected to find therapeutic and/or prophylactic benefit in the methods described herein, wherein pharmaceutical composition of Formula (I) and/or combinations with other therapeutics are combined with genetic testing to determine whether that individual is a carrier of a mutant gene that is known to be correlated with certain diseases or conditions.

The compounds described herein, such as compounds of Formula (I), and combination therapies can be administered before, during or after the occurrence of a disease or condition, and the timing of administering the composition(s) containing a compound can vary. Thus, for example, the compounds can be used as a prophylactic and can be administered continuously to subjects with a propensity to develop conditions or diseases in order to prevent the occurrence of the disease or condition. The compounds and compositions can be administered to a subject during or as soon as possible after the onset of the symptoms. The administration of the compounds can be initiated within the first 48 hours of the onset of the symptoms, in some embodiments within the first 48 hours of the onset of the symptoms, in other embodiments within the first 6 hours of the onset of the symptoms, and yet in other embodiments within 3 hours of the onset of the symptoms. The initial administration can be via any route practical, such as, for example, an intravenous injection, a bolus injection, infusion over 5 minutes to about 5 hours, a pill, a capsule, transdermal patch, buccal delivery, a solution, suspension, and the like, or combination thereof. A compound is preferably administered as soon as is practicable after the onset of a disease or condition is detected or suspected, and for a length of time necessary for the treatment of the disease, such as, for example, from about 1 day to about 3 months. The length of treatment can vary for each subject, and the length can be determined using known criteria. For example, the compound or a formulation containing the compound can be administered for at least 2 weeks, in some embodiments from about 1 month to about 5 years, and in other embodiments from about 1 month to about 3 years.

By way of example, therapies which combine compounds of Formula (I) with inhibitors of leukotriene synthesis or leukotriene receptor antagonists, either acting at the same or other points in the leukotriene synthesis pathway, could prove to be particularly useful for treating leukotriene-dependent or leukotriene mediated diseases or conditions. In addition, by way of example, therapies which combine compounds of Formula (I) with inhibitors of inflammation could prove to be particularly useful for treating leukotriene-dependent or leukotriene mediated diseases or conditions.

Anti-Inflammatory Agents

In another embodiment described herein, methods for treatment of leukotriene-dependent or leukotriene mediated conditions or diseases include administering to a patient compounds, pharmaceutical compositions, or medicaments described herein in combination with an anti-inflammatory agent including, but not limited to, non-steroidal anti-inflammatory drugs (NSAIDs) and corticosteroids (glucocorticoids).

NSAIDs include, but are not limited to: aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, COX-2 specific inhibitors (such as, but not limited to, celecoxib, rofecoxib, valdecoxib, parecoxib, etoricoxib, CS-502, JTE-522, L-745,337 and NS398).

Corticosteroids, include, but are not limited to: betamethasone (Celestone), prednisone (Deltasone), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone/cortisol, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone/prednisolone, rimexolone, tixocortol, triamcinolone, and ulobetasol.

Corticosteroids do not directly inhibit leukotriene production, therefore co-dosing with steroids could provide additional anti-inflammatory benefit.

Some commercially available anti-inflammatories include, but are not limited to: diclofenac and misoprostol (Arthrotec®), 5-aminosalicyclic acid (Asacol®, Salofalk®), antipyrine and benzocaine (Auralgan®), sulfasalazine (Azulfidine®), oxaprozin (Daypro®), etodolac (Lodine®), mefenamic acid (Ponstan®), methylprednisolone (Solumedrol®), aspirin (Bayer®, Bufferin®), indomethacin (Indocin®), rofecoxib (Vioxx®), celecoxib (Celebrex®), valdecoxib (Bextra®), etoricoxib (Arcoxia®), lumiracoxib (Prexige®), ibuprofen (Advil®, Motrin®), diclofenac (Voltaren®), ketoprofen (Orudis®), meloxicam (Mobic®), nabumetone (Relafen®), naproxen (Aleve®, Naprosyn®), piroxicam (Feldene®).

By way of example, asthma is a chronic inflammatory disease characterized by pulmonary eosinophilia and airway hyperresponsiveness. Zhao et al., Proteomics, Jul. 4, 2005. In patients with asthma, leukotrienes may be released from mast cells, eosinophils, and basophils. The leukotrienes are involved in contraction of airway smooth muscle, an increase in vascular permeability and mucus secretions, and have been reported to attract and activate inflammatory cells in the airways of asthmatics (Siegel et al., ed., Basic Neurochemistry, Molecular, Cellular and Medical Aspects, Sixth Ed., Lippincott Williams & Wilkins, 1999). Thus, in another embodiment described herein, the methods for treatment of respiratory diseases include administering to a patient compounds, pharmaceutical compositions, or medicaments described herein in combination with an anti-inflammatory agent.

Leukotriene Receptor Antagonists

In another embodiment described herein, methods for treatment of leukotriene-dependent or leukotriene mediated conditions or diseases include administering to a patient compounds, pharmaceutical compositions, or medicaments described herein in combination with leukotriene receptor antagonists including, but are not limited to, CysLT₁/CysLT₂ dual receptor antagonists and CysLT₁ receptor anatagonists. In another embodiment described herein, methods for treatment of leukotriene-dependent or leukotriene mediated conditions or diseases include administering to a patient compounds, pharmaceutical compositions, or medicaments described herein in combination with a CysLT₁/CysLT₂ dual receptor antagonist. CysLT₁/CysLT₂ dual receptor antagonists include, but are not limited to, BAY u9773 (4-((4S,5R,6E,8E,10Z,13Z)-1-carboxy-4-hydroxynonadeca-6,8,10,13-tetraen-5-ylthio)benzoic acid) (EP 00791576), DUO-LT (6-((4-(2-carbamoylacetyl)phenyl)sulfanyl)-6-(4-nonylphenyl)-5-oxohexanoic acid) (Galczenski et al, D38, Poster F4 presented at American Thoracic Society, May 2002, Tsuji et al, Org. Biomol. Chem., 3139-3141, 2003). For a particular patient, the most appropriate formulation or method of use of such combination treatments may depend on the type of leukotriene-dependent or leukotriene mediated disorder, the time period in which the 5-lipoxygenase inhibitor acts to treat the disorder and the time period in which the CysLT₁/CysLT₂ dual receptor antagonist acts to inhibit CysLT receptor activity. By way of example only, such combination treatments may be used for treating a patient suffering from a respiratory disorder.

In another embodiment described herein, methods for treatment of leukotriene-dependent or leukotriene mediated conditions or diseases include administering to a patient compounds, pharmaceutical compositions, or medicaments described herein in combination with a CysLT₁ receptor antagonist. CysLT₁ receptor antagonists include, but are not limited to, zafirlukast (Accolate®), montelukast (Singulair®), prankulast (ONO-1078), and derivatives or analogs thereof. Such combinations may be used to treat leukotriene-dependent or leukotriene mediated disorder, including respiratory disorders.

The co-administration of a 5-lipoxygenase or FLAP inhibitor described herein with a CysLT₁ receptor antagonist or a dual CysLT₁/CysLT₂ receptor antagonist may have therapeutic benefit over and above the benefit derived from the administration of a either a 5-lipoxygenase or FLAP inhibitor or a CysLT₁R antagonist alone. In the case that substantial inhibition of leukotriene production has undesired effects, partial inhibition of this pathway through the amelioration of the effects of the proinflammatory LTB₄ and cysteinyl leukotrienes combined with the block of the CysLT₁ receptor and/or dual CysLT₁/CysLT₂ receptor block may afford substantial therapeutic benefits, particularly for respiratory diseases.

Other Combination Therapies

In another embodiment described herein, methods for treatment of leukotriene-dependent or leukotriene mediated conditions or diseases, such as proliferative disorders, including cancer, include administration to a patient compounds, pharmaceutical compositions, or medicaments described herein in combination with at least one additional agent selected from among: alemtuzumab, arsenic trioxide, asparaginase (pegylated or non-), bevacizumab, cetuximab, platinum-based compounds such as cisplatin, cladribine, daunorubicin/doxorubicin/idarubicin, irinotecan, fludarabine, 5-fluorouracil, gemtuzumab, methotrexate, paclitaxel (Taxol), temozolomide, thioguanine, or classes of drugs including hormones (an antiestrogen, an antiandrogen, or gonadotropin releasing hormone analogues), interferons such as, but not limited to, alpha interferon; nitrogen mustards such as, but not limited to, busulfan, melphalan, and mechlorethamine; retinoids such as, but not limited to, tretinoin; topoisomerase inhibitors such as, but not limited to, irinotecan, and topotecan; tyrosine kinase inhibitors such as, but not limited to, gefinitinib, and imatinib; or agents to treat signs or symptoms induced by such therapy including, but not limited to, allopurinol, filgrastim, granisetron ondansetron/palonosetron, and dronabinol.

In another embodiment described herein, methods for treatment of leukotriene-dependent or leukotriene mediated conditions or diseases, such as the therapy of transplanted organs or tissues or cells, include administration to a patient compounds, pharmaceutical compositions, or medicaments described herein in combination with at least one additional agent selected from among azathioprine, a corticosteroid, cyclophosphamide, cyclosporin, dacluzimab, mycophenolate mofetil, OKT3, rapamycin, tacrolimus, and thymoglobulin.

In another embodiment described herein, methods for treatment of leukotriene-dependent or leukotriene mediated conditions or diseases, such as atherosclerosis, include administration to a patient compounds, pharmaceutical compositions, or medicaments described herein in combination with at least one additional agent selected from among HMG-CoA reductase inhibitors (e.g., statins in their lactonized or dihydroxy open acid forms and pharmaceutically acceptable salts and esters thereof, including but not limited to lovastatin; simvastatin; dihydroxy open-acid simvastatin, particularly the ammonium or calcium salts thereof; pravastatin, particularly the sodium salt thereof; fluvastatin, particularly the sodium salt thereof; atorvastatin, particularly the calcium salt thereof; nisvastatin, also referred to as NK-104; rosuvastatin); agents that have both lipid-altering effects and other pharmaceutical activities; HMG-CoA synthase inhibitors; cholesterol absorption inhibitors such as ezetimibe; cholesterol ester transfer protein (CETP) inhibitors, for example JTT-705 and CP529, 414; squalene epoxidase inhibitors; squalene synthetase inhibitors (also known as squalene synthase inhibitors); acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; probucol; niacin; bile acid sequestrants; LDL (low density lipoprotein) receptor inducers; platelet aggregation inhibitors, for example adenosine diphosphate (ADP) receptor (P2Y12 receptor) inhibitors such as clopidogrel (Plavix®), glycoprotein IIb/IIIa fibrinogen receptor antagonists and aspirin; human peroxisome proliferator activated receptor gamma (PPARγ) agonists, including the compounds commonly referred to as glitazones, for example troglitazone, pioglitazone and rosiglitazone and including those compounds included within the structural class known as thiazolidinediones as well as those PPARγ agonists outside the thiazolidinedione structural class; PPARα agonists such as clofibrate, fenofibrate including micronized fenofibrate, and gemfibrozil; PPAR dual α/γ agonists such as 5-[(2,4-dioxo-5-thiazolidinyl)methyl]-2-methoxy-N-[[4-(trifluoromethyl)phenyl]methyl]-benzamide, known as KRP-297; vitamin B6 (also known as pyridoxine) and the pharmaceutically acceptable salts thereof such as the HCl salt; vitamin B12 (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; calcium channel blockers such as nifedipine and diltiazam; endothelian antagonists; agents that enhance ABC1 gene expression; FXR and LXR ligands including both inhibitors and agonists; bisphosphonate compounds such as alendronate sodium; and cyclooxygenase-2 inhibitors, such as, for example, rofecoxib, celecoxib, etoricoxib, and lumiracoxib.

In another embodiment described herein, methods for treatment of leukotriene-dependent or leukotriene mediated conditions or diseases, such as the therapy of stroke, include administration to a patient compounds, pharmaceutical compositions, or medicaments described herein in combination with at least one additional agent selected from COX-2 inhibitors; nitric oxide synthase inhibitors, such as, but not limited to, N-(3-(aminomethyl)benzyl)acetamidine; Rho kinase inhibitors, such as, but not limited to, fasudil; angiotension II type-1 receptor antagonists, including, but not limited to, candesartan, losartan, irbesartan, eprosartan, telmisartan, and valsartan; glycogen synthase kinase 3 inhibitors; sodium or calcium channel blockers, including, but not limited to, crobenetine; p38 MAP kinase inhibitors, including, but not limited to, SKB 239063; thromboxane AX-synthetase inhibitors, including, but not limited to, isbogrel, ozagrel, ridogrel and dazoxiben; statins (HMG CoA reductase inhibitors), including, but not limited to, lovastatin, simvastatin, dihydroxy open-acid simvastatin, pravastatin, fluvastatin, atorvastatin, nisvastatin, and rosuvastatin; neuroprotectants, including free radical scavengers, calcium channel blockers, excitatory amino acid antagonists, growth factors, antioxidants, such as edaravone, vitamin C, vitamin E, 6-hydroxy-2,5,7,8-tetramethylchroman-2-carboxylic acid (TROLOX™), citicoline and minicycline, and reactive astrocyte inhibitors, such as (2R)-2-propyloctanoic acid; beta andrenergic blockers, such as, but not limited to, propranolol, nadolol, timolol, pindolol, labetalol, metoprolol, atenolol, esmolol and acebutolol; NMDA receptor antagonists, including memantine; NR2B antagonists, such as traxoprodil; 5-HT1A agonists; adenosine diphosphate (ADP) receptor (P2Y12 receptor) inhibitors such as clopidogrel (Plavix®); aspirin; receptor platelet fibrinogen receptor antagonists, including tirofiban and lamifiban; thrombin inhibitors; antithrombotics, such as argatroban; antihypertensive agents, such as enalapril; vasodilators, such as cyclandelate; nociceptin antagonists; DPIV antagonists; GABA 5 inverse agonists; and selective androgen receptor modulators.

In another embodiment described herein, methods for treatment of leukotriene-dependent or leukotriene mediated conditions or diseases, such as the therapy of pulmonary fibrosis, include administration to a patient compounds, pharmaceutical compositions, or medicaments described herein in combination with at least one additional agent selected from among anti-inflammatory agents, such as corticosteroids, azathioprine, and cyclophosphamide.

In another embodiment described herein, methods for treatment of leukotriene-dependent or leukotriene mediated conditions or diseases, such as the therapy of interstitial cystitis, include administration to a patient compounds, pharmaceutical compositions, or medicaments described herein in combination with at least one additional agent selected from among dimethylsulfoxide, omalizumab, and pentosan polysulfate.

In another embodiment described herein, methods for treatment of leukotriene-dependent or leukotriene mediated conditions or diseases, such as the therapy of disorders of bone, include administration to a patient compounds, pharmaceutical compositions, or medicaments described herein in combination with at least one additional agent selected from among minerals, vitamins, bisphosphonates, anabolic steroids, parathyroid hormone or analogs, and cathepsin K inhibitors.

In another embodiment, described herein, methods for treatment of leukotriene-dependent or leukotriene-mediated conditions or diseases, such as the therapy of painful disorders such as arthritis, including administration to a patient compounds, pharmaceutical compositions, topically applied gels or ointments or medicaments described herein in combination with at least one additional agent selected from among NSAIDs, opiods or anesthetics.

Diagnostic Methods for Patient Identification

For convenience, the diagnostic and/or patient identification methods and treatment methods resulting therefrom that are described in this section and other parts herein use a single formula, such as “Formula (I),” by way of example. In addition, the diagnostic and/or patient identification methods and treatment methods resulting therefrom that are described herein apply equally well to all formulae presented herein that fall within the scope of Formula (I). For example, the diagnostic and/or patient identification methods and treatment methods resulting therefrom that are described herein can be applied to compounds having the structure of any of Formulas (I) or (II), as well as to all of the specific compounds that fall within the scope of these generic formulae and any compounds specifically identifed herein.

The screening of “leukotriene-responsive patients” which may be selected for treatment with compounds described herein, such as compounds of Formula (I), or pharmaceutical compositions or medicaments described herein which include compounds of Formula (I), or other 5-lipoxygenase modulators, may be accomplished using techniques and methods described herein. Such techniques and methods include, by way of example, evaluation of gene haplotypes (genotype analysis), monitoring/measurement of biomarkers (phenotype analysis), monitoring/measurement of functional markers (phenotype analysis), which indicate patient response to known modulators of the leukotriene pathway, or any combination thereof.

Phenotype Analysis: Biomarkers

Patients who are under consideration for treatment with compounds described herein, such as compounds of Formula (I), or drug combinations described herein that include compounds described herein, such as compounds of Formula (I), may be screened for potential responsiveness to treatment based on leukotriene-driven inflammatory biomarker phenotypes.

Patient screening based on leukotriene-driven inflammatory biomarker phenotypes may be used as an alternative to, or it may be complimentary with, patient screening by leukotriene pathway gene haplotype detection. The term “biomarker” as used herein refers to a characteristic which can be measured and evaluated as an indicator of normal biological processes, pathological processes, or pharmacological responses to therapeutic intervention. Thus a biomarker may be any substance, structure or process which can be measured in the body, or its products, and which may influence or predict the incidence of outcome or disease. Biomarkers may be classified into markers of exposure, effect, and susceptibility. Biomarkers can be physiologic endpoints, by way of example blood pressure, or they can be analytical endpoints, by way of example, blood glucose, or cholesterol concentrations. Techniques, used to monitor and/or measure biomarkers include, but are not limited to, NMR, LC-MS, LC-MS/MS, GC-MS, GC-MS/MS, HPLC-MS, HPLC-MS/MS, FT-MS, FT-MS/MS, ICP-MS, ICP-MS/MS, peptide/protein sequencing, nucleic acid sequencing, electrophoresis techniques, immuno-assays, immuno-blotting, in-situ hybridization, fluorescence in-situ hybridization, PCR, radio-immuno assays, and enzyme-immuno assays. Single nucleotide polymorphisms (SNPs) have also been useful for the identification of biomarkers for propensity to certain diseases and also susceptibility or responsiveness to drugs such as chemotherapeutic agents and antiviral agents. These techniques, or any combination thereof, may be used to screen patients for leukotriene-dependent or leukotriene mediated diseases or conditions, wherein such patients may be beneficially treated with compounds described herein, such as compounds of Formula (I), or drug combinations described herein that include compounds described herein, such as compounds of Formula (I).

By way of example only, patients may be selected for treatment with compounds described herein, such as compounds of Formula (I), or drug combinations described herein that include compounds described herein, such as compounds of Formula (I), by screening for enhanced inflammatory blood biomarkers such as, but not limited to, stimulated LTB₄, LTC₄, LTE₄, myeloperoxidase (MPO), eosinophil peroxidase (EPO), C-reactive protein (CRP), soluble intracellular adhesion molecule (sICAM), monocyte chemoattractant protein (MCP-1), monocyte inflammatory protein (MIP-1α), interleukin-6 (IL-6), the TH2 T cell activators interleukin 4 (IL-4), and 13 (IL-13) and other inflammatory cytokines. In certain embodiments, patients with inflammatory respiratory diseases, including but not limited to, asthma and COPD, or with cardiovascular diseases, are selected as those most likely to be responsive to leukotriene synthesis inhibition using compounds described herein, such as compounds of Formula (I), by using a panel of leukotriene driven inflammatory biomarkers.

Phenotype Analysis: Functional Markers

Patients who are under consideration for treatment with compounds described herein, such as compounds of Formula (I), or drug combinations described herein that include compounds described herein, such as compounds of Formula (I), may be screened for response to known modulators of the leukotriene pathway. Patient screening by evaluation of functional markers as indicators of a patient's response to known modulators of the leukotriene pathway may be used as an alternative to, or it may be complimentary with, patient screening by leukotriene pathway gene haplotype detection (genotype analysis) and/or monitoring/measurement of leukotriene-driven inflammatory biomarker phenotypes. Functional markers may include, but are not limited to, any physical characteristics associated with a leukotriene dependent condition or disease, or knowledge of current or past drug treatment regimens.

By way of example only, the evaluation of lung volume and/or function may be used as a functional marker for leukotriene-dependent or leukotriene mediated diseases or conditions, such as respiratory diseases. Lung function tests may be used to screen patients, with such leukotriene-dependent or leukotriene mediated diseases or conditions, for treatment using compounds described herein, such as compounds of Formula (I) or pharmaceutical compostitons or medicaments which include compounds of Formula (I). Such tests include, but are not limited to, evaluation of lung volumes and capacities, such as tidal volume, inspiratory reserve volume, expiratory reserve volume, residual volume, inspiratory capacity, functional residual capacity, vital capacity, total lung capacity, respiratory minute volume, alveolar ventilation, timed vital capacity, and ventilatory capacity. Method of measurement of lung volumes and capacities include, but are not limited to, maximum expiratory flow volume curve, forced expiratory volume in 1 sec. (FEV1), peak expiratory flow rate. In addition, other lung function tests used as functional markers for patient evaluation described herein include, but are not limited to, respiratory muscle power, maximum inspiratory pressure, maximum expiratory pressure, transdiaphragmatic pressure, distribution of ventilation, single breath nitrogen test, pulmonary nitrogen washout, and gas transfer.

Additionally, the knowledge of a patients past or current treatment regimen may be used as a functional marker to assist in screening patients for treatment of leukotriene dependent conditions or diseases using compounds of Formula (I) or pharmaceutical compositions or medicaments that include compounds of Formula (I). By way of example only, such treatment regimens may include past or current treatment using zileuton (Zyflo®), montelukast (Singulair®), pranlukast, zafirlukast (Accolate®).

Also, patients who are under consideration for treatment with compounds described herein, such as compounds of Formula (I), or drug combinations described herein that include compounds described herein, such as compounds of Formula (I), may be screened for functional markers which include, but are not limited to, reduced eosinophil and/or basophil, and/or neutrophil, and/or monocyte and/or dendritic cell and/or lymphocyte recruitment, decreased mucosal secretion, decreased mucosal edema, and/or increased bronchodilation.

Methods for the identification of a patient in need of treatment for leukotriene-dependent or leukotriene mediated conditions or diseases, and exemplary, non-limiting treatment methods are shown in FIGS. 5-8, wherein a patient sample is analyzed and the information obtained is used to identify possible treatment methods. It is expected that one skilled in the art will use this information in conjunction with other patient information, including, but not limited to age, weight, sex, diet, and medical condition, to choose a treatment method. It is also expected that each piece of information will be given a particular weight in the decision process. In certain embodiments, the information obtained from the diagnostic methods described above and any other patient information, including, but not limited to age, weight, sex, diet, and medical condition, are incorporated into an algorithm used to elucidate a treatment method, wherein each piece of information will be given a particular weight in the decision process.

In certain embodiments a patient sample is analyzed for leukotriene gene haplotypes and the information obtained identifies a patient in need of treatment using various treatment methods. Such treatment methods include, but are not limited to, administering a therapeutic effective amount of a compound of Formula (I) or pharmaceutical composition or medicament which includes a compound of Formula (I), administering a therapeutic effective amount of a compound of Formula (I) or pharmaceutical composition or medicament which includes a compound of Formula (I), in combination with a therapeutic effective amount of a leukotriene receptor antagonist (by way of example, CysLT₁/CysLT₂ antagonist or CysLT₁ antagonist), or administering a therapeutic effective amount of a compound of Formula (I) or pharmaceutical composition or medicament which includes a compound of Formula (I), in combination with a therapeutic effective amount of another anti-inflammatory agent. In other embodiments a patient sample is analyzed for leukotriene gene haplotypes, and/or phenotype biomarkers, and/or phenotype functional marker responses to leukotriene modifying agents. The patient may then be treated using various treatment methods. Such treatment methods include, but are not limited to, administering a therapeutic effective amount of a compound of Formula (I) or pharmaceutical composition or medicament which includes a compound of Formula (I), administering a therapeutic effective amount of a compound of Formula (I) or pharmaceutical composition or medicament which includes a compound of Formula (I), in combination with a therapeutic effective amount of a leukotriene receptor antagonist (by way of example, CysLT₁/CysLT₂ antagonist or CysLT₁ antagonist), or administering a therapeutic effective amount of a compound of Formula (I) or pharmaceutical composition or medicament which includes a compound of Formula (I), in combination with a therapeutic effective amount of another anti-inflammatory agent. In still other embodiments a patient sample is analyzed for leukotriene gene haplotypes, and phenotype biomarkers, and phenotype functional marker responses to leukotriene modifying agents. The patient may then be treated using various treatment methods. Such treatment methods include, but are not limited to, administering a therapeutic effective amount of a 5-lipoxygenase inhibitor, or pharmaceutical composition or medicament that includes a 5-lipoxygenase inhibitor, administering a therapeutic effective amount of a 5-lipoxygenase inhibitor, or pharmaceutical composition or medicament which includes a 5-lipoxygenase inhibitor, in combination with a therapeutic effective amount of a leukotriene receptor antagonist (by way of example, CysLT₁/CysLT₂ antagonist or CysLT₁ antagonist), or administering a therapeutic effective amount of a 5-lipoxygenase inhibitor, or pharmaceutical composition or medicament that includes a 5-lipoxygenase inhibitor in combination with a therapeutic effective amount of another anti-inflammatory agent.

Kits/Articles of Manufacture

For use in the therapeutic applications described herein, kits and articles of manufacture are also described herein. Such kits can comprise a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease, disorder, or condition that would benefit by inhibition of 5-LO or in which 5-LO activity is a mediator or contributor to the symptoms or cause.

For example, the container(s) can include one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally include an identifying description or label or instructions relating to its use in the methods described herein.

A kit may include one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, the pharmaceutical compositions can be presented in a pack or dispenser device which can contain one or more unit dosage forms containing a compound provided herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a compound provided herein formulated in a compatible pharmaceutical carrier can also be prepared, placed in an appropriate container, and labeled for treatment of an indicated condition.

### EXAMPLES

The person skilled in the art may further appreciate various aspects and advantages of the present disclosure upon review of the following illustrative and non-limiting examples:

The starting material used for the synthesis of the compounds described herein may be synthesized or can be obtained from commercial sources, such as, but not limited to, Aldrich Chemical Co. (Milwaukee, Wis.), or Sigma Chemical Co. (St. Louis, Mo.). The compounds described herein, and other related compounds having different substituents can be synthesized using techniques and materials described herein as well as those that are known to those of skill in the art.

### Example 1

### Synthesis of 3-(4-Fluoro-phenyl)-7-[4-(1-hydroxy-1-trifluoromethyl-propyl)-[1,2,3]triazol-1-ylmethyl]-indolizine-2-carboxylic acid ethyl ester (Compound 1-4)

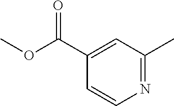

### Step 1: 2-Methyl-isonicotinic acid methyl ester

4-Bromo-2-methylpyridine (70 g, 407 mmol) and triethylamine (141 mL, 1.02 mol) were combined in DMF (400 mL) and MeOH (400 mL). The solution was purged with N₂ for 10 minutes, and then Pd(dppf)₂ (15 g, 20.35 mmol) was added. Carbon monoxide was bubbled through the mixture for 10 minutes, and then the reaction was stirred at 75° C. overnight. The mixture was concentrated and the residue was diluted with 1:1 EtOAc:hexanes (2 L) and washed 10 times with H₂O/brine. The aqueous layer was back-extracted 6 times, and the combined organic layers were dried, filtered, and concentrated. The residue was purified by silica gel chromatography to give the title compound.

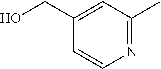

### Step 2: (2-Methyl-pyridin-4-yl)-methanol

A solution of 2-methyl-isonicotinic acid methyl ester (58.41 g, 384 mmol) in THF (750 mL) was cooled to −78° C. Lithium aluminum hydride (2 M in THF, 211.5 mL, 423 mmol) was added dropwise over 1 hour, and the reaction was slowly warmed to room temperature over 2 hours. The reaction was diluted with Et₂O (2 L) and cooled to 0° C. H₂O (16 mL) was added slowly, followed by 15% aqueous NaOH (16 mL), and then additional H₂O (48 mL), and the mixture was stirred for 3 hours at room temperature. MgSO₄ was then added and the mixture was stirred for another 1 hour, then filtered and concentrated to give the title compound.

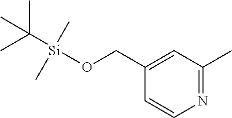 [SMILES: Cc1cc(CO[Si](C)(C)C(C)(C)C)ccn1]

### Step 3: 4-(tert-Butyl-dimethyl-silanyloxymethyl)-2-methyl-pyridine

(2-Methyl-pyridin-4-yl)-methanol (43.51 g, 354 mmol) was dissolved in CH₂Cl₂ (500 mL) and cooled to 0° C. tert-Butyldimethylsilyl chloride (53.32 g, 354 mmol) was added, followed by imidazole (36 g, 531 mmol), and the reaction was warmed to room temperature and stirred for 1 hour. The mixture was filtered through filter paper, and the filtrate was washed 3 times with H₂O/brine, then dried, filtered, and concentrated to give the title compound.

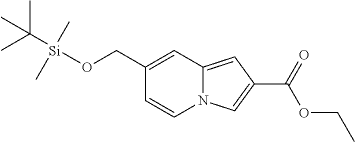 [SMILES: CCOC(=O)c1cc2cc(CO[Si](C)(C)C(C)(C)C)ccn2c1]

### Step 4: 7-(tert-Butyl-dimethyl-silanyloxymethyl)-indolizine-2-carboxylic acid ethyl ester

To 4-(tert-butyl-dimethyl-silanyloxymethyl)-2-methyl-pyridine (10 g, 42 mmol) in MeCN (200 mL) was added ethyl 3-bromopyruvate (6.4 mL, 46.2 mmol), followed by NaHCO₃ (6.7 g, 84 mmol), and the mixture was purged with N₂ for 10 minutes. The reaction was stirred at 140° C. in a sealed tube for 3 hours. The mixture was cooled and concentrated, and the residue was purified by silica gel chromatography (0-50% EtOAc in hexanes) to give the title compound.

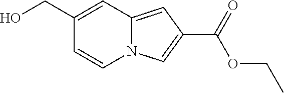 [SMILES: CCOC(=O)c1cc2cc(CO)ccn2c1]

### Step 5: 7-Hydroxymethyl-indolizine-2-carboxylic acid ethyl ester

7-(tert-Butyl-dimethyl-silanyloxymethyl)-indolizine-2-carboxylic acid ethyl ester (13.53 g, 40.5 mmol) in THF (400 mL) was treated with tetrabutylammonium fluoride (1 M in THF, 40.5 mL, 40.5 mmol), and the reaction was stirred at room temperature for 30 minutes. The mixture was diluted with 1:1 EtOAc:hexanes (500 mL) and washed 4 times with H₂O/brine and once with brine. The organic layer was dried, filtered, and concentrated, and the residue was purified by silica gel chromatography (100% EtOAc) to give the title compound.

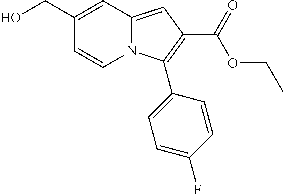 [SMILES: CCOC(=O)c1cc2cc(CO)ccn2c1-c1ccc(F)cc1]

### Step 6: 3-(4-Fluoro-phenyl)-7-hydroxymethyl-indolizine-2-carboxylic acid ethyl ester

7-Hydroxymethyl-indolizine-2-carboxylic acid ethyl ester (5 g, 22.7 mmol) was dissolved in NMP (45 mL), and the solution was purged with N₂ for 10 minutes. 1-Bromo-4-fluorobenzene (6.155 mL, 56.8 mmol) and potassium acetate (4.086 g, 45.4 mmol) were added while the solution was being purged, and then Pd(PPh₃)₂Cl₂ (1.5 g, 22.7 mmol) and H₂O (0.825 mL) were added, and the reaction was stirred at 100° C. in a sealed tube for 2 hours. The mixture was diluted with 1:1 EtOAc:hexanes (500 mL) and washed 4 times with H₂O/brine, and then dried, filtered, and concentrated. The residue was purified by silica gel chromatography (0-100% EtOAc in hexanes) to give the title compound, as well as some oxidized product. The oxidized product (0.688 g, 2.2 mmol) was dissolved in EtOH and treated with sodium borohydride (0.163 g, 4.4 mmol). Aqueous work-up provided the desired product, which was combined with the previously isolated product and repurified by silica gel chromatography (0-100% EtOAc in hexanes) to give the title compound.

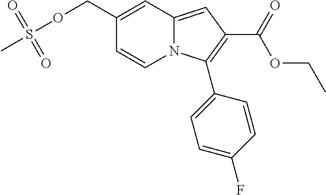 [SMILES: CCOC(=O)c1cc2cc(COS(C)(=O)=O)ccn2c1-c1ccc(F)cc1]

### Step 7: 3-(4-Fluoro-phenyl)-7-methanesulfonyloxymethyl-indolizine-2-carboxylic acid ethyl ester

3-(4-Fluoro-phenyl)-7-hydroxymethyl-indolizine-2-carboxylic acid ethyl ester (6.379 g, 20.3 mmol) was dissolved in CH₂Cl₂ (200 mL) and cooled to −10° C. Triethylamine (16.9 mL, 121.8 mmol) was added, followed by methanesulfonyl chloride (4.71 mL, 60.9 mmol), and the reaction was stirred for 5 minutes and then quenched with saturated aqueous NaHCO₃. The mixture was extracted with EtOAc, and the combined organic layers were washed with saturated aqueous NaHCO₃ and brine, and then dried, filtered, and concentrated to give the title compound.

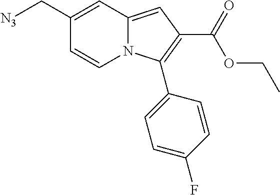 [SMILES: CCOC(=O)c1cc2cc(CN=[N+]=[N-])ccn2c1-c1ccc(F)cc1]

### Step 8: 7-Azidomethyl-3-(4-fluoro-phenyl)-indolizine-2-carboxylic acid ethyl ester

3-(4-Fluoro-phenyl)-7-methanesulfonyloxymethyl-indolizine-2-carboxylic acid ethyl ester (20.3 mmol) was dissolved in DMF (100 mL) and treated with sodium azide (1.979 g, 30.45 mmol). The reaction was stirred at room temperature for 10 minutes, and then diluted with 1:1 EtOAc:hexanes. The mixture was washed with H₂O/brine, and the aqueous layer was back-extracted with EtOAc. The combined organic layers were dried, filtered, and concentrated, and the residue was purified by silica gel chromatography (0-40% EtOAc in hexanes) to give the title compound.

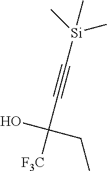

### Step 9: 3-Trifluoromethyl-1-trimethylsilanyl-pent-1-yn-3-ol

To (trimethylsilyl)acetylene (2.45 g, 25 mmol) in THF (100 mL) at −78° C. was added n-butyllithium (1.6 M in hexanes, 19 mL, 30 mmol) dropwise. After stirring for 1 hour at −78° C., 1,1,1-trifluoro-2-butanone (4 mL, 30 mmol) in THF (10 mL) was added dropwise, and the reaction was slowly warmed to room temperature. The mixture was diluted with Et₂O (300 mL) and washed with H₂O, and then dried, filtered, and concentrated to give the title compound.

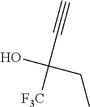

### Step 10: 3-Trifluoromethyl-pent-1-yn-3-ol

3-Trifluoromethyl-1-trimethylsilanyl-pent-1-yn-3-ol (3.67 g, 16.4 mmol) in MeCN (100 mL) was treated with cesium fluoride (2.62 g, 17.2 mmol), and the reaction was stirred at room temperature for 15 minutes. The mixture was diluted with Et₂O (300 mL) and washed with H₂O and brine. The combined aqueous layers were back-extracted with Et₂O, and the mixture was concentrated to ˜300 mL. THF (500 mL) was added, and the mixture was concentrated to ˜100 mL and then used directly in the following step.

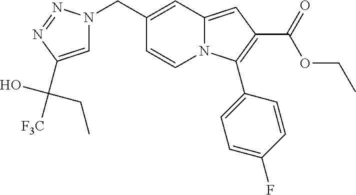 [SMILES: CCOC(=O)c1cc2cc(Cn3cc(C(O)(CC)C(F)(F)F)nn3)ccn2c1-c1ccc(F)cc1]

### Step 11: 3-(4-Fluoro-phenyl)-7-[4-(1-hydroxy-1-trifluoromethyl-propyl)-[1,2,3]triazol-1-ylmethyl]-indolizine-2-carboxylic acid ethyl ester

To 7-azidomethyl-3-(4-fluoro-phenyl)-indolizine-2-carboxylic acid ethyl ester (2.777 g, 8.2 mmol) and copper iodide (2.33 g, 12.3 mmol) was added a solution of 3-trifluoromethyl-pent 1-yn-3-ol (16.3 mmol) in THF (100 mL), followed by diisopropylethylamine (8.5 mL, 49.2 mmol), and the reaction was stirred at room temperature for 1.5 hours. The mixture was diluted with EtOAc and washed 4 times with saturated aqueous NH₄Cl, and then dried, filtered, and concentrated to give the title compound 1-4.

### Example 2

### Synthesis of 3-(4-Fluoro-phenyl)-7-[4-(1-hydroxy-1-trifluoromethyl-propyl)-[1,2,3]triazol-1-ylmethyl]-indolizine-2-carboxylic acid amide (Enantiomer A) (Compound 1-9)

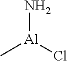

### Step 1: Methylchloroaluminum amide

NH₄Cl (0.535 g, 10 mmol) was dissolved in dry toluene (10 mL) at 0° C. Trimethylaluminum (2 M in toluene, 5.0 mL, 10 mmol) was added, and the reaction was warmed to room temperature and stirred for 1 hour to give the title compound, which was used directly as a 0.67 M solution in toluene.

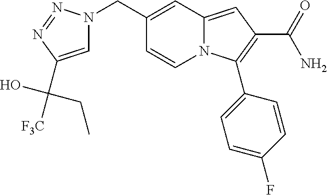 [SMILES: CCC(O)(c1cn(Cc2ccn3c(-c4ccc(F)cc4)c(C(N)=O)cc3c2)nn1)C(F)(F)F]

### Step 2: 3-(4-Fluoro-phenyl)-7-[4-(1-hydroxy-1-trifluoromethyl-propyl)-[1,2,3]triazol-1-ylmethyl]-indolizine-2-carboxylic acid amide (Enantiomer A)

Freshly prepared methylchloroaluminum amide (0.67 M in toluene, 0.45 mL, 0.3 mmol) was added to 3-(4-fluoro-phenyl)-7-[4-(1-hydroxy-1-trifluoromethyl-propyl)-[1,2,3]triazol-1-ylmethyl]-indolizine-2-carboxylic acid ethyl ester (0.050 g, 0.1 mmol), and the reaction was stirred at 75° C. overnight. The mixture was diluted with H₂O and extracted with EtOAc. The combined organic layers were washed twice with H₂O and once with brine, and then dried, filtered, and concentrated. The residue was purified by silica gel chromatography (0-100% EtOAc in hexanes) to give the title compound as a mixture of enantiomers. Chiral HPLC (using ChiralPak AD column, 50/50 hexanes/iPrOH) was used to separate the two enantiomers. The first product to elute from the column (17.67 minutes) is Enantiomer A (Compound 1-9) and the second product to elute from the column (30.19 minutes) is Enantiomer B (Compound 1-10). The first isolated compound was repurified by preparative HPLC to give the title compound 1-9.

### Example 3

### Synthesis of 3-(4-Fluoro-phenyl)-7-{[5-(1-hydroxy-1-trifluoromethyl-propyl)-[1,3,4]oxadiazol-2-ylamino]-methyl}-indolizine-2-carboxylic acid ethyl ester (Compound 1-11)

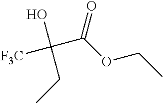

### Step 1: 2-Hydroxy-2-trifluoromethyl-butyric acid ethyl ester

To a solution of ethyl 3,3,3-trifluoropyruvate (50 g, 294 mmol) in Et₂O (500 mL) at −78° C. was added ethylmagnesium bromide (3 M in Et₂O, 98 mL, 294 mmol) over 90 minutes. The reaction was allowed to warm to −10° C. and then poured in saturated aqueous NH₄Cl (1 L). The aqueous layer was extracted 5 times with Et₂O, and the combined organic layers were dried over MgSO₄, filtered, and concentrated to give the title compound.

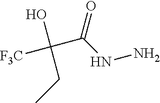

### Step 2: 2-Hydroxy-2-trifluoromethyl-butyric acid hydrazide

2-Hydroxy-2-trifluoromethyl-butyric acid ethyl ester (48 g, 224 mmol) and hydrazine hydrate (25 mL) were combined, and the reaction was stirred at 80° C. for 18 hours. The mixture was concentrated, and the residue was purified by silica gel chromatography (70% EtOAc in hexanes) to give the title compound as an orange oil.

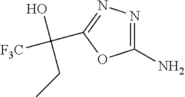

### Step 3: 2-(5-Amino-[1,3,4]oxadiazol-2-yl)-1,1,1-trifluoro-butan-2-ol

To 2-Hydroxy-2-trifluoromethyl-butyric acid hydrazide (28 g, 150 mmol) in H₂O (250 mL) was added NaHCO₃ (12.6 g, 150 mmol). Cyanogen bromide (15.93 g, 150 mmol) was added over 5 minutes, and the reaction was stirred at room temperature overnight, with a precipitate forming after 10 minutes. The mixture was filtered, and the isolated solids were washed with H₂O and dried to give

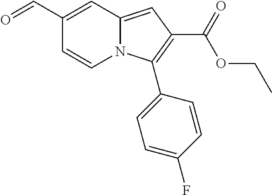 [SMILES: CCOC(=O)c1cc2cc(C=O)ccn2c1-c1ccc(F)cc1]

### Step 4: 3-(4-Fluoro-phenyl)-7-formyl-indolizine-2-carboxylic acid ethyl ester

3-(4-Fluoro-phenyl)-7-hydroxymethyl-indolizine-2-carboxylic acid ethyl ester (0.080 g, 0.26 mmol) in CH₂Cl₂ (17 mL) and MeCN (2 mL) was treated with N-methylmorpholine N-oxide (0.032 g, 0.27 mmol), followed by tetrapropylammonium perruthenate (0.003 g, 0.01 mmol), and the reaction was stirred overnight at room temperature. The mixture was concentrated, and the residue was purified by silica gel chromatography (50% EtOAc in hexanes) to give the title compound.

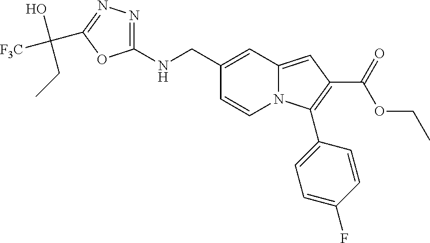 [SMILES: CCOC(=O)c1cc2cc(CNc3nnc(C(O)(CC)C(F)(F)F)o3)ccn2c1-c1ccc(F)cc1]

### Step 5: 3-(4-Fluoro-phenyl)-7-{[5-(1-hydroxy-1-trifluoromethyl-propyl)-[1,3,4]oxadiazol-2-ylamino]-methyl}-indolizine-2-carboxylic acid ethyl ester

3-(4-Fluoro-phenyl)-7-formyl-indolizine-2-carboxylic acid ethyl ester (0.066 g, 0.21 mmol), 2-(5-amino-[1,3,4]oxadiazol-2-yl)-1,1,1-trifluoro-butan-2-ol (0.053 g, 0.25 mmol), and pyridinium p-toluenesulfonate (0.005 g, 0.02 mmol) were combined in toluene (10 mL), and the reaction was stirred at 140° C. for 1 hour, using Dean-Stark apparatus. The mixture was cooled to room temperature and concentrated, and the residue was dissolved in EtOH and cooled to 0° C. Sodium borohydride (0.009 g, 0.23 mmol) was added, and the reaction was stirred at room temperature for 5-10 minutes. The mixture was quenched with H₂O (1 mL) and left overnight at room temperature. After concentrating, the crude material was purified twice by preparative HPLC to give the title compound 1-11.

### Example 4

### Synthesis of 3-(4-Fluoro-phenyl)-7-[4-(1-hydroxy-1-trifluoromethyl-propyl)-[1,2,3]triazol-1-ylmethyl]-indolizine-2-carbonitrile (Compound 1-22)

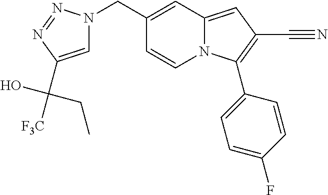 [SMILES: CCC(O)(c1cn(Cc2ccn3c(-c4ccc(F)cc4)c(C#N)cc3c2)nn1)C(F)(F)F]

### Step 1: 3-(4-Fluoro-phenyl)-7-[4-(1-hydroxy-1-trifluoromethyl-propyl)-[1,2,3]triazol-1-ylmethyl]-indolizine-2-carbonitrile

To 3-(4-fluoro-phenyl)-7-[4-(1-hydroxy-1-trifluoromethyl-propyl)-[1,2,3]triazol-1-ylmethyl]-indolizine-2-carboxylic acid amide (0.085 g, 0.18 mmol) in CH₂Cl₂ (4 mL) was added triethylamine (0.055 mL, 0.4 mmol), and the mixture was cooled to 0° C. Trifluoroacetic anhydride (0.033 mL, 0.2 mmol) was added, and the reaction was warmed to room temperature and stirred for 15 minutes. Analytical LCMS indicated that starting material was still present, so the reaction was cooled to 0° C., and additional triethylamine (0.025 mL, 0.3 mmol) and trifluoroacetic anhydride (0.033 mL, 0.2 mmol) were added. Once no starting material was seen by analytical LCMS, the mixture was quenched with H₂O (10 mL) and diluted with EtOAc (10 mL). The organic layer was separated and washed with brine, and the combined aqueous layers were back-extracted with EtOAc. The combined organic layers were dried, filtered, and concentrated, and the residue was purified by silica gel chromatography to give the title compound 1-22.

### Example 5

### Synthesis of 3-(4-Fluoro-phenyl)-7-[4-(1-hydroxy-1-trifluoromethyl-propyl)-[1,2,3]triazol-1-ylmethyl]-indolizine-2-carboxylic acid (Compound 1-5)

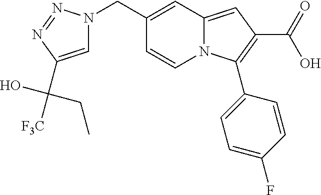 [SMILES: CCC(O)(c1cn(Cc2ccn3c(-c4ccc(F)cc4)c(C(=O)O)cc3c2)nn1)C(F)(F)F]

### Step 1: 3-(4-Fluoro-phenyl)-7-[4-(1-hydroxy-1-trifluoromethyl-propyl)-[1,2,3]triazol-1-ylmethyl]-indolizine-2-carboxylic acid

3-(4-Fluoro-phenyl)-7-[4-(1-hydroxy-1-trifluoromethyl-propyl)-[1,2,3]triazol-1-ylmethyl]-indolizine-2-carboxylic acid ethyl ester (1 g, 2.0 mmol) in MeOH (10 mL) was treated with 1 N aqueous LiOH (2 mL, 2.0 mmol), and the reaction was stirred at 40° C. over the weekend. Analytical LCMS indicated that starting material was still present, so additional 1 N aqueous LiOH (2 mL, 2.0 mmol) was added, and the reaction was stirred at 60° C. for 6 hours. Additional 1N aqueous LiOH (5 mL, 5.0 mmol) was added, and the reaction was complete after 30 minutes. The mixture was quenched with 1 N aqueous HCl and extracted with EtOAc. The combined organic layers were washed with H₂O/brine, dried, filtered, and concentrated to give the title compound 1-5.

### Example 6

### Synthesis of 3-(4-Fluoro-phenyl)-7-[4-(1-hydroxy-1-trifluoromethyl-propyl)-[1,2,3]triazol-1-ylmethyl]-indolizine-2-carboxylic acid methoxy-methyl-amide (Compound 1-51)

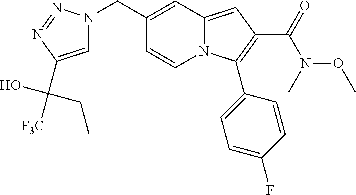 [SMILES: CCC(O)(c1cn(Cc2ccn3c(-c4ccc(F)cc4)c(C(=O)N(C)OC)cc3c2)nn1)C(F)(F)F]

### Step 1: 3-(4-Fluoro-phenyl)-7-[4-(1-hydroxy-1-trifluoromethyl-propyl)-[1,2,3]triazol-1-ylmethyl]-indolizine-2-carboxylic acid methoxy-methyl-amide

3-(4-Fluoro-phenyl)-7-[4-(1-hydroxy-1-trifluoromethyl-propyl)-[1,2,3]triazol-1-ylmethyl]-indolizine-2-carboxylic acid (0.94 g, 2.0 mmol) was dissolved in CH₂Cl₂ (20 mL). N, 0-Dimethylhydroxylamine hydrochloride (0.585 g, 6.0 mmol) was added, followed by EDC (0.573 g, 3.0 mmol), HOBt (0.405 g, 3.0 mmol), and diisopropylethylamine (2.8 mL, 16.0 mmol). The reaction was stirred at room temperature overnight, and then diluted with EtOAc and washed 3 times with H₂O/brine. The aqueous layer was back-extracted with EtOAc, and the combined organic layers were dried, filtered, and concentrated. The residue was purified by silica gel chromatography (0-100% EtOAc in hexanes) to give the title compound 1-51.

### Example 7

### Synthesis of 1-{3-(4-Fluoro-phenyl)-7-[4-(1-hydroxy-1-trifluoromethyl-propyl)-[1,2,3]triazol-1-ylmethyl]-indolizin-2-yl}-ethanone Compound 1-54)

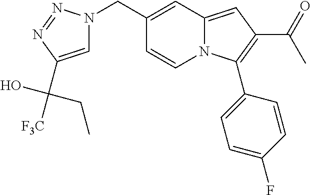 [SMILES: CCC(O)(c1cn(Cc2ccn3c(-c4ccc(F)cc4)c(C(C)=O)cc3c2)nn1)C(F)(F)F]

### Step 1: 1-{3-(4-Fluoro-phenyl)-7-[4-(1-hydroxy-1-trifluoromethyl-propyl)-[1,2,3]triazol-1-ylmethyl]-indolizin-2-yl}-ethanone

3-(4-Fluoro-phenyl)-7-[4-(1-hydroxy-1-trifluoromethyl-propyl)-[1,2,3]triazol-1-ylmethyl]-indolizine-2-carboxylic acid methoxy-methyl-amide (0.050 g, 0.10 mmol) was dissolved in CH₂Cl₂ and cooled to 0° C. under N₂. Methylmagnesium iodide (3 M in Et₂O, 0.066 mL, 0.198 mmol) was added dropwise, and the reaction was stirred for 1 hour. The mixture was quenched with H₂O and dilute acid, and extracted 3 times with EtOAc. The combined organic layers were dried, filtered, and concentrated, and the residue was purified by silica gel chromatography (0-100% EtOAc in hexanes) to give the title compound 1-54.

### Example 8

### Synthesis of 1-Chloro-3-(4-fluoro-phenyl)-7-[4-(1-hydroxy-1-trifluoromethyl-propyl)-[1,2,3]triazol-1-ylmethyl]-indolizine-2-carboxylic acid amide (Compound 1-61)

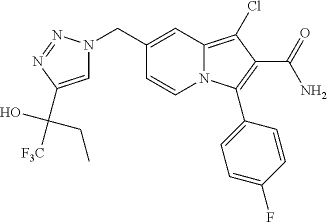 [SMILES: CCC(O)(c1cn(Cc2ccn3c(-c4ccc(F)cc4)c(C(N)=O)c(Cl)c3c2)nn1)C(F)(F)F]

### Step 1: 1-Chloro-3-(4-fluoro-phenyl)-7-[4-(1-hydroxy-1-trifluoromethyl-propyl)-[1,2,3]triazol-1-ylmethyl]-indolizine-2-carboxylic acid amide

3-(4-Fluoro-phenyl)-7-[4-(1-hydroxy-1-trifluoromethyl-propyl)-[1,2,3]triazol-1-ylmethyl]-indolizine-2-carboxylic acid amide (0.060 g, 0.13 mmol) and N-chlorosuccinimide (0.021 g, 0.16 mmol) were dissolved in MeCN (4 mL), and the reaction was stirred for 30 minutes at room temperature. Aqueous work-up followed by silica gel chromatography provided the title compound I-61, which was further purified by preparative HPLC.

### Example 9

### Synthesis of 7-[4-(1-Hydroxy-1-trifluoromethyl-propyl)-[1,2,3]triazol-1-ylmethyl]-3-(6-methyl-pyridin-3-yl)-indolizine-2-carbonitrile (Enantiomer B) (Compound 1-76)

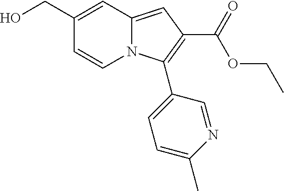 [SMILES: CCOC(=O)c1cc2cc(CO)ccn2c1-c1ccc(C)nc1]

### Step 1: 7-Hydroxymethyl-3-(6-methyl-pyridin-3-yl)-indolizine-2-carboxylic acid ethyl ester

To a solution of 7-hydroxymethyl-indolizine-2-carboxylic acid ethyl ester (0.252 g, 1.15 mmol) in NMP (2.3 mL) was added 5-bromo-2-methylpyridine (0.495 g, 2.87 mmol) and potassium acetate (0.207 g, 2.3 mmol). The mixture was purged with N₂ for 5 minutes, and then Pd(PPh₃)₂Cl₂ (0.080 g, 0.12 mmol) and H₂O (0.041 mL) were added, and the reaction was stirred at 100° C. in a sealed tube for 3.5 hours. The mixture was extracted 4 times with 1:1 EtOAc:hexanes (500 mL), and then dried, filtered, and concentrated. The residue was purified by silica gel chromatography (0-100% EtOAc in hexanes) to give the title compound.

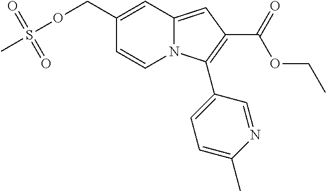 [SMILES: CCOC(=O)c1cc2cc(COS(C)(=O)=O)ccn2c1-c1ccc(C)nc1]

### Step 2: 7-Methanesulfonyloxymethyl-3-(6-methyl-pyridin-3-yl)-indolizine-2-carboxylic acid ethyl ester

Prepared according to the procedure described in Example 1, Step 7, using the following starting materials: 7-hydroxymethyl-3-(6-methyl-pyridin-3-yl)-indolizine-2-carboxylic acid ethyl ester and methanesulfonyl chloride.

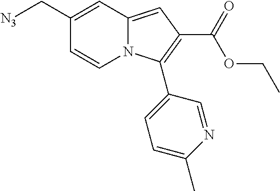 [SMILES: CCOC(=O)c1cc2cc(CN=[N+]=[N-])ccn2c1-c1ccc(C)nc1]

### Step 3: 7-Azidomethyl-3-(6-methyl-pyridin-3-yl)-indolizine-2-carboxylic acid ethyl ester

Prepared according to the procedure described in Example 1, Step 8, using the following starting materials: 7-methanesulfonyloxymethyl-3-(6-methyl-pyridin-3-yl)-indolizine-2-carboxylic acid ethyl ester and sodium azide.

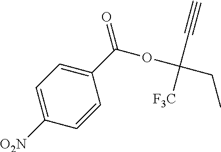 [SMILES: C#CC(CC)(OC(=O)c1ccc([N+](=O)[O-])cc1)C(F)(F)F]

### Step 4: 4-Nitro-benzoic acid 1-ethyl-1-trifluoromethyl-prop-2-ynyl ester (Enantiomer B)

A solution of 3-trifluoromethyl-1-trimethylsilanyl-pent-1-yn-3-ol (0.500 g, 2.23 mmol) in DMF (15 mL) was treated with sodium hydride (60% in mineral oil, 0.090 g, 2.25 mmol), followed by 4-nitrobenzoyl chloride (0.414 g, 2.23 mmol), and the reaction was stirred at room temperature. Aqueous work-up provided the title compound. The material was submitted to chiral HPLC separation to resolve the two enantiomers (Conditions: D-Star HPLC unit with a 5 cm ID×25 cm L CHIRALCEL® OD 20 mm column, hexanes:IPA::95:5, 160 mL/min, 25° C., >99.9 ee. The second isomer to elute from the column is Enantiomer B.

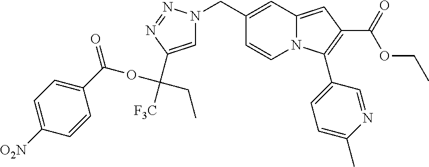 [SMILES: CCOC(=O)c1cc2cc(Cn3cc(C(CC)(OC(=O)c4ccc([N+](=O)[O-])cc4)C(F)(F)F)nn3)ccn2c1-c1ccc(C)nc1]

### Step 5: 3-(6-Methyl-pyridin-3-yl)-7-[4-[1-(4-nitro-benzoyloxy)-1-trifluoromethyl-propyl]-[1,2,3]triazol-1-ylmethyl]-indolizine-2-carboxylic acid ethyl ester (Enantiomer B)

Prepared according to the procedure described in Example 1, Step 11, using the following starting materials: 7-azidomethyl-3-(6-methyl-pyridin-3-yl)-indolizine-2-carboxylic acid ethyl ester and 4-nitro-benzoic acid 1-ethyl-1-trifluoromethyl-prop-2-ynyl ester.

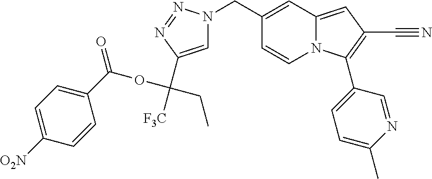 [SMILES: CCC(OC(=O)c1ccc([N+](=O)[O-])cc1)(c1cn(Cc2ccn3c(-c4ccc(C)nc4)c(C#N)cc3c2)nn1)C(F)(F)F]

### Step 6: 4-Nitro-benzoic acid 1-[1-[2-cyano-3-(6-methyl-pyridin-3-yl)-indolizin-7-ylmethyl]-1H-[1,2,3]triazol-4-yl]-1-trifluoromethyl-propyl ester (Enantiomer B)

Freshly prepared methylchloroaluminum amide (4 mL, 2.94 mmol) was added to 3-(6-methyl-pyridin-3-yl)-7-{4-[1-(4-nitro-benzoyloxy)-1-trifluoromethyl-propyl]-[1,2,3]triazol-1-ylmethyl}-indolizine-2-carboxylic acid ethyl ester (0.625 g, 0.98 mmol), and the reaction was stirred at 75° C. for 5 hours. The mixture was quenched with EtOAc and H₂O, and the organic layer was washed 4 times with saturated aqueous NaHCO₃. The combined organic layers were back-extracted twice with EtOAc, and the combined organic layers were dried, filtered, and concentrated. The crude material was purified by silica gel chromatograph y (0-100% EtOAc in hexanes, followed by 5% MeOH in CH₂Cl₂) to give a separable mixture of two compounds, of which the major compound isolated was the title compound. The minor compound, 4-nitro-benzoic acid 1-{1-[2-carbamoyl-3-(6-methyl-pyridin-3-yl)-indolizin-7-ylmethyl]-1H-[1,2,3]triazol-4-yl}-1-trifluoromethyl-propyl ester (Enantiomer B), was also isolated.

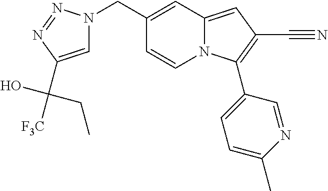 [SMILES: CCC(O)(c1cn(Cc2ccn3c(-c4ccc(C)nc4)c(C#N)cc3c2)nn1)C(F)(F)F]

### Step 7: 7-[4-(1-Hydroxy-1-trifluoromethyl-propyl)-[1,2,3]triazol-1-ylmethyl]-3-(6-methyl-pyridin-3-yl)-indolizine-2-carbonitrile (Enantiomer B)

4-Nitro-benzoic acid 1-{1-[2-cyano-3-(6-methyl-pyridin-3-yl)-indolizin-7-ylmethyl]-1H-[1,2,3]triazol-4-yl}-1-trifluoromethyl-propyl ester (0.293 g, 0.5 mmol) in MeOH (10 mL) was treated with 1 N aqueous LiOH (1 mL, 1.0 mmol), and the reaction was stirred at room temperature for 10 minutes. The mixture was extracted 4 times with EtOAc, and the combined organic layers were dried, filtered, and concentrated. The residue was purified by preparative HPLC to give the title compound.

### Example 10

### Synthesis of 7-[4-(1-Hydroxy-1-trifluoromethyl-propyl)-[1,2,3]triazol-1-ylmethyl]-3-(6-methoxy-pyridin-3-yl)-indolizine-2-carboxylic acid amide (Enantiomer B) (Compound 1-78)

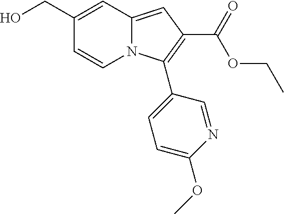 [SMILES: CCOC(=O)c1cc2cc(CO)ccn2c1-c1ccc(OC)nc1]

### Step 1: 7-Hydroxymethyl-3-(6-methoxy-pyridin-3-yl)-indolizine-2-carboxylic acid ethyl ester

Prepared according to the procedure described in Example 9, Step 1, using the following starting materials: 7-hydroxymethyl-indolizine-2-carboxylic acid ethyl ester and 5-bromo-2-methoxypyridine.

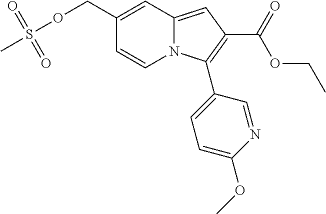 [SMILES: CCOC(=O)c1cc2cc(COS(C)(=O)=O)ccn2c1-c1ccc(OC)nc1]

### Step 2: 7-Methanesulfonyloxymethyl-3-(6-methoxy-pyridin-3-yl)-indolizine-2-carboxylic acid ethyl ester

Prepared according to the procedure described in Example 1, Step 7, using the following starting materials: 7-hydroxymethyl-3-(6-methoxy-pyridin-3-yl)-indolizine-2-carboxylic acid ethyl ester and methanesulfonyl chloride.

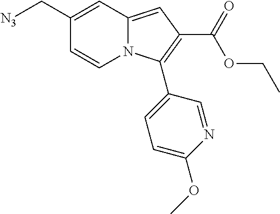 [SMILES: CCOC(=O)c1cc2cc(CN=[N+]=[N-])ccn2c1-c1ccc(OC)nc1]

### Step 3: 7-Azidomethyl-3-(6-methoxy-pyridin-3-yl)-indolizine-2-carboxylic acid ethyl ester

Prepared according to the procedure described in Example 1, Step 8, using the following starting materials: 7-methanesulfonyloxymethyl-3-(6-methoxy-pyridin-3-yl)-indolizine-2-carboxylic acid ethyl ester and sodium azide.

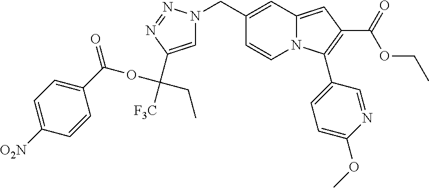 [SMILES: CCOC(=O)c1cc2cc(Cn3cc(C(CC)(OC(=O)c4ccc([N+](=O)[O-])cc4)C(F)(F)F)nn3)ccn2c1-c1ccc(OC)nc1]

### Step 4: 3-(6-Methoxy-pyridin-3-yl)-7-[4-[1-(4-nitro-benzoyloxy)-1-trifluoromethyl-propyl]-[1,2,3]triazol-1-ylmethyl]-indolizine-2-carboxylic acid ethyl ester (Enantiomer B)

Prepared according to the procedure described in Example 1, Step 11, using the following starting materials: 7-azidomethyl-3-(6-methoxy-pyridin-3-yl)-indolizine-2-carboxylic acid ethyl ester and 4-nitro-benzoic acid 1-ethyl 1-trifluoromethyl-prop-2-ynyl ester (Enantiomer B).

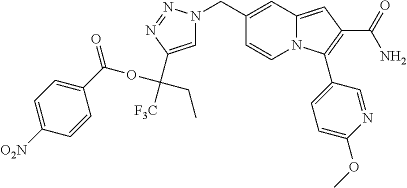 [SMILES: CCC(OC(=O)c1ccc([N+](=O)[O-])cc1)(c1cn(Cc2ccn3c(-c4ccc(OC)nc4)c(C(N)=O)cc3c2)nn1)C(F)(F)F]

### Step 5: 4-Nitro-benzoic acid 1-{1-[2-carbamoyl-3-(6-methoxy-pyridin-3-yl)-indolizin-7-ylmethyl]-1H-[1,2,3]triazol-4-yl}-1-trifluoromethyl-propyl ester (Enantiomer B)

Freshly prepared methylchloroaluminum amide (4 mL, 2.68 mmol) was added to 3-(6-methoxy-pyridin-3-yl)-7-{4-[1-(4-nitro-benzoyloxy)-1-trifluoromethyl-propyl]-[1,2,3]triazol-1-ylmethyl}-indolizine-2-carboxylic acid ethyl ester (Enantiomer B) (0.50 g, 0.77 mmol) in toluene (2.5 mL), and the reaction was stirred at 75° C. for 2.5 hours. Sodium sulfate decahydrate was added and the mixture was diluted with EtOAc and stirred until clarified, and then filtered. The filtrate was concentrated to give the title compound.

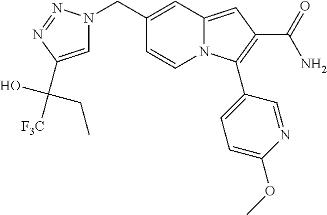 [SMILES: CCC(O)(c1cn(Cc2ccn3c(-c4ccc(OC)nc4)c(C(N)=O)cc3c2)nn1)C(F)(F)F]

### Step 6: 7-[4-(1-Hydroxy-1-trifluoromethyl-propyl)-[1,2,3]triazol-1-ylmethyl]-3-(6-methoxy-pyridin-3-yl)-indolizine-2-carboxylic acid amide (Enantiomer B)

Prepared according to the procedure described in Example 9, Step 7, using the following starting material: 4-nitro-benzoic acid 1-{1-[2-carbamoyl-3-(6-methoxy-pyridin-3-yl)-indolizin-7-ylmethyl]-1H-[1,2,3]triazol-4-yl}-1-trifluoromethyl-propyl ester (Enantiomer B).

### Example 11

### Synthesis of 7-[4-(1-Hydroxy-1-trifluoromethyl-propyl)-[1,2,3]triazol-1-ylmethyl]-3-(6-methoxy-pyridin-3-yl)-indolizine-2-carbonitrile (Enantiomer B) (Compound 1-79)

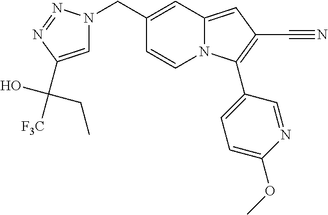 [SMILES: CCC(O)(c1cn(Cc2ccn3c(-c4ccc(OC)nc4)c(C#N)cc3c2)nn1)C(F)(F)F]

### Step 1: 7-[4-(1-Hydroxy-1-trifluoromethyl-propyl)-[1,2,3]triazol-1-ylmethyl]-3-(6-methoxy-pyridin-3-yl)-indolizine-2-carbonitrile (Enantiomer B)

Freshly prepared methylchloroaluminum amide (0.42 mL, 0.28 mmol) was added to a solution of 7-[4-(1-hydroxy-1-trifluoromethyl-propyl)-[1,2,3]triazol-1-ylmethyl]-3-(6-methoxy-pyridin-3-yl)-indolizine-2-carboxylic acid amide (Enantiomer B) (0.042 g, 0.09 mmol) in toluene, and the reaction was stirred at 75° C. for 2.5 hours. Sodium sulfate decahydrate was added, and the mixture was diluted with EtOAc and stirred until clarified, and then filtered. The filtrate was concentrated, and the residue was purified by preparative HPLC to give the title compound.

### Example 12

### Synthesis of 7-[4-(1-Hydroxy-1-trifluoromethyl-propyl)-[1,2,3]triazol-1-ylmethyl]-3-(2-methyl-pyridin-4-yl)-indolizine-2-carbonitrile (Enantiomer B) (Compound 1-80)

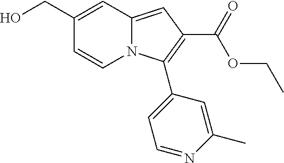 [SMILES: CCOC(=O)c1cc2cc(CO)ccn2c1-c1ccnc(C)c1]

### Step 1: 7-Hydroxymethyl-3-(2-methyl-pyridin-4-yl)-indolizine-2-carboxylic acid ethyl ester

Prepared according to the procedure described in Example 9, Step 1, using the following starting materials: 7-hydroxymethyl-indolizine-2-carboxylic acid ethyl ester and 4-bromo-2-methylpyridine.

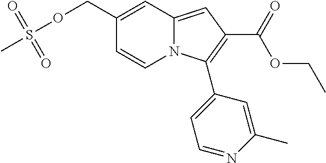 [SMILES: CCOC(=O)c1cc2cc(COS(C)(=O)=O)ccn2c1-c1ccnc(C)c1]

### Step 2: 7-Methanesulfonyloxymethyl-3-(2-methyl-pyridin-4-yl)-indolizine-2-carboxylic acid ethyl ester

Prepared according to the procedure described in Example 1, Step 7, using the following starting materials: 7-hydroxymethyl-3-(2-methyl-pyridin-4-yl)-indolizine-2-carboxylic acid ethyl ester

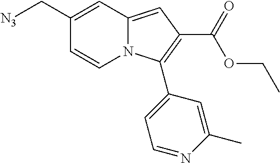 [SMILES: CCOC(=O)c1cc2cc(CN=[N+]=[N-])ccn2c1-c1ccnc(C)c1]

### Step 3: 7-Azidomethyl-3-(2-methyl-pyridin-4-yl)-indolizine-2-carboxylic acid ethyl ester

Prepared according to the procedure described in Example 1, Step 8, using the following starting materials: 7-methanesulfonyloxymethyl-3-(2-methyl-pyridin-4-yl)-indolizine-2-carboxylic acid ethyl ester and sodium azide.

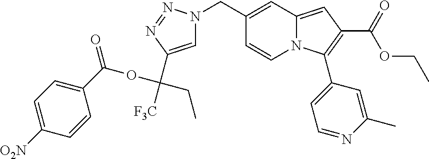 [SMILES: CCOC(=O)c1cc2cc(Cn3cc(C(CC)(OC(=O)c4ccc([N+](=O)[O-])cc4)C(F)(F)F)nn3)ccn2c1-c1ccnc(C)c1]

### Step 4: 3-(2-Methyl-pyridin-4-yl)-7-{4-[1-(4-nitro-benzoyloxy)-1-trifluoromethyl-propyl]-[1,2,3]triazol-1-ylmethyl}-indolizine-2-carboxylic acid ethyl ester (Enantiomer B)

Prepared according to the procedure described in Example 1, Step 11, using the following starting materials: 7-azidomethyl-3-(2-methyl-pyridin-4-yl)-indolizine-2-carboxylic acid ethyl ester and 4-nitro-benzoic acid 1-ethyl-1-trifluoromethyl-prop-2-ynyl ester (Enantiomer B).

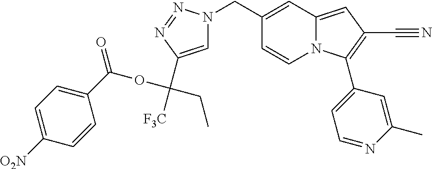 [SMILES: CCC(OC(=O)c1ccc([N+](=O)[O-])cc1)(c1cn(Cc2ccn3c(-c4ccnc(C)c4)c(C#N)cc3c2)nn1)C(F)(F)F]

### Step 5: 4-Nitro-benzoic acid 1-{1-[2-cyano-3-(2-methyl-pyridin-4-yl)-indolizin-7-ylmethyl]-1H-[1,2,3]triazol-4-yl}-1-trifluoromethyl-propyl ester (Enantiomer B)

Freshly prepared methylchloroaluminum amide (5.3 mL, 3.5 mmol) was added to 3-(2-methyl-pyridin-4-yl)-7-{4-[1-(4-nitro-benzoyloxy)-1-trifluoromethyl-propyl]-[1,2,3]triazol-1-ylmethyl}-indolizine-2-carboxylic acid ethyl ester (Enantiomer B) (0.758 g, 1.2 mmol), and the reaction was stirred at 75° C. for 4 hours, and at 85° C. overnight. The mixture was quenched with H₂O and extracted 3 times with EtOAc. The combined organic layers were dried, filtered, and concentrated, and the residue was purified by silica gel chromatography (50-100% EtOAc in hexanes) to give the title compound.

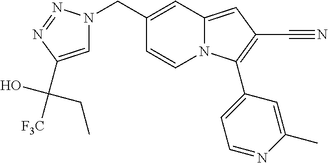 [SMILES: CCC(O)(c1cn(Cc2ccn3c(-c4ccnc(C)c4)c(C#N)cc3c2)nn1)C(F)(F)F]

### Step 6: 7-[4-(1-Hydroxy-1-trifluoromethyl-propyl)-[1,2,3]triazol-1-ylmethyl]-3-(2-methyl-pyridin-4-yl)-indolizine-2-carbonitrile (Enantiomer B)

Prepared according to the procedure described in Example 9, Step 7, using the following starting material: 4-nitro-benzoic acid 1-{1-[2-cyano-3-(2-methyl-pyridin-4-yl)-indolizin-7-ylmethyl]-1H-[1,2,3]triazol-4-yl}-1-trifluoromethyl-propyl ester (Enantiomer B).

### Example 13

### Synthesis of: 7-[4-(1-Hydroxy-1-trifluoromethyl-propyl)-[1,2,3]triazol-1-ylmethyl]-3-isopropyl-indolizine-2-carboxylic acid ethyl ester (Enantiomer B) (Compound 1-77)

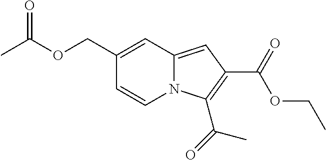 [SMILES: CCOC(=O)c1cc2cc(COC(C)=O)ccn2c1C(C)=O]

### Step 1: 7-Acetoxymethyl-3-acetyl-indolizine-2-carboxylic acid ethyl ester

7-Hydroxymethyl-indolizine-2-carboxylic acid ethyl ester (0.328 g, 1.5 mmol) and sodium acetate (0.366 g, 4.5 mmol) were combined in acetic anhydride (10 mL), and the reaction was stirred at 140° C. in a sealed tube overnight. The mixture was diluted with EtOAc, and the solution was washed 3 times with saturated aqueous NH₄Cl. The organic layer was dried, filtered, and concentrated, and the residue was purified by silica gel chromatography (0-100% EtOAc in hexanes) to give the title compound.

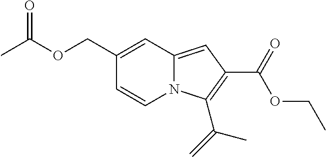 [SMILES: C=C(C)c1c(C(=O)OCC)cc2cc(COC(C)=O)ccn12]

### Step 2: 7-Acetoxymethyl-3-isopropenyl-indolizine-2-carboxylic acid ethyl ester

To 7-acetoxymethyl-3-acetyl-indolizine-2-carboxylic acid ethyl ester (0.445 g, 1.46 mmol) in THF at 0° C. was added Tebbe reagent (0.5 M in toluene, 3.2 mL, 1.6 mmol), and the reaction was stirred at 0° C. for 20 minutes. The mixture was quenched with H₂O and extracted with EtOAc, and the crude material was purified by silica gel chromatography to give the title compound.

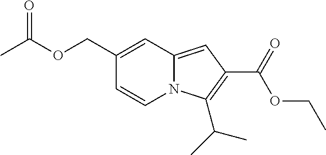 [SMILES: CCOC(=O)c1cc2cc(COC(C)=O)ccn2c1C(C)C]

### Step 3: 7-Acetoxymethyl-3-isopropyl-indolizine-2-carboxylic acid ethyl ester

7-Acetoxymethyl-3-isopropenyl-indolizine-2-carboxylic acid ethyl ester (0.130 g, 0.43 mmol) was dissolved in 5:1 EtOAc:MeOH (10 mL), and the solution was purged with N₂ for 5 minutes. 10% Palladium on carbon (0.100 g) was added, and the reaction was stirred under H₂ for 90 minutes. The mixture was filtered and partitioned between EtOAc and H₂O, and standard work-up provided the title compound, which was used directly in the next step.

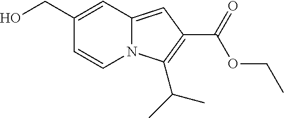 [SMILES: CCOC(=O)c1cc2cc(CO)ccn2c1C(C)C]

### Step 4: 7-Hydroxymethyl-3-isopropyl-indolizine-2-carboxylic acid ethyl ester

7-Acetoxymethyl-3-isopropyl-indolizine-2-carboxylic acid ethyl ester (0.120 g, 0.4 mmol) in MeOH (10 mL) was treated with potassium carbonate (0.014 g, 0.1 mmol), and the reaction was stirred at 50° C. for 30 minutes. The mixture was diluted with EtOAc (100 mL) and H₂O (2 mL), and standard work-up provided the title compound, which was used directly in the following step.

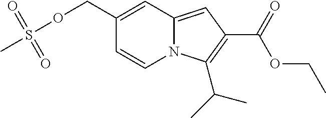 [SMILES: CCOC(=O)c1cc2cc(COS(C)(=O)=O)ccn2c1C(C)C]

### Step 5: 3-Isopropyl-7-methanesulfonyloxymethyl-indolizine-2-carboxylic acid ethyl ester

Prepared according to the procedure described in Example 1, Step 7, using the following starting materials: 7-hydroxymethyl-3-isopropyl-indolizine-2-carboxylic acid ethyl ester and methanesulfonyl chloride.

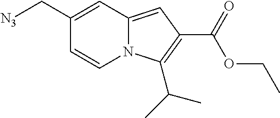 [SMILES: CCOC(=O)c1cc2cc(CN=[N+]=[N-])ccn2c1C(C)C]

### Step 6: 7-Azidomethyl-3-isopropyl-indolizine-2-carboxylic acid ethyl ester

Prepared according to the procedure described in Example 1, Step 8, using the following starting materials: 3-isopropyl-7-methanesulfonyloxymethyl-indolizine-2-carboxylic acid ethyl ester and sodium azide.

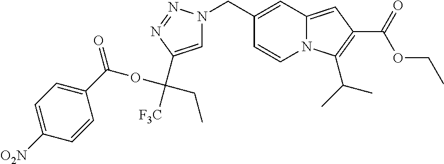 [SMILES: CCOC(=O)c1cc2cc(Cn3cc(C(CC)(OC(=O)c4ccc([N+](=O)[O-])cc4)C(F)(F)F)nn3)ccn2c1C(C)C]

### Step 7: 3-Isopropyl-7-[4-[1-(4-nitro-benzoyloxy)-1-trifluoromethyl-propyl]-[1,2,3]triazol-1-ylmethyl]-indolizine-2-carboxylic acid ethyl ester (Enantiomer B)

Prepared according to the procedure described in Example 1, Step 11, using the following starting materials: 7-azidomethyl-3-isopropyl-indolizine-2-carboxylic acid ethyl ester and 4-nitro-benzoic acid 1-ethyl-1-trifluoromethyl-prop-2-ynyl ester (Enantiomer B).

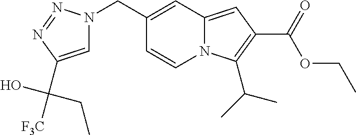 [SMILES: CCOC(=O)c1cc2cc(Cn3cc(C(O)(CC)C(F)(F)F)nn3)ccn2c1C(C)C]

### Step 8: 7-[4-(1-Hydroxy-1-trifluoromethyl-propyl)-[1,2,3]triazol-1-ylmethyl]-3-isopropyl-indolizine-2-carboxylic acid ethyl ester (Enantiomer B)

Prepared according to the procedure described in Example 9, Step 7, using the following starting material: 3-isopropyl-7-{4-[1-(4-nitro-benzoyloxy)-1-trifluoromethyl-propyl]-[1,2,3]triazol-1-ylmethyl}-indolizine-2-carboxylic acid ethyl ester (Enantiomer B).

### Example 14

### Synthesis of 7-[4-(1-Hydroxy-1-trifluoromethyl-propyl)-[1,2,3]triazol-1-ylmethyl]-3-isopropyl-indolizine-2-carbonitrile (Enantiomer B) (Compound 1-81)

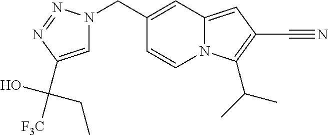 [SMILES: CCC(O)(c1cn(Cc2ccn3c(C(C)C)c(C#N)cc3c2)nn1)C(F)(F)F]

### Step 1: 7-[4-(1-Hydroxy-1-trifluoromethyl-propyl)-[1,2,3]triazol-1-ylmethyl]-3-isopropyl-indolizine-2-carbonitrile (Enantiomer B)

Prepared according to the procedure described in Example 12, Step 5, using the following starting materials: 7-[4-(1-hydroxy-1-trifluoromethyl-propyl)-[1,2,3]triazol-1-ylmethyl]-3-isopropyl-indolizine-2-carboxylic acid ethyl ester (Enantiomer B) and freshly prepared methylchloroaluminum amide.

### Example 15

### Synthesis of 7-[5-(1-Hydroxy-1-trifluoromethyl-propyl)-thiazol-2-ylsulfanyl]-indolizine-2-carboxylic acid ethyl ester (Compound 1-1)

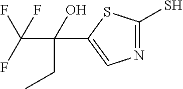

### Step 1: 1,1,1-Trifluoro-2-(2-mercapto-thiazol-5-yl)-butan-2-ol

Diisopropylamine (2.01 g, 19.9 mmol) was dissolved in THF (23 mL) and cooled to −78° C. n-Butyllithium (2.5 M in THF, 7.96 mL, 19.9 mmol) was slowly added and the reaction stirred for 15 minutes. 2-Mercaptothiazole (0.948 g, 7.93 mmol) in THF (5 mL) was added over 10 minutes. After stirring for 10 minutes, 1,1,1-trifluoro-butan-2-one (0.5 g, 3.97 mmol) was added. After stirring at −78° C. for 6 hours, the reaction was allowed to warm to room temperature and submitted to standard aqueous workup. The residue was purified via silica gel chromatography (0-50% EtOAc in hexanes)

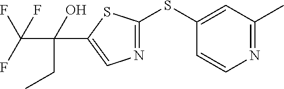 [SMILES: CCC(O)(c1cnc(Sc2ccnc(C)c2)s1)C(F)(F)F]

### Step 2: 1,1,1-Trifluoro-2-[2-(2-methyl-pyridin-4-ylsulfanyl)-thiazol-5-yl]-butan-2-ol

1,1,1-Trifluoro-2-(2-mercapto-thiazol-5-yl)-butan-2-ol (0.486 g, 2.0 mmol), 4-bromo-2-methyl-pyridine (0.344 g, 2.0 mmol), Xantphos (0.081 g, 0.14 mmol) and diisopropylethyl amine (0.560 mL, 3.2 mmol) were suspended in dioxane (15 mL). The mixture was purged with N₂ (g) for 5 minutes and then tris(dibenzylideneacetone) palladium(0) (0.055 g, 0.06 mmol) was added. The reaction was heated to 100° C. for 3 hours then allowed to cool to room temperature. The reaction was concentrated then purified on silica gel (0-100% EtOAc in hexanes) to afford the title compound.

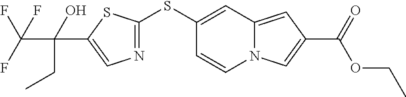 [SMILES: CCOC(=O)c1cc2cc(Sc3ncc(C(O)(CC)C(F)(F)F)s3)ccn2c1]

### Step 3: 7-[5-(1-Hydroxy-1-trifluoromethyl-propyl)-thiazol-2-ylsulfanyl]-indolizine-2carboxylic acid ethyl ester

Prepared according to the procedure described in Example 1, Step 4, using the following starting materials: 1,1,1-trifluoro-2-[2-(2-methyl-pyridin-4-ylsulfanyl)-thiazol-5-yl]-butan-2-ol and ethyl 3-bromopyruvate.

### Example 16

### Synthesis of 7-[5-(1-Hydroxy-1-trifluoromethyl-propyl)-thiazol-2-ylsulfanyl]-indolizine-2-carboxylic acid (Compound 1-2)

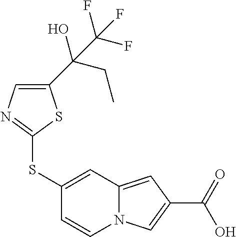 [SMILES: CCC(O)(c1cnc(Sc2ccn3cc(C(=O)O)cc3c2)s1)C(F)(F)F]

### Step 1: 7-[5-(1-Hydroxy-1-trifluoromethyl-propyl)-thiazol-2-ylsulfanyl]-indolizine-2-carboxylic acid

Prepared according to the procedure described in Example 9, Step 7, using the following starting material: 7-[5-(1hydroxy-1-trifluoromethyl-propyl)-thiazol-2-ylsulfanyl]-indolizine-2-carboxylic acid ethyl ester.

### Example 17

### Synthesis of 3-(4-Fluoro-phenyl)-7-[4-(1-hydroxy-1-trifluoromethyl-propyl)-[1,2,3]triazol-2-ylmethyl]-indolizine-2-carboxylic acid ethyl ester (Compound 1-3)

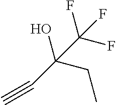

### Step 1: 3-Trifluoromethyl-pent-1-yn-3-ol

Prepared according to the procedure described in Example 3, Step 1, using the following starting materials: 1,1,1-trifluoro-butan-2-one and ethynyl magnesium bromide. The product was carried to the next step without further purification and analysis.

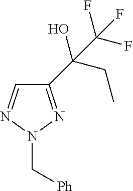 [SMILES: CCC(O)(c1cnn(Cc2ccccc2)n1)C(F)(F)F]

### Step 2: 2-(2-Benzyl-2H-[1,2,3]triazol-4-yl)-1,1,1-trifluoro-butan-2-ol

3-Trifluoromethyl-pent 1-yn-3-ol (from the previous step) was dissolved in t-BuOH (11 mL) and benzyl azide (0.650 mL, 5.18 mmol) was added. Sodium ascorbate (0.0532 g, 0.259 mmol) was added followed by copper(II)sulfate pentahydrate (0.1328 g, 0.518 mmol) and the reaction was stirred at room temperature for 3 days. The reaction mixture was submitted to standard aqueous workup and purified on silica gel (0-100% EtOAc in hexanes) to afford the title compound.

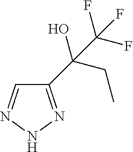

### Step 3: 1,1,1-Trifluoro-2-(2H-[1,2,3]triazol-4-yl)-butan-2-ol

2-(2-Benzyl-2H-[1,2,3]triazol-4-yl)-1,1,1-trifluoro-butan-2-ol (0.9252 g, 3.243 mmol) and palladium(II) hydroxide (0.0932 g, 10% by weight) were suspended in EtOH (19 mL) and placed in a Parr hydrogenator at 60 psi. After 5 hours, the reaction was filtered and concentrated to yield the title compound.

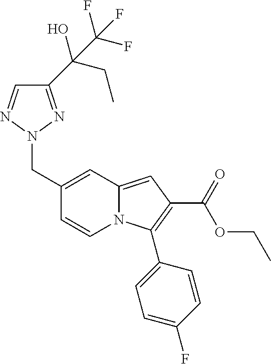 [SMILES: CCOC(=O)c1cc2cc(Cn3ncc(C(O)(CC)C(F)(F)F)n3)ccn2c1-c1ccc(F)cc1]

### Step 4: 3-(4-Fluoro-phenyl)-7-[4-(1-hydroxy-1-trifluoromethyl-propyl)-[1,2,3]triazol-2-ylmethyl]-indolizine-2-carboxylic acid ethyl ester

Prepared according to the procedure described in Example 9, Step 4, using the following starting materials: 3-(4-fluoro-phenyl)-7-methanesulfonyloxymethyl-indolizine-2-carboxylic acid ethyl ester and 1,1,1-trifluoro-2-(2H-[1,2,3]triazol-4-yl)-butan-2-ol.

### Example 18

### Synthesis of 3-(4-Fluoro-phenyl)-7-[4-(1-hydroxy-1-trifluoromethyl-propyl)-[1,2,3]triazol-1-ylmethyl]-indolizine-2-carboxylic acid amide (Compound 1-6)

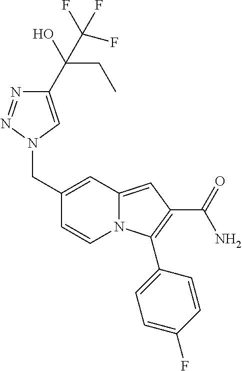 [SMILES: CCC(O)(c1cn(Cc2ccn3c(-c4ccc(F)cc4)c(C(N)=O)cc3c2)nn1)C(F)(F)F]

### Step 1: 3-(4-Fluoro-phenyl)-7-[4-(1-hydroxy-1-trifluoromethyl-propyl)-[1,2,3]triazol-1-ylmethyl]-indolizine-2-carboxylic acid amide

Prepared according to the procedure described in Example 9, Step 6, using the following starting material: 3-(4-fluoro-phenyl)-7-[4-(1-hydroxy-1-trifluoromethyl-propyl)-[1,2,3]triazol-1-ylmethyl]-indolizine-2-carboxylic acid ethyl ester.

### Example 19

### Synthesis of 1,1,1-Trifluoro-2-{1-[3-(4-fluoro-phenyl)-2-(1-hydroxy-1-methyl-ethyl)-indolizin-7-ylmethyl]-1H-[1,2,3]triazol-4-yl}-butan-2-ol (Compound 1-7)

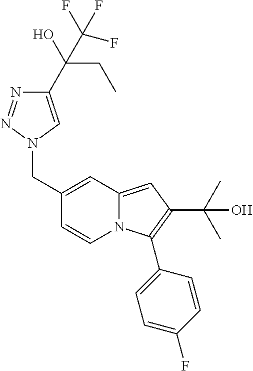 [SMILES: CCC(O)(c1cn(Cc2ccn3c(-c4ccc(F)cc4)c(C(C)(C)O)cc3c2)nn1)C(F)(F)F]

### Step 1: 1,1,1-Trifluoro-2-{1-[3-(4-fluoro-phenyl)-2-(1-hydroxy-1-methyl-ethyl)-indolizin-7-ylmethyl]-1H-[1,2,3]triazol-4-yl}-butan-2-ol

Prepared according to the procedure described in Example 3, Step 1, using the following starting materials: 3-(4-fluoro-phenyl)-7-[4-(1-hydroxy-1-trifluoromethyl-propyl)-[1,2,3]triazol-1-ylmethyl]-indolizine-2-carboxylic acid ethyl ester and methylmagnesium chloride (5 eq.).

### Example 20

### Synthesis of 3-(4-Fluoro-phenyl)-7-[5-(1-hydroxy-1-trifluoromethyl-propyl)-2-imino-[1,3,4]oxadiazol-3-ylmethyl]-indolizine-2-carboxylic acid ethyl ester (Compound 1-8)

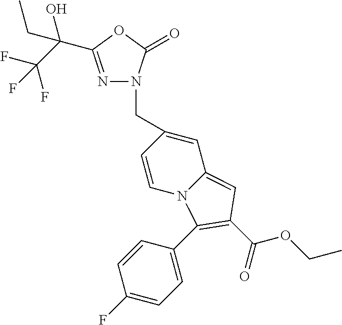 [SMILES: CCOC(=O)c1cc2cc(Cn3nc(C(O)(CC)C(F)(F)F)oc3=O)ccn2c1-c1ccc(F)cc1]

### Step 1: 3-(4-Fluoro-phenyl)-7-[5-(1-hydroxy-1-trifluoromethyl-propyl)-2-imino-[1,3,4]oxadiazol-3-ylmethyl]-indolizine-2-carboxylic acid ethyl ester

Prepared according to the procedure described in Example 9, Step 4, using the following starting materials: 3-(4-fluoro-phenyl)-7-methanesulfonyloxymethyl-indolizine-2-carboxylic acid ethyl ester and 2-(5-amino-[1,3,4]oxadiazol-2-yl)-1,1,1-trifluoro-butan-2-ol.

### Example 21

### Synthesis of 3-(4-Fluoro-phenyl)-7-[4-(1-hydroxy-1-trifluoromethyl-propyl)-[1,2,3]triazol-1-ylmethyl]-indolizine-2-carboxylic acid amide (Enantiomer B) (Compound 1-10)

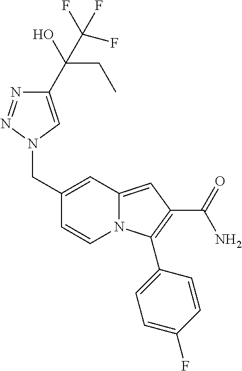 [SMILES: CCC(O)(c1cn(Cc2ccn3c(-c4ccc(F)cc4)c(C(N)=O)cc3c2)nn1)C(F)(F)F]

### Step 1: 3-(4-Fluoro-phenyl)-7-[4-(1-hydroxy-1-trifluoromethyl-propyl)-[1,2,3]triazol-1-ylmethyl]-indolizine-2-carboxylic acid amide (Enantiomer B)

Prepared according to the procedure described in Example 2, Step 2, where the second product to elute from the column the title compound, Enantiomer B (Compound 1-10). This product was again purified using preparatory HPLC to afford the title compound.

### Example 22

### Synthesis of 7-{[5-(1-Hydroxy-1-trifluoromethyl-propyl)-[1,3,4]oxadiazol-2-ylamino]-methyl}-3-phenyl-imidazo[1,2-a]pyridine-2-carboxylic acid ethyl ester (Compound 1-12)

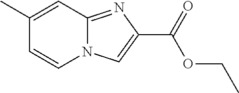

### Step 1: 7-Methyl-imidazo[1,2-a]pyridine-2-carboxylic acid ethyl ester

4-Methyl-pyridin-2-ylamine (2.16 g, 20 mmol) was dissolved in absolute EtOH (50 mL) and heated to 70° C. then 3-bromo-2-oxo-propionic acid ethyl ester (4.29 g, 22 mmol) was added dropwise. After refluxing for 2 hours, the reaction was concentrated to 20% volume then submitted to standard aqueous workup. The crude material was purified on silica gel (50-100% EtOAc in hexanes) to afford the title compound.

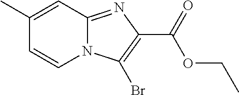 [SMILES: CCOC(=O)c1nc2cc(C)ccn2c1Br]

### Step 2: 3-Bromo-7-methyl-imidazo[1,2-a]pyridine-2-carboxylic acid ethyl ester

7-Methyl-imidazo[1,2-c]pyridine-2-carboxylic acid ethyl ester (2.1 g, 10.3 mmol) and N-bromosuccinimide (1.93 g, 10.8 mmol) were dissolved in EtOH (200 mL) at 0° C. After stirring for 30 minutes the reaction was submitted to standard aqueous workup to yield the title compound.

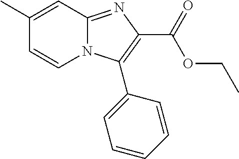 [SMILES: CCOC(=O)c1nc2cc(C)ccn2c1-c1ccccc1]

### Step 3: 7-Methyl-3-phenyl-imidazo[1,2-a]pyridine-2-carboxylic acid ethyl ester

3-Bromo-7-methyl-imidazo[1,2-a]pyridine-2-carboxylic acid ethyl ester (0.425 g, 1.5 mmol), phenylboronic acid (0.220 g, 1.8 mmol) and NaHCO₃ (s, 0.323 g, 5.75 mmol) were combined in a microwave tube then DME:H₂O (3:1, 8 mL) was added. The mixture was bubbled with N₂ (g) for 2 minutes then PdCl₂(PPh₃)₂ (0.053 g, 0.075 mmol) was added. The reaction vessel was sealed and heated by microwave at 140° C. for 10 minutes. An additional portion of phenylboronic acid (0.100 g, 0.82 mmol), NaHCO₃ (0.100 g, 1.78 mmol) and PdCl₂(PPh₃)₂ (0.020 g) were added and the reaction was heated in the microwave at 140° C. for 15 minutes. The reaction mixture was submitted to standard aqueous workup and purified on silica gel to afford the title compound.

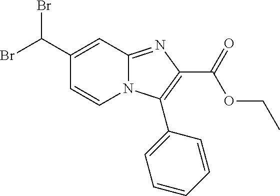 [SMILES: CCOC(=O)c1nc2cc(C(Br)Br)ccn2c1-c1ccccc1]

### Step 4: 7-Dibromomethyl-3-phenyl-imidazo[1,2-a]pyridine-2-carboxylic acid ethyl ester

7-Methyl-3-phenyl-imidazo[1,2-c]pyridine-2-carboxylic acid ethyl ester (0.350 g, 1.25 mmol) and N-bromosuccinimide (0.556 g, 3.13 mmol) were dissolved in carbon tetrachloride (15 mL). The solution was flushed with N₂ (g) then azobisisobutyronitrile (0.01 g, 0.06 mmol) was added and the reaction heated to reflux. After 1 hour, additional portions of N-bromosuccinimide (0.10 g) and azobisisobutyronitrile (0.02 g) were added and the reaction was heated at reflux overnight. The reaction mixture was cooled and then submitted to standard aqueous workup and purified on silica gel to afford the title compound.

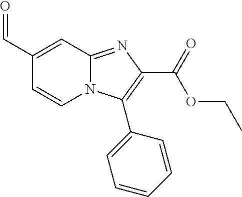 [SMILES: CCOC(=O)c1nc2cc(C=O)ccn2c1-c1ccccc1]

### Step 5: 7-Formyl-3-phenyl-imidazo[1,2-a]pyridine-2-carboxylic acid ethyl ester

7-Dibromomethyl-3-phenyl-imidazo[1,2-a]pyridine-2-carboxylic acid ethyl ester (0.119 g, 0.27 mmol) was dissolved in dioxane (2 mL). Silver nitrate (0.115 g, 0.68 mmol) in H₂O (1 mL) was added and the reaction was heated to 60° C. for 2 hours. After cooling, the reaction was submitted to standard aqueous workup to yield the title compound.

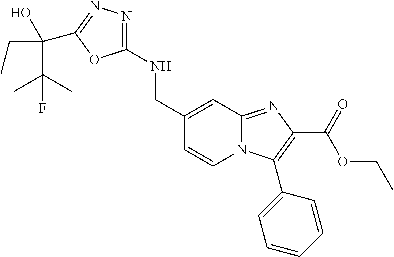 [SMILES: CCOC(=O)c1nc2cc(CNc3nnc(C(O)(CC)C(C)(C)F)o3)ccn2c1-c1ccccc1]

### Step 6: 7-{[5-(1-Hydroxy-1-trifluoromethyl-propyl)-[1,3,4]oxadiazol-2-ylamino]-methyl}-3-phenyl-imidazo[1,2-a]pyridine-2-carboxylic acid ethyl ester

Prepared according to the procedure described in Example 3, Step 5, using the following starting materials: 7-formyl-3-phenyl-imidazo[1,2-a]pyridine-2-carboxylic acid ethyl ester and 2-(5-amino-[1,3,4]oxadiazol-2-yl)-1,1,1-trifluoro-butan-2-ol.

### Example 23

### Synthesis of 3-(4-Fluoro-phenyl)-7-[4-(1-hydroxy-1-trifluoromethyl-propyl)-[1,2,3]triazol-1-ylmethyl]-imidazo[1,2-a]pyridine-2-carboxylic acid ethyl ester (Compound 1-13)

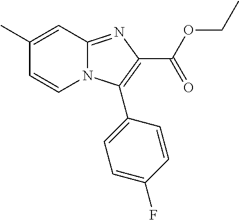 [SMILES: CCOC(=O)c1nc2cc(C)ccn2c1-c1ccc(F)cc1]

### Step 1: 3-(4-Fluoro-phenyl)-7-methyl-imidazo[1,2-a]pyridine-2-carboxylic acid ethyl ester

3-Bromo-7-methyl-imidazo[1,2-a]pyridine-2-carboxylic acid ethyl ester (10 g, 35.3 mmol), 4-fluorophenylboronic acid (7 g, 50 mmol) and NaHCO₃ (s, 15.2 g, 177 mmol) were combined in DME:H₂O (3:1, 400 mL). The mixture was bubbled with N₂ (g) for 10 minutes then PdCl₂(PPh₃)₂ (1.24 g, 1.77 mmol) was added. The reaction was heated at 75° C. overnight. The reaction mixture was submitted to standard aqueous workup to afford the title compound. During the workup, some of the product was insoluble and was filtered off, then recombined with the recovered material, and purified on silica gel for use in the next step.

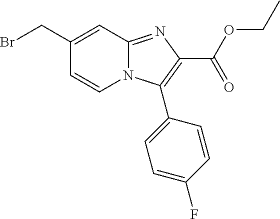 [SMILES: CCOC(=O)c1nc2cc(CBr)ccn2c1-c1ccc(F)cc1]

### Step 2: 7-Bromomethyl-3-(4-fluoro-phenyl)-imidazo[1,2-a]pyridine-2-carboxylic acid ethyl ester

3-(4-Fluoro-phenyl)-7-methyl-imidazo[1,2-a]pyridine-2-carboxylic acid ethyl ester (5.31 g, 17.8 mmol) was dissolved in benzene (200 mL) along with N-bromosuccinimide (3.17 g, 17.8 mmol) and benzoyl peroxide (0.216 g, 0.89 mmol). The reaction was heated to reflux overnight then cooled and submitted to standard aqueous workup. Purification on silica gel afforded the title compound.

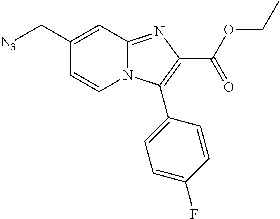 [SMILES: CCOC(=O)c1nc2cc(CN=[N+]=[N-])ccn2c1-c1ccc(F)cc1]

### Step 3: 7-Azidomethyl-3-(4-fluoro-phenyl)-imidazo[1,2-a]pyridine-2-carboxylic acid ethyl ester

Prepared according to the procedure described in Example 1, Step 8, using the following starting materials: 7-Bromomethyl-3-(4-fluoro-phenyl)-imidazo[1,2-c]pyridine-2-carboxylic acid ethyl ester and sodium azide.

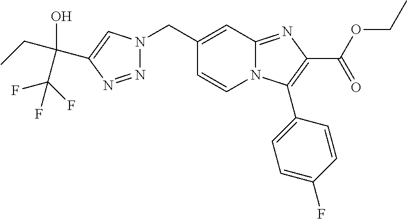 [SMILES: CCOC(=O)c1nc2cc(Cn3cc(C(O)(CC)C(F)(F)F)nn3)ccn2c1-c1ccc(F)cc1]

### Step 4: 3-(4-Fluoro-phenyl)-7-[4-(1-hydroxy-1-trifluoromethyl-propyl)-[1,2,3]triazol-1-ylmethyl]-imidazo[1,2-a]pyridine-2-carboxylic acid ethyl ester

Prepared according to the procedure described in Example 1, Step 11, using the following starting materials: 7-azidomethyl-3-(4-fluoro-phenyl)-imidazo[1,2-c]pyridine-2-carboxylic acid ethyl ester and 3-trifluoromethyl-pent-1-yn-3-ol.

### Example 24

### Synthesis of 3-(4-Fluoro-phenyl)-7-[4-(1-hydroxy-1-trifluoromethyl-propyl)-[1,2,3]triazol-1-ylmethyl]-imidazo[1,2-a]pyridine-2-carboxylic acid amide (Compound 1-14)

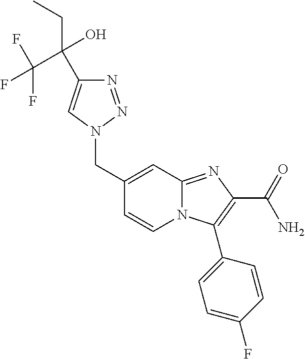 [SMILES: CCC(O)(c1cn(Cc2ccn3c(-c4ccc(F)cc4)c(C(N)=O)nc3c2)nn1)C(F)(F)F]

### Step 1: 3-(4-Fluoro-phenyl)-7-[4-(1-hydroxy-1-trifluoromethyl-propyl)-[1,2,3]triazol-1-ylmethyl]-imidazo[1,2-a]pyridine-2-carboxylic acid amide

Prepared according to the procedure described in Example 9, Step 6, using the following starting material: 3-(4-fluoro-phenyl)-7-[4-(1-hydroxy-1-trifluoromethyl-propyl)-[1,2,3]triazol-1-ylmethyl]-imidazo[1,2-a]pyridine-2-carboxylic acid ethyl ester.

### Example 25

### Synthesis of 1-Bromo-3-(4-fluoro-phenyl)-7-{[5-(1-hydroxy-1-trifluoromethyl-propyl)-[1,3,4]oxadiazol-2-ylamino]-methyl}-indolizine-2-carboxylic acid ethyl ester (Compound 1-15)

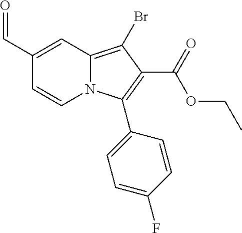 [SMILES: CCOC(=O)c1c(Br)c2cc(C=O)ccn2c1-c1ccc(F)cc1]

### Step 1: 1-Bromo-3-(4-fluoro-phenyl)-7-formyl-indolizine-2-carboxylic acid ethyl ester

Prepared according to the procedure described in Example 22, Step 2, using the following starting material: 3-(4-fluoro-phenyl)-7-formyl-indolizine-2-carboxylic acid ethyl ester.

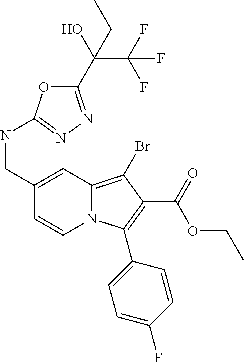 [SMILES: CCOC(=O)c1c(Br)c2cc(CNc3nnc(C(O)(CC)C(F)(F)F)o3)ccn2c1-c1ccc(F)cc1]

### Step 2: 1-Bromo-3-(4-fluoro-phenyl)-7-{[5-(1-hydroxy-1-trifluoromethyl-propyl)-[1,3,4]oxadiazol-2-ylamino]-methyl}-indolizine-2-carboxylic acid ethyl ester

Prepared according to the procedure described in Example 3, Step 5, using the following starting materials: 1-bromo-3-(4-fluoro-phenyl)-7-formyl-indolizine-2-carboxylic acid ethyl ester and 2-(5amino-[1,3,4]oxadiazol-2-yl)-1,1,1-trifluoro-butan-2ol.

### Example 26

### Synthesis of 3-(4-Fluoro-phenyl)-7-[4-(1-hydroxy-1-trifluoromethyl-propyl)-[1,2,3]triazol-1-ylmethyl]-indolizine-2-carbonitrile (Compound 1-16)

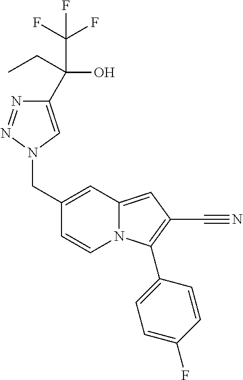 [SMILES: CCC(O)(c1cn(Cc2ccn3c(-c4ccc(F)cc4)c(C#N)cc3c2)nn1)C(F)(F)F]

### Step 1: 3-(4-Fluoro-phenyl)-7-[4-(1-hydroxy-1-trifluoromethyl-propyl)-[1,2,3]triazol-1-ylmethyl]-indolizine-2-carbonitrile

Prepared according to the procedure described in Example 9, Step 6, using the following starting material: 3-(4-fluoro-phenyl)-7-[4-(1-hydroxy-1-trifluoromethyl-propyl)-[1,2,3]triazol-1-ylmethyl]-indolizine-2-carboxylic acid ethyl ester. The reaction yielded a mixture of two separable products, of which the title compound was the minor product isolated.

### Example 27

### Synthesis of 1-Bromo-3-(4-fluoro-phenyl)-7-{[5-(1-hydroxy-1-trifluoromethyl-propyl)-[1,3,4]oxadiazol-2-ylamino]-methyl}-indolizine-2-carboxylic acid amide (Compound 1-17)

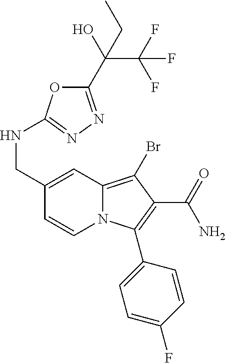 [SMILES: CCC(O)(c1nnc(NCc2ccn3c(-c4ccc(F)cc4)c(C(N)=O)c(Br)c3c2)o1)C(F)(F)F]

### Step 1: 1-Bromo-3-(4-fluoro-phenyl)-7-{[5-(1-hydroxy-1-trifluoromethyl-propyl)-[1,3,4]oxadiazol-2-ylamino]-methyl}-indolizine-2-carboxylic acid amide

Prepared according to the procedure described in Example 9, Step 6, using the following starting material: 1-bromo-3-(4-fluoro-phenyl)-7-{[5-(1-hydroxy-1-trifluoromethyl-propyl)-[1,3,4]oxadiazol-2-ylamino]-methyl}-indolizine-2-carboxylic acid ethyl ester. The reaction yielded a mixture of two separable products, of which the title compound was the major product isolated.

### Example 28

### Synthesis of 1-Bromo-3-(4-fluoro-phenyl)-7-{[5-(1-hydroxy-1-trifluoromethyl-propyl)-[1,3,4]oxadiazol-2-ylamino]-methyl}-indolizine-2-carbonitrile (Compound 1-18)

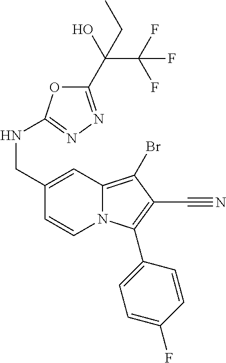 [SMILES: CCC(O)(c1nnc(NCc2ccn3c(-c4ccc(F)cc4)c(C#N)c(Br)c3c2)o1)C(F)(F)F]

### Step 1: 1-Bromo-3-(4-fluoro-phenyl)-7-{[5-(1-hydroxy-1-trifluoromethyl-propyl)-[1,3,4]oxadiazol-2-ylamino]-methyl}-indolizine-2-carbonitrile

Prepared according to the procedure described in Example 9, Step 6, using the following starting material: 1-bromo-3-(4-fluoro-phenyl)-7-{[5-(1-hydroxy-1-trifluoromethyl-propyl)-[1,3,4]oxadiazol-2-ylamino]-methyl}-indolizine-2-carboxylic acid ethyl ester. The reaction yielded a mixture of two separable products, of which the title compound was the minor product isolated.

### Example 29

### Synthesis of 3-(4-Fluoro-phenyl)-7-{[5-(1-hydroxy-1-trifluoromethyl-propyl)-[1,3,4]oxadiazol-2-ylamino]-methyl}-indolizine-2-carboxylic acid amide (Compound 1-19)

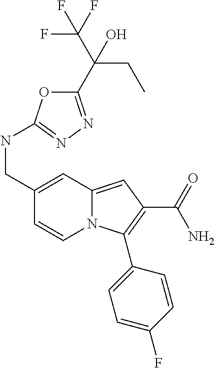 [SMILES: CCC(O)(c1nnc(NCc2ccn3c(-c4ccc(F)cc4)c(C(N)=O)cc3c2)o1)C(F)(F)F]

### Step 1: 3-(4-Fluoro-phenyl)-7-{[5-(1-hydroxy-1-trifluoromethyl-propyl)-[1,3,4]oxadiazol-2-ylamino]-methyl}-indolizine-2-carboxylic acid amide

Prepared according to the procedure described in Example 13, Step 3, using the following starting material: 1-bromo-3-(4-fluoro-phenyl)-7-{[5-(1-hydroxy-1-trifluoromethyl-propyl)-[1,3,4]oxadiazol-2-ylamino]-methyl}-indolizine-2-carboxylic acid amide.

### Example 30

### Synthesis of 3-(4-Fluoro-phenyl)-7-{[5-(1-hydroxy-1-trifluoromethyl-propyl)-[1,3,4]oxadiazol-2-ylamino]-methyl}-indolizine-2-carboxylic acid amide (Enantiomer A) (Compound 1-20)

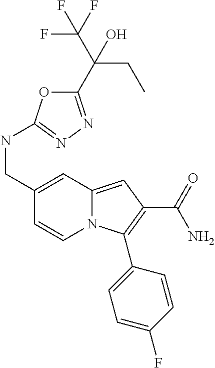 [SMILES: CCC(O)(c1nnc(NCc2ccn3c(-c4ccc(F)cc4)c(C(N)=O)cc3c2)o1)C(F)(F)F]

### Step 1: 3-(4-Fluoro-phenyl)-7-{[5-(1-hydroxy-1-trifluoromethyl-propyl)-[1,3,4]oxadiazol-2-ylamino]-methyl}-indolizine-2-carboxylic acid amide (Enantiomer A)

Prepared according to the procedure described in Example 29, Step 1, and after the reaction the racemic product was submitted to chiral preparatory HPLC (50% isopropanol in hexanes, 32 minutes). The first enantiomer to elute from the column is Enantiomer A.

### Example 31

### Synthesis of 3-(4-Fluoro-phenyl)-7-{[5-(1-hydroxy-1-trifluoromethyl-propyl)-[1,3,4]oxadiazol-2-ylamino]-methyl}-indolizine-2-carboxylic acid amide (Enantiomer B) (Compound 1-21)

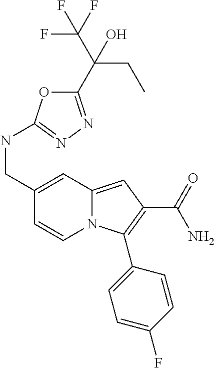 [SMILES: CCC(O)(c1nnc(NCc2ccn3c(-c4ccc(F)cc4)c(C(N)=O)cc3c2)o1)C(F)(F)F]

### Step 1: 3-(4-Fluoro-phenyl)-7-{[5-(1-hydroxy-1-trifluoromethyl-propyl)-[1,3,4]oxadiazol-2-ylamino]-methyl}-indolizine-2-carboxylic acid amide (Enantiomer B)

Prepared according to the procedure described in Example 29, Step 1, and after the reaction the racemic product was submitted to chiral preparatory HPLC (50% isopropanol in hexanes, 32 minutes). The second enantiomer to elute from the column is Enantiomer B.

### Example 32

### Synthesis of 1,1,1-Trifluoro-2-{1-[3-(4-fluoro-phenyl)-2-(2H-tetrazol-5-yl)-indolizin-7-ylmethyl]-1H-[1,2,3]triazol-4-yl}-butan-2-ol (Compound 1-23)

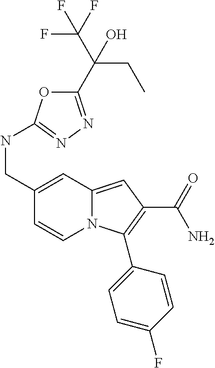 [SMILES: CCC(O)(c1nnc(NCc2ccn3c(-c4ccc(F)cc4)c(C(N)=O)cc3c2)o1)C(F)(F)F]

### Step 1: 1,1,1-Trifluoro-2-{1-[3-(4-fluoro-phenyl)-2-(2H-tetrazol-5-yl)-indolizin-7-ylmethyl]-1H-[1,2,3]triazol-4-yl}-butan-2-ol

3-(4-Fluoro-phenyl)-7-[4-(1-hydroxy-1-trifluoromethyl-propyl)-[1,2,3]triazol-1-ylmethyl]-indolizine-2-carbonitrile (0.070 g, 0.16 mmol) and dibutyltin oxide (0.004 g, 0.016 mmol) were dissolved in toluene in a scintillation vial. Trimethylsilyl azide (0.063 mL, 0.48 mmol) was added, the reaction vessel was sealed, and the reaction was heated to 85° C. overnight. After cooling the reaction as submitted to standard aqueous workup and purified via preparatory HPLC to afford the title compound.

### Example 33

### Synthesis of 7-[4-(1-Hydroxy-1-trifluoromethyl-propyl)-[1,2,3]triazol-1-ylmethyl]-3-pyridin-3-yl-indolizine-2-carboxylic acid ethyl ester (Compound 1-24)

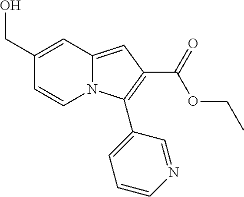 [SMILES: CCOC(=O)c1cc2cc(CO)ccn2c1-c1cccnc1]

### Step 1: 7-Hydroxymethyl-3-pyridin-3-yl-indolizine-2-carboxylic acid ethyl ester

Prepared according to the procedure described in Example 9, Step 1, using the following starting materials: 7-hydroxymethyl-indolizine-2-carboxylic acid ethyl ester and 3-bromo-pyridine.

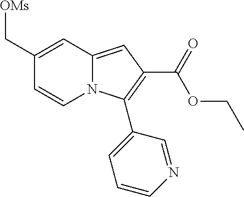 [SMILES: CCOC(=O)c1cc2cc(COS(C)(=O)=O)ccn2c1-c1cccnc1]

### Step 2: 7-Methanesulfonyloxymethyl-3-pyridin-3-yl-indolizine-2-carboxylic acid ethyl ester

Prepared according to the procedure described in Example 1, Step 7, using the following starting material: 7-hydroxymethyl-3-pyridin-3-yl-indolizine-2-carboxylic acid ethyl ester.

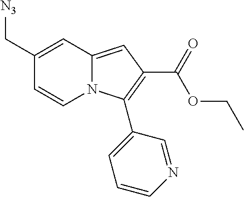 [SMILES: CCOC(=O)c1cc2cc(CN=[N+]=[N-])ccn2c1-c1cccnc1]

### Step 3: 7-Azidomethyl-3-pyridin-3-yl-indolizine-2-carboxylic acid ethyl ester

Prepared according to the procedure described in Example 1, Step 8, using the following starting material: 7-Methanesulfonyloxymethyl-3-pyridin-3-yl-indolizine-2-carboxylic acid ethyl ester.

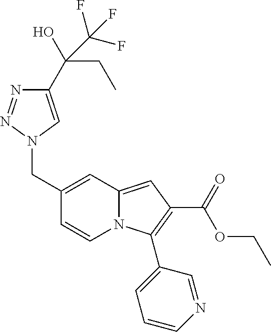 [SMILES: CCOC(=O)c1cc2cc(Cn3cc(C(O)(CC)C(F)(F)F)nn3)ccn2c1-c1cccnc1]

### Step 4: 7-[4-(1-Hydroxy-1-trifluoromethyl-propyl)-[1,2,3]triazol-1-ylmethyl]-3-pyridin-3-yl-indolizine-2-carboxylic acid ethyl ester

Prepared according to the procedure described in Example 1, Step 11, using the following starting materials: 7-azidomethyl-3-pyridin-3-yl-indolizine-2-carboxylic acid ethyl ester and 3-trifluoromethyl-pent-1-yn-3-ol.

### Example 34

### Synthesis of 7-[4-(1-Hydroxy-1-trifluoromethyl-propyl)-[1,2,3]triazol-1-ylmethyl]-3-pyridin-3-yl-indolizine-2-carboxylic acid amide (Compound 1-25)

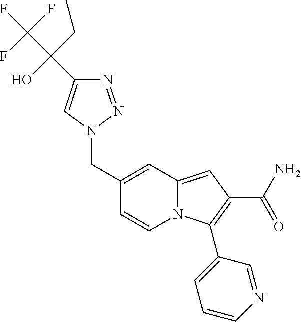 [SMILES: CCC(O)(c1cn(Cc2ccn3c(-c4cccnc4)c(C(N)=O)cc3c2)nn1)C(F)(F)F]

### Step 1: 7-[4-(1-Hydroxy-1-trifluoromethyl-propyl)-[1,2,3]triazol-1-ylmethyl]-3-pyridin-3-yl-indolizine-2-carboxylic acid amide

Prepared according to the procedure described in Example 9, Step 6, using the following starting material: 7-[4-(1-hydroxy-1-trifluoromethyl-propyl) [1,2,3]triazol-1-ylmethyl]-3-pyridin-3-yl-indolizine-2-carboxylic acid ethyl ester. The reaction yielded a mixture of two separable products, of which the title compound was the major product isolated.

### Example 35

### Synthesis of 7-[4-(1-Hydroxy-1-trifluoromethyl-propyl)-[1,2,3]triazol-1-ylmethyl]-3-pyridin-3-yl-indolizine-2-carbonitrile (Compound 1-26)

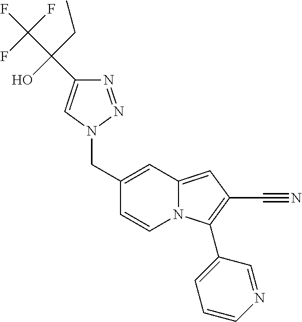 [SMILES: CCC(O)(c1cn(Cc2ccn3c(-c4cccnc4)c(C#N)cc3c2)nn1)C(F)(F)F]

### Step 1: 7-[4-(1-Hydroxy-1-trifluoromethyl-propyl)-[1,2,3]triazol-1-ylmethyl]-3-pyridin-3-yl-indolizine-2-carbonitrile

Prepared according to the procedure described in Example 9, Step 6, using the following starting material: 7-[4-(1-hydroxy-1-trifluoromethyl-propyl)-[1,2,3]triazol-1-ylmethyl]-3-pyridin-3-yl-indolizine-2-carboxylic acid ethyl ester. The reaction yielded a mixture of two separable products, of which the title compound was the minor product isolated.

### Example 36

### Synthesis of 3-(4-Chloro-phenyl)-7-[4-(1-hydroxy-1-trifluoromethyl-propyl)-[1,2,3]triazol-1-ylmethyl]-indolizine-2-carboxylic acid ethyl ester (Compound 1-27)

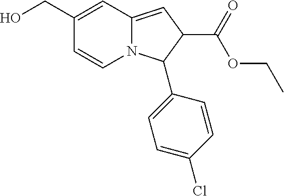 [SMILES: CCOC(=O)C1C=C2C=C(CO)C=CN2C1c1ccc(Cl)cc1]

### Step 1: 3-(4-Chloro-phenyl)-7-hydroxymethyl-indolizine-2-carboxylic acid ethyl ester

Prepared according to the procedure described in Example 9, Step 1, using the following starting materials: 7-hydroxymethyl-indolizine-2-carboxylic acid ethyl ester and 1-bromo-4-chloro-benzene.

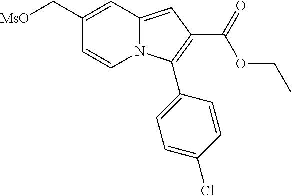 [SMILES: CCOC(=O)c1cc2cc(COS(C)(=O)=O)ccn2c1-c1ccc(Cl)cc1]

### Step 2: 3-(4-Chloro-phenyl)-7-methanesulfonyloxymethyl-indolizine-2-carboxylic acid ethyl ester

Prepared according to the procedure described in Example 1, Step 7, using the following starting material: 3-(4-chloro-phenyl)-7-hydroxymethyl-indolizine-2-carboxylic acid ethyl ester.

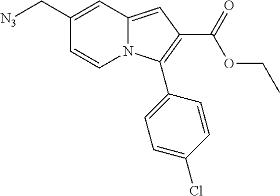 [SMILES: CCOC(=O)c1cc2cc(CN=[N+]=[N-])ccn2c1-c1ccc(Cl)cc1]

### Step 3: 7-Azidomethyl-3-(4-chloro-phenyl)-indolizine-2-carboxylic acid ethyl ester

Prepared according to the procedure described in Example 1, Step 8, using the following starting material: 3-(4-chloro-phenyl)-7-methanesulfonyloxymethyl-indolizine-2-carboxylic acid ethyl

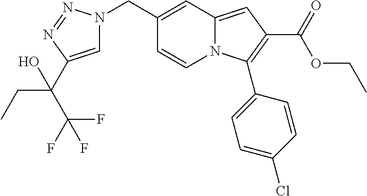 [SMILES: CCOC(=O)c1cc2cc(Cn3cc(C(O)(CC)C(F)(F)F)nn3)ccn2c1-c1ccc(Cl)cc1]

### Step 4: 3-(4-Chloro-phenyl)-7-[4-(1-hydroxy-1-trifluoromethyl-propyl)-[1,2,3]triazol-1-ylmethyl]-indolizine-2-carboxylic acid ethyl ester

Prepared according to the procedure described in Example 1, Step 11, using the following starting materials: 7-azidomethyl-3-(4-chloro-phenyl)-indolizine-2-carboxylic acid ethyl ester and 3-trifluoromethyl-pent-1-yn-3-ol.

### Example 37

### Synthesis of 3-(4-Chloro-phenyl)-7-[4-(1-hydroxy-1-trifluoromethyl-propyl)-[1,2,3]triazol-1-ylmethyl]-indolizine-2-carboxylic acid amide (Compound 1-28)

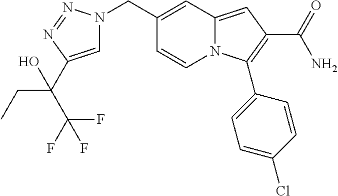 [SMILES: CCC(O)(c1cn(Cc2ccn3c(-c4ccc(Cl)cc4)c(C(N)=O)cc3c2)nn1)C(F)(F)F]

### Step 1: 3-(4-Chloro-phenyl)-7-[4-(1-hydroxy-1-trifluoromethyl-propyl)-[1,2,3]triazol-1-ylmethyl]-indolizine-2-carboxylic acid amide

Prepared according to the procedure described in Example 9, Step 6, using the following starting material: 3-(4-chloro-phenyl)-7-[4-(1-hydroxy-1-trifluoromethyl-propyl)-[1,2,3]triazol-1-ylmethyl]-indolizine-2-carboxylic acid ethyl ester.

### Example 38

### Synthesis of 3-(6-Fluoro-pyridin-3-yl)-7-[4-(1-hydroxy-1-trifluoromethyl-propyl)-[1,2,3]triazol-1-ylmethyl]-indolizine-2-carboxylic acid ethyl ester (Compound 1-29)

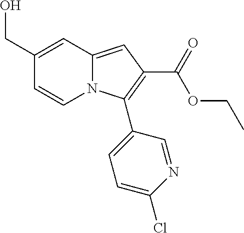 [SMILES: CCOC(=O)c1cc2cc(CO)ccn2c1-c1ccc(Cl)nc1]

### Step 1: 3-(6-Fluoro-pyridin-3-yl)-7-hydroxymethyl-indolizine-2-carboxylic acid ethyl ester

Prepared according to the procedure described in Example 9, Step 1, using the following starting materials: 7-hydroxymethyl-indolizine-2-carboxylic acid ethyl ester and 5-bromo-2-fluoro-pyridine.

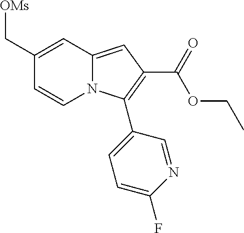 [SMILES: CCOC(=O)c1cc2cc(COS(C)(=O)=O)ccn2c1-c1ccc(F)nc1]

### Step 2: 3-(6-Fluoro-pyridin-3-yl)-7-methanesulfonyloxymethyl-indolizine-2-carboxylic acid ethyl ester

Prepared according to the procedure described in Example 1, Step 7, using the following starting material: 3-(6-fluoro-pyridin-3-yl)-7-hydroxymethyl-indolizine-2-carboxylic acid ethyl ester.

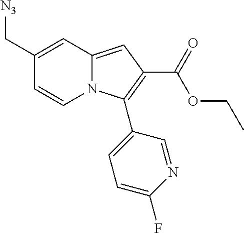 [SMILES: CCOC(=O)c1cc2cc(CN=[N+]=[N-])ccn2c1-c1ccc(F)nc1]

### Step 3: 7-Azidomethyl-3-(6-fluoro-pyridin-3-yl)-indolizine-2-carboxylic acid ethyl ester

Prepared according to the procedure described in Example 1, Step 8, using the following starting material: 3-(6-fluoro-pyridin-3-yl)-7-methanesulfonyloxymethyl-indolizine-2-carboxylic acid ethyl ester.

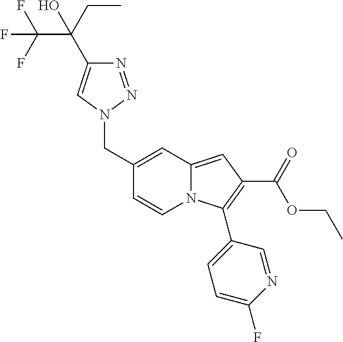 [SMILES: CCOC(=O)c1cc2cc(Cn3cc(C(O)(CC)C(F)(F)F)nn3)ccn2c1-c1ccc(F)nc1]

### Step 4: 3-(6-Fluoro-pyridin-3-yl)-7-[4-(1-hydroxy-1-trifluoromethyl-propyl)-[1,2,3]triazol-1-ylmethyl]-indolizine-2-carboxylic acid ethyl ester

Prepared according to the procedure described in Example 1, Step 11, using the following starting materials: 7-azidomethyl-3-(6-fluoro-pyridin-3-yl)-indolizine-2-carboxylic acid ethyl ester and 3-trifluoromethyl-pent-1-yn-3-ol.

### Example 39

### Synthesis of 1,1,1-Trifluoro-2-{1-[3-(4-fluoro-phenyl)-2-hydroxymethyl-indolizin-7-ylmethyl]-1H-[1,2,3]triazol-4-yl}-butan-2-ol (Compound 1-30)

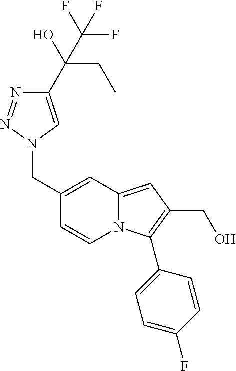 [SMILES: CCC(O)(c1cn(Cc2ccn3c(-c4ccc(F)cc4)c(CO)cc3c2)nn1)C(F)(F)F]

### Step 1: 1,1,1-Trifluoro-2-{1-[3-(4-fluoro-phenyl)-2-hydroxymethyl-indolizin-7-ylmethyl]-1H-[1,2,3]triazol-4-yl}-butan-2-ol

3-(4-Fluoro-phenyl)-7-[4-(1-hydroxy-1-trifluoromethyl-propyl)-[1,2,3]triazol-1-ylmethyl]-indolizine-2-carboxylic acid ethyl ester (0.150 g, 0.31 mmol) was dissolved in THF (3 mL) and cooled to −78° C. Diisobutylaluminal hydride (1 M in THF, 0.930 mL, 0.93 mmol) was added slowly and the reaction stirred at cryogenic temperature for 30 minutes. Monitoring the reaction by analytical LCMS, it was warmed to room temperature and stirred for 1 hour. The reaction was cooled to −78° C. and diisobutylaluminum hydride (0.62 mmol) was added. After stirring for 2 hours, additional diisobutylaluminum hydride (0.31 mmol) was added and the reaction was allowed to warm to room temperature. Standard aqueous workup yielded the title compound.

### Example 40

### Synthesis of 3-(6-Fluoro-pyridin-3-yl)-7-[4-(1-hydroxy-1-trifluoromethyl-propyl)-[1,2,3]triazol-1-ylmethyl]-indolizine-2-carboxylic acid amide (Compound 1-31)

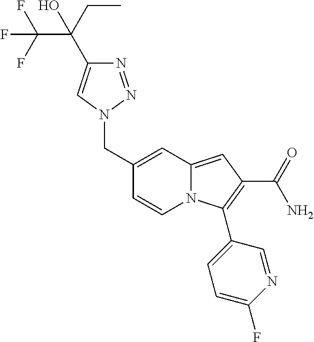 [SMILES: CCC(O)(c1cn(Cc2ccn3c(-c4ccc(F)nc4)c(C(N)=O)cc3c2)nn1)C(F)(F)F]

### Step 1: 3-(6-Fluoro-pyridin-3-yl)-7-[4-(1-hydroxy-1-trifluoromethyl-propyl)-[1,2,3]triazol-1-ylmethyl]-indolizine-2-carboxylic acid amide

Prepared according to the procedure described in Example 9, Step 6, using the following starting material: 3-(6-fluoro-pyridin-3-yl)-7-[4-(1-hydroxy-1-trifluoromethyl-propyl)-[1,2,3]triazol-1-ylmethyl]-indolizine-2-carboxylic acid ethyl ester. The reaction yielded a mixture of two separable products, of which the title compound was the major product isolated.

### Example 41

### Synthesis of 3-(6-Fluoro-pyridin-3-yl)-7-[4-(1-hydroxy-1-trifluoromethyl-propyl)-[1,2,3]triazol-1-ylmethyl]-indolizine-2-carbonitrile (Compound 1-32)

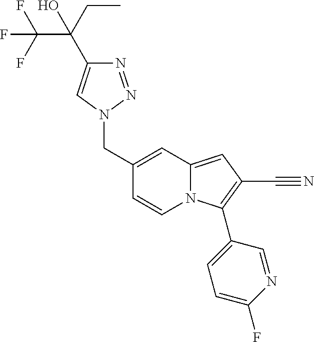 [SMILES: CCC(O)(c1cn(Cc2ccn3c(-c4ccc(F)nc4)c(C#N)cc3c2)nn1)C(F)(F)F]

### Step 1: 3-(6-Fluoro-pyridin-3-yl)-7-[4-(1-hydroxy-1-trifluoromethyl-propyl)-[1,2,3]triazol-1-ylmethyl]-indolizine-2-carbonitrile

Prepared according to the procedure described in Example 9, Step 6, using the following starting material: 3-(6-fluoro-pyridin-3-yl)-7-[4-(1-hydroxy-1-trifluoromethyl-propyl)-[1,2,3]triazol-1-ylmethyl]-indolizine-2-carboxylic acid ethyl ester. The reaction yielded a mixture of two separable products, of which the title compound was the minor product isolated.

### Example 42

### Synthesis of 3-(4-Fluoro-phenyl)-7-[4-(1-hydroxy-1-trifluoromethyl-propyl)-[1,2,3]triazol-1-ylmethyl]-indolizine-2-carbaldehyde (Compound 1-33)

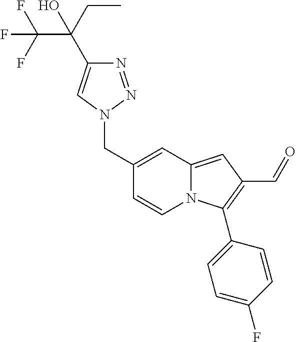 [SMILES: CCC(O)(c1cn(Cc2ccn3c(-c4ccc(F)cc4)c(C=O)cc3c2)nn1)C(F)(F)F]

### Step 1: 3-(4-Fluoro-phenyl)-7-[4-(1-hydroxy-1-trifluoromethyl-propyl)-[1,2,3]triazol-1-ylmethyl]-indolizine-2-carbaldehyde

Prepared according to the procedure described in Example 3, Step 4, using the following starting material: 1,1,1-trifluoro-2-{1-[3-(4-fluoro-phenyl)-2-hydroxymethyl-indolizin-7-ylmethyl]-1H-[1,2,3]triazol-4-yl}-butan-2-ol.

### Example 43

### Synthesis of 1,1,1-Trifluoro-2-{1-[3-(4-fluoro-phenyl)-2-methanesulfonylmethyl-indolizin-7-ylmethyl]-1H-[1,2,3]triazol-4-yl}-butan-2-ol (Compound 1-34)

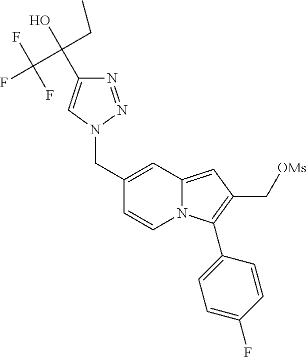 [SMILES: CCC(O)(c1cn(Cc2ccn3c(-c4ccc(F)cc4)c(COS(C)(=O)=O)cc3c2)nn1)C(F)(F)F]

### Step 1: Methanesulfonic acid 3-(4-fluoro-phenyl)-7-[4-(1-hydroxy-1-trifluoromethyl-propyl)-[1,2,3]triazol-1-ylmethyl]-indolizin-2-ylmethyl ester

Prepared according to the procedure described in Example 1, Step 7, using the following starting material: 1,1,1-trifluoro-2-{1-[3-(4-fluoro-phenyl)-2-hydroxymethyl-indolizin-7-ylmethyl]-1H-[1,2,3]triazol-4-yl}-butan-2-ol. The product was used directly in the next step.

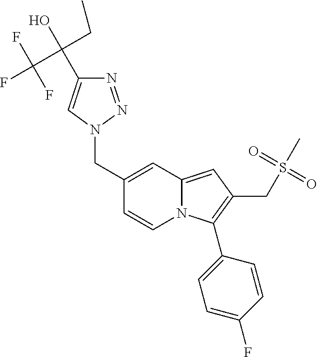 [SMILES: CCC(O)(c1cn(Cc2ccn3c(-c4ccc(F)cc4)c(CS(C)(=O)=O)cc3c2)nn1)C(F)(F)F]

### Step 2: 1,1,1-Trifluoro-2-{1-[3-(4-fluoro-phenyl)-2-methanesulfonylmethyl-indolizin-7-ylmethyl]-1H-[1,2,3]triazol-4-yl}-butan-2-ol

Methanesulfonic acid 3-(4-fluoro-phenyl)-7-[4-(1-hydroxy-1-trifluoromethyl-propyl)-[1,2,3]triazol-1-ylmethyl]-indolizin-2-ylmethyl ester (from the previous step) was dissolved in DMF (10 mL) and sodium methansulfinate (0.120 g, 1.15 mmol) was added. The reaction was heated to 60° C. for 2 hours then cooled and submitted to standard aqueous workup. The residue was purified on silica gel (0-100% EtOAc in hexanes) to give the title compound.

### Example 44

### Synthesis of 7-[4-(1-Hydroxy-1-trifluoromethyl-propyl)-[1,2,3]triazol-1-ylmethyl]-3-(6-methoxy-pyridin-3-yl)-indolizine-2-carboxylic acid ethyl ester (Compound 1-36)

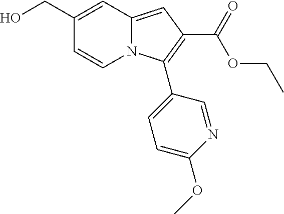 [SMILES: CCOC(=O)c1cc2cc(CO)ccn2c1-c1ccc(OC)nc1]

### Step 1: 7-Hydroxymethyl-3-(6-methoxy-pyridin-3-yl)-indolizine-2-carboxylic acid ethyl ester

Prepared according to the procedure described in Example 9, Step 1, using the following starting materials: 7-hydroxymethyl-indolizine-2-carboxylic acid ethyl ester and 5-bromo-2-methoxy-pyridine.

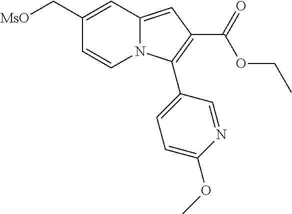 [SMILES: CCOC(=O)c1cc2cc(COS(C)(=O)=O)ccn2c1-c1ccc(OC)nc1]

### Step 2: 7-Methanesulfonyloxymethyl-3-(6-methoxy-pyridin-3-yl)-indolizine-2-carboxylic acid ethyl ester

Prepared according to the procedure described in Example 1, Step 7, using the following starting material: 7-hydroxymethyl-3-(6-methoxy-pyridin-3-yl)-indolizine-2-carboxylic acid ethyl

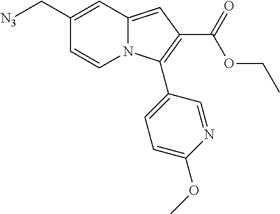 [SMILES: CCOC(=O)c1cc2cc(CN=[N+]=[N-])ccn2c1-c1ccc(OC)nc1]

### Step 3: 7-Azidomethyl-3-(6-methoxy-pyridin-3-yl)-indolizine-2-carboxylic acid ethyl ester

Prepared according to the procedure described in Example 1, Step 8, using the following starting material: 7-methanesulfonyloxymethyl-3-(6-methoxy-pyridin-3-yl)-indolizine-2-carboxylic

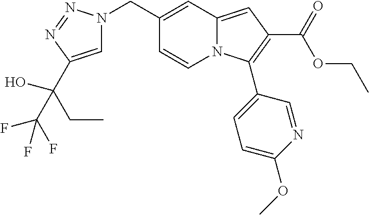 [SMILES: CCOC(=O)c1cc2cc(Cn3cc(C(O)(CC)C(F)(F)F)nn3)ccn2c1-c1ccc(OC)nc1]

### Step 4: 7-[4-(1-Hydroxy-1-trifluoromethyl-propyl)-[1,2,3]triazol-1-ylmethyl]-3-(6-methoxy-pyridin-3-yl)-indolizine-2-carboxylic acid ethyl ester

Prepared according to the procedure described in Example 1, Step 11, using the following starting materials: 7-azidomethyl-3-(6-methoxy-pyridin-3-yl)-indolizine-2-carboxylic acid ethyl ester and 3-trifluoromethyl-pent-1-yn-3-ol.

### Example 45

### Synthesis of 7-[4-(1-Hydroxy-1-trifluoromethyl-propyl)-[1,2,3]triazol-1-ylmethyl]-3-(6-methoxy-pyridin-3-yl)-indolizine-2-carboxylic acid amide (Compound 1-35)

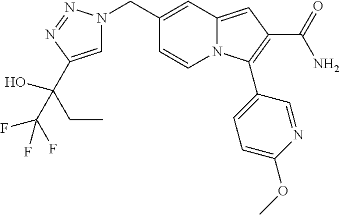 [SMILES: CCC(O)(c1cn(Cc2ccn3c(-c4ccc(OC)nc4)c(C(N)=O)cc3c2)nn1)C(F)(F)F]

### Step 1: 7-[4-(1-Hydroxy-1-trifluoromethyl-propyl)-[1,2,3]triazol-1-ylmethyl]-3-(6-methoxy-pyridin-3-yl)-indolizine-2-carboxylic acid amide

Prepared according to the procedure described in Example 9, Step 6, using the following starting material: 7-[4-(1-hydroxy-1-trifluoromethyl-propyl)-[1,2,3]triazol-1-ylmethyl]-3-(6-methoxy-pyridin-3-yl)-indolizine-2-carboxylic acid ethyl ester.

### Example 46

### Synthesis of 3-({3-(4-Fluoro-phenyl)-7-[4-(1-hydroxy-1-trifluoromethyl-propyl)-[1,2,3]triazol-1-ylmethyl]-indolizin-2-ylmethyl}-amino)-propionic acid ethyl ester (Compound 1-37)

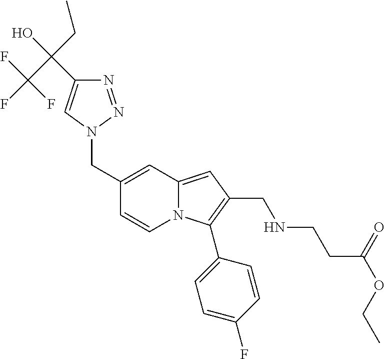 [SMILES: CCOC(=O)CCNCc1cc2cc(Cn3cc(C(O)(CC)C(F)(F)F)nn3)ccn2c1-c1ccc(F)cc1]

### Step 1: 3-({3-(4-Fluoro-phenyl)-7-[4-(1-hydroxy-1-trifluoromethyl-propyl)-[1,2,3]triazol-1-ylmethyl]-indolizin-2-ylmethyl}-amino)-propionic acid ethyl ester

3-(4-Fluoro-phenyl)-7-[4-(1-hydroxy-1-trifluoromethyl-propyl)-[1,2,3]triazol-1-ylmethyl]-indolizine-2-carbaldehyde (0.150 g, 0.34 mmol) was dissolved in DCE (8 mL) then β-alanine ethyl ester hydrochloride (0.7834 g, 0.51 mmol), acetic acid (0.0038 mL) and molecular sieves were added. The reaction stirred at room temperature for 1 hour then sodium triacetoxyborohydride (0.1081 g, 0.51 mmol) was added. After 30 minutes the reaction was filtered then submitted to standard aqueous workup and preparatory HPLC purification (10-100% ACN in H₂O) to yield the title compound.

### Example 47

### Synthesis of 1,1,1-Trifluoro-2-{1-[3-(4-fluoro-phenyl)-2-morpholin-4-ylmethyl-indolizin-7-ylmethyl]-1H-[1,2,3]triazol-4-yl}-butan-2-ol (Compound 1-38)

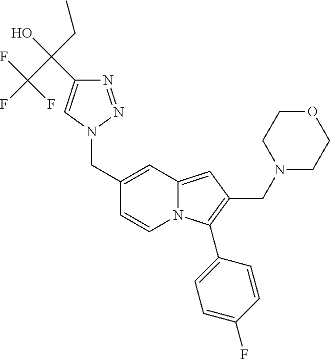 [SMILES: CCC(O)(c1cn(Cc2ccn3c(-c4ccc(F)cc4)c(CN4CCOCC4)cc3c2)nn1)C(F)(F)F]

### Step 1: 1,1,1-Trifluoro-2-{1-[3-(4-fluoro-phenyl)-2-morpholin-4-ylmethyl-indolizin-7-ylmethyl]-1H-[1,2,3]triazol-4-yl}-butan-2-ol

Prepared according to the procedure described in Example 46, Step 1, using the following starting materials: 3-(4-Fluoro-phenyl)-7-[4-(1-hydroxy-1-trifluoromethyl-propyl)-[1,2,3]triazol-1-ylmethyl]-indolizine-2-carbaldehyde and morpholine.

### Example 48

### Synthesis of (E)-3-{3-(4-Fluoro-phenyl)-7-[4-(1-hydroxy-1-trifluoromethyl-propyl)-[1,2,3]triazol-1-ylmethyl]-indolizin-2-yl}-acrylic acid methyl ester (Compound 1-39)

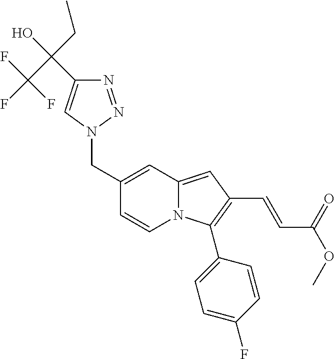 [SMILES: CCC(O)(c1cn(Cc2ccn3c(-c4ccc(F)cc4)c(/C=C/C(=O)OC)cc3c2)nn1)C(F)(F)F]

### Step 1: (E)-3-{3-(4-Fluoro-phenyl)-7-[4-(1-hydroxy-1-trifluoromethyl-propyl)-[1,2,3]triazol-1-ylmethyl]-indolizin-2-yl}-acrylic acid methyl ester

Trimethyl phosphonoacetate (0.233 mL, 1.6 mmol) was dissolved in THF (20 mL) and cooled to 0° C. Sodium hydride (60% in mineral oil, 0.066 g, 1.67 mmol) was added and the reaction stirred for 5 minutes. 3-(4-Fluoro-phenyl)-7-[4-(1-hydroxy-1-trifluoromethyl-propyl)-[1,2,3]triazol-1-ylmethyl]-indolizine-2-carbaldehyde (0.150 g, 0.34 mmol) in THF (10 mL) was added and the reaction slowly warmed to room temperature. When no starting material remained by analytical TLC, the reaction was submitted to standard aqueous workup. The residue was purified on silica gel (0-100% EtOAc in hexanes) to give the title compound.

### Example 49

### Synthesis of 3-({3-(4-Fluoro-phenyl)-7-[4-(1-hydroxy-1-trifluoromethyl-propyl)-[1,2,3]triazol-1-ylmethyl]-indolizin-2-ylmethyl}-amino)-propionic acid (Compound 1-40)

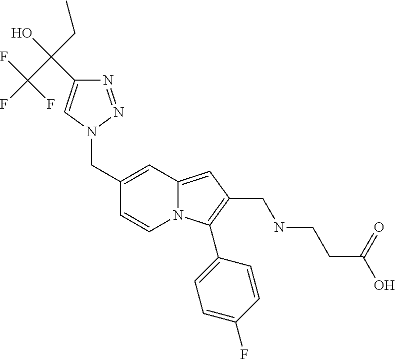 [SMILES: CCC(O)(c1cn(Cc2ccn3c(-c4ccc(F)cc4)c(CNCCC(=O)O)cc3c2)nn1)C(F)(F)F]

### Step 1: 3-({3-(4-Fluoro-phenyl)-7-[4-(1-hydroxy-1-trifluoromethyl-propyl)-[1,2,3]triazol-1-ylmethyl]-indolizin-2-ylmethyl}-amino)-propionic acid

Prepared according to the procedure described in Example 5, Step 1, using the following starting material: 3-({3-(4-fluoro-phenyl)-7-[4-(1-hydroxy-1-trifluoromethyl-propyl)-[1,2,3]triazol-1-ylmethyl]-indolizin-2-ylmethyl}-amino)-propionic acid ethyl ester.

### Example 50

### Synthesis of (E)-3-{3-(4-Fluoro-phenyl)-7-[4-(1-hydroxy-1-trifluoromethyl-propyl)-[1,2,3]triazol-1-ylmethyl]-indolizin-2-yl}-acrylic acid (Compound 1-41)

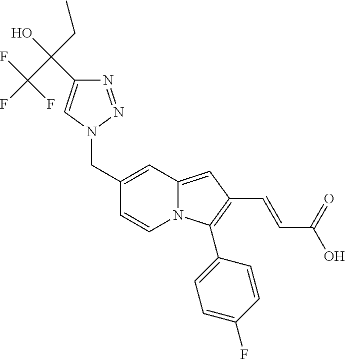 [SMILES: CCC(O)(c1cn(Cc2ccn3c(-c4ccc(F)cc4)c(/C=C/C(=O)O)cc3c2)nn1)C(F)(F)F]

### Step 1: (E)-3-{3-(4-Fluoro-phenyl)-7-[4-(1-hydroxy-1-trifluoromethyl-propyl)-[1,2,3]triazol-1-ylmethyl]-indolizin-2-yl}-acrylic acid

Prepared according to the procedure described in Example 5, Step 1, using the following starting material: (E)-3-{3-(4-fluoro-phenyl)-7-[4-(1-hydroxy-1-trifluoromethyl-propyl)-[1,2,3]triazol-1-ylmethyl]-indolizin-2-yl}-acrylic acid methyl ester.

### Example 51

### Synthesis of 3-({3-(4-Fluoro-phenyl)-7-[4-(1-hydroxy-1-trifluoromethyl-propyl)-[1,2,3]triazol-1-ylmethyl]-indolizin-2-ylmethyl}-amino)-propionitrile (Compound 1-42)

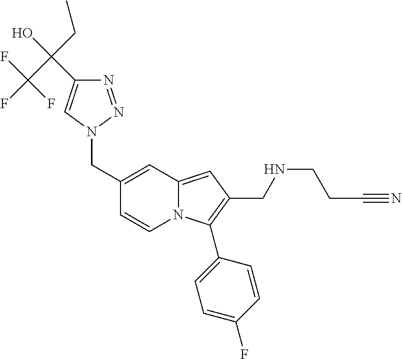 [SMILES: CCC(O)(c1cn(Cc2ccn3c(-c4ccc(F)cc4)c(CNCCC#N)cc3c2)nn1)C(F)(F)F]

### Step 1: 3-({3-(4-Fluoro-phenyl)-7-[4-(1-hydroxy-1-trifluoromethyl-propyl)-[1,2,3]triazol-1-ylmethyl]-indolizin-2-ylmethyl}-amino)-propionitrile

Prepared according to the procedure described in Example 9, Step 6, using the following starting material: 3-({3-(4-fluoro-phenyl)-7-[4-(1-hydroxy-1-trifluoromethyl-propyl)-[1,2,3]triazol-1-ylmethyl]-indolizin-2-ylmethyl}-amino)-propionic acid ethyl ester.

### Example 52

### Synthesis of (E)-3-{3-(4-Fluoro-phenyl)-7-[4-(1-hydroxy-1-trifluoromethyl-propyl)-[1,2,3]triazol-1-ylmethyl]-indolizin-2-yl}-acrylamide (Compound 1-43)

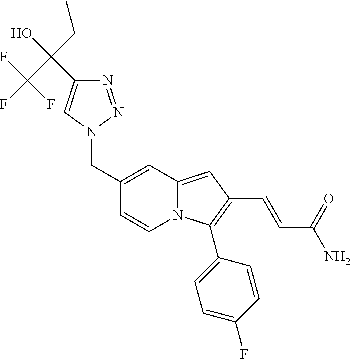 [SMILES: CCC(O)(c1cn(Cc2ccn3c(-c4ccc(F)cc4)c(/C=C/C(N)=O)cc3c2)nn1)C(F)(F)F]

### Step 1: (E)-3-{3-(4-Fluoro-phenyl)-7-[4-(1-hydroxy-1-trifluoromethyl-propyl)-[1,2,3]triazol-1-ylmethyl]-indolizin-2-yl}-acrylamide

Prepared according to the procedure described in Example 9, Step 6, using the following starting material: (E)-3-{3-(4-fluoro-phenyl)-7-[4-(1-hydroxy-1-trifluoromethyl-propyl)-[1,2,3]triazol-1-ylmethyl]-indolizin-2-yl}-acrylic acid methyl ester.

### Example 53

### Synthesis of 1,1,1-Trifluoro-2-{1-[3-(4-fluoro-phenyl)-2-methoxymethyl-indolizin-7-ylmethyl]-1H-[1,2,3]triazol-4-yl}-butan-2-ol (Compound 1-44)

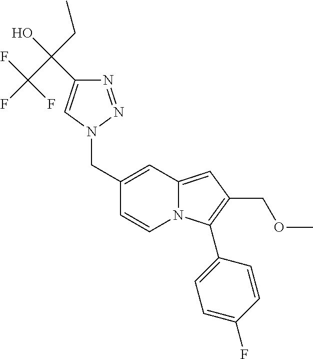 [SMILES: CCC(O)(c1cn(Cc2ccn3c(-c4ccc(F)cc4)c(COC)cc3c2)nn1)C(F)(F)F]

### Step 1: 1,1,1-Trifluoro-2-{1-[3-(4-fluoro-phenyl)-2-methoxymethyl-indolizin-7-ylmethyl]-1H-[1,2,3]triazol-4-yl}-butan-2-ol

Prepared according to the procedure described in Example 9, Step 4, using the following starting materials: 1,1,1-Trifluoro-2-{1-[3-(4-fluoro-phenyl)-2-hydroxymethyl-indolizin-7-ylmethyl]-1H-[1,2,3]triazol-4-yl}-butan-2-ol and iodomethane. After the reaction, the residue was purified by silica gel chromatography (0-100% EtOAc in hexanes) to give a separable mixture of two products, of which the major product was the title compound.

### Example 54

### Synthesis of 3-(4-Fluoro-phenyl)-2-methoxymethyl-7-[4-(1-methoxy-1-trifluoromethyl-propyl)-[1,2,3]triazol-1-ylmethyl]-indolizine (Compound 1-45)

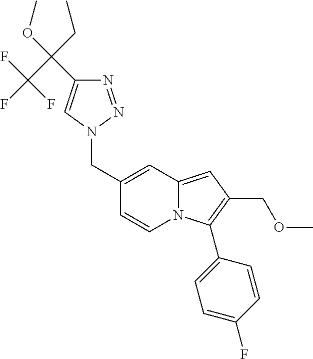 [SMILES: CCC(OC)(c1cn(Cc2ccn3c(-c4ccc(F)cc4)c(COC)cc3c2)nn1)C(F)(F)F]

### Step 1: 3-(4-Fluoro-phenyl)-2-methoxymethyl-7-[4-(1-methoxy-1-trifluoromethyl-propyl)-[1,2,3]triazol-1-ylmethyl]-indolizine

Prepared according to the procedure described in Example 9, Step 4, using the following starting materials: 1,1,1-Trifluoro-2-{1-[3-(4-fluoro-phenyl)-2-hydroxymethyl-indolizin-7-ylmethyl]-1H-[1,2,3]triazol-4-yl}-butan-2-ol and iodomethane. After the reaction, the residue was purified by silica gel chromatography (0-100% EtOAc in hexanes) to give a separable mixture of two products, of which the minor product was the title compound.

### Example 55

### Synthesis of 3-(3-Fluoro-phenyl)-7-[4-(1-hydroxy-1-trifluoromethyl-propyl)-[1,2,3]triazol-1-ylmethyl]-indolizine-2-carboxylic acid ethyl ester (Compound 1-46)

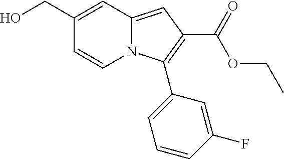 [SMILES: CCOC(=O)c1cc2cc(CO)ccn2c1-c1cccc(F)c1]

### Step 1: 3-(3-Fluoro-phenyl)-7-hydroxymethyl-indolizine-2-carboxylic acid ethyl ester

Prepared according to the procedure described in Example 9, Step 1, using the following starting materials: 7-hydroxymethyl-indolizine-2-carboxylic acid ethyl ester and 1-bromo-3-fluoro-benzene.

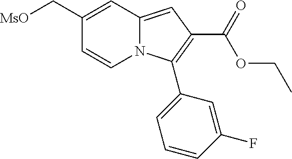 [SMILES: CCOC(=O)c1cc2cc(COS(C)(=O)=O)ccn2c1-c1cccc(F)c1]

### Step 2: 3-(3-Fluoro-phenyl)-7-methanesulfonyloxymethyl-indolizine-2-carboxylic acid ethyl ester

Prepared according to the procedure described in Example 1, Step 7, using the following starting material: 3-(3-fluoro-phenyl)-7-hydroxymethyl-indolizine-2-carboxylic acid ethyl ester.

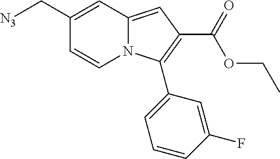 [SMILES: CCOC(=O)c1cc2cc(CN=[N+]=[N-])ccn2c1-c1cccc(F)c1]

### Step 3: 7-Azidomethyl-3-(3-fluoro-phenyl)-indolizine-2-carboxylic acid ethyl ester

Prepared according to the procedure described in Example 1, Step 8, using the following starting material: 3-(3-fluoro-phenyl)-7-methanesulfonyloxymethyl-indolizine-2-carboxylic acid ethyl ester.

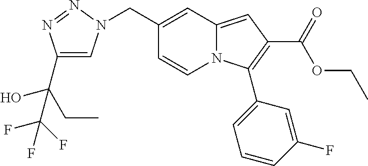 [SMILES: CCOC(=O)c1cc2cc(Cn3cc(C(O)(CC)C(F)(F)F)nn3)ccn2c1-c1cccc(F)c1]

### Step 4: 3-(3-Fluoro-phenyl)-7-[4-(1-hydroxy-1-trifluoromethyl-propyl)-[1,2,3]triazol-1-ylmethyl]-indolizine-2-carboxylic acid ethyl ester

Prepared according to the procedure described in Example 1, Step 11, using the following starting materials: 7-azidomethyl-3-(3-fluoro-phenyl)-indolizine-2-carboxylic acid ethyl ester and 3-trifluoromethyl-pent-1-yn-3-ol.

### Example 56

### Synthesis of 7-[4-(1-Hydroxy-1-trifluoromethyl-propyl)-[1,2,3]triazol-1-ylmethyl]-3-(3-methanesulfonyl-phenyl)-indolizine-2-carboxylic acid ethyl ester (Compound 1-47)

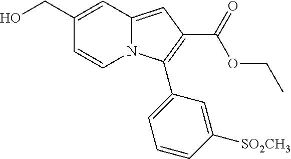 [SMILES: CCOC(=O)c1cc2cc(CO)ccn2c1-c1cccc(S(C)(=O)=O)c1]

### Step 1: 7-Hydroxymethyl-3-(3-methanesulfonyl-phenyl)-indolizine-2-carboxylic acid ethyl ester

Prepared according to the procedure described in Example 9, Step 1, using the following starting materials: 7-hydroxymethyl-indolizine-2-carboxylic acid ethyl ester and 1-bromo-3-methanesulfonyl-benzene.

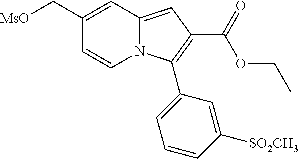 [SMILES: CCOC(=O)c1cc2cc(COS(C)(=O)=O)ccn2c1-c1cccc(S(C)(=O)=O)c1]

### Step 2: 7-Methanesulfonyloxymethyl-3-(3-methanesulfonyl-phenyl)-indolizine-2-carboxylic acid ethyl ester

Prepared according to the procedure described in Example 1, Step 7, using the following starting material: 7-hydroxymethyl-3-(3-methanesulfonyl-phenyl)-indolizine-2-carboxylic acid ethyl ester.

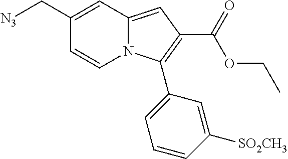 [SMILES: CCOC(=O)c1cc2cc(CN=[N+]=[N-])ccn2c1-c1cccc(S(C)(=O)=O)c1]

### Step 3: 7-Azidomethyl-3-(3-methanesulfonyl-phenyl)-indolizine-2-carboxylic acid ethyl ester

Prepared according to the procedure described in Example 1, Step 8, using the following starting material: 7-methanesulfonyloxymethyl-3-(3-methanesulfonyl-phenyl)-indolizine-2-carboxylic acid ethyl ester.

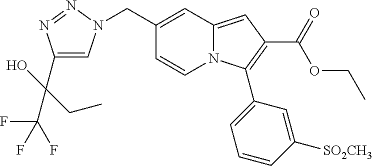 [SMILES: CCOC(=O)c1cc2cc(Cn3cc(C(O)(CC)C(F)(F)F)nn3)ccn2c1-c1cccc(S(C)(=O)=O)c1]

### Step 4: 7-[4-(1-Hydroxy-1-trifluoromethyl-propyl)-[1,2,3]triazol-1-ylmethyl]-3-(3-methanesulfonyl-phenyl)-indolizine-2-carboxylic acid ethyl ester

Prepared according to the procedure described in Example 1, Step 11, using the following starting materials: 7-azidomethyl-3-(3-methanesulfonyl-phenyl)-indolizine-2-carboxylic acid ethyl ester and 3-trifluoromethyl-pent-1-yn-3-ol.

### Example 57

### Synthesis of 3-(3-Fluoro-phenyl)-7-[4-(1-hydroxy-1-trifluoromethyl-propyl)-[1,2,3]triazol-1-ylmethyl]-indolizine-2-carboxylic acid amide (Compound 1-48)

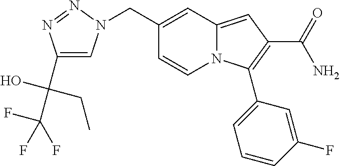 [SMILES: CCC(O)(c1cn(Cc2ccn3c(-c4cccc(F)c4)c(C(N)=O)cc3c2)nn1)C(F)(F)F]

### Step 1: 3-(3-Fluoro-phenyl)-7-[4-(1-hydroxy-1-trifluoromethyl-propyl)-[1,2,3]triazol-1-ylmethyl]-indolizine-2-carboxylic acid amide

Prepared according to the procedure described in Example 9, Step 6, using the following starting material: 3-(3-fluoro-phenyl)-7-[4-(1-hydroxy-1-trifluoromethyl-propyl)-[1,2,3]triazol-1-ylmethyl]-indolizine-2-carboxylic acid ethyl ester.

### Example 58

### Synthesis of 7-[4-(1-Hydroxy-1-trifluoromethyl-propyl)-[1,2,3]triazol-1-ylmethyl]-3-(3-methanesulfonyl-phenyl)-indolizine-2-carbonitrile (Compound 1-49)

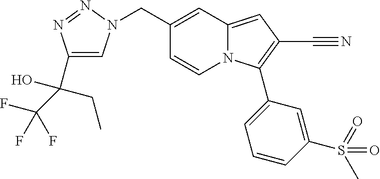 [SMILES: CCC(O)(c1cn(Cc2ccn3c(-c4cccc(S(C)(=O)=O)c4)c(C#N)cc3c2)nn1)C(F)(F)F]

### Step 1: 7-[4-(1-Hydroxy-1-trifluoromethyl-propyl)-[1,2,3]triazol-1-ylmethyl]-3-(3-methanesulfonyl-phenyl)-indolizine-2-carbonitrile

Prepared according to the procedure described in Example 9, Step 6, using the following starting material: 7-[4-(1-hydroxy-1-trifluoromethyl-propyl)-[1,2,3]triazol-1-ylmethyl]-3-(3-methanesulfonyl-phenyl)-indolizine-2-carboxylic acid ethyl ester. The reaction yielded a mixture of two separable products, of which the title compound was the minor product isolated.

### Example 59

### Synthesis of (1,1-Dioxo-1(λ)6-thiazolidin-3-yl)-{3-(4-fluoro-phenyl)-7-[4-(1-hydroxy-1-trifluoromethyl-propyl)-[1,2,3]triazol-1-ylmethyl]-indolizin-2-yl}-methanone (Compound 1-50)

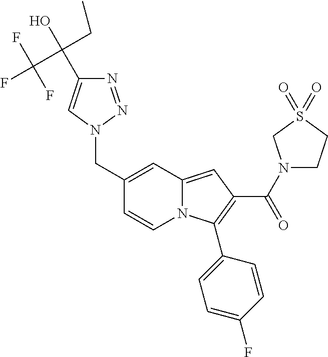 [SMILES: CCC(O)(c1cn(Cc2ccn3c(-c4ccc(F)cc4)c(C(=O)N4CCS(=O)(=O)C4)cc3c2)nn1)C(F)(F)F]

### Step 1: (1,1-Dioxo-1(λ)6-thiazolidin-3-yl)-{3-(4-fluoro-phenyl)-7-[4-(1-hydroxy-1-trifluoromethyl-propyl)-[1,2,3]triazol-1-ylmethyl]-indolizin-2-yl}-methanone

Prepared according to the procedure described in Example 6, Step 1, using the following starting materials: 3-(4-fluoro-phenyl)-7-[4-(1-hydroxy-1-trifluoromethyl-propyl)-[1,2,3]triazol-1-ylmethyl]-indolizine-2-carboxylic acid and thiazolidine 1,1-dioxide hydrochloride.

### Example 60

### Synthesis of 7-[4-(1-Hydroxy-1-trifluoromethyl-propyl)-[1,2,3]triazol-1-ylmethyl]-3-quinolin-3-yl-indolizine-2-carboxylic acid ethyl ester (Compound 1-52)

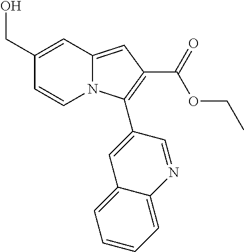 [SMILES: CCOC(=O)c1cc2cc(CO)ccn2c1-c1cnc2ccccc2c1]

### Step 1: 7-Hydroxymethyl-3-quinolin-3-yl-indolizine-2-carboxylic acid ethyl ester

Prepared according to the procedure described in Example 9, Step 1, using the following starting materials: 7-hydroxymethyl-indolizine-2-carboxylic acid ethyl ester and 3-bromo-quinoline.

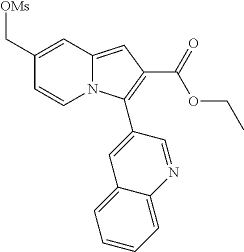 [SMILES: CCOC(=O)c1cc2cc(COS(C)(=O)=O)ccn2c1-c1cnc2ccccc2c1]

### Step 2: 7-Methanesulfonyloxymethyl-3-quinolin-3-yl-indolizine-2-carboxylic acid ethyl ester

Prepared according to the procedure described in Example 1, Step 7, using the following starting material: 7-hydroxymethyl-3-quinolin-3-yl-indolizine-2-carboxylic acid ethyl ester.

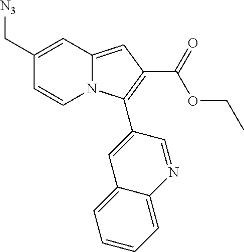 [SMILES: CCOC(=O)c1cc2cc(CN=[N+]=[N-])ccn2c1-c1cnc2ccccc2c1]

### Step 3: 7-Azidomethyl-3-quinolin-3-yl-indolizine-2-carboxylic acid ethyl ester

Prepared according to the procedure described in Example 1, Step 8, using the following starting material: 7-methanesulfonyloxymethyl-3-quinolin-3-yl-indolizine-2-carboxylic acid ethyl ester.

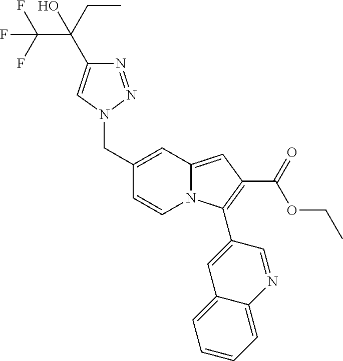 [SMILES: CCOC(=O)c1cc2cc(Cn3cc(C(O)(CC)C(F)(F)F)nn3)ccn2c1-c1cnc2ccccc2c1]

### Step 4: 7-[4-(1-Hydroxy-1-trifluoromethyl-propyl)-[1,2,3]triazol-1-ylmethyl]-3-quinolin-3-yl-indolizine-2-carboxylic acid ethyl ester

Prepared according to the procedure described in Example 1, Step 11, using the following starting materials: 7-azidomethyl-3-quinolin-3-yl-indolizine-2-carboxylic acid ethyl ester and 3-trifluoromethyl-pent-1-yn-3-ol.

### Example 61

### Synthesis of 7-[4-(1-Hydroxy-1-trifluoromethyl-propyl)-[1,2,3]triazol-1-ylmethyl]-3-quinolin-3-yl-indolizine-2-carbonitrile (Compound 1-53)

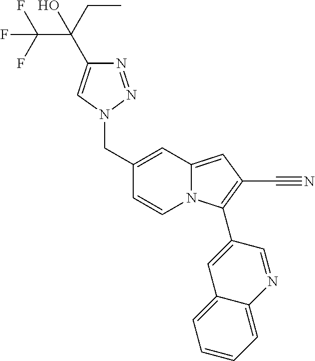 [SMILES: CCC(O)(c1cn(Cc2ccn3c(-c4cnc5ccccc5c4)c(C#N)cc3c2)nn1)C(F)(F)F]

### Step 1: 7-[4-(1-Hydroxy-1-trifluoromethyl-propyl)-[1,2,3]triazol-1-ylmethyl]-3-quinolin-3-yl-indolizine-2-carbonitrile

Prepared according to the procedure described in Example 9, Step 6, using the following starting material: 7-[4-(1-hydroxy-1-trifluoromethyl-propyl)-[1,2,3]triazol-1-ylmethyl]-3-quinolin-3-yl-indolizine-2-carboxylic acid ethyl ester. The reaction yielded a mixture of two separable products, of which the title compound was the minor product isolated.

### Example 62

### Synthesis of 3-(4-Fluoro-phenyl)-7-[4-(1-hydroxy-1-trifluoromethyl-propyl)-[1,2,3]triazol-1-ylmethyl]-indolizine-2-carboxylic acid ethylamide (Compound 1-55)

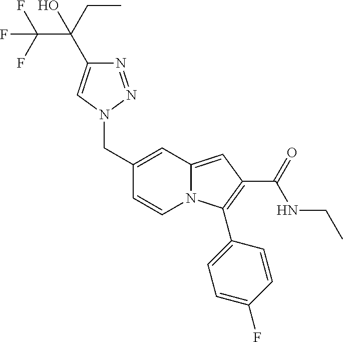 [SMILES: CCNC(=O)c1cc2cc(Cn3cc(C(O)(CC)C(F)(F)F)nn3)ccn2c1-c1ccc(F)cc1]

### Step 1: 3-(4-Fluoro-phenyl)-7-[4-(1-hydroxy-1-trifluoromethyl-propyl)-[1,2,3]triazol-1-ylmethyl]-indolizine-2-carboxylic acid ethylamide

Prepared according to the procedure described in Example 6, Step 1, using the following starting materials: 3-(4-fluoro-phenyl)-7-[4-(1-hydroxy-1-trifluoromethyl-propyl)-[1,2,3]triazol-1-ylmethyl]-indolizine-2-carb oxylic acid and ethylamine.

### Example 63

### Synthesis of 3-(6-Ethoxy-pyridin-3-yl)-7-[4-(1-hydroxy-1-trifluoromethyl-propyl)-[1,2,3]triazol-1-ylmethyl]-indolizine-2-carboxylic acid ethyl ester (Compound 1-56)

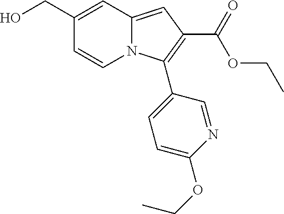 [SMILES: CCOC(=O)c1cc2cc(CO)ccn2c1-c1ccc(OCC)nc1]

### Step 1: 3-(6-Ethoxy-pyridin-3-yl)-7-hydroxymethyl-indolizine-2-carboxylic acid ethyl ester

Prepared according to the procedure described in Example 9, Step 1, using the following starting materials: 7-hydroxymethyl-indolizine-2-carboxylic acid ethyl ester and 5-bromo-2-ethoxy-pyridine.

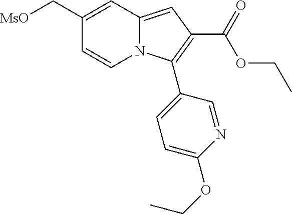 [SMILES: CCOC(=O)c1cc2cc(COS(C)(=O)=O)ccn2c1-c1ccc(OCC)nc1]

### Step 2: 3-(6-Ethoxy-pyridin-3-yl)-7-methanesulfonyloxymethyl-indolizine-2-carboxylic acid ethyl ester

Prepared according to the procedure described in Example 1, Step 7, using the following starting material: 3-(6-ethoxy-pyridin-3-yl)-7-hydroxymethyl-indolizine-2-carboxylic acid ethyl ester.

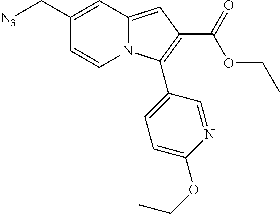 [SMILES: CCOC(=O)c1cc2cc(CN=[N+]=[N-])ccn2c1-c1ccc(OCC)nc1]

### Step 3: 7-Azidomethyl-3-(6-ethoxy-pyridin-3-yl)-indolizine-2-carboxylic acid ethyl ester

Prepared according to the procedure described in Example 1, Step 8, using the following starting material: 3-(6-Ethoxy-pyridin-3-yl)-7-methanesulfonyloxymethyl-indolizine-2-carboxylic acid ethyl ester.

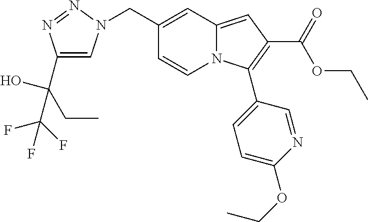 [SMILES: CCOC(=O)c1cc2cc(Cn3cc(C(O)(CC)C(F)(F)F)nn3)ccn2c1-c1ccc(OCC)nc1]

### Step 4: 3-(6-Ethoxy-pyridin-3-yl)-7-[4-(1-hydroxy-1-trifluoromethyl-propyl)-[1,2,3]triazol-1-ylmethyl]-indolizine-2-carboxylic acid ethyl ester

Prepared according to the procedure described in Example 1, Step 11, using the following starting materials: 7-azidomethyl-3-(6-ethoxy-pyridin-3-yl)-indolizine-2-carboxylic acid ethyl ester and 3-trifluoromethyl-pent-1-yn-3-ol.

### Example 64

### Synthesis of 7-[4-(1-Hydroxy-1-trifluoromethyl-propyl)-[1,2,3]triazol-1-ylmethyl]-3-(6-methoxy-pyridin-3-yl)-indolizine-2-carbonitrile (Compound 1-57)

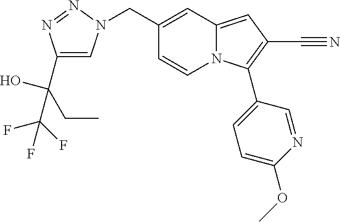 [SMILES: CCC(O)(c1cn(Cc2ccn3c(-c4ccc(OC)nc4)c(C#N)cc3c2)nn1)C(F)(F)F]

### Step 1: 7-[4-(1-Hydroxy-1-trifluoromethyl-propyl)-[1,2,3]triazol-1-ylmethyl]-3-(6-methoxy-pyridin-3-yl)-indolizine-2-carbonitrile

Prepared according to the procedure described in Example 9, Step 6, using the following starting material: 7-[4-(1-hydroxy-1-trifluoromethyl-propyl)-[1,2,3]triazol-1-ylmethyl]-3-(6-methoxy-pyridin-3-yl)-indolizine-2-carboxylic acid ethyl ester.

### Example 65

### Synthesis of 3-(6-Ethoxy-pyridin-3-yl)-7-[4-(1-hydroxy-1-trifluoromethyl-propyl)-[1,2,3]triazol-1-ylmethyl]-indolizine-2-carboxylic acid amide (Compound 1-58)

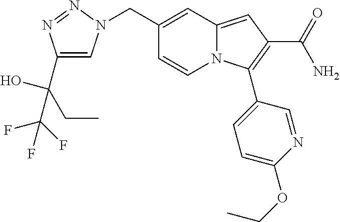 [SMILES: CCOc1ccc(-c2c(C(N)=O)cc3cc(Cn4cc(C(O)(CC)C(F)(F)F)nn4)ccn23)cn1]

### Step 1: 3-(6-Ethoxy-pyridin-3-yl)-7-[4-(1-hydroxy-1-trifluoromethyl-propyl)-[1,2,3]triazol-1-ylmethyl]-indolizine-2-carboxylic acid amide

Prepared according to the procedure described in Example 9, Step 6, using the following starting material: 3-(6-ethoxy-pyridin-3-yl)-7-[4-(1-hydroxy-1-trifluoromethyl-propyl)-[1,2,3]triazol-1-ylmethyl]-indolizine-2-carboxylic acid ethyl ester. The reaction yielded a mixture of two separable products, of which the title compound was the major product isolated.

### Example 66

### Synthesis of 3-(6-Ethoxy-pyridin-3-yl)-7-[4-(1-hydroxy-1-trifluoromethyl-propyl)-[1,2,3]triazol-1-ylmethyl]-indolizine-2-carbonitrile (Compound 1-59)

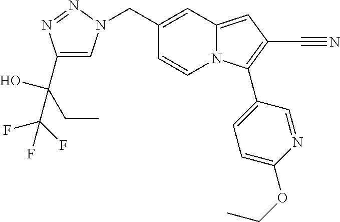 [SMILES: CCOc1ccc(-c2c(C#N)cc3cc(Cn4cc(C(O)(CC)C(F)(F)F)nn4)ccn23)cn1]

### Step 1: 3-(6-Ethoxy-pyridin-3-yl)-7-[4-(1-hydroxy-1-trifluoromethyl-propyl)-[1,2,3]triazol-1-ylmethyl]-indolizine-2-carbonitrile

Prepared according to the procedure described in Example 9, Step 6, using the following starting material: 3-(6-ethoxy-pyridin-3-yl)-7-[4-(1-hydroxy-1-trifluoromethyl-propyl)-[1,2,3]triazol-1-ylmethyl]-indolizine-2-carboxylic acid ethyl ester. The reaction yielded a mixture of two separable products, of which the title compound was the minor product isolated.

### Example 67

### Synthesis of 2-[1-[2-Ethylaminomethyl-3-(4-fluoro-phenyl)-indolizin-7-ylmethyl]-1H-[1,2,3]triazol-4-yl]-1,1,1-trifluoro-butan-2-ol (Compound 1-60)

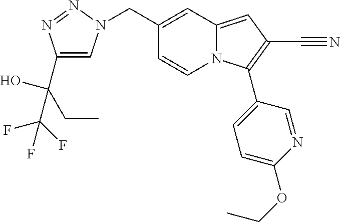 [SMILES: CCOc1ccc(-c2c(C#N)cc3cc(Cn4cc(C(O)(CC)C(F)(F)F)nn4)ccn23)cn1]

### Step 1: 2-{1-[2-Ethylaminomethyl-3-(4-fluoro-phenyl)-indolizin-7-ylmethyl]-1H-[1,2,3]triazol-4-yl}-1,1,1-trifluoro-butan-2-ol

Prepared according to the procedure described in Example 46, Step 1, using the following starting materials: 3-(4-fluoro-phenyl)-7-[4-(1-hydroxy-1-trifluoromethyl-propyl)-[1,2,3]triazol-1-ylmethyl]-indolizine-2-carbaldehyde and ethylamine.

### Example 68

### Synthesis of 7-[4-(1-Hydroxy-1-trifluoromethyl-propyl)-[1,2,3]triazol-1-ylmethyl]-3-(1-methyl-6-oxo-1,6-dihydro-pyridin-3-yl)-indolizine-2-carboxylic acid ethyl ester (Compound

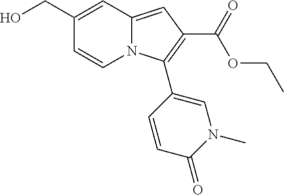 [SMILES: CCOC(=O)c1cc2cc(CO)ccn2c1-c1ccc(=O)n(C)c1]

### Step 1: 7-Hydroxymethyl-3-(1-methyl-6-oxo-1,6-dihydro-pyridin-3-yl)-indolizine-2-carboxylic acid ethyl ester

Prepared according to the procedure described in Example 9, Step 1, using the following starting materials: 7-hydroxymethyl-indolizine-2-carboxylic acid ethyl ester and 5-bromo-1-methyl-1H-pyridin-2-one

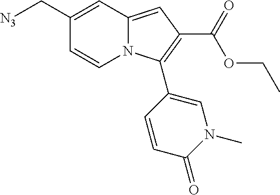 [SMILES: CCOC(=O)c1cc2cc(CN=[N+]=[N-])ccn2c1-c1ccc(=O)n(C)c1]

### Step 2: 7-Azidomethyl-3-(1-methyl-6-oxo-1,6-dihydro-pyridin-3-yl)-indolizine-2-carboxylic acid ethyl ester

7-Hydroxymethyl-3-(1-methyl-6-oxo-1,6-dihydro-pyridin-3-yl)-indolizine-2-carboxylic acid ethyl ester (0.120 g, 0.37 mmol), sodium azide (0.0335 g, 052 mmol) and then phosphorous trichloride (0.0162 g, 0.41 mmol) were dissolved in CCl₄:DMF (1:4, 1 mL). The reaction was heated to 90° C. and monitored by analytical LCMS. When the reaction was complete it was submitted to standard aqueous workup to afford the title compound, which was used in the next step without further purification.

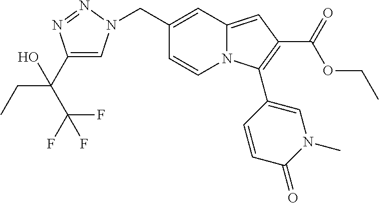 [SMILES: CCOC(=O)c1cc2cc(Cn3cc(C(O)(CC)C(F)(F)F)nn3)ccn2c1-c1ccc(=O)n(C)c1]

### Step 3: 7-[4-(1-Hydroxy-1-trifluoromethyl-propyl)-[1,2,3]triazol-1-ylmethyl]-3-(1-methyl-6-oxo-1,6-dihydro-pyridin-3-yl)-indolizine-2-carboxylic acid ethyl ester

Prepared according to the procedure described in Example 1, Step 11, using the following starting materials: 7-azidomethyl-3-(1-methyl-6-oxo-1,6-dihydro-pyridin-3-yl)-indolizine-2-carboxylic acid ethyl ester and 3-trifluoromethyl-pent-1-yn-3-ol.

### Example 69

### Synthesis of 7-[4-(1-Hydroxy-1-trifluoromethyl-propyl)-[1,2,3]triazol-1-ylmethyl]-3-(2-methyl-pyridin-4-yl)-indolizine-2-carboxylic acid ethyl ester (Compound 1-63)

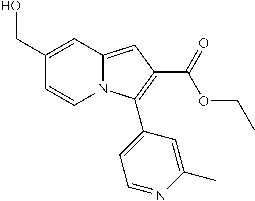 [SMILES: CCOC(=O)c1cc2cc(CO)ccn2c1-c1ccnc(C)c1]

### Step 1: 7-Hydroxymethyl-3-(2-methyl-pyridin-4-yl)-indolizine-2-carboxylic acid ethyl ester

Prepared according to the procedure described in Example 9, Step 1, using the following starting materials: 7-hydroxymethyl-indolizine-2-carboxylic acid ethyl ester and 4-bromo-2-methyl-pyridine.

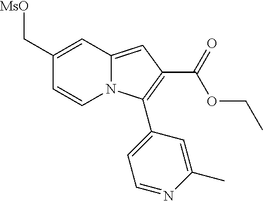 [SMILES: CCOC(=O)c1cc2cc(COS(C)(=O)=O)ccn2c1-c1ccnc(C)c1]

### Step 2: 7-Methanesulfonyloxymethyl-3-(2-methyl-pyridin-4-yl)-indolizine-2-carboxylic acid ethyl ester

Prepared according to the procedure described in Example 1, Step 7, using the following starting material: 7-hydroxymethyl-3-(2-methyl-pyridin-4-yl)-indolizine-2-carboxylic acid ethyl ester.

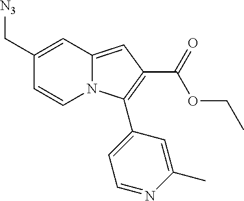 [SMILES: CCOC(=O)c1cc2cc(CN=[N+]=[N-])ccn2c1-c1ccnc(C)c1]

### Step 3: 7-Azidomethyl-3-(2-methyl-pyridin-4-yl)-indolizine-2-carboxylic acid ethyl ester

Prepared according to the procedure described in Example 1, Step 8, using the following starting material: 7-methanesulfonyloxymethyl-3-(2-methyl-pyridin-4-yl)-indolizine-2-carboxylic acid

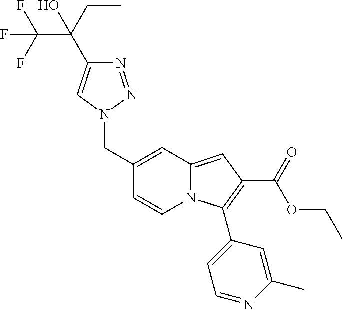 [SMILES: CCOC(=O)c1cc2cc(Cn3cc(C(O)(CC)C(F)(F)F)nn3)ccn2c1-c1ccnc(C)c1]

### Step 4: 7-[4-(1-Hydroxy-1-trifluoromethyl-propyl)-[1,2,3]triazol-1-ylmethyl]-3-(2-methyl-pyridin-4-yl)-indolizine-2-carboxylic acid ethyl ester

Prepared according to the procedure described in Example 1, Step 11, using the following starting materials: 7-azidomethyl-3-(2-methyl-pyridin-4-yl)-indolizine-2-carboxylic acid ethyl ester and 3-trifluoromethyl-pent-1-yn-3-ol.

### Example 70

### Synthesis of 7-[4-(1-Hydroxy-1-trifluoromethyl-propyl)-[1,2,3]triazol-1-ylmethyl]-3-(2-methyl-pyridin-4-yl)-indolizine-2-carboxylic acid amide (Compound 1-64)

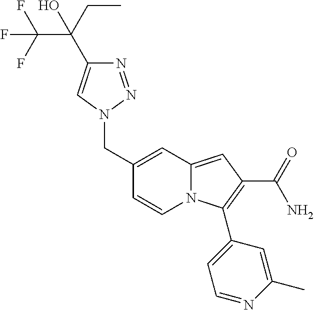 [SMILES: CCC(O)(c1cn(Cc2ccn3c(-c4ccnc(C)c4)c(C(N)=O)cc3c2)nn1)C(F)(F)F]

### Step 1: 7-[4-(1-Hydroxy-1-trifluoromethyl-propyl)-[1,2,3]triazol-1-ylmethyl]-3-(2-methyl-pyridin-4-yl)-indolizine-2-carboxylic acid amide

Prepared according to the procedure described in Example 9, Step 6, using the following starting material: 7-[4-(1-hydroxy-1-trifluoromethyl-propyl)-[1,2,3]triazol-1-ylmethyl]-3-(2-methyl-pyridin-4-yl)-indolizine-2-carboxylic acid ethyl ester. The reaction yielded a mixture of two separable products, of which the title compound was the major product isolated.

### Example 71

### Synthesis of 7-[4-(1-Hydroxy-1-trifluoromethyl-propyl)-[1,2,3]triazol-1-ylmethyl]-3-(2-methyl-pyridin-4-yl)-indolizine-2-carbonitrile (Compound 1-65)

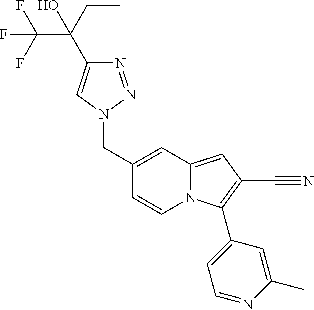 [SMILES: CCC(O)(c1cn(Cc2ccn3c(-c4ccnc(C)c4)c(C#N)cc3c2)nn1)C(F)(F)F]

### Step 1: 7-[4-(1-Hydroxy-1-trifluoromethyl-propyl)-[1,2,3]triazol-1-ylmethyl]-3-(2-methyl-pyridin-4-yl)-indolizine-2-carbonitrile

Prepared according to the procedure described in Example 9, Step 6, using the following starting material: 7-[4-(1-hydroxy-1-trifluoromethyl-propyl)-[1,2,3]triazol-1-ylmethyl]-3-(2-methyl-pyridin-4-yl)-indolizine-2-carboxylic acid ethyl ester. The reaction yielded a mixture of two separable products, of which the title compound was the minor product isolated.

### Example 72

### Synthesis of 3-Acetyl-7-[4-(1-hydroxy-1-trifluoromethyl-propyl)-[1,2,3]triazol-1-ylmethyl]-indolizine-2-carboxylic acid methyl ester (Compound 1-66)

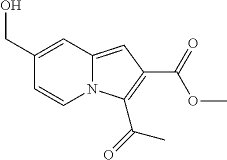 [SMILES: COC(=O)c1cc2cc(CO)ccn2c1C(C)=O]

### Step 1: 3-Acetyl-7-hydroxymethyl-indolizine-2-carboxylic acid methyl ester

Prepared according to the procedure described in Example 13, Step 4, using the following starting material: 7-acetoxymethyl-3-acetyl-indolizine-2-carboxylic acid ethyl ester.

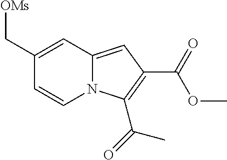 [SMILES: COC(=O)c1cc2cc(COS(C)(=O)=O)ccn2c1C(C)=O]

### Step 2: 3-Acetyl-7-methanesulfonyloxymethyl-indolizine-2-carboxylic acid methyl ester

Prepared according to the procedure described in Example 1, Step 7, using the following starting material: 3-acetyl-7-hydroxymethyl-indolizine-2-carboxylic acid methyl ester.

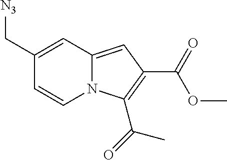 [SMILES: COC(=O)c1cc2cc(CN=[N+]=[N-])ccn2c1C(C)=O]

### Step 3: 3-Acetyl-7-azidomethyl-indolizine-2-carboxylic acid methyl ester

Prepared according to the procedure described in Example 1, Step 8, using the following starting material: 3-acetyl-7-methanesulfonyloxymethyl-indolizine-2-carboxylic acid methyl ester.

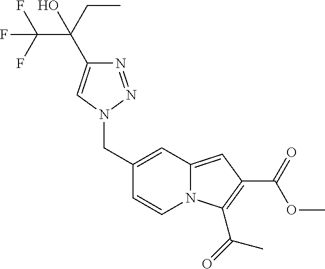 [SMILES: CCC(O)(c1cn(Cc2ccn3c(C(C)=O)c(C(=O)OC)cc3c2)nn1)C(F)(F)F]

### Step 4: 3-Acetyl-7-[4-(1-hydroxy-1-trifluoromethyl-propyl)-[1,2,3]triazol-1-ylmethyl]-indolizine-2-carboxylic acid methyl ester

Prepared according to the procedure described in Example 1, Step 11, using the following starting materials: 3-acetyl-7-azidomethyl-indolizine-2-carboxylic acid methyl ester and 3-trifluoromethyl-pent-1-yn-3-ol.

### Example 73

### Synthesis of 3-Acetyl-7-[4-(1-hydroxy-1-trifluoromethyl-propyl)-[1,2,3]triazol-1-ylmethyl]-indolizine-2-carboxylic acid amide (Compound 1-67)

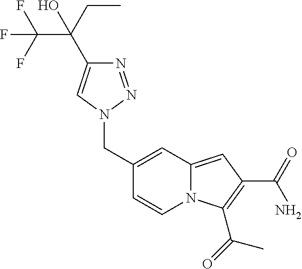 [SMILES: CCC(O)(c1cn(Cc2ccn3c(C(C)=O)c(C(N)=O)cc3c2)nn1)C(F)(F)F]

### Step 1: 3-Acetyl-7-[4-(1-hydroxy-1-trifluoromethyl-propyl)-[1,2,3]triazol-1-ylmethyl]-indolizine-2-carboxylic acid amide

Prepared according to the procedure described in Example 9, Step 6, using the following starting material: 3-acetyl-7-[4-(1-hydroxy-1-trifluoromethyl-propyl)-[1,2,3]triazol-1-ylmethyl]-indolizine-2-carboxylic acid methyl ester.

### Example 74

### Synthesis of 7-[4-(1-Hydroxy-1-trifluoromethyl-propyl)-[1,2,3]triazol-1-ylmethyl]-3-(1-methyl-6-oxo-1,6-dihydro-pyridin-3-yl)-indolizine-2-carboxylic acid amide (Compound 1-68)

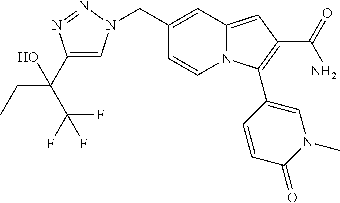 [SMILES: CCC(O)(c1cn(Cc2ccn3c(-c4ccc(=O)n(C)c4)c(C(N)=O)cc3c2)nn1)C(F)(F)F]

### Step 1: 7-[4-(1-Hydroxy-1-trifluoromethyl-propyl)-[1,2,3]triazol-1-ylmethyl]-3-(1-methyl-6-oxo-1,6-dihydro-pyridin-3-yl)-indolizine-2-carboxylic acid amide

Prepared according to the procedure described in Example 9, Step 6, using the following starting material: 7-[4-(1-hydroxy-1-trifluoromethyl-propyl)-[1,2,3]triazol-1-ylmethyl]-3-(1-methyl-6-oxo-1,6-dihydro-pyridin-3-yl)-indolizine-2-carboxylic acid ethyl ester. The reaction yielded a mixture of two separable products, of which the title compound was the major product isolated.

### Example 75

### Synthesis of 7-[4-(1-Hydroxy-1-trifluoromethyl-propyl)-[1,2,3]triazol-1-ylmethyl]-3-(6-methyl-pyridin-3-yl)-indolizine-2-carboxylic acid ethyl ester (Compound 1-69)

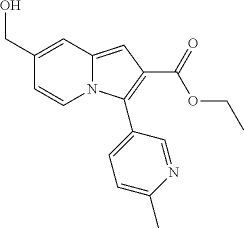 [SMILES: CCOC(=O)c1cc2cc(CO)ccn2c1-c1ccc(C)nc1]

### Step 1: 7-Hydroxymethyl-3-(6-methyl-pyridin-3-yl)-indolizine-2-carboxylic acid ethyl ester

Prepared according to the procedure described in Example 9, Step 1, using the following starting materials: 7-hydroxymethyl-indolizine-2-carboxylic acid ethyl ester and 5-bromo-2-methyl-pyridine.

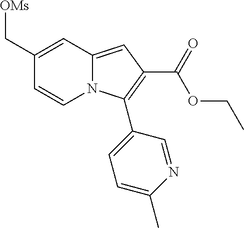 [SMILES: CCOC(=O)c1cc2cc(COS(C)(=O)=O)ccn2c1-c1ccc(C)nc1]

### Step 2: 7-Methanesulfonyloxymethyl-3-(6-methyl-pyridin-3-yl)-indolizine-2-carboxylic acid ethyl ester

Prepared according to the procedure described in Example 1, Step 7, using the following starting material: 7-hydroxymethyl-3-(6-methyl-pyridin-3-yl)-indolizine-2-carboxylic acid ethyl ester.

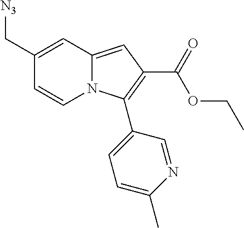 [SMILES: CCOC(=O)c1cc2cc(CN=[N+]=[N-])ccn2c1-c1ccc(C)nc1]

### Step 3: 7-Azidomethyl-3-(6-methyl-pyridin-3-yl)-indolizine-2-carboxylic acid ethyl ester

Prepared according to the procedure described in Example 1, Step 8, using the following starting material: 7-methanesulfonyloxymethyl-3-(6-methyl-pyridin-3-yl)-indolizine-2-carboxylic acid

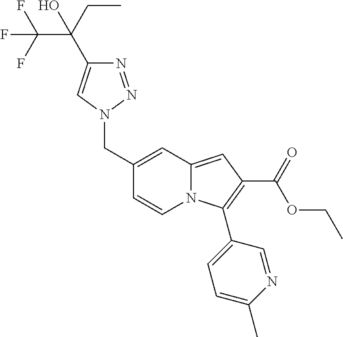 [SMILES: CCOC(=O)c1cc2cc(Cn3cc(C(O)(CC)C(F)(F)F)nn3)ccn2c1-c1ccc(C)nc1]

### Step 4: 7-[4-(1-Hydroxy-1-trifluoromethyl-propyl)-[1,2,3]triazol-1-ylmethyl]-3-(6-methyl-pyridin-3-yl)-indolizine-2-carboxylic acid ethyl ester

Prepared according to the procedure described in Example 1, Step 11, using the following starting materials: 7-azidomethyl-3-(6-methyl-pyridin-3-yl)-indolizine-2-carboxylic acid ethyl ester and 3-trifluoromethyl-pent-1-yn-3-ol.

### Example 76

### Synthesis of 7-[4-(1-Hydroxy-1-trifluoromethyl-propyl)-[1,2,3]triazol-1-ylmethyl]-3-(1-methyl-6-oxo-1,6-dihydro-pyridin-3-yl)-indolizine-2-carbonitrile (Compound 1-70)

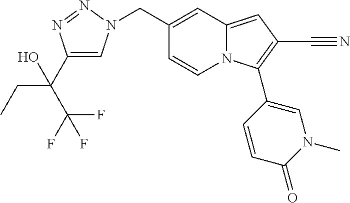 [SMILES: CCC(O)(c1cn(Cc2ccn3c(-c4ccc(=O)n(C)c4)c(C#N)cc3c2)nn1)C(F)(F)F]

### Step 1: 7-[4-(1-Hydroxy-1-trifluoromethyl-propyl)-[1,2,3]triazol-1-ylmethyl]-3-(1-methyl-6-oxo-1,6-dihydro-pyridin-3-yl)-indolizine-2-carbonitrile

Prepared according to the procedure described in Example 9, Step 6, using the following starting material: 7-[4-(1-hydroxy-1-trifluoromethyl-propyl)-[1,2,3]triazol-1-ylmethyl]-3-(1-methyl-6-oxo-1,6-dihydro-pyridin-3-yl)-indolizine-2-carb oxylic acid ethyl ester. The reaction yielded a mixture of two separable products, of which the title compound was the minor product isolated.

### Example 77

### Synthesis of 7-[4-(1-Hydroxy-1-trifluoromethyl-propyl)-[1,2,3]triazol-1-ylmethyl]-3-(6-methyl-pyridin-3-yl)-indolizine-2-carboxylic acid amide (Compound 1-71)

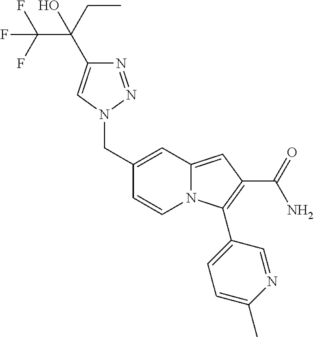 [SMILES: CCC(O)(c1cn(Cc2ccn3c(-c4ccc(C)nc4)c(C(N)=O)cc3c2)nn1)C(F)(F)F]

### Step 1: 7-[4-(1-Hydroxy-1-trifluoromethyl-propyl)-[1,2,3]triazol-1-ylmethyl]-3-(6-methyl-pyridin-3-yl)-indolizine-2-carboxylic acid amide

Prepared according to the procedure described in Example 9, Step 6, using the following starting material: 7-[4-(1-hydroxy-1-trifluoromethyl-propyl)-[1,2,3]triazol-1-ylmethyl]-3-(6-methyl-pyridin-3-yl)-indolizine-2-carboxylic acid ethyl ester. The reaction yielded a mixture of two separable products, of which the title compound was the major product isolated.

### Example 78

### Synthesis of 7-[4-(1-Hydroxy-1-trifluoromethyl-propyl)-[1,2,3]triazol-1-ylmethyl]-3-(6-methyl-pyridin-3-yl)-indolizine-2-carbonitrile (Compound 1-72)

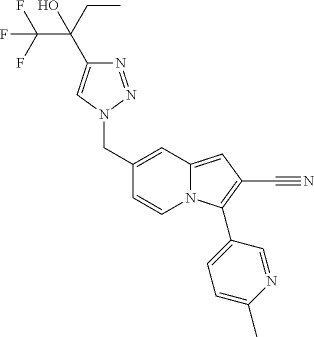 [SMILES: CCC(O)(c1cn(Cc2ccn3c(-c4ccc(C)nc4)c(C#N)cc3c2)nn1)C(F)(F)F]

### Step 1: 7-[4-(1-Hydroxy-1-trifluoromethyl-propyl)-[1,2,3]triazol-1-ylmethyl]-3-(6-methyl-pyridin-3-yl)-indolizine-2-carbonitrile

Prepared according to the procedure described in Example 9, Step 6, using the following starting material: 7-[4-(1-hydroxy-1-trifluoromethyl-propyl)-[1,2,3]triazol-1-ylmethyl]-3-(6-methyl-pyridin-3-yl)-indolizine-2-carboxylic acid ethyl ester. The reaction yielded a mixture of two separable products, of which the title compound was the minor product isolated.

### Example 79

### Synthesis of 7-[4-(1-Hydroxy-1-trifluoromethyl-propyl)-[1,2,3]triazol-1-ylmethyl]-3-pyrimidin-5-yl-indolizine-2-carboxylic acid ethyl ester (Compound 1-73)

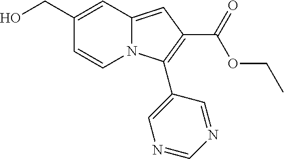 [SMILES: CCOC(=O)c1cc2cc(CO)ccn2c1-c1cncnc1]

### Step 1: 7-Hydroxymethyl-3-pyrimidin-5-yl-indolizine-2-carboxylic acid ethyl ester

Prepared according to the procedure described in Example 9, Step 1, using the following starting materials: 7-hydroxymethyl-indolizine-2-carboxylic acid ethyl ester and 5-bromo-pyrimidine.

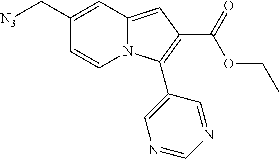 [SMILES: CCOC(=O)c1cc2cc(CN=[N+]=[N-])ccn2c1-c1cncnc1]

### Step 2: 7-Azidomethyl-3-pyrimidin-5-yl-indolizine-2-carboxylic acid ethyl ester

Prepared according to the procedure described in Example 68, Step 2, using the following starting material: 7-hydroxymethyl-3-pyrimidin-5-yl-indolizine-2-carboxylic acid ethyl ester.

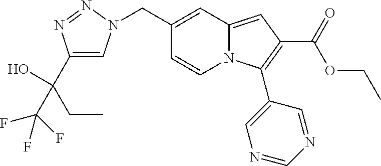 [SMILES: CCOC(=O)c1cc2cc(Cn3cc(C(O)(CC)C(F)(F)F)nn3)ccn2c1-c1cncnc1]

### Step 3: 7-[4-(1-Hydroxy-1-trifluoromethyl-propyl)-[1,2,3]triazol-1-ylmethyl]-3-pyrimidin-5-yl-indolizine-2-carboxylic acid ethyl ester

Prepared according to the procedure described in Example 1, Step 11, using the following starting materials: 7-azidomethyl-3-pyrimidin-5-yl-indolizine-2-carboxylic acid ethyl ester and 3-trifluoromethyl-pent-1-yn-3-ol.

### Example 80

### Synthesis of 7-[4-(1-Hydroxy-1-trifluoromethyl-propyl)-[1,2,3]triazol-1-ylmethyl]-3-(1-methyl-1H-pyrazol-4-yl)-indolizine-2-carboxylic acid ethyl ester (Compound 1-74)

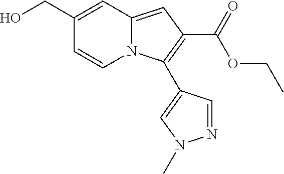 [SMILES: CCOC(=O)c1cc2cc(CO)ccn2c1-c1cnn(C)c1]

### Step 1: 7-Hydroxymethyl-3-(1-methyl-1H-pyrazol-4-yl)-indolizine-2-carboxylic acid ethyl ester

Prepared according to the procedure described in Example 9, Step 1, using the following starting materials: 7-hydroxymethyl-indolizine-2-carboxylic acid ethyl ester and 4-bromo-1-methyl-1H-pyrazole.

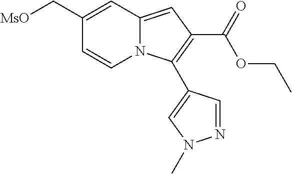 [SMILES: CCOC(=O)c1cc2cc(COS(C)(=O)=O)ccn2c1-c1cnn(C)c1]

### Step 2: 7-Methanesulfonyloxymethyl-3-(1-methyl-1H-pyrazol-4-yl)-indolizine-2-carboxylic acid ethyl ester

Prepared according to the procedure described in Example 1, Step 7, using the following starting material: 7-hydroxymethyl-3-(1-methyl-1H-pyrazol-4-yl)-indolizine-2-carboxylic acid ethyl

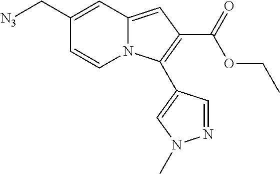 [SMILES: CCOC(=O)c1cc2cc(CN=[N+]=[N-])ccn2c1-c1cnn(C)c1]

### Step 3: 7-azidomethyl-3-(1-methyl-1H-pyrazol-4-yl)-indolizine-2-carboxylic acid ethyl ester

Prepared according to the procedure described in Example 1, Step 8, using the following starting material: 7-methanesulfonyloxymethyl-3-(1-methyl-1H-pyrazol-4-yl)-indolizine-2-carboxylic acid ethyl ester.

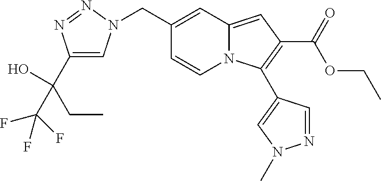 [SMILES: CCOC(=O)c1cc2cc(Cn3cc(C(O)(CC)C(F)(F)F)nn3)ccn2c1-c1cnn(C)c1]

### Step 4: 7-[4-(1-Hydroxy-1-trifluoromethyl-propyl)-[1,2,3]triazol-1-ylmethyl]-3-(1-methyl-1H-pyrazol-4-yl)-indolizine-2-carboxylic acid ethyl ester

Prepared according to the procedure described in Example 1, Step 11, using the following starting materials: 7-azidomethyl-3-(1-methyl-1H-pyrazol-4-yl)-indolizine-2-carboxylic acid ethyl ester and 3-trifluoromethyl-pent-1-yn-3-ol.

### Example 81

### Synthesis of 4-Nitro-benzoic acid 1-[1-[2-carbamoyl-3-(6-methyl-pyridin-3-yl)-indolizin-7-ylmethyl]-1H-[1,2,3]triazol-4-yl]-1-trifluoromethyl-propyl ester (Enantiomer B) (Compound 1-75)

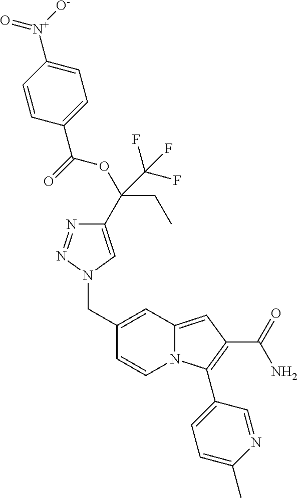 [SMILES: CCC(OC(=O)c1ccc([N+](=O)[O-])cc1)(c1cn(Cc2ccn3c(-c4ccc(C)nc4)c(C(N)=O)cc3c2)nn1)C(F)(F)F]

### Step 1: 4-Nitro-benzoic acid 1-[1-[2-carbamoyl-3-(6-methyl-pyridin-3-yl)-indolizin-7-ylmethyl]-1H-[1,2,3]triazol-4-yl]-1-trifluoromethyl-propyl ester (Enantiomer B)

Prepared according to the procedure described in Example 9, Step 6, where the minor product isolated was the title compound.

### Example 82

### Synthesis of 7-[4-(1-Hydroxy-1-trifluoromethyl-propyl)-[1,2,3]triazol-1-ylmethyl]-3-(6-methoxy-pyridin-3-yl)-1-(2,2,2-trifluoro-acetyl)-indolizine-2-carboxylic acid amide (Enantiomer B) (Compound 1-82)

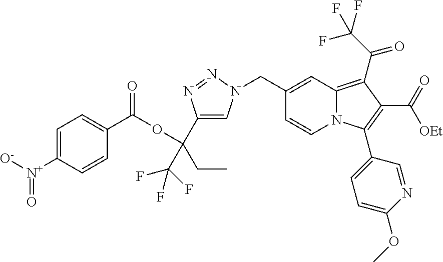 [SMILES: CCOC(=O)c1c(C(=O)C(F)(F)F)c2cc(Cn3cc(C(CC)(OC(=O)c4ccc([N+](=O)[O-])cc4)C(F)(F)F)nn3)ccn2c1-c1ccc(OC)nc1]

### Step 1: 3-(6-Methoxy-pyridin-3-yl)-7-{4-[1-(4-nitro-benzoyloxy)-1-trifluoromethyl-propyl]-[1,2,3]triazol-1-ylmethyl}-1-(2,2,2-trifluoro-acetyl)-indolizine-2-carboxylic acid ethyl ester (Enantiomer B)

3-(6-Methoxy-pyridin-3-yl)-7-{4-[1-(4-nitro-benzoyloxy)-1-trifluoromethyl-propyl]-[1,2,3]triazol-1-ylmethyl}-indolizine-2-carboxylic acid ethyl ester (Enantiomer B) (0.075 g, 0.115 mmol) was dissolved in CH₂Cl₂ (1.15 mL). Trifluoroacetic anhydride (0.032 mL, 0.223 mmol) was added and the reaction stirred at room temperature overnight. The reaction mixture was submitted to standard aqueous workup to yield the title compound.

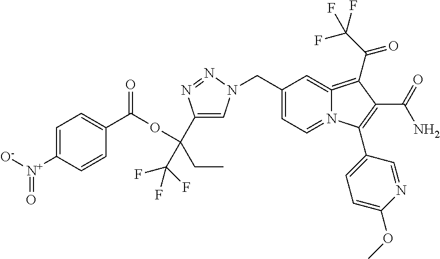

### Step 2: 4-Nitro-benzoic acid 1-{1-[2-carbamoyl-3-(6-methoxy-pyridin-3-yl)-1-(2,2,2-trifluoro-acetyl)-indolizin-7-ylmethyl]-1H-[1,2,3]triazol-4-yl}-1-trifluoromethyl-propyl ester (Enantiomer B)

Prepared according to the procedure described in Example 9, Step 6, using the following starting material: 3-(6-methoxy-pyridin-3-yl)-7-{4-[1-(4-nitro-benzoyloxy)-1-trifluoromethyl-propyl]-[1,2,3]triazol-1-ylmethyl}-1-(2,2,2-trifluoro-acetyl)-indolizine-2-carboxylic acid ethyl ester (Enantiomer B).

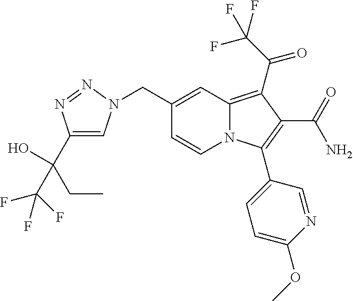 [SMILES: CCC(O)(c1cn(Cc2ccn3c(-c4ccc(OC)nc4)c(C(N)=O)c(C(=O)C(F)(F)F)c3c2)nn1)C(F)(F)F]

### Step 3: 7-[4-(1-Hydroxy-1-trifluoromethyl-propyl)-[1,2,3]triazol-1-ylmethyl]-3-(6-methoxy-pyridin-3-yl)-1-(2,2,2-trifluoro-acetyl)-indolizine-2-carboxylic acid amide (Enantiomer B)

Prepared according to the procedure described in Example 9, Step 7, using the following starting material: 4-Nitro-benzoic acid 1-{1-[2-carbamoyl-3-(6-methoxy-pyridin-3-yl)-1-(2,2,2-trifluoro-acetyl)-indolizin-7-ylmethyl]-1H-[1,2,3]triazol-4-yl}-1-trifluoromethyl-propyl ester (Enantiomer B).

### Example 83

### Synthesis of 4-Nitro-benzoic acid 1-{1-[2-carbamoyl-3-(6-methoxy-pyridin-3-yl)-indolizin-7-ylmethyl]-1H-[1,2,3]triazol-4-yl}-1-trifluoromethyl-propyl ester (Enantiomer B) (Compound 1-83)

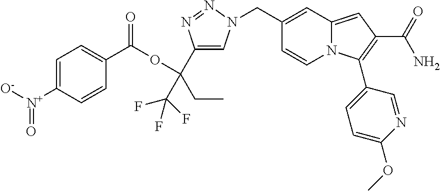 [SMILES: CCC(OC(=O)c1ccc([N+](=O)[O-])cc1)(c1cn(Cc2ccn3c(-c4ccc(OC)nc4)c(C(N)=O)cc3c2)nn1)C(F)(F)F]

Prepared according to the procedure described in Example 10, Step 5.

### Example 84

### Synthesis of 3-(6-Methoxy-pyridin-3-yl)-7-[4-[1-(4-nitro-benzoyloxy)-1-trifluoromethyl-propyl]-[1,2,3]triazol-1-ylmethyl]-1-(2,2,2-trifluoro-acetyl)-indolizine-2-carboxylic acid ethyl ester (Enantiomer B) (Compound 1-84)

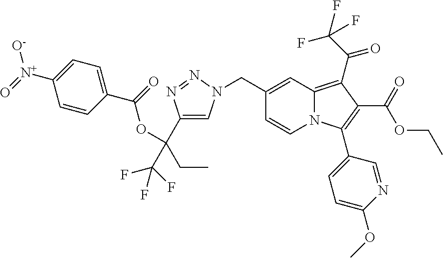 [SMILES: CCOC(=O)c1c(C(=O)C(F)(F)F)c2cc(Cn3cc(C(CC)(OC(=O)c4ccc([N+](=O)[O-])cc4)C(F)(F)F)nn3)ccn2c1-c1ccc(OC)nc1]

Prepared according to the procedure described in Example 82, Step 1.

### Example 85

### Synthesis of 3-(6-Methoxy-pyridin-3-yl)-7-[4-[1-(4-nitro-benzoyloxy)-1-trifluoromethyl-propyl]-[1,2,3]triazol-1-ylmethyl]-indolizine-2-carboxylic acid ethyl ester (Enantiomer B) (Compound 1-85)

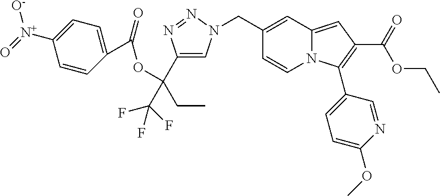 [SMILES: CCOC(=O)c1cc2cc(Cn3cc(C(CC)(OC(=O)c4ccc([N+](=O)[O-])cc4)C(F)(F)F)nn3)ccn2c1-c1ccc(OC)nc1]

Prepared according to the procedure described in Example 10, Step 4.

### Example 86

### Synthesis of 7-[4-[1-(4-Nitro-benzoyloxy)-1-trifluoromethyl-propyl]-[1,2,3]triazol-1-ylmethyl]-indolizine-2-carboxylic acid ethyl ester (Enantiomer B) (Compound 1-86)

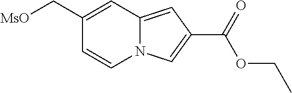 [SMILES: CCOC(=O)c1cc2cc(COS(C)(=O)=O)ccn2c1]

### Step 1: 7-Methanesulfonyloxymethyl-indolizine-2-carboxylic acid ethyl ester

Prepared according to the procedure described in Example 1, Step 7, using the following starting material: 7-hydroxymethyl-indolizine-2-carboxylic acid ethyl ester.

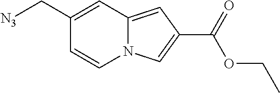 [SMILES: CCOC(=O)c1cc2cc(CN=[N+]=[N-])ccn2c1]

### Step 2: 7-Azidomethyl-indolizine-2-carboxylic acid ethyl ester

Prepared according to the procedure described in Example 1, Step 8, using the following starting material: 7-methanesulfonyloxymethyl-indolizine-2-carboxylic acid ethyl ester.

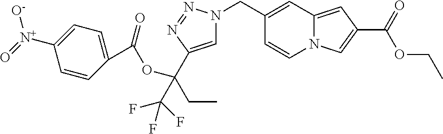 [SMILES: CCOC(=O)c1cc2cc(Cn3cc(C(CC)(OC(=O)c4ccc([N+](=O)[O-])cc4)C(F)(F)F)nn3)ccn2c1]

### Step 3: 7-{4-[1-(4-Nitro-benzoyloxy)-1-trifluoromethyl-propyl]-[1,2,3]triazol-1-ylmethyl}-indolizine-2-carboxylic acid ethyl ester (Enantiomer B)

Prepared according to the procedure described in Example 1, Step 11, using the following starting materials: 7-azidomethyl-indolizine-2-carboxylic acid ethyl ester and 4-nitro-benzoic acid 1-ethyl-1-trifluoromethyl-prop-2-ynyl ester (Enantiomer B).

### Example 87

### Synthesis of 7-{4-[1-(4-Nitro-benzoyloxy)-1-trifluoromethyl-propyl]-[1,2,3]triazol-1-ylmethyl}-indolizine-2-carboxylic acid ethyl ester (Enantiomer B) (Compound 1-87)

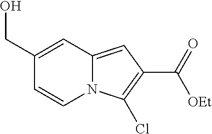 [SMILES: CCOC(=O)c1cc2cc(CO)ccn2c1Cl]

### Step 1: 3-Chloro-7-hydroxymethyl-indolizine-2-carboxylic acid ethyl ester

Prepared according to the procedure described in Example 8, Step 1, using the following starting material: 7-hydroxymethyl-indolizine-2-carboxylic acid ethyl ester.

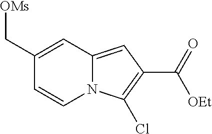 [SMILES: CCOC(=O)c1cc2cc(COS(C)(=O)=O)ccn2c1Cl]

### Step 2: 3-Chloro-7-methanesulfonyloxymethyl-indolizine-2-carboxylic acid ethyl ester

Prepared according to the procedure described in Example 1, Step 7, using the following starting material: 3-chloro-7-hydroxymethyl-indolizine-2-carboxylic acid ethyl ester.

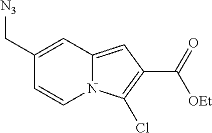 [SMILES: CCOC(=O)c1cc2cc(CN=[N+]=[N-])ccn2c1Cl]

### Step 3: 7-Azidomethyl-3-chloro-indolizine-2-carboxylic acid ethyl ester

Prepared according to the procedure described in Example 1, Step 8, using the following starting material: 3-chloro-7-methanesulfonyloxymethyl-indolizine-2-carboxylic acid ethyl ester.

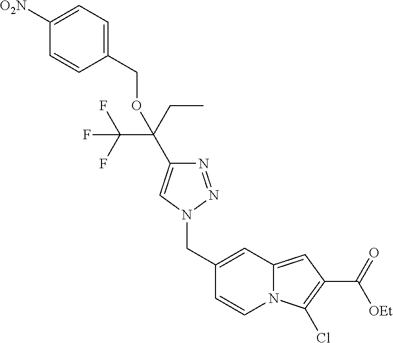 [SMILES: CCOC(=O)c1cc2cc(Cn3cc(C(CC)(OCc4ccc([N+](=O)[O-])cc4)C(F)(F)F)nn3)ccn2c1Cl]

### Step 4: 3-Chloro-7-{4-[1-(4-nitro-benzyloxy)-1-trifluoromethyl-propyl]-[1,2,3]triazol-1-ylmethyl}-indolizine-2-carboxylic acid ethyl ester (Enantiomer B)

Prepared according to the procedure described in Example 1, Step 11, using the following starting materials: 7-azidomethyl-3-chloro-indolizine-2-carboxylic acid ethyl ester and 4-nitro-benzoic acid 1-ethyl-1-trifluoromethyl-prop-2-ynyl ester (Enantiomer B).

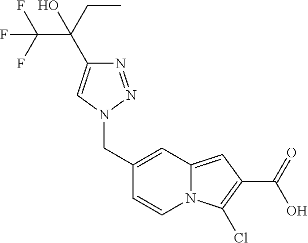 [SMILES: CCC(O)(c1cn(Cc2ccn3c(Cl)c(C(=O)O)cc3c2)nn1)C(F)(F)F]

### Step 5: 3-Chloro-7-[4-(1-hydroxy-1-trifluoromethyl-propyl)-[1,2,3]triazol-1-ylmethyl]-indolizine-2-carboxylic acid (Enantiomer B)

Prepared according to the procedure described in Example 9, Step 7, using the following starting material: 3-chloro-7-[4-(1-hydroxy-1-trifluoromethyl-propyl)-[1,2,3]triazol-1-ylmethyl]-indolizine-2-carboxylic acid (Enantiomer B).

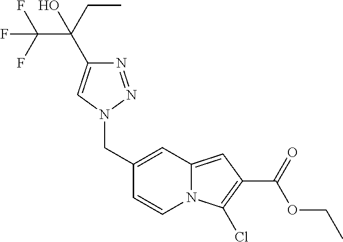 [SMILES: CCOC(=O)c1cc2cc(Cn3cc(C(O)(CC)C(F)(F)F)nn3)ccn2c1Cl]

### Step 6: 3-Chloro-7-[4-(1-hydroxy-1-trifluoromethyl-propyl)-[1,2,3]triazol-1-ylmethyl]-indolizine-2-carboxylic acid ethyl ester (Enantiomer B)

3-Chloro-7-[4-(1-hydroxy-1-trifluoromethyl-propyl)-[1,2,3]triazol-1-ylmethyl]-indolizine-2-carboxylic acid (Enantiomer B) (0.650 g, 1.6 mmol) was dissolved in EtOH (50 mL) and conc. H₂SO₄ (2 mL) was added. The reaction was heated to reflux overnight then cooled to room temperature and concentrated to remove all EtOH. The residue was submitted to standard aqueous workup then purified on silica gel (0-100% EtOAc in hexanes) to afford the title compound.

### Example 88

### Synthesis of 3-Chloro-7-[4-(1-hydroxy-1-trifluoromethyl-propyl)-[1,2,3]triazol-1-ylmethyl]-indolizine-2-carbonitrile (Enantiomer B) (Compound 1-88)

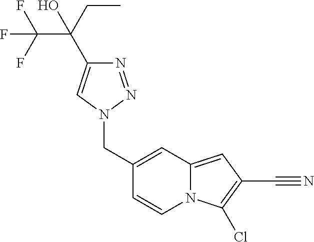 [SMILES: CCC(O)(c1cn(Cc2ccn3c(Cl)c(C#N)cc3c2)nn1)C(F)(F)F]

Prepared according to the procedure described in Example 9, Step 6, using the following starting material: 3-chloro-7-[4-(1-hydroxy-1-trifluoromethyl-propyl)-[1,2,3]triazol-1-ylmethyl]-indolizine-2-carboxylic acid ethyl ester (Enantiomer B). The reaction yielded a mixture of two separable products, of which the title compound was the major product isolated.

### Example 89

### Synthesis of 3-Chloro-7-[4-(1-hydroxy-1-trifluoromethyl-propyl)-[1,2,3]triazol-1-ylmethyl]-indolizine-2-carboxylic acid amide (Enantiomer B) (Compound 1-89)

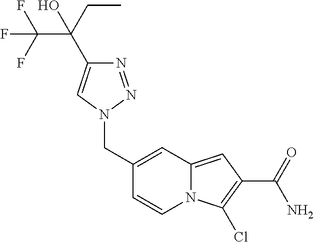 [SMILES: CCC(O)(c1cn(Cc2ccn3c(Cl)c(C(N)=O)cc3c2)nn1)C(F)(F)F]

Prepared according to the procedure described in Example 9, Step 6, using the following starting material: 3-chloro-7-[4-(1-hydroxy-1-trifluoromethyl-propyl)-[1,2,3]triazol-1-ylmethyl]-indolizine-2-carboxylic acid ethyl ester (Enantiomer B). The reaction yielded a mixture of two separable products, of which the title compound was the minor product isolated.

### Example 90

### Human 5-Lipoxygenase Activity Inhibition Assay

A non-limiting example of a 5-lipoxygenase activity inhibition assay is as follows:

Human polymorphonuclear leukocytes are prepared from blood by Ficoll-Hypaque separation, lysed and centrifuged at 100,00×g. The 100,000×g supernatant containing 5-lipoxygenase is added to tubes, containing 100 mM TrisCl pH 8.0, 2 mM ATP, 2 mM calcium and incubated with 20-200 μM arachidonic acid in a final volume of 100 μL for 1-10 minutes at 37° C. Reaction is terminated by the addition of an equal volume of ice cold methanol, centrifuged at 10,000×g for 10 minutes and supernatant analyzed by reverse phase HPLC for formation of 5-HETE/5HPETE monitoring absorbance at 235 nm.

### Example 91

### Human Leukocyte LTB₄ Inhibition Assay

A non-limiting example of a human leukocyte inhibition assay is as follows; Blood was drawn from consenting human volunteers into heparanized tubes and 3% dextran 0.9% saline ⅓ rd volume added. After sedimentation of red blood cells a hypotonic lysis of remaining red blood cells was performed and leukocytes sedimented at 1200 rpm. The pellet was resuspended at 1.25×10⁵ cells/ml and aliquoted into wells containing 1 μL 100% DMSO (vehicle) or 2.5 μL drug in 100% DMSO. Samples were incubated for 5 minutes at 37° C. and 2 μL calcium ionophore A23817 (from a 50 mM DMSO stock diluted just prior to the assay in Hanks balanced salt solution (Invitrogen)) to 1.25 mM) was added, solutions mixed and incubated for 30 minutes at 37° C. Samples were centrifuged at 1,200 rpm (−200×g) for 10 minutes at 4° C., plasma removed and a 1:4 dilution assayed for LTB₄ concentration using ELISA (Assay Designs). Drug concentrations to achieve 50% inhibition (IC₅₀'s) of vehicle LTB₄ were determined by nonlinear regression (Graphpad Prism) of % inhibition versus log drug concentration.

Illustrative biological data for representative compounds disclosed herein is presented in Table 2.

TABLE 2

| Compound # | LTB4 HLA IC₅₀ |
|---|---|
| Compound 1-1 | A |
| Compound 1-2 | C |
| Compound 1-3 | A |
| Compound 1-4 | A |
| Compound 1-5 | B |
| Compound 1-6 | A |
| Compound 1-7 | A |
| Compound 1-8 | A |
| Compound 1-9 | A |
| Compound 1-10 | A |
| Compound 1-11 | A |
| Compound 1-12 | A |
| Compound 1-13 | A |
| Compound 1-14 | B |
| Compound 1-15 | A |
| Compound 1-16 | A |
| Compound 1-17 | A |
| Compound 1-18 | A |
| Compound 1-19 | A |
| Compound 1-20 | A |
| Compound 1-21 | A |
| Compound 1-22 | A |
| Compound 1-23 | C |
| Compound 1-24 | A |
| Compound 1-25 | C |
| Compound 1-26 | A |
| Compound 1-27 | A |
| Compound 1-28 | A |
| Compound 1-29 | A |
| Compound 1-30 | A |
| Compound 1-31 | C |
| Compound 1-32 | A |
| Compound 1-33 | A |
| Compound 1-34 | A |
| Compound 1-35 | B |
| Compound 1-36 | A |
| Compound 1-37 | A |
| Compound 1-38 | B |
| Compound 1-39 | A |
| Compound 1-40 | C |
| Compound 1-41 | B |
| Compound 1-42 | A |
| Compound 1-43 | A |
| Compound 1-44 | A |
| Compound 1-45 | A |
| Compound 1-46 | A |
| Compound 1-47 | A |
| Compound 1-48 | A |
| Compound 1-49 | B |
| Compound 1-50 | C |
| Compound 1-51 | A |
| Compound 1-52 | A |
| Compound 1-53 | A |
| Compound 1-54 | A |
| Compound 1-55 | B |
| Compound 1-56 | A |
| Compound 1-57 | A |
| Compound 1-58 | C |
| Compound 1-59 | A |
| Compound 1-60 | C |
| Compound 1-61 | A |
| Compound 1-62 | B |
| Compound 1-63 | A |
| Compound 1-64 | C |
| Compound 1-65 | A |
| Compound 1-66 | A |
| Compound 1-67 | C |
| Compound 1-68 | C |
| Compound 1-69 | A |
| Compound 1-70 | C |
| Compound 1-71 | C |
| Compound 1-72 | A |
| Compound 1-73 | A |
| Compound 1-74 | A |
| Compound 1-75 | C |
| Compound 1-76 | A |
| Compound 1-77 | A |
| Compound 1-78 | A |
| Compound 1-79 | A |
| Compound 1-80 | A |
| Compound 1-81 | A |
| Compound 1-82 | C |
| Compound 1-83 | B |
| Compound 1-84 | B |
| Compound 1-85 | B |
| Compound 1-86 | A |
| Compound 1-87 | A |
| Compound 1-88 | A |
| Compound 1-89 | C |

Note:
Compound 1-9 and Compound 1-10 are enantiomers; Compound 1-20 and Compound 1-21 are enantiomers. A < 0.3 μM; 0.3 μM < B < 1.0 μM; C > 1.0 μM

### Example 92

### Human Blood LTB₄ Inhibition Assay

A non-limiting example of such a human blood LTB₄ inhibition assay is as follows: Blood is drawn from consenting human volunteers into heparinized tubes and 148 μL aliquots added to wells containing 1.5 μL 100% DMSO (vehicle) or 1.5 μL drug in 100% DMSO. Samples are incubated for 15 minutes at 37° C. then 2 μL calcium ionophore A23817 (from a 50 mM DMSO stock diluted just prior to the assay in Hanks balanced salt solution (Invitrogen)) to 1.25 mM) is added, solutions mixed and incubated for 30 minutes at 37° C. Samples were centrifuged at 1,500 rpm (−300×g) for 10 minutes at 4° C., plasma removed and a 1:100 dilution assayed for LTB₄ concentration using ELISA (Assay Designs). Drug concentrations to achieve 50% inhibition (IC₅₀'s) of vehicle LTB₄ are determined by nonlinear regression (Graphpad Prism) of % inhibition versus log drug concentration.

### Example 93

### Rat Peritoneal Inflammation and Edema Assay

A non-limiting example of such a rat peritoneal inflammation and edema assay is as follows:

The in vivo efficacy of leukotriene biosynthesis inhibitors against inflammation and vascular edema is assessed using a rat model of peritoneal inflammation. Male Sprague-Dawley rats (weighing 200-300 grams) receive a single intraperitoneal (i.p.) injection of 3 ml saline containing zymosan (5 mg/mL) followed immediately by an intravenous (i.v.) injection of Evans blue dye (2 mL of 1.5% solution). Compounds are administered orally (3 mL/kg in 0.5% methylcellulose vehicle) 2 to 4 hours prior to zymosan injection. One to two hours after zymosan injection, rats are euthanized, and the peritoneal cavity was flushed with 10 mL phosphate buffered saline solution (PBS). The resulting fluid is centrifuged at 1,200 rpm for 10 minutes. Vascular edema is assesses by quantifying the amount of Evans blue dye in the supernatant using a spectrophotometer (Absorbance 610 nm). LTB₄ and cysteinyl leukotriene concentrations in the supernatant are determined by ELISA. Drug concentrations to achieve 50% inhibition of plasma leakage (Evans blue dye) and inhibition of peritoneal LTB₄ and cysteinyl leukotrienes could be calculated by nonlinear regression (Graphpad Prism) of % inhibition versus log drug concentration.

### Example 94

### Rat Ionophore Lung Lavage

A non-limiting example of such a rat ionophore lung lavage assay is as follows: A rat ionophore lung lavage model (see Smith et al., J.P.E.T., 1995, 275, 1332-1338) is utilized to determine efficacy of leukotriene biosynthesis inhibitors in the target tissue for respiratory therapy. Male Sprague-Dawley rats (weighing 200-300 grams) are administered compound orally (3 ml/kg in 0.5% methylcellulose vehicle) 2 to 4 hours prior to lung lavage. LTB₄ and cysteinyl leukotrienes are stimulated within the lung via an intra-tracheal instillation of 10 ml PBS containing 20 μg/mL calcium ionophore. After a 3-min period the fluid is withdrawn from the lung and was centrifuged at 1,200 rpm for 10 minutes. LTB₄ and cysteinyl leukotriene concentrations in the supernatant are determined by ELISA. Drug concentrations to achieve 50% inhibition of lung LTB₄ and cysteinyl leukotrienes are calculated by nonlinear regression (Graphpad Prism) of % inhibition versus log drug concentration.

### Example 95

### Induction of Joint Inflammation

Animals are treated systmeically with either vehicle or active compound and then subject to joint inflammation 30 min to 5 hours following dose. Joint inflammation is induced by intra articular (i.a.) injection of zymosan (500 μg/cavity in 25 μl sterile saline), by the insertion of a 27-G needle through the suprapatellar ligament into the knee joint cavity. Control animals received an i.a. injection of an equal volume of sterile saline. Knee-joint swelling is evaluated by measurement of the transverse diameters of injected knee joints using a digital caliper Animals are then euthanized by an excess of CO2 at specific time points after i.a. inject of zymosan (1 hr to 24 hr). Knee synovial cavities are washed with 300 μl PBS containing EDTA (10 mM) by the insertion of a 21-G needle into mouse knee joints, and the synovial fluid recovered by aspiration. Total leukocyte counts are recorded as well as cell differentials. Synovial fluid is centrifuged at 1,200 rpm for 10 minutes. LTB4 and cysteinyl leukotriene concentrations in the supernatant are determined by ELISA.

### Example 96

### Pharmacokinetic Analysis

A non-limiting example of such a pharmacokinetic analysis is as follows: Catheterized rats (SD-JVC from Charles River) are fasted over night (n=2/group) and then dosed with compound either at 2 mg/kg IV or 10 mg/kg PO. For IV dosing, compounds were given in solution using 10% EtOH/40% PEG/50% H2O as vehicle and blood samples are drawn at 5, 15 and 30 mins, 1, 2, 4, 6, 8 hours. For PO dosing, compounds are given in solution using 25% Hydroxypropyl-beta-cyclodextrin in water as vehicle and blood samples were drawn at 30 mins, 1, 2, 4, 6, 8 hours PO. Samples are then analyzed by HPLC-MS/MS.

### Example 97a

### Parenteral Composition

To prepare a parenteral pharmaceutical composition suitable for administration by injection, 100 mg of a water-soluble salt of a compound described herein is dissolved in DMSO and then mixed with 10 mL of 0.9% sterile saline. The mixture is incorporated into a dosage unit form suitable for administration by injection.

### Example 97b

### Oral Composition

To prepare a pharmaceutical composition for oral delivery, 100 mg of a compound described herein is mixed with 750 mg of starch. The mixture is incorporated into an oral dosage unit for, such as a hard gelatin capsule, which is suitable for oral administration.

### Example 97c

### Sublingual (Hard Lozenge) Composition

To prepare a pharmaceutical composition for buccal delivery, such as a hard lozenge, mix 100 mg of a compound described herein, with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 97d

### Inhalation Composition

To prepare a pharmaceutical composition for inhalation delivery, 20 mg of a compound described herein is mixed with 50 mg of anhydrous citric acid and 100 mL of 0.9% sodium chloride solution. The mixture is incorporated into an inhalation delivery unit, such as a nebulizer, which is suitable for inhalation administration.

### Example 97e

### Rectal Gel Composition

To prepare a pharmaceutical composition for rectal delivery, 100 mg of a compound described herein is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

### Example 97f

### Topical Gel Composition

To prepare a pharmaceutical topical gel composition, 100 mg of a compound described herein is mixed with 1.75 g of hydroxypropyl celluose, 10 mL of propylene glycol, 10 mL of isopropyl myristate and 100 mL of purified alcohol USP. The resulting gel mixture is then incorporated into containers, such as tubes, which are suitable for topical administration.

### Example 97g

### Ophthalmic Solution Composition

To prepare a pharmaceutical ophthalmic solution composition, 100 mg of a compound described herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

The examples and embodiments described herein are for illustrative purposes only and various modifications or changes suggested to persons skilled in the art are to be included within the spirit and purview of this application and scope of the appended claims.

1. A compound of Formula (I), or a pharmaceutically acceptable salt, or a pharmaceutically acceptable N-oxide thereof: 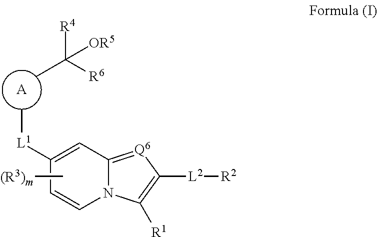
wherein:
A is a 5-membered N-containing heteroaryl;
Q⁶ is N or CR⁷;
R¹ is H, —C(═O)R⁸, substituted or unsubstituted C₁-C₆alkyl, substituted or unsubstituted C₁-C₆-fluoroalkyl, substituted or unsubstituted C₃-C₆cycloalkyl, substituted or unsubstituted C₁-C₆heteroalkyl, substituted or unsubstituted C₂-C₆cycloheteroalkyl, substituted or unsubstituted aryl, substituted or unsubstituted heteroaryl;
R² is halo, —CO₂R⁹, tetrazole, —C(═O)N(R⁹)₂, —CN, —CH(═O), —SR⁸, —S(O)R⁸, —S(O)₂R⁸, —S(O)₂N(R⁹)₂, —OR⁸, —N(R⁹)₂, —C(═O)R⁸, substituted or unsubstituted C₁-C₆alkyl, substituted or unsubstituted C₃-C₆cycloalkyl, substituted or unsubstituted C₁-C₆heteroalkyl, substituted or unsubstituted C₂-C₆cycloheteroalkyl, substituted or unsubstituted aryl, or substituted or unsubstituted heteroaryl;
each R³ is independently halo, —CO₂R⁹, —C(═O)N(R⁹)₂, —CN, tetrazole, —CH(═O), —SR⁸, —S(O)R⁸, —S(O)₂R⁸, substituted or unsubstituted alkyl, substituted or unsubstituted heteroalkyl, —OR⁸, —SR⁸, —N(R⁹)₂, or —C(═O)R⁸;
R⁴ is C₁-C₆haloalkyl, C₁-C₆alkyl, C₃-C₆cycloalkyl, or C₁-C₆heteroalkyl;
R⁵ is H, C₁-C₆alkyl, or —C(═O)R¹⁰;
R⁶ is H, C₁-C₆alkyl, C₃-C₆cycloalkyl, C₁-C₆heteroalkyl, or C₁-C₆haloalkyl;
R⁷ is H, halo, —CO₂R⁹, —C(═O)R⁸, —C(═O)N(R⁹)₂, —CN, —CH(═O), —SR⁸, —S(O)R⁸, —S(O)₂R⁸, —OR⁸, —N(R⁹)₂, substituted or unsubstituted C₁-C₆alkyl, substituted or unsubstituted C₁-C₆heteroalkyl, substituted or unsubstituted aryl, or substituted or unsubstituted heteroaryl;
each R⁸ is independently substituted or unsubstituted C₁-C₆alkyl, substituted or unsubstituted C₁-C₆-fluoroalkyl, substituted or unsubstituted C₁-C₆heteroalkyl, substituted or unsubstituted aryl, or substituted or unsubstituted heteroaryl;
each R⁹ is independently H, substituted or unsubstituted C₁-C₆alkyl, substituted or unsubstituted C₁-C₆-fluoroalkyl, substituted or unsubstituted C₁-C₆heteroalkyl, substituted or unsubstituted aryl, or substituted or unsubstituted heteroaryl;
or two R⁹ taken together with the nitrogen to which they are bound form a substituted or unsubstituted heterocycle;
each R¹⁰ substituted or unsubstituted C₁-C₆alkyl, substituted or unsubstituted C₁-C₆heteroalkyl, substituted or unsubstituted aryl, or substituted or unsubstituted heteroaryl;
or two R¹⁰ taken together with the carbon to which they are bound form a substituted or unsubstituted carbocycle, or substituted or unsubstituted heterocycle;
L¹ is —O—, —NR⁸—, —S—, —C₁-C₃alkylene-, —OC₁-C₃alkylene-, —C₁-C₃alkylene-O—, —C₁-C₃alkylene-NR⁸—, —NR⁸—C₁-C₃alkylene-, —C₁-C₃alkylene-S—, —S—C₁-C₃alkylene-, or —C₁-C₃heteroalkylene-;
L² is a bond, C₁-C₆alkylene, or C₁-C₆heteroalkylene; and
m is 0,1, 2 or 3.

2. The compound of claim 1, or a pharmaceutically acceptable salt, or a pharmaceutically acceptable N-oxide thereof, wherein:
A is pyrrolyl, oxazolyl, thiazolyl, imidazolyl, pyrazolyl, isoxazolyl, isothiazolyl, triazolyl, or thiadiazolyl;
R⁴ is C₁-C₄-fluoroalkyl, C₁-C₄alkyl, or C₃-C₆cycloalkyl;
R⁵ is H, C₁-C₄alkyl, or —C(═O)R¹⁰;
R⁶ is C₁-C₆alkyl, C₃-C₆cycloalkyl, or C₁-C₄fluoroalkyl; and
m is 0 or 1.

3. The compound of claim 2, or a pharmaceutically acceptable salt, or a pharmaceutically acceptable N-oxide thereof, wherein:
A is triazolyl, oxadiazolyl or thiazolyl;
R¹ is H, —C(═O)R⁸, C₁-C₆alkyl, C₃-C₆cycloalkyl, C₁-C₆heteroalkyl, substituted or unsubstituted C₂-C₆cycloheteroalkyl, substituted or unsubstituted phenyl, substituted or unsubstituted naphthyl, substituted or unsubstituted monocyclic heteroaryl, substituted or unsubstituted bicyclic heteroaryl;
L¹ is —O—, —S—, —C₁-C₃alkylene-, —C₁-C₃alkylene-NH—, or —NH—C₁-C₃alkylene-; and
L² is a bond, C₁-C₄alkylene, or C₁-C₄heteroalkylene.

4. The compound of claim 3, or a pharmaceutically acceptable salt, or a pharmaceutically acceptable N-oxide thereof, wherein: 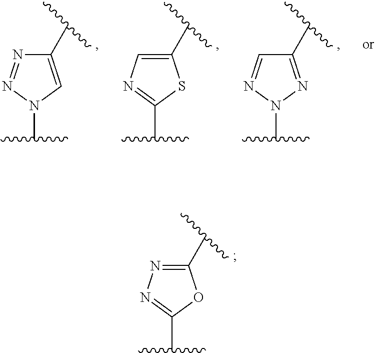 [SMILES: *c1cn(*)nn1 ; *c1cnc(*)s1 ; *c1cnn(*)n1 ; *c1nnc(*)o1]
A is
L² is a bond, C₁-C₄alkylene, or C₁-C₄heteroalkylene; and
R² is halo, —CO₂R⁹, tetrazole, —C(═O)N(R⁹)₂, —CN, —CH(═O), —SR⁸, —S(O)R⁸, —S(O)₂R⁸, —S(O)₂N(R⁹)₂, —OR⁸, —N(R⁹)₂, or —C(═O)R⁸.

5. The compound of claim 4, or a pharmaceutically acceptable salt, or a pharmaceutically acceptable N-oxide thereof, wherein:
A is 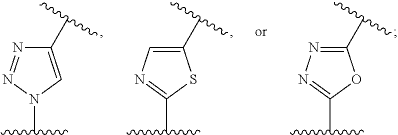 [SMILES: *c1cn(*)nn1 ; *c1cnc(*)s1 ; *c1nnc(*)o1]
R⁴ is C₁-C₄fluoroalkyl; and
R⁶ is C₁-C₄alkyl.

6. The compound of claim 5, or a pharmaceutically acceptable salt, or a pharmaceutically acceptable N-oxide thereof, wherein the compound has one of the following structures:

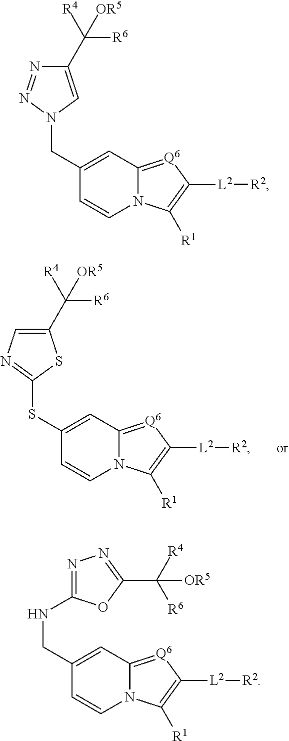

7. The compound of claim 6, or a pharmaceutically acceptable salt, or a pharmaceutically acceptable N-oxide thereof, wherein:
R¹ is H, —C(═O)R⁸, C₁-C₆alkyl, C₃-C₆cycloalkyl, C₁-C₆heteroalkyl, substituted or unsubstituted C₂-C₆cycloheteroalkyl, substituted or unsubstituted phenyl, substituted or unsubstituted naphthyl, substituted or unsubstituted monocyclic heteroaryl, substituted or unsubstituted bicyclic heteroaryl; and
L² is a bond, —CH₂—, —CH₂CH₂—, —CH═CH—, or —CH₂NH(CH₂)₂—.

8. The compound of claim 7, or a pharmaceutically acceptable salt, or a pharmaceutically acceptable N-oxide thereof, wherein:
R¹ is C₁-C₆alkyl or C₃-C₆cycloalkyl; and
R² is —CO₂R⁹, —C(═O)N(R⁹)₂, —CN, —S(O)₂R⁸, —S(O)₂N(R⁹)₂, or —C(═O)R⁸.

9. The compound of claim 7, or a pharmaceutically acceptable salt, or a pharmaceutically acceptable N-oxide thereof, wherein:
R¹ is H, —C(═O)R⁸, —CH₃, —CH₂CH₃, —CH(CH₃)₂, —C(═CH₂)CH₃, cyclopropyl, cyclobutyl, cyclopentyl, cyclopentenyl, cyclohexyl, cyclohexenyl, substituted or unsubstituted phenyl, substituted or unsubstituted naphthyl, substituted or unsubstituted monocyclic heteroaryl selected from substituted or unsubstituted furanyl, substituted or unsubstituted thienyl, substituted or unsubstituted pyrrolyl, substituted or unsubstituted oxazolyl, substituted or unsubstituted thiazolyl, substituted or unsubstituted imidazolyl, substituted or unsubstituted triazolyl, substituted or unsubstituted tetrazolyl, substituted or unsubstituted pyrazolyl, substituted or unsubstituted isoxazolyl, substituted or unsubstituted isothiazolyl, substituted or unsubstituted 1,3,4-thiadiazolyl, substituted or unsubstituted pyridinyl, substituted or unsubstituted pyridazinyl, substituted or unsubstituted pyrimidinyl, substituted or unsubstituted pyrazinyl or substituted or unsubstituted quinolinyl; and
R² is —CO₂R⁹, —C(═O)N(R⁹)₂, —CN, —S(O)₂R⁸, —S(O)₂N(R⁹)₂, or —C(═O)R⁸.

10. The compound of claim 7, or a pharmaceutically acceptable salt, or a pharmaceutically acceptable N-oxide thereof, wherein:
R¹ is a substituted or unsubstituted phenyl, or a substituted or unsubstituted monocyclic heteroaryl; and
R² is —CO₂R⁹, —C(═O)N(R⁹)₂, —CN, —S(O)₂R⁸, —S(O)₂N(R⁹)₂, or —C(═O)R⁸.

11. The compound of claim 9, or a pharmaceutically acceptable salt, or a pharmaceutically acceptable N-oxide thereof, wherein:
R¹ is a substituted or unsubstituted phenyl, substituted or unsubstituted pyridinyl, substituted or unsubstituted pyridazinyl, substituted or unsubstituted pyrimidinyl or substituted or unsubstituted pyrazinyl.

12. The compound of claim 9, or a pharmaceutically acceptable salt, or a pharmaceutically acceptable N-oxide thereof, wherein:
R¹ is a substituted or unsubstituted phenyl or a substituted or unsubstituted pyridinyl.

13. The compound of claim 6, or a pharmaceutically acceptable salt, or a pharmaceutically acceptable N-oxide thereof, wherein:
Q⁶ is N;
L² is a bond, —CH₂—, —CH₂CH₂—, or —CH═CH—.
R⁴ is C₁-C₄-fluoroalkyl;
R⁶ is C₁-C₆alkyl;
R⁸ is C₁-C₆alkyl; and
each R⁹ is independently H or C₁-C₆alkyl.

14. The compound of claim 6, or a pharmaceutically acceptable salt, or a pharmaceutically acceptable N-oxide thereof, wherein:
Q⁶ is CR⁷;
L² is a bond, —CH₂—, —CH₂CH₂—, or —CH═CH—.
R⁴ is C₁-C₄-fluoroalkyl;
R⁶ is C₁-C₆alkyl;
R⁷ is H, halo, —C(═O)R⁸, or C₁-C₆alkyl;
R⁸ is C₁-C₆alkyl; and
each R⁹ is independently H or C₁-C₆alkyl.

15. The compound of claim 1, wherein the compound is: 7-[5-(1-Hydroxy-1-trifluoromethyl-propyl)-thiazol-2-ylsulfanyl]-indolizine-2-carboxylic acid ethyl ester (Compound 1-1); 7-[5-(1-Hydroxy-1-trifluoromethyl-propyl)-thiazol-2-ylsulfanyl]-indolizine-2-carboxylic acid (Compound 1-2); 3-(4-Fluoro-phenyl)-7-[4-(1-hydroxy-1-trifluoromethyl-propyl)-[1,2,3]triazol-2-ylmethyl]-indolizine-2-carboxylic acid ethyl ester (Compound 1-3); 3-(4-Fluoro-phenyl)-7-[4-(1-hydroxy-1-tri fluoromethyl-propyl)-[1,2,3]triazol-1-ylmethyl]-indolizine-2-carboxylic acid ethyl ester (Compound 1-4); 3-(4-Fluoro-phenyl)-7-[4-(1-hydroxy-1-trifluoromethyl-propyl)-[1,2,3]triazol-1-ylmethyl]-indolizine-2-carboxylic acid (Compound 1-5); 3-(4-Fluoro-phenyl)-7-[4-(1-hydroxy-1-trifluoromethyl-propyl)-[1,2,3]triazol-1-ylmethyl]-indolizine-2-carboxylic acid amide (Compound 1-6); 1,1,1-Trifluoro-2-{1-[3-(4-fluoro-phenyl)-2-(1-hydroxy-1-methyl-ethyl)-indolizin-7-ylmethyl]-1H-[1,2,3]triazol-4-yl}-butan-2-ol (Compound 1-7); 3-(4-Fluoro-phenyl)-7-[5-(1-hydroxy-1-trifluoromethyl-propyl)-2-imino-[1,3,4]oxadiazol-3-ylmethyl]-indolizine-2-carboxylic acid ethyl ester (Compound 1-8); 3-(4-Fluoro-phenyl)-7-[4-(1-hydroxy-1-trifluoromethyl-propyl)-[1,2,3]triazol-1-ylmethyl]-indolizine-2-carboxylic acid amide (Compound 1-9); 3-(4-Fluoro-phenyl)-7-[4-(1-hydroxy-1-trifluoromethyl-propyl)-[1,2,3]triazol-1-ylmethyl]-indolizine-2-carboxylic acid amide (Compound 1-10); 3-(4-Fluoro-phenyl)-7-{[5-(1-hydroxy-1-trifluoromethyl-propyl)-[1,3,4]oxadiazol-2-ylamino]-methyl}-indolizine-2-carboxylic acid ethyl ester (Compound 1-11); 7-{[5-(1-Hydroxy-1-trifluoromethyl-propyl)-[1,3,4]oxadiazol-2-ylamino]-methyl}-3-phenyl-imidazo[1,2-a]pyridine-2-carboxylic acid ethyl ester (Compound 1-12); 3-(4-Fluoro-phenyl)-7-[4-(1-hydroxy-1-trifluoromethyl-propyl)-[1,2,3]triazol-1-ylmethyl]-imidazo[1,2-a]pyridine-2-carboxylic acid ethyl ester (Compound 1-13); 3-(4-Fluoro-phenyl)-7-[4-(1-hydroxy-1-trifluoromethyl-propyl)-[1,2,3]triazol-1-ylmethyl]-imidazo[1,2-c]pyridine-2-carboxylic acid amide (Compound 1-14); 1-Bromo-3-(4-fluoro-phenyl)-7-{[5-(1-hydroxy-1-trifluoromethyl-propyl)-[1,3,4]oxadiazol-2-ylamino]-methyl}-indolizine-2-carboxylic acid ethyl ester (Compound 1-15); 3-(4-Fluoro-phenyl)-7-[4-(1-hydroxy-1-tri fluoromethyl-propyl)-[1,2,3]triazol-1-ylmethyl]-indolizine-2-carbonitrile (Compound 1-16); 1-Bromo-3-(4-fluoro-phenyl)-7-{[5-(1-hydroxy-1-trifluoromethyl-propyl)-[1,3,4]oxadiazol-2-ylamino]-methyl}-indolizine-2-carboxylic acid amide (Compound 1-17); 1-Bromo-3-(4-fluoro-phenyl)-7-{[5-(1-hydroxy-1-trifluoromethyl-propyl)-[1,3,4]oxadiazol-2-ylamino]-methyl}-indolizine-2-carbonitrile (Compound 1-18); 3-(4-Fluoro-phenyl)-7-{[5-(1-hydroxy-1-trifluoromethyl-propyl)-[1,3,4]oxadiazol-2-ylamino]-methyl}-indolizine-2-carboxylic acid amide (Compound 1-19); 3-(4-Fluoro-phenyl)-7-{[5-(1-hydroxy-1-trifluoromethyl-propyl)-[1,3,4]oxadiazol-2-ylamino]-methyl}-indolizine-2-carboxylic acid amide (Compound 1-20); 3-(4-Fluoro-phenyl)-7-{[5-(1-hydroxy-1-trifluoromethyl-propyl)-[1,3,4]oxadiazol-2-ylamino]-methyl}-indolizine-2-carboxylic acid amide (Compound 1-21); 3-(4-Fluoro-phenyl)-7-[4-(1-hydroxy-1-trifluoromethyl-propyl)-[1,2,3]triazol-1-ylmethyl]-indolizine-2-carbonitrile (Compound 1-22); 1,1,1-Trifluoro-2-{1-[3-(4-fluoro-phenyl)-2-(2H-tetrazol-5-yl)-indolizin-7-ylmethyl]-1H-[1,2,3]triazol-4-yl}-butan-2-ol (Compound 1-23); 7-[4-(1-Hydroxy-1-trifluoromethyl-propyl)-[1,2,3]triazol-1-ylmethyl]-3-pyridin-3-yl-indolizine-2-carboxylic acid ethyl ester (Compound 1-24); 7-[4-(1-Hydroxy-1-trifluoromethyl-propyl)-[1,2,3]triazol-1-ylmethyl]-3-pyridin-3-yl-indolizine-2-carboxylic acid amide (Compound 1-25); 7-[4-(1-Hydroxy-1-trifluoromethyl-propyl)-[1,2,3]triazol-1-ylmethyl]-3-pyridin-3-yl-indolizine-2-carbonitrile (Compound 1-26); 3-(4-Chloro-phenyl)-7-[4-(1-hydroxy-1-trifluoromethyl-propyl)-[1,2,3]triazol-1-ylmethyl]-indolizine-2-carboxylic acid ethyl ester (Compound 1-27); 3-(4-Chloro-phenyl)-7-[4-(1-hydroxy-1-trifluoromethyl-propyl)-[1,2,3]triazol-1-ylmethyl]-indolizine-2-carboxylic acid amide (Compound 1-28); 3-(6-Fluoro-pyridin-3-yl)-7-[4-(1-hydroxy-1-trifluoromethyl-propyl)-[1,2,3]triazol-1-ylmethyl]-indolizine-2-carboxylic acid ethyl ester (Compound 1-29); 1,1,1-Trifluoro-2-{1-[3-(4-fluoro-phenyl)-2-hydroxymethyl-indolizin-7-ylmethyl]-1H-[1,2,3]triazol-4-yl}-butan-2-ol (Compound 1-30); 3-(6-Fluoro-pyridin-3-yl)-7-[4-(1-hydroxy-1-trifluoromethyl-propyl)-[1,2,3]triazol-1-ylmethyl]-indolizine-2-carboxylic acid amide (Compound 1-31); 3-(6-Fluoro-pyridin-3-yl)-7-[4-(1-hydroxy-1-trifluoromethyl-propyl)-[1,2,3]triazol-1-ylmethyl]-indolizine-2-carbonitrile (Compound 1-32); 3-(4-Fluoro-phenyl)-7-[4-(1-hydroxy-1-trifluoromethyl-propyl)-[1,2,3]triazol-1-ylmethyl]-indolizine-2-carbaldehyde (Compound 1-33); 1,1,1-Trifluoro-2-{1-[3-(4-fluoro-phenyl)-2-methanesulfonylmethyl-indolizin-7-ylmethyl]-1H-[1,2,3]triazol-4-yl}-butan-2-ol (Compound 1-34); 7-[4-(1-Hydroxy-1-trifluoromethyl-propyl)-[1,2,3]triazol-1-ylmethyl]-3-(6-methoxy-pyridin-3-yl)-indolizine-2-carboxylic acid amide (Compound 1-35); 7-[4-(1-Hydroxy-1-trifluoromethyl-propyl)-[1,2,3]triazol-1-ylmethyl]-3-(6-methoxy-pyridin-3-yl)-indolizine-2-carboxylic acid ethyl ester (Compound 1-36); 3-({3-(4-Fluoro-phenyl)-7-[4-(1-hydroxy-1-tri fluoromethyl-propyl)-[1,2,3]triazol-1-ylmethyl]-indolizin-2-ylmethyl}-amino)-propionic acid ethyl ester (Compound 1-37); 1,1,1-Trifluoro-2-{1-[3-(4-fluoro-phenyl)-2-morpholin-4-ylmethyl-indolizin-7-ylmethyl]-1H-[1,2,3]triazol-4-yl}-butan-2-ol (Compound 1-38); (E)-3-{3-(4-Fluoro-phenyl)-7-[4-(1-hydroxy-1-trifluoromethyl-propyl)-[1,2,3]triazol-1-ylmethyl]-indolizin-2-yl}-acrylic acid methyl ester (Compound 1-39); 3-({3-(4-Fluoro-phenyl)-7-[4-(1-hydroxy-1-trifluoromethyl-propyl)-[1,2,3]triazol-1-ylmethyl]-indolizin-2-ylmethyl}-amino)-propionic acid (Compound 1-40); (E)-3-{3-(4-Fluoro-phenyl)-7-[4-(1-hydroxy-1-trifluoromethyl-propyl)-[1,2,3]triazol-1-ylmethyl]-indolizin-2-yl}-acrylic acid (Compound 1-41); 3-({3-(4-Fluoro-phenyl)-7-[4-(1-hydroxy-1-trifluoromethyl-propyl)-[1,2,3]triazol-1-ylmethyl]-indolizin-2-ylmethyl}-amino)-propionitrile (Compound 1-42); (E)-3-{3-(4-Fluoro-phenyl)-7-[4-(1-hydroxy-1-trifluoromethyl-propyl)-[1,2,3]triazol-1-ylmethyl]-indolizin-2-yl}-acrylamide (Compound 1-43); 1,1,1-Trifluoro-2-{1-[3-(4-fluoro-phenyl)-2-methoxymethyl-indolizin-7-ylmethyl]-1H-[1,2,3]triazol-4-yl}-butan-2-ol (Compound 1-44); 3-(4-Fluoro-phenyl)-2-methoxymethyl-7-[4-(1-methoxy-1-trifluoromethyl-propyl)-[1,2,3]triazol-1-ylmethyl]-indolizine (Compound 1-45); 3-(3-Fluoro-phenyl)-7-[4-(1-hydroxy-1-trifluoromethyl-propyl)-[1,2,3]triazol-1-ylmethyl]-indolizine-2-carboxylic acid ethyl ester (Compound 1-46); 7-[4-(1-Hydroxy-1-trifluoromethyl-propyl)-[1,2,3]triazol-1-ylmethyl]-3-(3-methanesulfonyl-phenyl)-indolizine-2-carboxylic acid ethyl ester (Compound 1-47); 3-(3-Fluoro-phenyl)-7-[4-(1-hydroxy-1-trifluoromethyl-propyl)-[1,2,3]triazol-1-ylmethyl]-indolizine-2-carboxylic acid amide (Compound 1-48); 7-[4-(1-Hydroxy-1-trifluoromethyl-propyl)-[1,2,3]triazol-1-ylmethyl]-3-(3-methanesulfonyl-phenyl)-indolizine-2-carbonitrile (Compound 1-49); (1,1-Dioxo-1λ⁶-thiazolidin-3-yl)-{3-(4-fluoro-phenyl)-7-[4-(1-hydroxy-1-trifluoromethyl-propyl)-[1,2,3]triazol-1-ylmethyl]-indolizin-2-yl}-methanone (Compound 1-50); 3-(4-Fluoro-phenyl)-7-[4-(1-hydroxy-1-trifluoromethyl-propyl)-[1,2,3]triazol-1-ylmethyl]-indolizine-2-carboxylic acid methoxy-methyl-amide (Compound 1-51); 7-[4-(1-Hydroxy-1-tri fluoromethyl-propyl)-[1,2,3]triazol-1-ylmethyl]-3-quinolin-3-yl-indolizine-2-carboxylic acid ethyl ester (Compound 1-52); 7-[4-(1-Hydroxy-1-trifluoromethyl-propyl)-[1,2,3]triazol-1-ylmethyl]-3-quinolin-3-yl-indolizine-2-carbonitrile (Compound 1-53); 1-{3-(4-Fluoro-phenyl)-7-[4-(1-hydroxy-1-trifluoromethyl-propyl)-[1,2,3]triazol-1-ylmethyl]-indolizin-2-yl}-ethanone (Compound 1-54); 3-(4-Fluoro-phenyl)-7-[4-(1-hydroxy-1-trifluoromethyl-propyl)-[1,2,3]triazol-1-ylmethyl]-indolizine-2-carboxylic acid ethylamide (Compound 1-55); 3-(6-Ethoxy-pyridin-3-yl)-7-[4-(1-hydroxy-1-trifluoromethyl-propyl)-[1,2,3]triazol-1-ylmethyl]-indolizine-2-carboxylic acid ethyl ester (Compound 1-56); 7-[4-(1-Hydroxy-1-trifluoromethyl-propyl)-[1,2,3]triazol-1-ylmethyl]-3-(6-methoxy-pyridin-3-yl)-indolizine-2-carbonitrile (Compound 1-57); 3-(6-Ethoxy-pyridin-3-yl)-7-[4-(1-hydroxy-1-trifluoromethyl-propyl)-[1,2,3]triazol-1-ylmethyl]-indolizine-2-carboxylic acid amide (Compound 1-58); 3-(6-Ethoxy-pyridin-3-yl)-7-[4-(1-hydroxy-1-trifluoromethyl-propyl)-[1,2,3]triazol-1-ylmethyl]-indolizine-2-carbonitrile (Compound 1-59); 2-{1-[2-Ethylaminomethyl-3-(4-fluoro-phenyl)-indolizin-7-ylmethyl]-1H-[1,2,3]triazol-4-yl}-1,1,1-trifluoro-butan-2-ol (Compound 1-60); 1-Chloro-3-(4-fluoro-phenyl)-7-[4-(1-hydroxy-1-trifluoromethyl-propyl)-[1,2,3]triazol-1-ylmethyl]-indolizine-2-carboxylic acid amide (Compound 1-61); 7-[4-(1-Hydroxy-1-trifluoromethyl-propyl)-[1,2,3]triazol-1-ylmethyl]-3-(1-methyl-6-oxo-1,6-dihydro-pyridin-3-yl)-indolizine-2-carboxylic acid ethyl ester (Compound 1-62); 7-[4-(1-Hydroxy-1-trifluoromethyl-propyl)-[1,2,3]triazol-1-ylmethyl]-3-(2-methyl-pyridin-4-yl)-indolizine-2-carboxylic acid ethyl ester (Compound 1-63); 7-[4-(1-Hydroxy-1-trifluoromethyl-propyl)-[1,2,3]triazol-1-ylmethyl]-3-(2-methyl-pyridin-4-yl)-indolizine-2-carboxylic acid amide (Compound 1-64); 7-[4-(1-Hydroxy-1-trifluoromethyl-propyl)-[1,2,3]triazol-1-ylmethyl]-3-(2-methyl-pyridin-4-yl)-indolizine-2-carbonitrile (Compound 1-65); 3-Acetyl-7-[4-(1-hydroxy-1-trifluoromethyl-propyl)-[1,2,3]triazol-1-ylmethyl]-indolizine-2-carboxylic acid methyl ester (Compound 1-66); 3-Acetyl-7-[4-(1-hydroxy-1-trifluoromethyl-propyl)-[1,2,3]triazol-1-ylmethyl]-indolizine-2-carboxylic acid amide (Compound 1-67); 7-[4-(1-Hydroxy-1-trifluoromethyl-propyl)-[1,2,3]triazol-1-ylmethyl]-3-(1-methyl-6-oxo-1,6-dihydro-pyridin-3-yl)-indolizine-2-carboxylic acid amide (Compound 1-68); 7-[4-(1-Hydroxy-1-trifluoromethyl-propyl)-[1,2,3]triazol-1-ylmethyl]-3-(6-methyl-pyridin-3-yl)-indolizine-2-carboxylic acid ethyl ester (Compound 1-69); 7-[4-(1-Hydroxy-1-trifluoromethyl-propyl)-[1,2,3]triazol-1-ylmethyl]-3-(1-methyl-6-oxo-1,6-dihydro-pyridin-3-yl)-indolizine-2-carbonitrile (Compound 1-70); 7-[4-(1-Hydroxy-1-trifluoromethyl-propyl)-[1,2,3]triazol-1-ylmethyl]-3-(6-methyl-pyridin-3-yl)-indolizine-2-carboxylic acid amide (Compound 1-71); 7-[4-(1-Hydroxy-1-trifluoromethyl-propyl)-[1,2,3]triazol-1-ylmethyl]-3-(6-methyl-pyridin-3-yl)-indolizine-2-carbonitrile (Compound 1-72); 7-[4-(1-Hydroxy-1-trifluoromethyl-propyl)-[1,2,3]triazol-1-ylmethyl]-3-pyrimidin-5-yl-indolizine-2-carboxylic acid ethyl ester (Compound 1-73); 7-[4-(1-Hydroxy-1-trifluoromethyl-propyl)-[1,2,3]triazol-1-ylmethyl]-3-(1-methyl-1H-pyrazol-4-yl)-indolizine-2-carboxylic acid ethyl ester (Compound 1-74); 4-Nitro-benzoic acid 1-{1-[2-carbamoyl-3-(6-methyl-pyridin-3-yl)-indolizin-7-ylmethyl]-1H-[1,2,3]triazol-4-yl}-1-trifluoromethyl-propyl ester (Enantiomer B) (Compound 1-75); 7-[4-(1-Hydroxy-1-trifluoromethyl-propyl)-[1,2,3]triazol-1-ylmethyl]-3-(6-methyl-pyridin-3-yl)-indolizine-2-carbonitrile (Enantiomer B) (Compound 1-76); 7-[4-(1-Hydroxy-1-trifluoromethyl-propyl)-[1,2,3]triazol-1-ylmethyl]-3-isopropyl-indolizine-2-carboxylic acid ethyl ester (Enantiomer B) (Compound 1-77); 7-[4-(1-Hydroxy-1-trifluoromethyl-propyl)-[1,2,3]triazol-1-ylmethyl]-3-(6-methoxy-pyridin-3-yl)-indolizine-2-carboxylic acid amide (Enantiomer B) (Compound 1-78); 7-[4-(1-Hydroxy-1-trifluoromethyl-propyl)-[1,2,3]triazol-1-ylmethyl]-3-(6-methoxy-pyridin-3-yl)-indolizine-2-carbonitrile (Enantiomer B) (Compound 1-79); 7-[4-(1-Hydroxy-1-trifluoromethyl-propyl)-[1,2,3]triazol-1-ylmethyl]-3-(2-methyl-pyridin-4-yl)-indolizine-2-carbonitrile (Enantiomer B) (Compound 1-80); 7-[4-(1-Hydroxy-1-trifluoromethyl-propyl)-[1,2,3]triazol-1-ylmethyl]-3-isopropyl-indolizine-2-carbonitrile (Enantiomer B) (Compound 1-81); 7-[4-(1-Hydroxy-1-trifluoromethyl-propyl)-[1,2,3]triazol-1-ylmethyl]-3-(6-methoxy-pyridin-3-yl)-1-(2,2,2-trifluoro-acetyl)-indolizine-2-carboxylic acid amide (Enantiomer B) (Compound 1-82); 4-Nitro-benzoic acid 1-{1-[2-carbamoyl-3-(6-methoxy-pyridin-3-yl)-indolizin-7-ylmethyl]-1H-[1,2,3]triazol-4-yl}-1-trifluoromethyl-propyl ester (Enantiomer B) (Compound 1-83); 3-(6-Methoxy-pyridin-3-yl)-7-{4-[1-(4-nitro-benzoyloxy)-1-trifluoromethyl-propyl]-[1,2,3]triazol-1-ylmethyl}-1-(2,2,2-trifluoro-acetyl)-indolizine-2-carboxylic acid ethyl ester (Enantiomer B) (Compound 1-84); 3-(6-Methoxy-pyridin-3-yl)-7-{4-[1-(4-nitro-benzoyloxy)-1-trifluoromethyl-propyl]-[1,2,3]triazol-1-ylmethyl}-indolizine-2-carboxylic acid ethyl ester (Enantiomer B) (Compound 1-85); 7-{4-[1-(4-Nitro-benzoyloxy)-1-trifluoromethyl-propyl]-[1,2,3]triazol-1-ylmethyl}-indolizine-2-carboxylic acid ethyl ester (Enantiomer B) (Compound 1-86); 3-Chloro-7-[4-(1-hydroxy-1-trifluoromethyl-propyl)-[1,2,3]triazol-1-ylmethyl]-indolizine-2-carboxylic acid ethyl ester (Enantiomer B) (Compound 1-87); 3-Chloro-7-[4-(1-hydroxy-1-trifluoromethyl-propyl)-[1,2,3]triazol-1-ylmethyl]-indolizine-2-carbonitrile (Enantiomer B) (Compound 1-88); or 3-Chloro-7-[4-(1-hydroxy-1-trifluoromethyl-propyl)-[1,2,3]triazol-1-ylmethyl]-indolizine-2-carboxylic acid amide (Enantiomer B) (Compound 1-89); or a pharmaceutically acceptable salt, or a pharmaceutically acceptable N-oxide thereof.

16. A pharmaceutical composition comprising a compound, pharmaceutically acceptable salt, or a pharmaceutically acceptable N-oxide, of claim 1 and at least one pharmaceutically acceptable excipient.

17. The pharmaceutical composition of claim 16, wherein the pharmaceutical composition is formulated for intravenous injection, oral administration, inhalation, nasal administration, topical administration, ophthalmic administration or otic administration.

18. The pharmaceutical composition of claim 16, wherein the pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

19. A method of treating a leukotriene dependent or leukotriene-mediated disease or condition in a mammal in need thereof comprising administering to the patient a therapeutically effective amount of the compound, pharmaceutically acceptable salt; or a pharmaceutically acceptable N-oxide of claim 1.

20. The method of claim 19, wherein the disease or condition is inflammation.

21. The method of claim 19, wherein the disease or condition is a respiratory disease or cardiovascular disease.

22. The method of claim 19, wherein the disease or condition is asthma, atherosclerosis, chronic obstructive pulmonary disease, pulmonary hypertension, interstitial lung fibrosis, rhinitis, aortic aneurysm, myocardial infarction, stroke, osteoarthritis, rheumatoid arthritis, ankylosing spondylitis, NSAID-induced gastrointestinal tract lesions, cancer, metabolic disorder, or pain.

23. The method of claim 22, wherein the pain is acute pain, chronic pain, nociceptive pain, neuropathic pain, inflammatory pain, non-inflammatory pain, central pain, or peripheral pain.
